(19) United States
(12) Patent Application Publication
(10) Pub. No.: US 2013/0129668 A1
(43) Pub. Date: May 23, 2013

(54) Diagnosis and treatment of arthritis using epigenetics

(71) Applicants: Gary Firestein, Del Mar, CA (US); Kazuhisa Nakano, San Diego, CA (US)

(72) Inventors: Gary Firestein, Del Mar, CA (US); Kazuhisa Nakano, San Diego, CA (US)

(73) Assignee: The Regents of the University of California, Oakland, CA (US)

(21) Appl. No.: 13/573,233

(22) Filed: Aug. 31, 2012

Related U.S. Application Data
(60) Provisional application No. 61/530,072, filed on Sep. 1, 2011.

(51) Int. Cl. C12Q 1/68

(52) U.S. Cl. CPC C12Q 1/6883
USPC 424/85.1; 435/6.11; 514/16.6; 514/44 A; 514/44 R

(57) ABSTRACT

Embodiments of the present invention include methods, compositions and kits for evaluating a diagnosis, prognosis, or response to treatment of a subject with a disorder such as rheumatoid arthritis or osteoarthritis. Some embodiments include identifying a therapeutic agent for treating a disorder such as rheumatoid arthritis or osteoarthritis.

### RELATED APPLICATIONS

This application claims the benefit of U.S. Provisional Application No. 61/530,072 entitled “DIAGNOSIS AND TREATMENT OF ARTHRITIS USING EPIGENETICS” filed on Sep. 1, 2011, which is incorporated herein by reference in its entirety.

### REFERENCE TO SEQUENCE LISTING

The present application is being filed along with a Sequence Listing in electronic format. The Sequence Listing is provided as a file entitled UCSD055_{—}001A.TXT, created Aug. 24, 2012, which is approximately 135,386 KB in size. The information in the electronic format of the Sequence Listing is incorporated herein by reference in its entirety.

### FIELD OF THE INVENTION

Embodiments of the present invention include methods, compositions and kits for evaluating a diagnosis, prognosis, or response to treatment of a subject with a disorder such as rheumatoid arthritis or osteoarthritis. Some embodiments include identifying a therapeutic agent for treating a disorder such as rheumatoid arthritis or osteoarthritis.

### BACKGROUND OF THE INVENTION

Rheumatoid arthritis is an immune-mediated disease marked by symmetric inflammation in diarthrodial joints and destruction of the extracellular matrix. Genomics has rapidly advanced our understanding of susceptibility and severity of RA, and many associated polymorphisms in key genes have been described. However, identical twins have a concordance rate of only 12-15% suggesting that other influences can affect either the onset or progression of disease, such as epigenetic regulation of gene expression. One of the most widely studied epigenetic mechanisms, especially in oncology, is DNA methylation, which plays a key role regulating and silencing gene expression and could potentially contribute to immune dysregulation.

The pathogenesis of Rheumatoid arthritis (RA) is complex and involves numerous cell types that contribute through adaptive and innate immune responses (Firestein G S. Evolving concepts of rheumatoid arthritis. Nature, 423:356-361, 2003). Fibroblast-like synoviocytes (FLS), which form the synovial intimal lining, play an integral role by producing key cytokines (e.g., IL-6), small molecule mediators (e.g., prostanoids), and proteases (e.g., metalloproteinases). While osteoclasts are the primary effectors of bone erosions in arthritis, FLS are responsible for cartilage damage by virtue of their ability to adhere to and invade cartilage extracellular matrix. This capacity requires homotypic aggregation mediated by the adhesion molecule cadherin-11, which directs intimal lining formation and supports an invasive phenotype (Kiener H P et al. Cadherin 11 promotes invasive behavior of fibroblast-like synoviocytes. Arthritis Rheum. 2009 May; 60(5):1305-10). A well-defined relationship has long been recognized between synovial tissue histology and function and peripheral blood cell characteristics in diseases like RA, most likely because the circulating cells actively traffic between the synovium, lymph nodes, and peripheral blood (Malone D G et al. Immune function in severe, active rheumatoid arthritis. A relationship between peripheral blood mononuclear cell proliferation to soluble antigens and synovial tissue immunohistologic characteristics. J Clin Invest., 74(4):1173-1185, 1984; Firestein, G S. Etiology and pathogenesis of rheumatoid arthritis, In: Textbook of Rheumatology, G S Firestein, et al. eds., Elsevier, Philadelphia, 8th edition, 2009, pp. 1035-86). Understanding the precise molecular mechanisms that regulate FLS and peripheral blood cell activation in inflammatory arthritis could provide insights into the pathogenesis of RA and lead to novel therapeutic strategies.

### SUMMARY OF THE INVENTION

Some embodiments of the methods, compositions and kits provided herein include a method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising determining whether at least 2 nucleic acid loci or at least 2 genes in a sample from said subject have methylation states indicative of rheumatoid arthritis, osteoarthritis, a rheumatoid arthritis or osteoarthritis prognosis, or a response to treatment for rheumatoid arthritis or osteoarthritis.

Some embodiments also include comprising comparing the methylation states of the at least 2 loci or at least 2 genes in the sample from said subject with the methylation states of the loci or genes in normal tissue, tissue from a subject without said known rheumatoid arthritis or osteoarthritis prognosis, or tissue from a subject without said known response to treatment for rheumatoid arthritis or osteoarthritis.

In some embodiments, an increase or decrease in the extent of methylation of at least 2 loci or at least 2 genes compared to the extent of methylation of the loci or genes in normal tissue, tissue from a subject without said known rheumatoid arthritis or osteoarthritis prognosis, or tissue from a subject without said known response to treatment for rheumatoid arthritis or osteoarthritis is indicative of the presence or absence of rheumatoid arthritis, osteoarthritis, a rheumatoid arthritis or osteoarthritis prognosis, or response to treatment for rheumatoid arthritis or osteoarthritis for the subject.

In some embodiments, the methylation states of said at least 2 loci or at least 2 genes are determined in fibroblasts from said subject. In some embodiments, the methylation state of said at least 2 loci or at least 2 genes is determined in synoviocytes from said subject. In some embodiments, the methylation states of said at least 2 loci or at least 2 genes are determined in fibroblast-like synoviocytes from said subject. In some embodiments, the methylation states of said at least 2 loci or at least 2 genes are determined in rheumatoid arthritis fibroblast-like synoviocytes from said subject. In some embodiments, the methylation states of said at least 2 loci or at least 2 genes are determined in osteoarthritis fibroblast-like synoviocytes from said subject.

In some embodiments, the methylation states of said at least 2 loci or at least 2 genes are determined in macrophage from said subject.

In some embodiments, the methylation states of said at least 2 loci or at least 2 genes are determined in peripheral blood cells from said subject. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

In some embodiments, the at least 2 loci are selected from the group consisting of SEQ ID NO.s 1-485512. In some embodiments, the at least 2 loci are selected from the group consisting of the loci listed in TABLE 6. In some embodiments, the at least 2 loci are selected from the group consisting of the loci listed in TABLE 7. In some embodiments, the at least 2 loci are selected from the group consisting of the loci listed in TABLE 8. In some embodiments, the at least 2 loci are selected from the group consisting of the loci listed in TABLE 3.

In some embodiments, the methylation states of at least 5 nucleic acid loci or at least 5 genes in said sample are determined. In some embodiments, the methylation states of at least 10 nucleic acid loci or at least 10 genes in said sample are determined. In some embodiments, the methylation states of at least 20 nucleic acid loci or at least 20 genes in said sample are determined. In some embodiments, the methylation states of at least 50 nucleic acid loci or at least 50 genes in said sample are determined. In some embodiments, the methylation states of at least 100 nucleic acid loci or at least 100 genes are determined.

In some embodiments, the subject is a mammal. In some embodiments, the subject is a human.

In some embodiments, the nucleic acid loci are selected from the group consisting of the loci listed in TABLE 6. In some embodiments, the nucleic acid loci are selected from the group consisting of the loci listed in TABLE 7. In some embodiments, the nucleic acid loci are selected from the group consisting of the loci listed in TABLE 8.

Some embodiments of the methods, compositions and kits provided herein include a method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising determining the methylation states of a plurality of nucleic acid loci or the methylation states of a plurality of genes in a sample of said subject to obtain a methylation profile; and determining whether said methylation profile is indicative of rheumatoid arthritis, osteoarthritis, a rheumatoid arthritis or osteoarthritis prognosis, or a response to treatment for rheumatoid arthritis or osteoarthritis.

Some embodiments also include comparing the methylation state of the plurality of nucleic acid loci or the methylation state of the plurality of genes in the sample from said subject with the methylation state of the plurality of nucleic acid loci or the methylation state of the plurality of genes in normal tissue, tissue from a subject without said known rheumatoid arthritis or osteoarthritis prognosis, or tissue from a subject without said known response to treatment for rheumatoid arthritis or osteoarthritis.

In some embodiments, an increase or decrease in the extent of methylation of plurality of nucleic acid loci or of the methylation state of the plurality of genes compared to the extent of methylation of the plurality of nucleic acid loci or the extent of methylation of the plurality of genes in normal tissue, tissue from a subject without said known rheumatoid arthritis or osteoarthritis prognosis, or tissue from a subject without said known response to treatment for rheumatoid arthritis or osteoarthritis is indicative of the presence or absence of rheumatoid arthritis, osteoarthritis, a rheumatoid arthritis or osteoarthritis prognosis, or response to treatment for rheumatoid arthritis or osteoarthritis for the subject.

In some embodiments, the methylation state of said plurality of nucleic acid loci or said plurality of genes is determined in fibroblasts from said subject. In some embodiments, the methylation state of said plurality of nucleic acid loci or said plurality of genes is determined in synoviocytes from said subject. In some embodiments, the methylation state of said plurality of nucleic acid loci or said plurality of genes is determined in fibroblast-like synoviocytes from said subject. In some embodiments, the methylation state of said plurality of nucleic acid loci or said plurality of genes is determined in rheumatoid arthritis fibroblast-like synoviocytes from said subject. In some embodiments, the methylation state of said plurality of nucleic acid loci or said plurality of genes is determined in osteoarthritis fibroblast-like synoviocytes from said subject.

In some embodiments, the methylation state of said plurality of nucleic acid loci or said plurality of genes is determined in macrophage from said subject.

In some embodiments, the methylation state of said plurality of nucleic acid loci or said plurality of genes is determined in peripheral blood cells from said subject. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

In some embodiments, the plurality of nucleic acid loci is selected from the group consisting of SEQ ID NO.s 1-485512. In some embodiments, the plurality of nucleic acid loci is selected from the group consisting of the loci listed in TABLE 6. In some embodiments, the plurality of nucleic acid loci is selected from the group consisting of the loci listed in TABLE 7. In some embodiments, the plurality of nucleic acid loci is selected from the group consisting of the loci listed in TABLE 8.

In some embodiments, the plurality genes is selected from the group consisting of the loci listed in TABLE 3.

In some embodiments, the methylation states of at least 5 nucleic acid loci or at least 5 genes in said sample are determined. In some embodiments, the methylation states of at least 10 nucleic acid loci or at least 10 genes in said sample are determined. In some embodiments, the methylation states of at least 20 nucleic acid loci or at least 20 genes in said sample are determined. In some embodiments, the methylation states of at least 50 nucleic acid loci or at least 50 genes in said sample are determined. In some embodiments, the methylation states of at least 100 nucleic acid loci or at least 100 genes are determined.

In some embodiments, the subject is a mammal. In some embodiments, the subject is a human.

Some embodiments of the methods, compositions and kits provided herein include a method of identifying a methylation profile indicative of rheumatoid arthritis, osteoarthritis, a rheumatoid arthritis or osteoarthritis prognosis, or a response to treatment for rheumatoid arthritis or osteoarthritis comprising determining the methylation states of a plurality of nucleic acid loci or the methylation states of a plurality of genes in a sample from a test subject with rheumatoid arthritis, osteoarthritis, a particular rheumatoid arthritis or osteoarthritis prognosis, or a particular response to treatment for rheumatoid arthritis or osteoarthritis; determining the methylation states of said plurality of nucleic acid loci or said plurality of genes in a sample from a control subject without rheumatoid arthritis, without osteoarthritis, without said rheumatoid arthritis or osteoarthritis prognosis or without said response to treatment for rheumatoid arthritis or osteoarthritis; and identifying loci or genes which are hypermethylated or hypomethylated in said sample from said test subject relative to said sample from said control subject to identify said methylation profile indicative of rheumatoid arthritis, osteoarthritis a rheumatoid arthritis or osteoarthritis prognosis, or a response to treatment for rheumatoid arthritis or osteoarthritis.

Some embodiments also include storing data representing said loci or said genes which are hypermethylated or hypomethylated in said sample from said test subject relative to said sample from said control subject on a non-transitory computer readable medium.

Some embodiments also include comparing the methylation states of a plurality of nucleic acid loci or the methylation states of a plurality of genes in a sample from a test subject with rheumatoid arthritis, osteoarthritis, a particular rheumatoid arthritis or osteoarthritis prognosis, or a particular response to treatment for rheumatoid arthritis or osteoarthritis with the methylation states of said plurality of nucleic acid loci or the methylation states of said plurality of genes in a sample from a control subject without rheumatoid arthritis, without osteoarthritis, without said rheumatoid arthritis or osteoarthritis prognosis or without said response to treatment for rheumatoid arthritis or osteoarthritis.

In some embodiments, the methylation state of said plurality of nucleic acid loci or said plurality of genes in a sample from said test subject or said control subject is determined in fibroblasts from said test subject or said control subject. In some embodiments, the methylation state of said plurality of nucleic acid loci or said plurality of genes in a sample from said test subject or said control subject is determined in synoviocytes from said test subject or said control subject. In some embodiments, the methylation state of said plurality of nucleic acid loci or said plurality of genes in a sample from said test subject or control subject is determined in fibroblast-like synoviocytes from said test subject or said control subject. In some embodiments, the methylation state of said plurality of nucleic acid loci or said plurality of genes in a sample from said test subject or control subject is determined in rheumatoid arthritis fibroblast-like synoviocytes from said test subject or control subject. In some embodiments, the methylation state of said plurality of nucleic acid loci or said plurality of genes in a sample from said test subject or control subject is determined in osteoarthritis fibroblast-like synoviocytes from said test subject or control subject.

In some embodiments, the methylation state of said plurality of nucleic acid loci or said plurality of genes in a sample from said test subject or control subject is determined in macrophage from said test subject or control subject.

In some embodiments, the methylation state of said plurality of nucleic acid loci or said plurality of genes in a sample from said test subject or control subject is determined in peripheral blood cells from said test subject or control subject. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

In some embodiments, the methylation states of at least 5 nucleic acid loci or at least 5 genes are determined. In some embodiments, the methylation states of at least 10 nucleic acid loci or at least 10 genes are determined. In some embodiments, the methylation states of at least 20 nucleic acid loci or at least 20 genes are determined. In some embodiments, the methylation states of at least 50 nucleic acid loci or at least 50 genes are determined. In some embodiments, the methylation states of at least 100 nucleic acid loci or at least 100 genes are determined.

In some embodiments, the subject is a mammal. In some embodiments, the subject is a human.

Some embodiments of the methods, compositions and kits provided herein include a method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising accessing first data representing the methylation status of nucleic acid loci or the methylation status of at least one gene which are differentially methylated in individuals with rheumatoid arthritis, osteoarthritis a rheumatoid arthritis or osteoarthritis prognosis, or a response to treatment for rheumatoid arthritis or osteoarthritis wherein said data is stored on a non-transitory computer readable medium; instructing a computer to compare said first data to second data representing the methylation status of said nucleic acid loci or said at least one gene in a sample taken from said subject, wherein said data representing the methylation status of said nucleic acid loci or said at least one gene in a sample taken from said subject is stored on a non-transitory computer readable medium; and instructing said computer to provide an output indicating whether said comparison indicates that said subject has rheumatoid arthritis or osteoarthritis, has a positive or negative prognosis for rheumatoid arthritis or osteoarthritis prognosis, or indicates a positive or negative prediction for the subject's response to treatment for rheumatoid arthritis or osteoarthritis.

Some embodiments also include diagnosing rheumatoid arthritis or osteoarthritis, determining a rheumatoid arthritis or osteoarthritis prognosis, or determining or predicting a response to treatment for rheumatoid arthritis or osteoarthritis in said subject if said first data representing the methylation status of said nucleic acid loci or said at least one gene in a sample taken from said subject are significantly similar to said second data representing nucleic acid loci or genes which are differentially methylated in individuals with rheumatoid arthritis, osteoarthritis a rheumatoid arthritis or osteoarthritis prognosis, or a response to treatment for rheumatoid arthritis or osteoarthritis.

Some embodiments of the methods, compositions and kits provided herein include a method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising: determining the methylation states of at least 2 loci selected from the group consisting of SEQ ID NO.s 1-485512 in a sample obtained from the subject.

Some embodiments also include comparing the methylation states of the at least 2 loci in the sample with the methylation states of the loci in normal tissue, tissue from a subject with a known prognosis, or tissue from a subject with a known response to treatment.

In some embodiments, an increase or decrease in the extent of methylation of at least 2 loci compared to the extent of methylation of the loci in normal tissue, tissue from a subject with a known prognosis, or tissue from a subject with a known response to treatment is indicative of the presence or absence of rheumatoid arthritis, prognosis, or response to treatment for the subject.

In some embodiments, the methylation states of said at least 2 loci are determined in fibroblasts from said subject. In some embodiments, the methylation states of said at least 2 loci are determined in synoviocytes from said subject. In some embodiments, the methylation states of said at least 2 loci are determined in fibroblast-like synoviocytes from said subject. In some embodiments, the methylations state of said at least 2 loci are determined in rheumatoid arthritis fibroblast-like synoviocytes from said subject. In some embodiments, the methylation states of said at least 2 loci are determined in osteoarthritis fibroblast-like synoviocytes from said subject.

In some embodiments, the methylation states of said at least 2 loci are determined in macrophage from said subject.

In some embodiments, the methylation states of said at least 2 loci are determined in peripheral blood cells from said subject. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

In some embodiments, the at least 2 loci are selected from the group consisting of the loci listed in TABLE 6. In some embodiments, the at least 2 loci are selected from the group consisting of the loci listed in TABLE 7. In some embodiments, the at least 2 loci are selected from the group consisting of the loci listed in TABLE 8. In some embodiments, the methylation states of at least 5 loci selected from the group consisting of the loci listed in TABLE 6, TABLE 7, and TABLE 8 are determined. In some embodiments, the methylation states of at least 10 loci selected from the group consisting of the loci listed in TABLE 6, TABLE 7, and TABLE 8 are determined. In some embodiments, the methylation states of at least 20 loci selected from the group consisting of the loci listed in TABLE 6, TABLE 7, and TABLE 8 are determined. In some embodiments, the methylation states of at least 50 loci selected from the group consisting of the loci listed in TABLE 6, TABLE 7, and TABLE 8 are determined. In some embodiments, the methylation states of at least 100 loci selected from the group consisting of the loci listed in TABLE 6, TABLE 7, and TABLE 8 are determined.

In some embodiments, the subject is a mammal. In some embodiments, the subject is a human.

Some embodiments of the methods, compositions and kits provided herein include a method for identifying a therapeutic agent for treating rheumatoid arthritis or osteoarthritis comprising contacting a cell with a test agent; and determining the methylation states in the contacted cell of at least 2 loci which have differential extents of methylation in individuals with rheumatoid arthritis or osteoarthritis relative to individuals without rheumatoid arthritis or osteoarthritis.

Some embodiments also include comparing the methylation states of the at least 2 loci in the contacted cell with the methylation states of the loci in a cell which was not contacted with the test agent; and selecting a test agent that increases or decreases the extent of methylation of the at least 2 loci in the cell contacted with the test agent compared to the extent of methylation of the at least 2 loci in a cell which was not contacted with the test agent such that the extent of methylation of the at least 2 loci in the cell contacted with the test agent is a methylation states associated with the absence of rheumatoid arthritis or osteoarthritis or with a reduction in the symptoms associated with rheumatoid arthritis osteoarthritis.

In some embodiments, the at least 2 loci are selected from the group consisting of SEQ ID NO.s 1-485512. In some embodiments, the at least 2 loci are selected from the group consisting of the loci listed in TABLE 6. In some embodiments, the at least 2 loci are selected from the group consisting of the loci listed in TABLE 7. In some embodiments, the at least 2 loci are selected from the group consisting of the loci listed in TABLE 8.

In some embodiments, the methylation states of at least 5 loci selected from the group consisting of the loci listed in TABLE 6, TABLE 7, and TABLE 8 are determined. In some embodiments, the methylation states of at least 10 loci selected from the group consisting of the loci listed in TABLE 6, TABLE 7, and TABLE 8 are determined. In some embodiments, the methylation states of at least 20 loci selected from the group consisting of the loci listed in TABLE 6, TABLE 7, and TABLE 8 are determined. In some embodiments, the methylation states of at least 50 loci selected from the group consisting of the loci listed in TABLE 6, TABLE 7, and TABLE 8 are determined. In some embodiments, the methylation states of at least 100 loci selected from the group consisting of the loci listed in TABLE 6, TABLE 7, and TABLE 8 are determined.

In some embodiments, the cell comprises a fibroblast. In some embodiments, the cell comprises a synoviocyte. In some embodiments, the cell comprises a fibroblast-like synoviocyte. In some embodiments, the cell comprises a rheumatoid arthritis fibroblast-like synoviocyte. In some embodiments, the cell comprises an osteoarthritis fibroblast-like synoviocyte.

In some embodiments, the cell comprises a macrophage.

In some embodiments, the cell comprises a peripheral blood cell. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the cell is mammalian. In some embodiments, the cell is a human.

In some embodiments, a sample comprises the cell, the sample selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

Some embodiments of the methods, compositions and kits provided herein include a kit for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising: a reagent for determining the methylation states of at least 2 loci which have differential extents of methylation in individuals with rheumatoid arthritis or osteoarthritis relative to individuals without rheumatoid arthritis or osteoarthritis.

In some embodiments, the at least 2 loci are selected from the group consisting of SEQ ID NO.s 1-485512. In some embodiments, the at least 2 loci are selected from the group consisting of the loci listed in TABLE 6, TABLE 7, and TABLE 8.

Some embodiments also include at least one polynucleotide primer comprising a sequence hybridizing to at least a portion of the at least 2 loci selected from the group consisting of the loci listed in TABLE 6, TABLE 7, and TABLE 8.

In some embodiments, the reagent comprises a restriction enzyme.

In some embodiments, the at least 2 loci are selected from the group consisting of the loci listed in TABLE 6. In some embodiments, the at least 2 loci are selected from the group consisting of the loci listed in TABLE 7. In some embodiments, the at least 2 loci are selected from the group consisting of the loci listed in TABLE 7. In some embodiments, the reagent can determine the methylation states of at least 5 loci selected from the group consisting of SEQ ID NO.s 1-485512. In some embodiments, the reagent can determine the methylation states of at least 5 loci selected from the group consisting of the loci listed in TABLE 6, TABLE 7, and TABLE 8. In some embodiments, the reagent can determine the methylation states of at least 10 loci selected from the group consisting of the loci listed in TABLE 6, TABLE 7, and TABLE 8. In some embodiments, the reagent can determine the methylation states of at least 20 loci selected from the group consisting of the loci listed in TABLE 6, TABLE 7, and TABLE 8. In some embodiments, the reagent can determine the methylation states of at least 50 loci selected from the group consisting of the loci listed in TABLE 6, TABLE 7, and TABLE 8. In some embodiments, the reagent can determine the methylation states of at least 100 loci selected from the group consisting of the loci listed in TABLE 6, TABLE 7, and TABLE 8.

Some embodiments of the methods, compositions and kits provided herein include a method for determining whether an individual suffers from rheumatoid arthritis or osteoarthritis comprising determining the methylation states of at least 2 loci which have differential extents of methylation in individuals with rheumatoid arthritis or osteoarthritis relative to individuals without rheumatoid arthritis or osteoarthritis, wherein the individual is diagnosed with rheumatoid arthritis if the methylation state of said at least 2 loci are associated with rheumatoid arthritis and the individual is diagnosed with osteoarthritis if the methylation states of said at least 2 loci are associated with osteoarthritis.

In some embodiments, the at least 2 loci are selected from the group consisting of SEQ ID NO.s 1-485512. In some embodiments, the at least 2 loci are selected from the group consisting of the loci listed in TABLE 6. In some embodiments, the at least 2 loci are selected from the group consisting of the loci listed in TABLE 7. In some embodiments, the at least 2 loci are selected from the group consisting of the loci listed in TABLE 8.

In some embodiments, the methylation states of at least 5 loci selected from the group consisting of the loci listed in TABLE 6, TABLE 7, and TABLE 8 are determined. In some embodiments, the methylation states of at least 10 loci selected from the group consisting of the loci listed in TABLE 6, TABLE 7, and TABLE 8 are determined. In some embodiments, the methylation states of at least 20 loci selected from the group consisting of the loci listed in TABLE 6, TABLE 7, and TABLE 8 are determined. In some embodiments, the methylation states of at least 50 loci selected from the group consisting of the loci listed in TABLE 6, TABLE 7, and TABLE 8 are determined. In some embodiments, the methylation states of at least 100 loci selected from the group consisting of the loci listed in TABLE 6, TABLE 7, and TABLE 8 are determined.

Some embodiments of the methods, compositions and kits provided herein include a method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising: determining the methylation states of at least 2 genes selected from the group consisting of the genes listed in TABLE 3 in a sample obtained from the subject.

Some embodiments also include comparing the methylation states of the at least 2 genes in the sample with the methylation states of the genes in normal tissue, tissue from a subject with a known prognosis, or tissue from a subject with a known response to treatment.

In some embodiments, an increase or decrease in the extent of methylation of at least 2 genes compared to the extent of methylation of the of the genes in normal tissue, tissue from a subject with a known prognosis, or tissue from a subject with a known response to treatment is indicative of the presence or absence of rheumatoid arthritis, prognosis, or response to treatment for the subject.

In some embodiments, the methylation states of said at least 2 loci are determined in fibroblasts from said subject. In some embodiments, the methylation states of said at least 2 loci are determined in synoviocytes from said subject. In some embodiments, the methylation states of said at least 2 loci are determined in fibroblast-like synoviocytes from said subject. In some embodiments, the methylation states of said at least 2 loci are determined in rheumatoid arthritis fibroblast-like synoviocytes from said subject. In some embodiments, the methylation states of said at least 2 loci are determined in osteoarthritis fibroblast-like synoviocytes from said subject.

In some embodiments, the methylation states of said at least 2 loci are determined in macrophage from said subject.

In some embodiments, the methylation states of said at least 2 loci are determined in peripheral blood cells from said subject. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

In some embodiments, the methylation states of at least 5 genes selected from the group consisting of the genes listed in TABLE 3 are determined. In some embodiments, the methylation states of at least 10 genes selected from the group consisting of the genes listed in TABLE 3 are determined. In some embodiments, the methylation states of at least 20 genes selected from the group consisting of the genes listed in TABLE 3 are determined. In some embodiments, the methylation states of at least 50 genes selected from the group consisting of the genes listed in TABLE 3 are determined.

In some embodiments, the subject is a mammal. In some embodiments, the subject is a human.

Some embodiments of the methods, compositions and kits provided herein include a method for identifying a therapeutic agent for treating rheumatoid arthritis or osteoarthritis comprising contacting a cell with a test agent; and determining the methylation states of at least 2 genes selected from the group consisting of the genes listed in TABLE 3 in the contacted cell.

Some embodiments also include comparing the methylation states of the at least 2 genes in the contacted cell with the methylation states of the genes in a cell which was not contacted with the test agent; and selecting a test agent that increases or decreases the extent of methylation of the at least 2 genes in the cell contacted with the test agent compared to the extent of methylation of the at least 2 genes in a cell which was not contacted with the test agent such that the extent of methylation of the at least 2 genes in the cell contacted with the test agent is a methylation states associated with the absence of rheumatoid arthritis or osteoarthritis or with a reduction in the symptoms associated with rheumatoid arthritis or osteoarthritis.

In some embodiments, the methylation states of at least 5 genes selected from the group consisting of the genes listed in TABLE 3 are determined. In some embodiments, the methylation states of at least 10 genes selected from the group consisting of the genes listed in TABLE 3 are determined. In some embodiments, the methylation states of at least 20 genes selected from the group consisting of the genes listed in TABLE 3 are determined. In some embodiments, the methylation states of at least 50 genes selected from the group consisting of the genes listed in TABLE 3 are determined.

In some embodiments, the cell comprises a fibroblast. In some embodiments, the cell comprises a synoviocyte. In some embodiments, the cell comprises a fibroblast-like synoviocyte. In some embodiments, the cell comprises a rheumatoid arthritis fibroblast-like synoviocyte. In some embodiments, the cell comprises an osteoarthritis fibroblast-like synoviocyte.

In some embodiments, the cell comprises a macrophage.

In some embodiments, the cell comprises a peripheral blood cell. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the cell is mammalian. In some embodiments, the cell is a human.

In some embodiments, a sample comprises the cell, the sample selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

Some embodiments of the methods, compositions and kits provided herein include a kit for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising: a reagent for determining the methylation states of at least 2 genes selected from the group consisting of the genes listed in TABLE 3.

Some embodiments also include at least one polynucleotide primer comprising a sequence hybridizing to at least a portion of the at least 2 genes selected from the group consisting of the genes listed in TABLE 3.

In some embodiments, the reagent comprises a restriction enzyme.

In some embodiments, the reagent can determine the methylation states of at least 5 genes selected from the group consisting of the genes listed in TABLE 3. In some embodiments, the reagent can determine the methylation states of at least 10 genes selected from the group consisting of the genes listed in TABLE 3. In some embodiments, the reagent can determine the methylation states of at least 20 genes selected from the group consisting of the genes listed in TABLE 3. In some embodiments, the reagent can determine the methylation states of at least 50 genes selected from the group consisting of the genes listed in TABLE 3.

Some embodiments of the methods, compositions and kits provided herein include a method for identifying a therapeutic agent for treating rheumatoid arthritis or osteoarthritis comprising contacting a cell with a test agent; and determining the methylation states of at least 2 genes selected from a gene encoding a protein that acts in a pathway that includes a protein encoded by a gene that is differentially methylated in a rheumatoid arthritis cell or that is differentially methylated in a osteoarthritis cell compared to a normal cell.

Some embodiments also include comparing the methylation states of the at least 2 genes in the contacted cell with the methylation states of the genes in a cell which was not contacted with the test agent; and selecting a test agent that increases or decreases the extent of methylation of the at least 2 genes in the cell contacted with the test agent compared to the extent of methylation of the at least 2 genes in a cell which was not contacted with the test agent such that the extent of methylation of the at least 2 genes in the cell contacted with the test agent are methylation states associated with the absence of rheumatoid arthritis or methylation states associated with the absence of osteoarthritis with a reduction in the symptoms associated with rheumatoid arthritis.

In some embodiments, the pathway is selected from the group consisting of the pathways listed in TABLE 5.

In some embodiments, the methylation states of at least 5 genes selected from genes encoding proteins that act in a pathway that includes a protein encoded by a gene that is differentially methylated in a rheumatoid arthritis cell compared to a normal cell or that is differentially methylated in a osteoarthritis cell compared to a normal cell. In some embodiments, the methylation states of at least 10 genes selected from genes encoding proteins that act in a pathway that includes a protein encoded by a gene that is differentially methylated in a rheumatoid arthritis cell compared to a normal cell or that is differentially methylated in a osteoarthritis cell compared to a normal cell.

In some embodiments, the cell comprises a fibroblast. In some embodiments, the cell comprises a synoviocyte. In some embodiments, the cell comprises a fibroblast-like synoviocyte. In some embodiments, the cell comprises a rheumatoid arthritis fibroblast-like synoviocyte. In some embodiments, the cell comprises an osteoarthritis fibroblast-like synoviocyte.

In some embodiments, the cell comprises a macrophage.

In some embodiments, the cell comprises a peripheral blood cell. In some embodiments, the peripheral blood cell is selected from the group consisting of white blood cell, neutrophil, eosinophil, basophil, lymphocyte, B cell, plasma cell, T cell, natural killer cell, monocyte, and dendritic cell.

In some embodiments, the cell is mammalian. In some embodiments, the cell is a human.

In some embodiments, a sample comprises the cell, the sample selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

Some embodiments of the methods, compositions and kits provided herein include a method for identifying therapeutic agents for treating rheumatoid arthritis or osteoarthritis comprising contacting a cell with a test agent; and determining the activity of a protein encoded by a gene differentially methylated in a rheumatoid arthritis cell or that is differentially methylated in an osteoarthritis cell compared to a normal cell.

Some embodiments also include comparing the activity of the protein in the contacted cell with the activity of the protein in a cell which was not contacted with the test agent; and selecting a test agent that increases or decreases the activity of protein in the cell contacted with the test agent compared to the activity of the protein in a cell which was not contacted with the test agent such that the activity of the protein in the cell contacted with the test agent is an activity associated with the absence of rheumatoid arthritis or an activity associated with the absence of osteoarthritis with a reduction in the symptoms associated with rheumatoid arthritis.

In some embodiments, the protein is encoded by a gene selected from the group consisting of the genes listed in TABLE 3.

In some embodiments, the cell comprises a fibroblast. In some embodiments, the cell comprises a synoviocyte. In some embodiments, the cell comprises a fibroblast-like synoviocyte. In some embodiments, the cell comprises a rheumatoid arthritis fibroblast-like synoviocyte. In some embodiments, the cell comprises an osteoarthritis fibroblast-like synoviocyte.

In some embodiments, the cell comprises a macrophage.

In some embodiments, the cell comprises a peripheral blood cell. In some embodiments, the peripheral blood cell is selected from the group consisting of white blood cell, including neutrophil, eosinophil, basophil, lymphocyte, B cell, plasma cell, T cell, natural killer cell, monocyte, and dendritic cell.

In some embodiments, the cell is mammalian. In some embodiments, the cell is a human.

Some embodiments of the methods, compositions and kits provided herein include a method of determining the methylation status of a plurality of human nucleic acid loci comprising contacting a nucleic acid sample from a human subject with a reagent capable of providing an indication of the methylation status of said loci, wherein said loci comprise at least 5 which have differential extents of methylation in individuals with rheumatoid arthritis or osteoarthritis relative to individuals without rheumatoid arthritis or osteoarthritis.

In some embodiments, said reagent is a restriction enzyme.

In some embodiments, said reagent is a primer.

In some embodiments, said reagent is a probe.

In some embodiments, said reagent comprises sodium bisulfate.

In some embodiments, the at least 5 loci are selected from the group consisting of SEQ ID NO.s 1-485512. In some embodiments, the at least 5 loci are selected from the group consisting of the loci listed in TABLE 6, TABLE 7, and TABLE 8.

Some embodiments of the methods, compositions and kits provided herein include a nucleic acid array consisting essentially of nucleic acids useful for diagnosing rheumatoid arthritis or osteoarthritis, determining a prognosis of rheumatoid arthritis or osteoarthritis, or determining or predicting a response to treatment of a subject being evaluated for or suffering from rheumatoid arthritis or osteoarthritis, wherein said nucleic acids comprise at least 5 loci which have differential extents of methylation in individuals with rheumatoid arthritis or osteoarthritis relative to individuals without rheumatoid arthritis or osteoarthritis.

In some embodiments, said nucleic acids comprise at least 5 loci selected from the group consisting of SEQ ID NO.s 1-485512. In some embodiments, said nucleic acids comprise at least 5 loci selected from the group consisting of the loci listed in TABLE 6, TABLE 7, and TABLE 8. In some embodiments, said nucleic acids comprise at least 10 loci selected from the group consisting of the loci listed in TABLE 6, TABLE 7, and TABLE 8. In some embodiments, said nucleic acids comprise at least 20 loci selected from the group consisting of the loci listed in TABLE 6, TABLE 7, and TABLE 8. In some embodiments, said nucleic acids comprise at least 50 loci selected from the group consisting of the loci listed in TABLE 6, TABLE 7, and TABLE 8.

Some embodiments of the methods, compositions and kits provided herein include a method of ameliorating rheumatoid arthritis or osteoarthritis comprising evaluating the methylation status of a plurality of human nucleic acid loci in a nucleic acid sample from a human subject having symptoms of rheumatoid arthritis or osteoarthritis, wherein said loci comprise at least 5 loci which have differential extents of methylation in individuals with rheumatoid arthritis or osteoarthritis relative to individuals without rheumatoid arthritis or osteoarthritis; and administering a treatment for rheumatoid arthritis or osteoarthritis if said at least 5 loci have a methylation status indicative of rheumatoid arthritis or osteoarthritis.

In some embodiments, said nucleic acids comprise at least 5 loci selected from the group consisting of SEQ ID NO.s 1-485512. In some embodiments, said nucleic acids comprise at least 5 loci selected from the group consisting of the loci listed in TABLE 6, TABLE 7, and TABLE 8. In some embodiments, said nucleic acids comprise at least 10 loci selected from the group consisting of the loci listed in TABLE 6, TABLE 7, and TABLE 8. In some embodiments, said nucleic acids comprise at least 20 loci selected from the group consisting of the loci listed in TABLE 6, TABLE 7, and TABLE 8. In some embodiments, said nucleic acids comprise at least 50 loci selected from the group consisting of the loci listed in TABLE 6, TABLE 7, and TABLE 8.

Some embodiments of the methods, compositions and kits provided herein include a mixture comprising a plurality of human nucleic acid loci from a human subject having symptoms indicative of potential rheumatoid arthritis or osteoarthritis and a reagent capable of providing an indication of the methylation status of said loci, wherein said loci comprise at least 5 loci which have differential extents of methylation in individuals with rheumatoid arthritis or osteoarthritis relative to individuals without rheumatoid arthritis or osteoarthritis.

In some embodiments, said nucleic acids comprise at least 5 loci selected from the group consisting of SEQ ID NO.s 1-485512. In some embodiments, said nucleic acids comprise at least 5 loci selected from the group consisting of the loci listed in TABLE 6, TABLE 7, and TABLE 8. In some embodiments, said nucleic acids comprise at least 10 loci selected from the group consisting of the loci listed in TABLE 6, TABLE 7, and TABLE 8. In some embodiments, said nucleic acids comprise at least 20 loci selected from the group consisting of the loci listed in TABLE 6, TABLE 7, and TABLE 8. In some embodiments, said nucleic acids comprise at least 50 loci selected from the group consisting of the loci listed in TABLE 6, TABLE 7, and TABLE 8

### BRIEF DESCRIPTION OF THE DRAWINGS

FIG. 1 depicts DNA methyltranserases (DNMTs) function. DNMTs transfer methyl groups from SAM to deoxycytodine. DNMT function can be affected by changes in methyl donors in the diet or by DNMT inhibitors, like 5-aza-2′-deoxycytidine (5-azaC). The analog 5-azacytidine is also incorporated into RNA and can interfere with many other cellular processes.

FIG. 2 depicts an ILLUMINA array analysis of DNA methylation in RA and OA FLS, and the hierarchical clustering and heatmap of differentially methylated loci. The methylation levels at the 1,859 significantly differentially methylated loci were used for hierarchical clustering. The clustering of the sample is shown by the dendrogram at the top and the clustering of the loci is shown by the dendrogram on the left. The methylation levels at the loci are shown in the heatmap.

FIG. 3 depicts hypomethylation of several genes in RA FLS basal expression compared to OA FLS. Gene expression was determined by PCR in 6 to 13 separate OA and RA FLS lines for 7 genes that were significantly hypomethylated (CHI3L1, COL1A1, MYEF2, ITG4A, SYNJ2, STK24, MAP3K5). As a group, expression of hypomethylated genes in RA was significantly greater than OA (P<0.01); expression of genes that were normally methylated in RA was similar to OA, e.g., AXIN, IKKE, TBK1, NANOG, POU5F1, MAP2K6, IRF3.

FIG. 4 depicts an analysis of the focal adhesion pathway, with differentially methylated genes (ECM=extracellular matrix). Differentially methylated genes are present in key locations, most notably RTK, ITGA and ITGB.

FIG. 5 depicts a Cytoscape analysis to evaluate the networks affected by differential methylation and determine the most likely targets for subsequent analysis.

FIG. 6 depicts basal expression of DNMTs. DNMT expression was examined in resting cultured FLS. Synoviocytes were isolated from RA and OA synovium at the time of total joint replacement. 4^{th} through 6^{th} passage cells were evaluated by qPCR (graph shows relative expression units using the standard curve method).

FIG. 7 depicts decreased DNMT1 gene expression after IL-1 stimulation. FLS were stimulated with IL-1 for 24 hr and DNMT expression was determined by qPCR.

FIG. 8 depicts the decrease in DNMT function in FLS in response to IL-1. FLS were stimulated with 1 ng/ml of IL-1 for 14 days and extracts were assayed using the DNMT Activity/Inhibition Assay (Active Motif Co., Carlsbad, Calif.), which is an ELISA-based method that measures methylation of a CpG-enriched DNA substrate. A significant decline in total DNMT function (p<0.05) was observed.

### DETAILED DESCRIPTION

Abnormalities in DNA methylation have been implicated in autoimmunity. The mechanism of the aggressive rheumatoid phenotype is uncertain, although several studies implicate abnormal tumor suppressor gene structure and function. Differential DNA methylation might lead to altered gene expression, synoviocyte function, and peripheral blood cell function. Global DNA methylation patterns in RA FLS were evaluated and compared to FLS derived from individuals with non-inflammatory joint disease. The data show that the RA FLS display a DNA methylome signature that distinguishes them from osteoarthritis (OA) FLS, with differentially methylated genes that are critical to cell trafficking, inflammation, and cell-extracellular matrix interactions. This signature could define risk factors for developing RA or represent imprinting due to the synovial milieu. A relationship exists between the immunopathologic status of the synovial milieu and peripheral blood mononuclear cell immune function (See e.g., Malone, et al., J Clin Invest., 74(4):1173-1185, 1984; Firestein, G S. Etiology and pathogenesis of rheumatoid arthritis, In: Textbook of Rheumatology, G S Firestein, et al. eds., Elsevier, Philadelphia, 8th edition, 2009, pp. 1035-86). Peripheral blood mononuclear cells in patients with rheumatoid arthritis demonstrate global methylation abnormalities that parallel those found in cultured fibroblast-like synoviocytes, confirming that peripheral blood cells reflect synovial biology and synoviocyte function. Methylation of the IL-10 promoter has been studied in patients with RA (Fu L. H. et al., Methylation status of the IL-10 gene promoter in the peripheral blood mononuclear cells of rheumatoid arthritis patients. Yi Chuan. 2007 November; 29(11):1357-61). However, no previous data has identified patterns of specific combinations of loci or a distinctive methylome signature that can be used to diagnose RA or provide information on the potential response to therapy (Liu C C, et al. Global DNA methylation, DNMT1, and MBD2 in patients with rheumatoid arthritis. Immunol Lett. 2011; 135:96-9; Karouzakis E, et al. DNA hypomethylation in rheumatoid arthritis synovial fibroblasts. Arthritis Rheum. 2009; 60:3613-22).

Data implicating low DNA methylation in FLS is especially intriguing in the context of RA, where pathogenic FLS exhibit many features of partially transformed cells. DNMT regulation and the methylation status of FLS were examined in view of the association of DNA hypomethylation and an aggressive phenotype in cancer. As shown in this application, differences in basal RA vs. OA DNMT expression previously described were not confirmed. This may have been because conditions in the examples of this application were rigorously controlled by discontinuing methotrexate treatment up to a month before surgery. Furthermore, it was found that IL-1 significantly decreased DNMT1, DNMT3a, and DNMT3b gene expression within hours. The change in DNMT expression was accompanied by decreased DNMT function in nuclear extracts and global hypomethylation. More striking, as described in this application, an ILLUMINA methylation array study of RA and OA cells showed 100% concordance between DNA methylation patterns and the presence of RA, indicating that RA cells are imprinted with a distinctive methylation pattern that contributes to the pathogenesis of disease.

The unique pattern of DNA methylation in RA or OA, either taken in toto or when considered based on individual loci, genes, or pathways with differential methylation, has several implications. The pattern can be used for several applications, including: diagnosis of RA or OA; assessment of disease activity and prognosis of RA or OA; identification of novel therapeutic targets useful for the development of novel therapies for RA or OA; and the development of novel therapies that increase or decrease DNA methylation and alter the pattern, such as through DNMT inhibitors or activators.

Embodiments of the present invention include methods, compositions and kits for evaluating a diagnosis, prognosis, or response to treatment of a subject with a disorder such as rheumatoid arthritis or osteoarthritis. Some embodiments include identifying a therapeutic agent for treating a disorder such as rheumatoid arthritis or osteoarthritis.

Like peripheral blood mononuclear cells, rheumatoid FLS exhibit an abnormal phenotype that contributes to disease pathogenesis (Firestein, G S. Invasive fibroblast-like synoviocytes in rheumatoid arthritis: Passive responders or transformed aggressors? Arthritis Rheum 39:1781-1790, 1996). Functional studies suggest that RA FLS are imprinted in situ and maintain these features after many passages in tissue culture. For example, RA FLS, unlike OA or normal synoviocytes, adhere to and invade cartilage explants in SCID mice (Müller-Ladner U et al. Synovial fibroblasts of patients with rheumatoid arthritis attach to and invade normal human cartilage when engrafted into SCID mice. Am J. Pathol. 1996 November; 149(5):1607-15). RA FLS can grow under anchorage-independent conditions and are less susceptible to contact inhibition. Apoptosis of RA synoviocytes in situ appears to be defective, which contributes to intimal lining hyperplasia (Baier A et al. Apoptosis in rheumatoid arthritis. Curr Opin Rheumatol. 2003 May; 15(3):274-9). Data in a SCID mouse model show that RA synoviocytes can migrate from one site to another, thereby serving as a mechanism to spread the RA phenotype and cartilage damage to distant joints (Lefèvre S et al. Synovial fibroblasts spread rheumatoid arthritis to unaffected joints. Nat. Med. 2009 December; 15(12):1414-20). Despite these findings, RA FLS are not truly transformed, as they senesce in culture after 10 to 15 passages.

Several mechanisms have been implicated in the rheumatoid phenotype. For instance, resistance to apoptosis can be due, in part, to defective expression of Phosphatase and tensin homolog (PTEN) expression, high levels of sentrin, or preferential shunting of stressed cells to DNA repair rather than programmed cell death (Pap T et al. Activation of synovial fibroblasts in rheumatoid arthritis: lack of Expression of the tumour suppressor PTEN at sites of invasive growth and destruction. Arthritis Res. 2000; 2(1):59-64; Franz J K et al. Expression of sentrin, a novel antiapoptotic molecule, at sites of synovial invasion in rheumatoid arthritis. Arthritis Rheum. 2000 March; 43(3):599-607; and You X et al. PUMA-mediated apoptosis in fibroblast-like synoviocytes does not require p53. Arthritis Res Ther. 2006; 8(6):R157). Somatic mutations of regulatory genes have been identified in cultured FLS, including the p53 tumor suppressor gene (Firestein G S et al. Somatic mutations in the p53 tumor suppressor gene in rheumatoid arthritis synovium. Proc Natl Acad Sci USA, 94: 10895-10900, 1997; Igarashi H et al. TP53 mutations coincide with the ectopic expression of activation-induced cytidine deaminase in the fibroblast-like synoviocytes derived from a fraction of patients with rheumatoid arthritis. Clin Exp Immunol. 2010 Jul. 1; 161(1):71-80; Inazuka M et al. Analysis of p53 tumour suppressor gene somatic mutations in rheumatoid arthritis synovium. Rheumatology (Oxford). 2000 March; 39(3):262-6; and Rème T et al. Mutations of the p53 tumour suppressor gene in erosive rheumatoid synovial tissue. Clin Exp Immunol. 1998 February; 111(2):353-8). Similar somatic mutations have also been described in peripheral blood mononuclear cells and synovial cells, confirming that these cell populations share many structural DNA characteristics that are either caused by RA or a result of the toxic synovial microenvironment (Cannons J L, et al. HPRT-mutant T cells in the peripheral blood and synovial tissue of patients with rheumatoid arthritis. Arthritis Rheum. 1998; 41:1772-82).

The abnormal cells are more invasive and produce increased amounts of cytokines and metalloproteinases. Microdissection of rheumatoid synovium shows islands of mutant cells residing in the intimal lining that produce prodigious amount of IL-6 (Yamanishi Y et al. p53 regulates apoptosis, synovitis and joint destruction in collagen-induced arthritis. Amer J Pathol, 160:123-30, 2002). Microsatellite instability has also been identified in RA synovium, in part due to decreased DNA repair function (Lee S—H et al. Microsatellite instability and suppressed DNA repair enzyme expression in rheumatoid arthritis. J Immunol, 170:2214-20, 2003). Somatic mutations in several other genes, including mitochondrial DNA and structural proteins like vimentin, have also been reported (Bang H et al. Mutation and citrullination modifies vimentin to a novel autoantigen for rheumatoid arthritis. Arthritis Rheum. 2007 August; 56(8):2503-11; Da Sylva T R et al. Somatic mutations in the mitochondria of rheumatoid arthritis synoviocytes. Arthritis Res Ther. 2005; 7(4):R844-51). Most of these and other aggressive features appear to be result of imprinting by rheumatoid synovial environment. Thus, they serve as amplifying mechanisms that alters the natural history of disease and enhance extracellular matrix destruction and cytokine production, leading to a signature in the systemic circulation due to cellular trafficking that can be detected in peripheral blood cells, for example.

While the focus on gene sequences, mutations, and aggressive behavior has provided useful information, other mechanisms can change cell phenotype. Epigenetics, for instance, can profoundly influence cell activation and gene expression and include DNA methylation, histone modification, and microRNAs. Histone acetylation by histone acetyltransferases (HATs) can remodel chromatin and enhance gene expression while deacetylation by histone deacetylases (HDACs) has the opposite effect. The histone deacetylase HDAC1 is overexpressed in RA FLS and HDAC inhibitors decrease synoviocyte proliferation in culture and joint damage in collagen-induced arthritis (Horiuchi M et al. Expression and function of histone deacetylases in rheumatoid arthritis synovial fibroblasts. J. Rheumatol. 2009 August; 36(8):1580-9; Saouaf S J et al. Deacetylase inhibition increases regulatory T cell function and decreases incidence and severity of collagen-induced arthritis. Exp Mol Pathol. 2009; 87(2):99-104). MicroRNAs are another epigenetic mechanism that contribute to DNA accessibility and chromatin remodeling by directly targeting individual genes. Expression of some individual microRNAs like microRNA-124a, are decreased in RA compared with OA cells, leading to enhanced chemokine expression (Nakamachi Y et al. MicroRNA-124a is a key regulator of proliferation and monocyte chemoattractant protein 1 secretion in fibroblast-like synoviocytes from patients with rheumatoid arthritis. Arthritis Rheum 60:1294, 2009; Stanczyk J et al. Altered expression of microRNA-203 in rheumatoid arthritis synovial fibroblasts and its role in fibroblast activation. Arthritis Rheum. 2011 February; 63 (2): 373-81).

DNA methylation is especially relevant to RA in terms of epigenetic mechanisms by virtue of its role in neoplasia as well as embryonic growth and development. Normal ontogeny relies on a carefully orchestrated sequence of DNA methylation to repress regulatory genes by methylating cytosine in CpG islands after they have completed their programmed role in early development (Christophersen N S and Helin K. Epigenetic control of embryonic stem cell fate. J Exp Med. Oct. 25, 2010; 207(11):2287-95). Methylation abnormalities have been associated with a variety of diseases, most notably cancer where hypomethylation and renewed expression of embryonic genes can allow cells to de-differentiate and escape from normal homeostatic controls (Kulis M and Esteller M. DNA methylation and cancer. Adv Genet. 2010; 70:27-56). Hypermethylation has also been associated with certain malignancies (Ren J et al. DNA hypermethylation as a chemotherapy target. Cell Signal. Feb. 21, 2011).

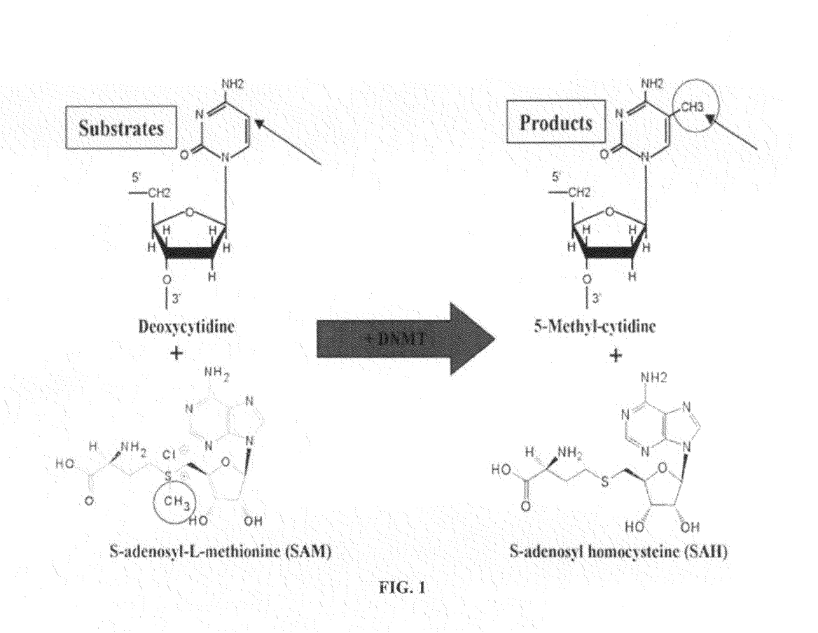

DNA methyltransferases (DNMTs) are responsible for initiating and maintaining CpG methylation in the human genome by converting cytosine to methylcytosine (FIG. 1) (Turek-Plewa J and Jagodzinski P P. The role of mammalian DNA methyltransferases in the regulation of gene expression. Cell Mol Biol Lett. 2005; 10(4):631-47). In mammalian cells, DNMT1, DNMT3a, and DNMT3b are the primary enzymes responsible for CpG methylation. DNMT3a and DNMT3b mainly regulate de novo methylation while DNMT1 maintains methylation, especially during cell division. Thus, DNMT1 plays a greater role perpetuating methylation patterns in proliferating cells. Decreased DNMT expression is associated with global hypomethylation as well as suppressed methylation of individual genes that participate in malignant transformation (Shukla V et al. BRCA1 affects global DNA methylation through regulation of DNMT1. Cell Res. 2010 November; 20(11):1201-15). DNMT expression and DNA methylation is not fixed but can be influenced by the environment and modify gene expression throughout life. The DNMTs can also maintain the methylation pattern during cell division, thereby allowing daughter cells to reflect the environmental influences of the parent cells.

DNMTs can also permit vertical transmission of parental DNA methylation (Ko Y G et al. Stage-by-stage change in DNA methylation status of Dnmt1 locus during mouse early development. J Biol. Chem. 2005 Mar. 11; 280(10):9627-34). This process allows relatively rapid responses to environmental stress that can persist over many cell divisions and even across generations (Rosenfeld. Animal models to study environmental epigenetics. Biol Reprod. 2010, 82:473-88; Kaati G et al. Transgenerational response to nutrition, early life circumstances and longevity. Eur J Hum Genet. 2007 July; 15(7):784-90). For instance, pregnant mice fed a diet rich in methyl donors like folate have increased levels of DNA methylation for at least 2 subsequent generations. Methylation of the Runx3 gene, in particular, is increased by the high methyl donor diet and leads to enhanced Th2 lymphocyte differentiation and increased airway reactivity in murine asthma (Hollingsworth J W et al. In utero supplementation with methyl donors enhances allergic airway disease in mice. J Clin Invest. 2008 October; 118(10):3462-9). Increased disease severity and airway remodeling can even be observed in F2 progeny and demonstrates how the environment can have multigenerational effects (Miller R L. Prenatal maternal diet affects asthma risk in offspring. J Clin Invest. 2008. 118:3265-8).

Abnormalities in DNA methylation have been implicated in autoimmunity. In addition to the murine model of airway disease, T cells can be affected by DNA methylation and influence Th2 differentiation (Gamper C J et al. Identification of DNA methyltransferase 3a as a T cell receptor-induced regulator of Th1 and Th2 differentiation. J Immunol. 2009 Aug. 15; 183(4):2267-76). The DNMT inhibitor 5′-aza-2′-deoxycytidine (5-azaC) (Fandy T E. Development of DNA methyltransferase inhibitors for the treatment of neoplastic diseases. Curr Med. Chem. 2009; 16(17):2075-85) affects expression of many T cell genes, including IFNα, IL-4, CD70, and LFA-1. 5-azaC enhances autoreactivity and induces robust responses to normally sub-threshold stimulation (Richardson B. DNA methylation and autoimmune disease. Clin Immunol. 2003 October; 109(1):72-9). T and B cell interactions are also affected, in part due to altered expression of surface receptors like CD70 (Oelke K et al. Overexpression of CD70 and overstimulation of IgG synthesis by lupus T cells and T cells treated with DNA methylation inhibitors. Arthritis Rheum. 2004 June; 50(6):1850-60). Decreased DNA methylation in Th1 and Th2 cell genomic DNA is also associated with production of anti-dsDNA antibodies in vivo (Richardson B et al. Murine models of lupus induced by hypomethylated T cells. Methods Mol Med. 2004; 102:285-94). Hypomethylation has been described in peripheral blood mononuclear cells in patients with RA (Liu C C et al. Global DNA methylation, DNMT1, and MBD2 in patients with rheumatoid arthritis. Immunol Lett. Mar. 30, 2011; 135(1-2):96-9. Epub Oct. 16, 2010). Despite some evidence of abnormal global methylation, there is no information on specific loci, patterns of loci, genes, or pathways that are abnormally methylated in rheumatoid arthritis.

Evidence of hypomethylation is not restricted to adaptive immunity in rheumatic disease. Like peripheral blood cells, global genomic hypomethylation was recently reported in RA cultured fibroblast-like synoviocytes (FLS) compared with Osteoarthritis (OA) FLS (Karouzakis E et al. DNA hypomethylation in rheumatoid arthritis synovial fibroblasts. Arthritis Rheum. 2009 December; 60(12):3613-22). This observation was associated with relatively low levels of DNMT1 gene expression that were unaffected by in vitro stimulation with IL-1 or TNF. DNMT3a or DNMT3b were not examined, and no studies were performed to determine whether DNMT function was abnormal. Culturing FLS in the presence of 5-azaC for 3 months to block DNA methylation increased expression of several genes implicated in RA. However, it is not certain which genes were hypomethylated and which ones were affected secondary to decreased methylation of other regulatory genes.

DNMT regulation and the methylation status of FLS were examined in view of the association of DNA hypomethylation and an aggressive phenotype in cancer. As shown in this application, it was found that IL-1 significantly decreased DNMT1, DNMT3a, and DNMT3b gene expression within hours. The change in DNMT expression was accompanied by decreased DNMT function in nuclear extracts and global hypomethylation. More striking, an ILLUMINA methylation array study of RA and OA cells showed 100% concordance between DNA methylation patterns and the presence of RA.

The unique patterns of DNA methylation in RA or OA have several implications. The patterns can be used for several applications, including: diagnosis of RA or OA; assessment of disease activity and prognosis of RA or OA; identification of novel therapeutic targets useful for the development of novel therapies for RA or OA; and the development of novel therapies that increase or decrease DNA methylation and alter the pattern, such as though DNMT inhibitors or activators.

### DEFINITIONS

As used herein, “methylation” refers to cytosine methylation at positions C5 or N4 of cytosine, the N6 position of adenine or other types of nucleic acid methylation. In particular embodiments, “methylation” refers to cytosine methylation at positions C5 of cytosine, namely, 5-methly cytosine. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively.

A “methylation profile” refers to a set of data representing the methylation states of two or more loci within a molecule of DNA from e.g., the genome of an individual or cells or tissues from an individual. The profile can indicate the methylation state of every cytosine base in an individual, can comprise information regarding a subset of the base pairs (e.g., the methylation state of specific restriction enzyme recognition sequence) in a genome, or can comprise information regarding regional methylation density of each locus.

As used herein, “methylation status” refers to the presence, absence and/or quantity of methylation at a particular nucleotide, or nucleotides within a portion of DNA. Determination of the methylation status of a particular DNA sequence (e.g., a locus, a DNA biomarker or DNA region as described herein) can involve determination of the methylation state of every cytosine in the sequence or can involve determination of the methylation state of a subset of the cytosines (such as the methylation state of cytosines in one or more specific restriction enzyme recognition sequences) within the sequence, or can involve determining regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value.

As used herein, “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC(N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC (see, e.g., U.S. Pat. No. 5,405,760), McrA, MrrA, BisI, GlaI and DpnI. One of skill in the art will appreciate that any methylation-dependent restriction enzyme, including homologs and orthologs of the restriction enzymes described herein, is also suitable for use in the present embodiments.

As used herein, “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., Nucleic Acids Res. 22(17):3640-59 (1994) and http://rebase.neb.com. Suitable methylation-sensitive restriction enzymes that do not cleave DNA at or near their recognition sequence when a cytosine within the recognition sequence is methylated at position C5 include, e.g., Aat I I, Aci I, Acd I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N6 include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

### Evaluating a Diagnosis, Prognosis, or Response to Treatment

Some embodiments provided herein relate to methods for diagnosing, determining a prognosis, or determining or predicting a response to treatment. As used herein, diagnosing can include determining whether a methylation status of 1 or more loci is indicative of a disorder, such as rheumatoid arthritis. As used herein, determining a prognosis can include determining whether methylation status of 1 or more loci is indicative of a likelihood of improvement in symptoms of a disorder, such as rheumatoid arthritis. As used herein, determining or predicting a response to treatment can include determining whether methylation status of 1 or more loci after treatment is more similar to a normal status before treatment or earlier in the treatment regimen. In some embodiments of the methods and compositions provided herein, the 1 or more locus or 1 or more gene is a locus or gene with no known association with RA.

In some such embodiments, the methylation state of at least 1 locus or at least one gene selected from a locus or gene described herein in a sample obtained from a subject is determined. Examples of loci for which the methylation state may be evaluated include the loci listed in TABLE 6, TABLE 7, and TABLE 8. In some embodiments, loci for which the methylation state may be evaluated include SEQ ID NO.s:1-485512. In each sequence provided in SEQ ID NO.s 1-485512, the “C” which is potentially methylated is at position 61. Examples of genes for which the methylation state may be evaluated include the loci listed in TABLE 3. The nucleic acid sequences of the loci listed in TABLE 6, TABLE 7, and TABLE 8 and the genes listed in TABLE 3 are incorporated herein by reference. In some embodiments, additional loci and genes useful for the methods and compositions provided herein can be further identified using the methods described herein. In some embodiments, additional loci and genes useful for the methods and compositions provided herein are identified by conducting methylation analyses in additional samples, thereby providing an increased number of data points which could assist in the identification of further genes or loci having statistically significant differences in their methylation states. The sample can comprise an in vivo sample, an in vitro sample, or an ex vivo sample. It will be understood, that in some embodiments of the compositions or methods provided herein, a sample or cell can be in vivo. In some embodiments of the compositions or methods provided herein, a sample or cell can be ex vivo. Methods to determine the methylation state of at least one locus or at least one gene are well known in the art and examples are provided herein. In some embodiments, the subject is a mammal, such as a human. In some embodiments, the methylation states of at least about 1, 2, 3, 4, 5, 10, 20, 30, 40, 50, 60, 70, 80, 90, 100, 200, 300, 400, 500 loci or more than 500 loci are determined. Some embodiments also include comparing the methylation state of the at least 1 locus in the sample with the methylation state of the locus in a normal cell, cell from a subject with a known prognosis, or cell from a subject with a known response to treatment. In some embodiments, the methylation states of at least about 1, 2, 3, 4, 5, 10, 20, 30, 40, 50, 60, 70, 80, 90, 100, 200, 300, 400, 500 genes or more than 500 genes are determined. Some embodiments also include comparing the methylation state of the at least 1 gene in the sample with the methylation state of the gene in a normal cell, cell from a subject with a known prognosis, or cell from a subject with a known response to treatment.

In some embodiments, an increase or decrease in the methylation state of the at least 1 locus compared to the methylation state of the locus in normal cell, cell from a subject with a known prognosis, or cell from a subject with a known response to treatment is indicative of the diagnosis, prognosis, or response to treatment for the subject. In some embodiments, an increase or decrease in the methylation state of a locus selected from the group consisting of the loci listed in TABLE 6 is indicative of the diagnosis, prognosis, or response to treatment for the subject. In some embodiments, an increase or decrease in the methylation state of a locus selected from the group consisting of the loci listed in TABLE 7 is indicative of the diagnosis, prognosis, or response to treatment for the subject. In some embodiments, an increase or decrease in the methylation state of a locus selected from the group consisting of the loci listed in TABLE 8 is indicative of the diagnosis, prognosis, or response to treatment for the subject. In some embodiments, an increase or decrease in the methylation state of the at least 1 gene compared to the methylation state of the gene in a normal cell, cell from a subject with a known prognosis, or tissue from a subject with a known response to treatment is indicative of the diagnosis, prognosis, or response to treatment for the subject. In some embodiments, an increase or decrease in the methylation state of a gene selected from the group consisting of the genes listed in TABLE 3 is indicative of the diagnosis, prognosis, or response to treatment for the subject. In some embodiments, the increase or decrease in methylation occurs in a cell, such as a synoviocyte, such as a fibroblast-like synoviocyte, for example, a rheumatoid arthritis fibroblast-like synoviocyte or an osteoarthritis fibroblast-like synoviocyte. In some embodiments, the cell comprises macrophage. In some embodiments, the cell comprises a peripheral blood cell. As used herein, ‘peripheral blood cell’ can include a cellular component of blood which contains DNA. Examples of peripheral blood cells include white blood cells, including neutrophils, eosinophils, basophils, lymphocytes, B cell, plasma cells, T cells, natural killer cells, monocytes, and dendritic cells. In some embodiments, the cell is mammalian, e.g., human. In some embodiments, the loci and genes which are differentially methylated in fibroblast-like synoviocytes from individuals with rheumatoid arthritis or osteoarthritis and the loci and genes which are differentially methylated in individuals with rheumatoid or osteoarthritis in cell types other than fibroblast-like synoviocytes may partially overlap. However, it is likely that there will be loci and genes which exhibit differential methylation in individuals with rheumatoid arthritis or osteoarthritis in one cell type which are not differentially methylated in other cell types from individuals with rheumatoid arthritis or osteoarthritis. Such differences in differential methylation may be a reflection of the fact that methylation patterns vary among different cell lineages. For example, differentially methylated loci and genes identified in T cells and in B cells types from individuals with rheumatoid arthritis or osteoarthritis can include loci and genes that are different in each cell type. Differentially methylated loci and genes in different cell types from individuals with rheumatoid arthritis or osteoarthritis can be identified using the methods described herein.

Some embodiments include methods of ameliorating rheumatoid arthritis or osteoarthritis in a subject. Some such embodiments include evaluating the methylation status of a plurality of human nucleic acid loci in a nucleic acid sample from a human subject having symptoms of rheumatoid arthritis or osteoarthritis. In some embodiments, the loci comprise at least about 5 loci, at least about 10 loci, at least about 15 loci, at least about 20 loci, at least about 25 loci, at least about 50 loci, and at least about 100 loci. In some embodiments the loci are selected from the group consisting of the loci listed in TABLE 6, TABLE 7, or TABLE 8. In some embodiments, the loci may be selected from SEQ ID NO.s:1-485512. Some embodiments also include administering a treatment for rheumatoid arthritis or osteoarthritis if the loci have a methylation status indicative of rheumatoid arthritis or osteoarthritis.

Some embodiments include a mixture comprising a plurality of human nucleic acid loci from a human subject having symptoms indicative of potential rheumatoid arthritis or osteoarthritis and a reagent capable of providing an indication of the methylation status of said loci. In some embodiments, the loci comprise at least about 5 loci, at least about 10 loci, at least about 15 loci, at least about 20 loci, at least about 25 loci, at least about 50 loci, and at least about 100 loci. In some embodiments the loci are selected from the group consisting of the loci listed in TABLE 6, TABLE 7, or TABLE 8. In some embodiments, the loci are selected from SEQ ID NO.s:1-485512

### Methods to Determine Methylation State of a Locus

In some embodiments, the methylation state of more than one DNA region, e.g., gene, locus or portion thereof is determined. In some embodiments, the methylation status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or more than 97 of the DNA regions is determined.

In some embodiments, the methylation state of a DNA region or portion thereof is determined and then normalized (e.g., compared) to the methylation state of a control locus. Typically the control locus will have a known, relatively constant, methylation status. For example, the control sequence can be previously determined to have no, some or a high amount of methylation, thereby providing a relative constant value to control for error in detection methods, etc., unrelated to the presence or absence of a disorder. In some embodiments, the control locus is endogenous, i.e., is part of the genome of the individual sampled. For example, in mammalian cells, the testes-specific histone 2B gene (hTH2B in human) gene is known to be methylated in all somatic tissues except testes. Alternatively, the control locus can be an exogenous locus, i.e., a DNA sequence spiked into the sample in a known quantity and having a known methylation status.

A DNA region comprises a nucleic acid including one or more methylation sites of interest (e.g., a cytosine, a “microarray feature,” or an amplicon amplified from select primers) and flanking nucleic acid sequences (i.e., “wingspan”) of up to 4 kilobases (kb) in either or both of the 3′ or 5′ direction from the amplicon. This range corresponds to the lengths of DNA fragments obtained by randomly fragmenting the DNA before screening for differential methylation between DNA in two or more samples (e.g., carrying out methods used to initially identify differentially methylated loci). In some embodiments, the wingspan of the one or more DNA regions is about 0.5 kb, 0.75 kb, 1.0 kb, 1.5 kb, 2.0 kb, 2.5 kb, 3.0 kb, 3.5 kb or 4.0 kb in both 3′ and 5′ directions relative to the sequence represented by the microarray feature. The DNA region of interest can comprise and/or be immediately adjacent to a locus selected from the group consisting of the loci listed in TABLE 6, TABLE 7, or TABLE 8, or a gene selected from a gene listed in TABLE 3. In some embodiments, the locus may be selected from SEQ ID NO.s:1-485512. The nucleic acid sequences of the loci listed in TABLE 6, TABLE 7, and TABLE 8 are available, for example, in the Illumina CpG database, and included in SEQ ID NO.s:1-485512.

The methylation sites in a DNA region can reside in non-coding transcriptional control sequences (e.g., promoters, enhancers, etc.) or in coding sequences, including introns and exons of the loci listed in TABLE 6, TABLE 7, or TABLE 8, and genes listed in TABLE 3. In some embodiments, the methods comprise detecting the methylation status in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the translational start site) of one or more of the locus identified in TABLE 6, TABLE 7, TABLE 8, or TABLE 3. In some embodiments, the locus may be selected from SEQ ID NO.s:1-485512.

Any method for detecting DNA methylation can be used in the methods provided herein. In some embodiments, an array can be used to determine the methylation state of at least one locus, such as the ILLUMINA HumanMethylation 450 BeadChip. DNA is treated with bisulfite to convert unmethylated cytosines to uracil, methylated cytosines are protected and remain cytosine. After the conversion step, a determination step is performed to identify whether a base at a particular locus was converted. Methylation status of the interrogated site is calculated as the ratio of the signal from a methylated probe relative to the sum of both methylated and unmethlylated probes. This value, known as β, ranges continuously from 0 (unmethlylated) to 1 (fully methylated). Arrays, such as the ILLUMINA HumanMethylation 450 BeadChip, include genes and CpG islands and other sequences.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. patent application Ser. Nos. 10/971,986; 11/071,013; and 10/971,339. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. As used herein, ‘methylation density’ can refer to the number of methylated C-residues within a region. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. patent application Ser. No. 10/971,986.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol. Biotechnol. 20(2):163-79 (2002). Amplifications may be monitored in “real time.”

Additional methods for detecting DNA methylation can involve genomic sequencing before and after treatment of the DNA with bisulfite. See, e.g., Frommer et al., Proc. Natl. Acad. Sci. USA 89:1827-1831 (1992). When sodium bisulfite is contacted to DNA, unmethylated cytosine is converted to uracil, while methylated cytosine is not modified.

In some embodiments, restriction enzyme digestion of PCR products amplified from bisulfite-converted DNA is used to detect DNA methylation. See, e.g., Sadri & Hornsby, Nucl. Acids Res. 24:5058-5059 (1996); Xiong & Laird, Nucleic Acids Res. 25:2532-2534 (1997).

In some embodiments, a MethyLight assay is used alone or in combination with other methods to detect DNA methylation (see, Eads et al., Cancer Res. 59:2302-2306 (1999)). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably-labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect DNA methylation (see, Gonzalgo & Jones, Nucleic Acids Res. 25:2529-2531 (1997)). The Ms-SNuPE technique is a quantitative method for assessing methylation differences at specific CpG sites based on bisulfite treatment of DNA, followed by single-nucleotide primer extension (Gonzalgo & Jones, supra). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA. See, Herman et al., Proc. Natl. Acad. Sci. USA 93:9821-9826, (1996); U.S. Pat. No. 5,786,146.

Additional methylation detection methods include, but are not limited to, methylated CpG island amplification (see, Toyota et al., Cancer Res. 59:2307-12 (1999)) and those described in, e.g., U.S. Patent Publication 2005/0069879; Rein, et al. Nucleic Acids Res. 26 (10): 2255-64 (1998); Olek, et al. Nat. Genet. 17(3): 275-6 (1997); and PCT Publication No. WO 00/70090.

Some embodiments provided herein include methods of determining the methylation status of a plurality of human nucleic acid loci. Some such embodiments include contacting a nucleic acid sample from a human subject with a reagent capable of providing an indication of the methylation status of said loci. In some embodiments, the loci comprise at least about 5 loci, at least about 10 loci, at least about 15 loci, at least about 20 loci, at least about 25 loci, at least about 50 loci, and at least about 100 loci. In some embodiments the loci are selected from the group consisting of the loci listed in TABLE 6, TABLE 7, or TABLE 8. In some embodiments, the reagent is a restriction enzyme. In some embodiments, the reagent is a primer. In some embodiments, the reagent is a probe. In some embodiments, the reagent comprises sodium bisulfite.

### Identifying Therapeutic Agents

Some embodiments provided herein relate to methods for identifying therapeutic agents. Some such embodiments for identifying therapeutic agents which may be used to treat rheumatoid arthritis or osteoarthritis, can include contacting a cell with a test agent; and determining the methylation state of at least 1 locus selected from the loci listed in TABLE 6, TABLE 7, or TABLE 8 or at least one gene listed in Table 3 in the contacted cell. In some embodiments, the at least one locus may be selected from SEQ ID NO.s:1-485512. Some methods also include comparing the methylation state of the at least 1 locus, or at least 1 gene in the contacted cell with the methylation state of the locus or the gene in the cell not contacted with the test agent, and selecting a test agent that increases or decreases the methylation state of the at least 1 locus or the at least 1 gene in the cell contacted with the test agent compared to the methylation state of the locus or the gene in a cell not contacted with the test agent. For example, if a locus, a gene group of loci or group of genes are hypermethylated in individuals with RA, agents which reduce the level of methylation at the locus, the gene, group of loci or group of genes may be useful as therapeutic agents. Likewise, if a locus, a gene, group of loci or group of genes are hypomethylated in individuals with RA, agents which increase the level of methylation at the locus, the gene group of loci or group of genes may be useful as therapeutic agents. Likewise, agents which produce a methylation profile in cells contacted with the agent having a greater similarity to the methylation profile of individuals who do not suffer from RA relative to the methylation profile in cells which have not been contacted with the agent may be useful as therapeutic agents. Examples of test agents and potential therapeutic agents include small molecules (including but not limited to organic chemical compounds which have been obtained from natural sources or synthesized), nucleic acids (including but not limited to antisense nucleic acids, ribozymes, or siRNAs), peptides and proteins (including but not limited to cytokines TNF-α, and DMNTs).

In some embodiments, at least 1 locus is selected from the group consisting of the loci listed in TABLE 6. In some embodiments, at least 1 locus is selected from the group consisting of the loci listed in TABLE 7. In some embodiments, the at least one locus may be selected from SEQ ID NO.s:1-485512. In some embodiments, at least 1 gene is selected from the group consisting of the gene listed in TABLE 3.

In some embodiments, the methylation states of at least about 5, 10, 20, 30, 40, 50, 60, 70, 80, 90, or 100 loci selected from the group consisting of the loci listed in TABLE 6, TABLE 7, or TABLE 8 are determined. In some embodiments, the methylation states of at least about 5, 10, 20, 30, 40, 50, 60, 70, 80, or 100 loci selected from the group consisting of the loci of SEQ ID NO.s:1-485512. In some embodiments, the methylation states of at least about 1, 5, 10, 20, 30, 40, 50 genes selected from the group consisting of the genes listed in TABLE 3 are determined

In some embodiments, the cell comprises a synoviocyte, such as a fibroblast-like synoviocyte, for example, a rheumatoid arthritis fibroblast-like synoviocyte or an osteoarthritis fibroblast-like synoviocyte. In some embodiments, the cell comprises a macrophage. In some embodiments, the cell comprises a peripheral blood cell. In some embodiments, the peripheral blood cell is selected from the group consisting of white blood cell, including neutrophil, eosinophil, basophil, lymphocyte, B cell, plasma cell, T cell, natural killer cell, monocyte, and dendritic cell. In some embodiments, the cell is mammalian, e.g., human.

More embodiments of methods for identifying therapeutic reagents include identifying agents that modulate methylation of genes encoding proteins that act in the same pathway as other proteins encoded by genes that are differentially methylated in rheumatoid arthritis or osteoarthritis or agents which modulate the activity of proteins in the same pathway as proteins encoded by genes which are differentially methylated in rheumatoid arthritis or osteoarthritis. Some methods for identifying a therapeutic agent for treating rheumatoid arthritis or osteoarthritis include contacting a cell with a test agent, and determining the methylation state of at least 1 gene selected from a gene encoding a protein that acts in a pathway that includes a protein encoded by a gene that is differentially methylated in a rheumatoid arthritis cell or osteoarthritis cell compared to a normal cell. Some methods also include comparing the methylation state of the at least 1 gene in the contacted cell with the methylation state of the gene in a cell which was not contacted with the test agent, selecting a test agent that increases or decreases the extent of methylation of the at least 1 gene in the cell contacted with the test agent compared to the extent of methylation of the at least 1 gene in a cell which was not contacted with the test agent such that the extent of methylation of the at least 1 gene in the cell contacted with the test agent is a methylation state associated with the absence of rheumatoid arthritis or osteoarthritis or with a reduction in the symptoms associated with rheumatoid arthritis or osteoarthrities. In some embodiments, the pathway is selected from focal adhesion, glycosphingolipid biosynthesis—lacto and neolacto series, arrhythmogenic right ventricular cardiomyopathy (ARVC), ECM-receptor interaction, amoebiasis, leukocyte transendothelial migration, protein digestion and absorption, cell adhesion molecules (CAMs), nitrogen metabolism, ErbB signaling pathway, African trypanosomiasis, primary bile acid biosynthesis, Fc epsilon RI signaling pathway, mTOR signaling pathway, and adipocytokine signaling pathway.

### Kits

Some embodiments provided herein relate to kits. Some such kits can be useful for diagnosing, determining a prognosis, or determining a response to treatment of a subject with a disorder, such as rheumatoid arthritis, comprising: a reagent for determining the methylation state of at least one locus selected from the group consisting of the loci listed in TABLE 6, TABLE 7, or TABLE 8. In some embodiments, the at least one locus may be selected from SEQ ID NO.s:1-485512. In some embodiments, the kit also includes at least one polynucleotide primer comprising a sequence hybridizing to at least a portion of the at least one locus selected from the group consisting of the loci listed in TABLE 6, TABLE 7, or TABLE 8. In some embodiments, the locus may be selected from SEQ ID NO.s:1-485512. In some embodiments, the kit can include one or more of methylation-dependent restriction enzymes, methylation-sensitive restriction enzymes, amplification (e.g., PCR) reagents, probes and/or primers. In some embodiments a reagent can determine the methylation states of at least about 1, 5, 10, 20, 30, 40, 50, 60, 70, 80, 90, 100 or more than 100 loci selected from the group consisting of the loci listed in TABLE 6, TABLE 7, or TABLE 8. In some embodiments, the at least about 1, 5, 10, 20, 30, 40, 50, 60, 70, 80, 90, 100 or more than 100 loci may be selected from SEQ ID NO.s:1-485512.

Some embodiments include kits for diagnosing, determining a prognosis, or determining or predicting a response to treatment of a subject with rheumatoid arthritis or osteoarthritis, comprising a reagent for determining the methylation state of at least one gene selected from the group consisting of the genes listed in TABLE 3. Some kits also include at least one polynucleotide primer comprising a sequence hybridizing to at least a portion of the at least one gene selected from the group consisting of the genes listed in TABLE 3. In some embodiments, the reagent comprises a restriction enzyme. In some embodiments a reagent can determine the methylation states of at least about 1, 5, 10, 20, 30, 40, 50, or more genes selected from the group consisting of the genes listed in TABLE 3.

Some embodiments include a nucleic acid array consisting essentially of nucleic acids useful for diagnosing rheumatoid arthritis or osteoarthritis, determining a prognosis of rheumatoid arthritis or osteoarthritis, or determining or predicting a response to treatment of a subject being evaluated for or suffering from rheumatoid arthritis or osteoarthritis. In some such embodiments, the nucleic acids comprise at least about 5 loci, at least about 10 loci, at least about 15 loci, at least about 20 loci, at least about 25 loci, at least about 50 loci, and at least about 100 loci. In some embodiments the loci are selected from the group consisting of the loci listed in TABLE 6, TABLE 7, or TABLE 8. In some embodiments, the loci may be selected from SEQ ID NO.s:1-485512.

### Computer-Based Methods

The calculations for the methods described herein can involve computer-based calculations and tools. For example, a methylation value for a DNA region or portion thereof can be compared by a computer to a threshold value, as described herein. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

Some embodiments include methods for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising accessing first data representing nucleic acid loci which are differentially methylated in individuals with rheumatoid arthritis, osteoarthritis a rheumatoid arthritis or osteoarthritis prognosis, or a response to treatment for rheumatoid arthritis or osteoarthritis wherein said data is stored on a non-transitory computer readable medium. Some embodiments also include instructing a computer to compare said first data to second data representing the methylation status of said nucleic acid loci in a sample taken from said subject, wherein said data representing the methylation status of said nucleic acid loci in a sample taken from said subject is stored on a non-transitory computer readable medium. Some embodiments also include diagnosing rheumatoid arthritis or osteoarthritis, determining a rheumatoid arthritis or osteoarthritis prognosis, or determining or predicting a response to treatment for rheumatoid arthritis or osteoarthritis in said subject if said first data representing the methylation status of said nucleic acid loci in a sample taken from said subject are significantly similar to said second data representing nucleic acid loci which are differentially methylated in individuals with rheumatoid arthritis, osteoarthritis a rheumatoid arthritis or osteoarthritis prognosis, or a response to treatment for rheumatoid arthritis or osteoarthritis.

While the present invention has been described in some detail for purposes of clarity and understanding, one skilled in the art will appreciate that various changes in form and detail can be made without departing from the true scope of the invention.

### EXAMPLES

### Example 1

### Methylation of Loci in RA and OA Patients

### Methods: Fibroblast-Like Synoviocytes

FLS were isolated from synovial tissues obtained from RA and OA patients at the time of joint replacement as described previously. The diagnosis of RA conformed to the American College of Rheumatology 1987 revised criteria. The protocol was approved by the UCSD Human Subjects Research Protection Program. Synovial tissues were minced and incubated with 0.5 mg/ml collagenase VIII (Sigma) in serum-free RPMI (Mediatech, VA) for 1.5 h at 37° C., filtered through a 0.22 μm cell strainer, extensively washed, and cultured in DMEM supplemented with 10% FCS (endotoxin content<0.006 ng/ml; Gemini Biosciences, CA), penicillin, streptomycin, gentamicin and L-glutamine in a humidified 5% CO₂ incubator. After overnight culture, nonadherent cells were removed, and adherent cells were trypsinized, split at a 1:3 ratio, and cultured. Synoviocytes were used from passage 4 through 9, when FLS were a homogeneous population with <1% CD11b, <1% phagocytic, and <1% FcR II positive cells.

Patient Phenotype

Synovial tissues were obtained at the time of clinically indicated total knee or hip joint replacement surgery except for one patient with RA who had wrist surgery. The mean ages of RA and OA patients were 53±9 and 68±16, respectively. Additional information on four patients (2 RA and 2 OA) was limited because the samples were de-identified. The erythrocyte sedimentation rates for the remaining RA and OA patients were 38±15 and 19±10, respectively. Of the 4 RA patients with clinical information, 3 were seropositive for serum rheumatoid factor or anti-CCP antibody and all were treated with low dose prednisone, 2 with methotrexate, 2 with a TNF blocker, and 1 with leflunomide. OA was mainly treated with acetaminophin and narcotics for pain.

Isolation of Genomic DNA and qPCR Analysis

RA and OA FLS were grown to 80% confluence and harvested. Genomic DNA of 10⁶ FLS was isolated using the MagMAX™ DNA Multi-Sample Kit (Applied Biosystems). DNA quality and quantity was assessed with a NanoDrop ND-2000 spectrometer (NanoDrop Technologies, Wilmington, Del., USA). mRNA from cultured FLS was isolated using RNA-STAT (Tel-Stat, TX) and cDNA was prepared, according to manufacturer's instructions using GeneAmp 2400 (Applied Biosystems). Quantitative real-time PCR was performed using Assays On Demand (Applied Biosystems) to determine relative mRNA levels using the GeneAmp 5700 Sequence Detection System (Applied Biosystems) as described previously. Standard curves for human MMP1 and GAPDH were generated. Sample Ct values were used to calculate the number of cell equivalents in the test samples. The data were then normalized to GAPDH expression to obtain relative cell equivalents.

Infinium HumanMethylation450 Chip Analysis

Genomic DNA was isolated from female RA and OA FLS as described. The Infinium HumanMethylation450 chip was processed as described by the manufacturer (Illumina, San Diego, Calif.). This chip covers 96% of RefSeq genes and provides comprehensive gene region coverage, targeting multiple sites with promoter, 5□ UTR, 1st exon, gene body and 3□ UTR. Initial analysis was performed with the Genome Studio methylation module, and then further analysed as described herein. The methylation level of a loci is measured as:

β=M/(U+M+100)

M is the fluorescence level of the methylation probe and U is the methylation level of the unmethylated probe. A constant value of 100 is added to prevent division by a small number (or 0) when background subtraction was used. The β values varied from 0 (completely unmethylated) to 1 (completely methylated). To measure the difference in methylation at a loci between OA and RA the average β levels were compared.

Method for Determining Enrichment of Multiple Methylated Loci

To avoid taking a single P-value cut-off when identifying genes that are enriched with differentially methylated (differentially methylated) loci, a variable cut-off scheme was used. Loci were ranked by their differentially methylated P-values. Then a P-value cut-off was taken. Those loci with P-value beneath the cut-off were taken to be differentially methylated and the rest were taken as not being differentially methylated. While doing this all P-value cut-offs beneath 0.05 were tried. Then for each gene, with loci beneath the P-value cut-off, the level which its loci were enriched beneath the cut-off was calculated as its enrichment factor (EF).

EF=(number of loci from gene A beneath cut-off/total loci from gene A)/(total number of loci beneath cut-off/total number of loci)

If EF is greater than 1 then it means the genes loci are enriched with differentially methylated loci. For those genes with an EF greater than 1, a P-value for the level of enrichment was calculated using the hypergeometric distribution. The resulting P-values were corrected with the Benjamini-Hochberg correction. Genes with enrichment P-values beneath <0.05 were recorded. If a gene was found to be enriched at multiple loci differentially methylated levels then only the level with the lower enrichment P-value was reported.

### Pathway and Gene Ontology Analyses

Pathway enrichment was carried out using the KEGG human pathways and modules (www.genome.jp/kegg/download). The enrichment analysis of 1859 loci was performed by mapping pathway to loci via the loci gene annotations, the EF of loci being enriched in KEGG pathway as calculated. If EF was greater than 1 then P-value for the level of enrichment was calculated using the hypergeometric distribution. The resulting P-values were corrected with the Benjamini-Hochberg correction. As the KEGG pathways represent groups of related bimolecular pathways a P-value cut-off of <0.1 was used as it would allow enrichment within individual bimolecular pathways to be identified. A P-value cut-off of <0.1 has been used previously during KEGG pathway analysis (Xu et al. 2010-Revealing parasite influence in metabolic pathways in Apicomplexa infected patients, BMC Bioinformatics 2010, 11(Suppl 11):S13; Shen et al. 2006—Sepsis Plasma Protein Profiling with Immunodepletion, Three-Dimensional Liquid Chromatography Tandem Mass Spectrometry, and Spectrum Counting, J. Proteome Res., 2006, 5 (11), pp 3154-3160, each incorporated by reference in their entireties). Gene ontology (GO) analysis was carried using human GO term associations (www.geneontology.org). GO term enrichment analysis was carried out using model-based gene set analysis which uses probabilistic inference to identify the active GO terms (Bauer, et al. 2010 GOing Bayesian: model-based gene set analysis of genome-scale data. Nucleic Acids Res. 2010; 38:3523-32, incorporated by reference in its entirety). This approach naturally deals with overlapping GO categories and avoids the need for multiple testing correction. Marginal probability values>0.50 were considered significantly enriched.

### Initial Probe and Data Filtering

The DNA methylome in RA and control (OA) FLS was evaluated. The Infinium HumanMethylation450 chip (Illumina, Inc.) was used to determine the methylation status of 485,512 loci from FLS isolated from 11 female patients at the time of total joint replacement surgery (6 RA; 5 OA). Loci were removed from subsequent analysis if any of the probes for a locus could not be disguised from background with a P-value<0.01 or if enough beads present upon the chip for accurate measurement of their methylation level. After filtering, 476,331 loci were available for further analysis.

### Global Methylation Status

To assess global methylation status of RA and OA FLS, the methylation scores over all filtered loci within a sample were summed. The difference between the two samples was assessed using Student's t-test. Initial analysis included all loci and was then repeated for only loci located in promoters. There were no significant differences between RA and OA (P-values 0.528 and 0.627, respectively). Therefore, globally hypo- or hypermethylation is not associated with RA when compared to OA. These results were confirmed using an antibody-based method to determine methylcytosine levels (Active Motif, Inc., Carlsbad, Calif.) where global methylation were similar for RA and OA (RA=0.85±0.32; OA=1.00±0.24; n=10 RA and 9 OA lines; P>0.05).

### Analysis of Differentially Methylated Individual Loci

Although global methylation are not different for RA and OA, there were differentially methylated (differentially methylated) loci that cluster to the two diseases. To identify the autosomal loci that are differentially methylated between OA and RA two filters were used: (i) an average difference in methylation level was greater than 0.1; and (ii) a P-value<0.05 that calculated using the Student's t-test and corrected for multiple testing with the Benjamini-Hochberg adjustment. 1,859 loci in 1206 different genes were identified as significantly differentially methylated in RA FLS. TABLE 1 shows example numbers of hypermethylated and hypomethylated sites.

TABLE 1

| | No. of loci | No. of loci |
|---|---|---|
| | hypomethylated | hypermethylated |
| | in RA | in RA |
| differentially methylated loci* | 732 | 1127 |
| differentially methylated genes** | 63 | 144 |
| differentially methylated KEGG | 10 | 6 |
| pathways | | |
| differentially methylated GO | 138 | 242 |
| terms | | |

*differentially methylated = Differentially methylated; Difference between OA and RA > 0.1, with P < 0 after Benjamini-Hochberg adjustment.
**6 additional genes had both hypo- and hypermethylated loci (ADAMTS2, C7orf50, DIP2C, FRMD SH3PXD2A)

Examples of genes identified with statistically significant differences between RA and OA at an individual locus using the ILLUMINA dataset are shown in TABLE 2. A mean value of OA methylation minus RA methylation; A P-value cut-off of <0.05 was used for determining significance. Where the RA minus OA value was positive the locus in RA was hypermethylated; where the RA minus OA value was negative, the locus in RA was hypomethylated.

TABLE 2

| | RA minus OA | Gene | Description |
|---|---|---|---|
| Hyper- | −0.34417 | PHLPP1 | PH domain and leucine rich repeat protein phosphatase 1 |
| methylated* | −0.34108 | RXRA | retinoid X receptor, alpha |
| | −0.30861 | CYFIP1 | cytoplasmic FMR1 interacting protein 1 |
| | −0.30363 | ADAMTS2 | ADAM metallopeptidase with thrombospondin type 1 motif, 2 |
| | −0.2964 | CD55 | CD55 molecule, decay accelerating factor for complement |
| | −0.27459 | CABLES1 | Cdk5 and Abl enzyme substrate 1 |
| | −0.26569 | HAS1 | hyaluronan synthase 1 |
| | −0.25362 | ITGB8 | integrin, beta 8 |
| | −0.25333 | HBEGF | heparin-binding EGF-like growth factor |
| | −0.23299 | PTPN14 | protein tyrosine phosphatase, non-receptor type 14 |
| | −0.23228 | COL4A2 | collagen, type IV, alpha 2 |
| | −0.23122 | COL4A1 | collagen, type IV, alpha 1 |
| | −0.21314 | TGFBR2 | transforming growth factor, beta receptor II (70/80 kDa) |
| | −0.20915 | EGF | epidermal growth factor |
| | −0.20692 | ITGBL1 | integrin, beta-like 1 (with EGF-like repeat domains) |
| | −0.20006 | TIMP2 | TIMP metallopeptidase inhibitor 2 |
| | −0.19768 | MAP3K1 | mitogen-activated protein kinase kinase kinase 1 |
| | −0.19005 | FOXO1 | forkhead box O1 |
| Hypo- | 0.365 | CHI3L1 | Chitinase 3-like 1 (cartilage glycoprotein-39) |
| methylated | 0.337 | WISP3; | WNT1 inducible signaling pathway protein 3 |
| | | WISP3 | |
| | 0.333 | STK24 | serine/threonine kinase 24 |
| | 0.329 | MMP20 | matrix metallopeptidase 20 |
| | 0.322 | EGFLAM | EGF-like, fibronectin type III and laminin G domain |
| | 0.318 | PTGIS | prostaglandin I2 (prostacyclin) synthase |
| | 0.301 | DCBLD1 | discoidin, CUB and LCCL domain containing 1 |
| | 0.299 | MAP3K5 | mitogen-activated protein kinase kinase kinase 5 (ASK1) |
| | 0.297 | MGMT | O-6-methylguaninE−DNA methyltransferase |
| | 0.282 | CDK14 | Cyclin-dependent kinase 14 |
| | 0.272 | STAT3 | signal transducer and activator of transcription 3 |
| | 0.270 | ADAM32 | ADAM metallopeptidase domain 32 |
| | 0.264 | RASGRF2 | Ras protein-specific guanine nucleotidE−releasing factor 2 |
| | 0.253 | CASP1 | caspase 1, apoptosis-related cysteine peptidase |
| | 0.225 | P2RX6 | purinergic receptor P2X, ligand-gated ion channel, 6 |
| | 0.195 | TRAF2 | TNF receptor-associated factor 2 |
| | 0.185 | MEFV | Mediterranean fever |

CpG methylation was significantly different in a number of genes implicated in RA. Several genes implicated in inflamation and immune responses are differentially methylated in RA.

A list of 1,859 loci is shown in TABLE 6 and TABLE 7. TABLE 6 and TABLE 7 list loci which are differentially methylated in RA compared to OA; a positive OA-RA value represents a loci which is hypomethylated in RA FLS (TABLE 6), a negative OA-RA value represents a loci which is hypermethylated in RA FLS (TABLE 7).

Permutation analysis was carried out to assess the significance of loci identified as differentially methylated. The 11 samples were randomly assigned to OA and RA while maintaining the same number of OA and RA labels, i.e., 5 OA and 6 RA. The permutation analysis was repeated 1,000 times. The average number of significant loci during the permutation analysis was 4.9, compared to 1,859 for the correct disease identification. The permutation analysis strongly supports these loci as truly differentially methylated and not as a result of random chance.

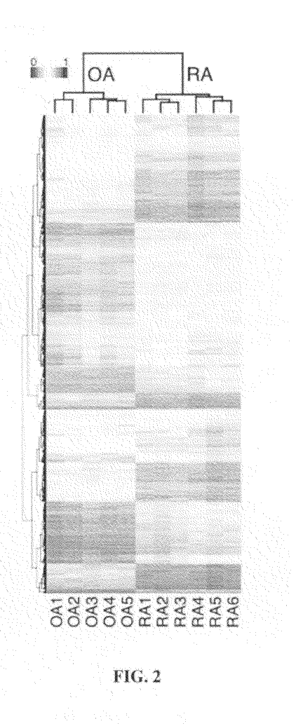

To assess the ability of the 1,859 loci to distinguish OA from RA, the methylation patterns of the loci across the 11 samples were hierarchically clustered (FIG. 2). The clustering of the samples distinguishes OA from RA, which clearly segregate according to disease type. The clustering of the loci also shows that groups of loci have similar patterns of differentially methylated across the samples.

Analysis of Genes with Multiple Differentially Methylated Loci

To examine genes that were significantly differentially methylated between OA and RA, genes that were enriched with multiple differentially methylated loci were identified (see Methods). This analysis was carried using all 1,859 differentially methylated loci identified in differentially methylated loci section. The data demonstrated that many genes have multiple CpGs that are hyper- or hypo-methylated. For example, COL1A1 has 41 loci, and 4 are hypomethylated in RA (P<10⁻⁶) giving a “relative enrichment” of 79-fold compared to OA. Relative enrichment for MEFV (pyrin) hypomethylation is nearly 200-fold. Of interest, 4 of 16 loci in the TNF promoter are hypermethylated in RA (relative enrichment-451; P<10⁻⁹), suggesting that TNF regulation might be under the control of DNMTs in some patients. 207 genes were either enriched for multiple hyper- or hypomethylated loci (TABLE 1). Representative examples of hypomethylated- (hypo-) and hypermethylated- (hyper-) differentially methylated genes are shown in TABLE 3.

TABLE 3

| | | No. of | | | |
|---|---|---|---|---|---|
| | | differentially | Total | | |
| | | methylated | loci | | Enrich- |
| Status | | loci beneath | on | P-value for | ment |
| in RA | Gene | cut-off | array | enrichment | factor |
| hypo | LOC146880 | 9 | 32 | 9.96E−18 | 245.34 |
| hypo | SH3D20 | 6 | 15 | 5.58E−14 | 487.22 |
| hypo | TACC2 | 7 | 70 | 4.60E−11 | 87.23 |
| hypo | KCNJ9 | 4 | 15 | 1.95E−08 | 251.27 |
| hypo | FUT4 | 4 | 20 | 4.90E−08 | 200.26 |
| hypo | ARHGAP27 | 4 | 25 | 2.45E−07 | 130.45 |
| hypo | OLFML3 | 3 | 9 | 2.97E−07 | 439.51 |
| hypo | ACTN1 | 4 | 42 | 3.73E−07 | 116 |
| hypo | TRIM15 | 4 | 82 | 3.79E−07 | 115.32 |
| hypo | PDZRN3 | 4 | 50 | 9.44E−07 | 88.29 |
| hypo | COL1A1 | 4 | 41 | 1.26E−06 | 79.55 |
| hypo | CD93 | 3 | 15 | 2.44E−06 | 200.26 |
| hypo | ITGA4 | 3 | 18 | 4.52E−06 | 157.79 |
| hypo | NFE2L3 | 3 | 19 | 7.74E−06 | 128.74 |
| hypo | HRNBP3 | 5 | 189 | 8.88E−06 | 25.05 |
| hypo | POMC | 3 | 26 | 1.00E−05 | 115.53 |
| hypo | SKAP1 | 3 | 27 | 1.09E−05 | 111.26 |
| hypo | T | 3 | 31 | 1.21E−05 | 106.8 |
| hypo | RHOJ | 2 | 9 | 2.16E−05 | 525.33 |
| hypo | COL22A1 | 3 | 36 | 2.26E−05 | 83.44 |
| hypo | BOC | 3 | 35 | 2.32E−05 | 81.15 |
| hypo | SCUBE1 | 2 | 23 | 6.91E−05 | 273.55 |
| hypo | ROBO4 | 2 | 15 | 7.76E−05 | 252.46 |
| hypo | HUNK | 2 | 16 | 8.36E−05 | 236.68 |
| hypo | AUTS2 | 3 | 114 | 8.36E−05 | 49.83 |
| hypo | FPR2 | 2 | 11 | 9.00E−05 | 221.46 |
| hypo | CASZ1 | 4 | 174 | 9.41E−05 | 21.76 |
| hypo | MEFV | 2 | 11 | 9.64E−05 | 200.66 |
| hypo | MXRA8 | 2 | 18 | 9.64E−05 | 210.38 |
| hypo | ARMC3 | 2 | 12 | 9.64E−05 | 203.01 |
| hypo | STX1B | 2 | 15 | 9.64E−05 | 199.51 |
| hypo | USP54 | 2 | 9 | 9.64E−05 | 193.85 |
| hypo | PLCH2 | 3 | 64 | 0.000112844 | 40.89 |
| hypo | FXYD7 | 2 | 17 | 0.000112844 | 176.04 |
| hypo | INSM1 | 2 | 13 | 0.000113996 | 169.79 |
| hypo | MYEF2 | 2 | 13 | 0.000113996 | 169.79 |
| hypo | P2RY6 | 2 | 21 | 0.000156705 | 142.51 |
| hypo | CA4 | 2 | 15 | 0.000227225 | 116.31 |
| hypo | AXIN2 | 2 | 37 | 0.000287729 | 102.35 |
| hypo | SPSB1 | 2 | 46 | 0.000287729 | 102.78 |
| hypo | KANK4 | 2 | 21 | 0.000316358 | 95.36 |
| hypo | APCDD1 | 2 | 20 | 0.000419791 | 81.53 |
| hypo | B3GNTL1 | 3 | 158 | 0.000474472 | 23.13 |
| hypo | ANGPT1 | 2 | 24 | 0.000585029 | 67.95 |
| hypo | PRRX1 | 2 | 27 | 0.000625725 | 64.62 |
| hypo | COL6A1 | 2 | 33 | 0.000696287 | 60.68 |
| hypo | LRIG1 | 2 | 46 | 0.000726192 | 59.28 |
| hypo | GLIS1 | 2 | 50 | 0.000828923 | 54.54 |
| hypo | ST14 | 2 | 43 | 0.000921733 | 51.33 |
| hypo | SYT7 | 2 | 54 | 0.001010062 | 48.83 |
| hypo | PHC2 | 2 | 34 | 0.001014634 | 47.96 |
| hypo | PLXNC1 | 2 | 34 | 0.001014634 | 47.96 |
| hypo | PKNOX2 | 2 | 63 | 0.001243393 | 43.29 |
| hypo | ODZ4 | 2 | 148 | 0.001280632 | 42.51 |
| hypo | MYO7A | 2 | 45 | 0.001280907 | 42.08 |
| hypo | EBF2 | 2 | 47 | 0.001382296 | 40.29 |
| hypo | NXN | 3 | 193 | 0.002031781 | 12.67 |
| hypo | PRKAR1B | 3 | 224 | 0.002535422 | 11.68 |
| hypo | SYNJ2 | 2 | 68 | 0.002651843 | 27.84 |
| hypo | MAML2 | 2 | 70 | 0.002780925 | 27.05 |
| hypo | GAS7 | 2 | 72 | 0.003896102 | 22.65 |
| hypo | MYT1L | 2 | 170 | 0.006150038 | 17.6 |
| hypo | SMOC2 | 2 | 164 | 0.01010976 | 13.46 |
| hyper | LOC90834 | 8 | 11 | 7.31E−18 | 375.88 |
| hyper | BRD1 | 8 | 42 | 1.65E−12 | 98.45 |
| hyper | C6orf25 | 5 | 42 | 1.90E−09 | 208.6 |
| hyper | PCSK6 | 7 | 65 | 2.09E−09 | 55.66 |
| hyper | LOC100292680 | 4 | 13 | 2.07E−08 | 291.99 |
| hyper | PPM1L | 5 | 37 | 6.18E−08 | 88.51 |
| hyper | FERMT3 | 4 | 21 | 1.05E−07 | 180.76 |
| hyper | TES | 4 | 21 | 3.42E−07 | 132.58 |
| hyper | PHYHD1 | 3 | 9 | 1.09E−06 | 316.32 |
| hyper | IGSF6 | 3 | 6 | 1.21E−06 | 275.53 |
| hyper | UACA | 3 | 22 | 2.44E−06 | 238.95 |
| hyper | TNF | 4 | 27 | 2.66E−06 | 71.05 |
| hyper | PRMT7 | 3 | 24 | 2.98E−06 | 219.03 |
| hyper | SEC14L3 | 3 | 9 | 3.07E−06 | 197 |
| hyper | HSD3B7 | 3 | 17 | 3.07E−06 | 208.59 |
| hyper | APP | 2 | 19 | 3.30E−06 | 2002.59 |
| hyper | MYPN | 3 | 11 | 3.44E−06 | 189.82 |
| hyper | HBEGF | 3 | 10 | 3.74E−06 | 177.3 |
| hyper | SLC22A1 | 3 | 13 | 3.74E−06 | 182.93 |
| hyper | LOC100271831 | 3 | 9 | 5.20E−06 | 151.33 |
| hyper | GPR1 | 3 | 14 | 6.06E−06 | 149.15 |
| hyper | NINJ2 | 4 | 39 | 6.06E−06 | 53.01 |
| hyper | GDPD3 | 3 | 10 | 6.90E−06 | 136.2 |
| hyper | C21orf34 | 3 | 16 | 8.70E−06 | 130.5 |
| hyper | KHDRBS3 | 3 | 17 | 1.24E−05 | 115.58 |
| hyper | TRERF1 | 4 | 50 | 1.49E−05 | 41.35 |
| hyper | COL4A2 | 5 | 139 | 1.53E−05 | 22.99 |
| hyper | MIRLET7C | 2 | 4 | 2.05E−05 | 591 |
| hyper | SPATA18 | 3 | 27 | 3.95E−05 | 77.34 |
| hyper | SEMA6D | 3 | 22 | 5.67E−05 | 65.4 |
| hyper | METTL9 | 3 | 25 | 5.67E−05 | 66.13 |
| hyper | TIMP2 | 4 | 64 | 5.67E−05 | 28.37 |
| hyper | KIAA1949 | 4 | 100 | 6.11E−05 | 27.84 |
| hyper | NRM | 2 | 43 | 8.35E−05 | 299.71 |
| hyper | NSMCE2 | 3 | 32 | 0.000105867 | 51.66 |
| hyper | SLC9A1 | 3 | 27 | 0.000109957 | 50.44 |
| hyper | C17orf99 | 2 | 10 | 0.000113832 | 236.4 |
| hyper | MIR1301 | 2 | 6 | 0.000143314 | 197 |
| hyper | PLXNA2 | 3 | 45 | 0.00015859 | 43.66 |
| hyper | GALNT12 | 2 | 9 | 0.00018091 | 176.15 |
| hyper | STX2 | 3 | 38 | 0.000181317 | 40.8 |
| hyper | CPT1A | 3 | 59 | 0.000185452 | 40.31 |
| hyper | EHD4 | 3 | 36 | 0.000185452 | 39.97 |
| hyper | MYH15 | 2 | 14 | 0.000191884 | 168.86 |
| hyper | FGL2 | 2 | 6 | 0.000226795 | 144.6 |
| hyper | RPH3AL | 4 | 154 | 0.000226795 | 18.08 |
| hyper | SCARA5 | 3 | 39 | 0.000253629 | 34.92 |
| hyper | BTC | 2 | 14 | 0.000270028 | 135.57 |
| hyper | CETN1 | 2 | 11 | 0.00029116 | 126.55 |
| hyper | NPL | 2 | 8 | 0.000332725 | 113.5 |
| hyper | LARP4 | 2 | 20 | 0.000332725 | 118.2 |
| hyper | NADK | 3 | 47 | 0.000334772 | 30.61 |
| hyper | ITPK1 | 3 | 65 | 0.000345734 | 30.23 |
| hyper | ZEB2 | 3 | 59 | 0.000346791 | 30.05 |
| hyper | CCDC25 | 2 | 12 | 0.000360628 | 106.54 |
| hyper | TMOD4 | 2 | 9 | 0.000360628 | 106.58 |
| hyper | EGF | 2 | 13 | 0.000360628 | 107.08 |
| hyper | SLC1A2 | 2 | 32 | 0.000360628 | 109.52 |
| hyper | ADCK5 | 2 | 22 | 0.000364838 | 107.45 |
| hyper | RNF165 | 2 | 16 | 0.000413716 | 99.09 |
| hyper | CSNK1G2 | 3 | 53 | 0.000425925 | 27.15 |
| hyper | ERRFI1 | 2 | 20 | 0.000448119 | 94.9 |
| hyper | ESCO2 | 2 | 14 | 0.000464905 | 91.32 |
| hyper | CHST11 | 3 | 55 | 0.000464905 | 26.16 |
| hyper | MUC6 | 3 | 56 | 0.00047946 | 25.69 |
| hyper | ITGB8 | 2 | 13 | 0.00050411 | 84.78 |
| hyper | ST5 | 3 | 71 | 0.00050411 | 24.97 |
| hyper | FANCA | 2 | 28 | 0.000525004 | 84.43 |
| hyper | MYF6 | 2 | 11 | 0.00055928 | 78.87 |
| hyper | SETD1A | 2 | 30 | 0.000588655 | 78.8 |
| hyper | RUFY3 | 2 | 17 | 0.000595625 | 77.05 |
| hyper | MYOM1 | 2 | 12 | 0.000597351 | 75.67 |
| hyper | ARL4D | 2 | 15 | 0.000638325 | 73.47 |
| hyper | NXPH2 | 2 | 21 | 0.000774146 | 66.29 |
| hyper | MGAT3 | 2 | 18 | 0.000776579 | 65.67 |
| hyper | AKT3 | 2 | 30 | 0.000841298 | 63.26 |
| hyper | RELL1 | 2 | 23 | 0.000896152 | 60.52 |
| hyper | CARS | 2 | 23 | 0.000896152 | 60.52 |
| hyper | TRAPPC9 | 4 | 215 | 0.000916221 | 11 |
| hyper | LZTS1 | 2 | 22 | 0.000916221 | 59.54 |
| hyper | PPP2R5C | 2 | 59 | 0.000924708 | 59.4 |
| hyper | DLX3 | 2 | 16 | 0.000963235 | 56.75 |
| hyper | AMBRA1 | 2 | 28 | 0.000986075 | 56.62 |
| hyper | SDCBP2 | 2 | 24 | 0.001039634 | 54.58 |
| hyper | LMBRD1 | 2 | 17 | 0.00105958 | 53.41 |
| hyper | ARHGAP1 | 2 | 18 | 0.00106039 | 53.29 |
| hyper | TBCD | 5 | 352 | 0.001086318 | 7.34 |
| hyper | ACSL1 | 2 | 30 | 0.001086318 | 52.85 |
| hyper | PLCB3 | 2 | 26 | 0.001238237 | 49.17 |
| hyper | MAP3K1 | 2 | 24 | 0.001394061 | 45.92 |
| hyper | LRRFIP1 | 2 | 76 | 0.001408026 | 46.11 |
| hyper | BAI2 | 2 | 36 | 0.001513586 | 44.04 |
| hyper | ZNRF1 | 2 | 26 | 0.001594799 | 42.39 |
| hyper | ITGBL1 | 2 | 23 | 0.001628294 | 41.71 |
| hyper | SPPL2B | 2 | 38 | 0.001642638 | 41.72 |
| hyper | PIGV | 2 | 23 | 0.001779686 | 39.48 |
| hyper | KLF12 | 2 | 40 | 0.001784969 | 39.63 |
| hyper | FOXO1 | 2 | 32 | 0.002026288 | 36.94 |
| hyper | C1QTNF1 | 2 | 39 | 0.002125118 | 35.69 |
| hyper | NCK2 | 2 | 53 | 0.002125118 | 35.81 |
| hyper | CYTH1 | 2 | 37 | 0.002143776 | 35.4 |
| hyper | APOLD1 | 2 | 34 | 0.002203548 | 34.76 |
| hyper | HTRA1 | 2 | 38 | 0.002233352 | 34.47 |
| hyper | CCDC146 | 2 | 28 | 0.002665635 | 30.98 |
| hyper | CRYL1 | 2 | 42 | 0.002665635 | 31.19 |
| hyper | COL4A1 | 2 | 71 | 0.003503044 | 26.73 |
| hyper | GPR133 | 3 | 165 | 0.003819221 | 10.75 |
| hyper | PHACTR2 | 2 | 40 | 0.004172106 | 23.98 |
| hyper | ANK3 | 2 | 38 | 0.004173533 | 23.89 |
| hyper | RPTOR | 5 | 432 | 0.004520935 | 5.02 |
| hyper | CACNA2D3 | 2 | 76 | 0.005405541 | 20.86 |
| hyper | LHFPL2 | 2 | 44 | 0.005447591 | 20.64 |
| hyper | TG | 2 | 47 | 0.00554287 | 20.41 |
| hyper | KANK2 | 2 | 45 | 0.005602318 | 20.18 |
| hyper | SRPK2 | 2 | 45 | 0.005602318 | 20.18 |
| hyper | PITPNM2 | 2 | 48 | 0.005684708 | 19.98 |
| hyper | ADAM12 | 2 | 52 | 0.005720838 | 19.88 |
| hyper | MICAL3 | 2 | 47 | 0.005961209 | 19.32 |
| hyper | FILIP1L | 2 | 45 | 0.005961209 | 19.28 |
| hyper | GALNS | 2 | 73 | 0.006100028 | 19.07 |
| hyper | ARHGAP26 | 2 | 61 | 0.006687473 | 18.07 |
| hyper | ARHGEF7 | 2 | 74 | 0.006937548 | 17.7 |
| hyper | WIPI2 | 2 | 64 | 0.007267014 | 17.22 |
| hyper | PTPN14 | 2 | 59 | 0.00801136 | 16.26 |
| hyper | CUX1 | 2 | 215 | 0.00801136 | 16.3 |
| hyper | SORBS2 | 2 | 81 | 0.00806007 | 16.17 |
| hyper | HPCAL1 | 2 | 60 | 0.008172002 | 15.99 |
| hyper | DNMT3A | 2 | 77 | 0.00881667 | 15.35 |
| hyper | TNS3 | 2 | 79 | 0.009173569 | 14.96 |
| hyper | SLC6A3 | 2 | 69 | 0.009173569 | 14.98 |
| hyper | PTPRG | 2 | 96 | 0.01090562 | 13.64 |
| hyper | MIR548G | 2 | 65 | 0.01127773 | 13.35 |
| hyper | ASAP2 | 2 | 70 | 0.01183843 | 12.97 |
| hyper | C3orf26 | 2 | 67 | 0.01183843 | 12.95 |
| hyper | NAV1 | 2 | 68 | 0.01212126 | 12.76 |
| hyper | CREB5 | 2 | 82 | 0.01424866 | 11.7 |
| hyper | CTBP2 | 2 | 102 | 0.02110821 | 9.4 |
| hyper | TCERG1L | 2 | 105 | 0.02218092 | 9.14 |
| hyper | SHANK2 | 3 | 262 | 0.02489354 | 4.97 |
| hyper | CMIP | 2 | 113 | 0.02508337 | 8.49 |
| hyper | BAT2 | 2 | 161 | 0.02704674 | 8.14 |
| hyper | EIF2C2 | 2 | 117 | 0.02919953 | 7.76 |
| hyper | RPS6KA2 | 2 | 244 | 0.02919953 | 7.78 |
| hyper | PRKCZ | 2 | 263 | 0.03328032 | 7.22 |

### Correlation of Gene Expression and Methylation Status

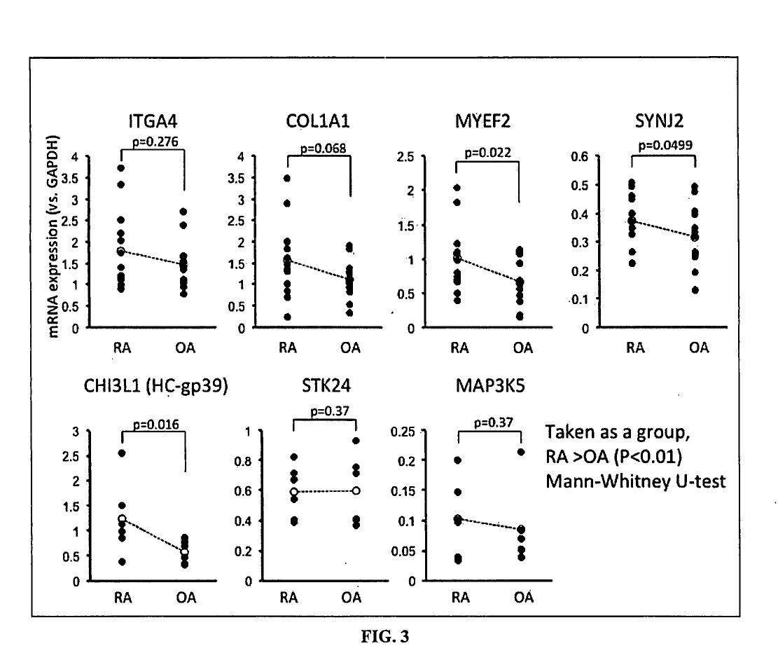

Functional analysis of the differentially methylated was performed by evaluating expression of several representative genes with hypo- and normally methylated loci in RA FLS by qPCR. FIG. 3 shows the results for several hypomethylated genes. These genes included ITGA4, COL1A1, MYEF2, SYNJ2, CHI3L1, STK24, and MAP3K5. As a group, expression of genes with hypomethylated loci was significantly greater in RA than OA FLS (n=7 genes; P<0.01). On the other hand, expression of normally methylated genes was similar in RA and OA FLS (n=7 genes; P>0.10).

Higher expression in RA was demonstrated for most hypomethylated genes, and the differences were significant when the genes were evaluated as a group (P<0.01 for RA vs. OA). Some genes, such as STK24, were not significantly different in RA and could reflect more than methylation in vitro. Nevertheless, these data demonstrated that methylation is reflected in gene expression patterns in RA and could contribute to production of inflammatory mediators in RA.

### Pathway and Gene Ontology Analyses

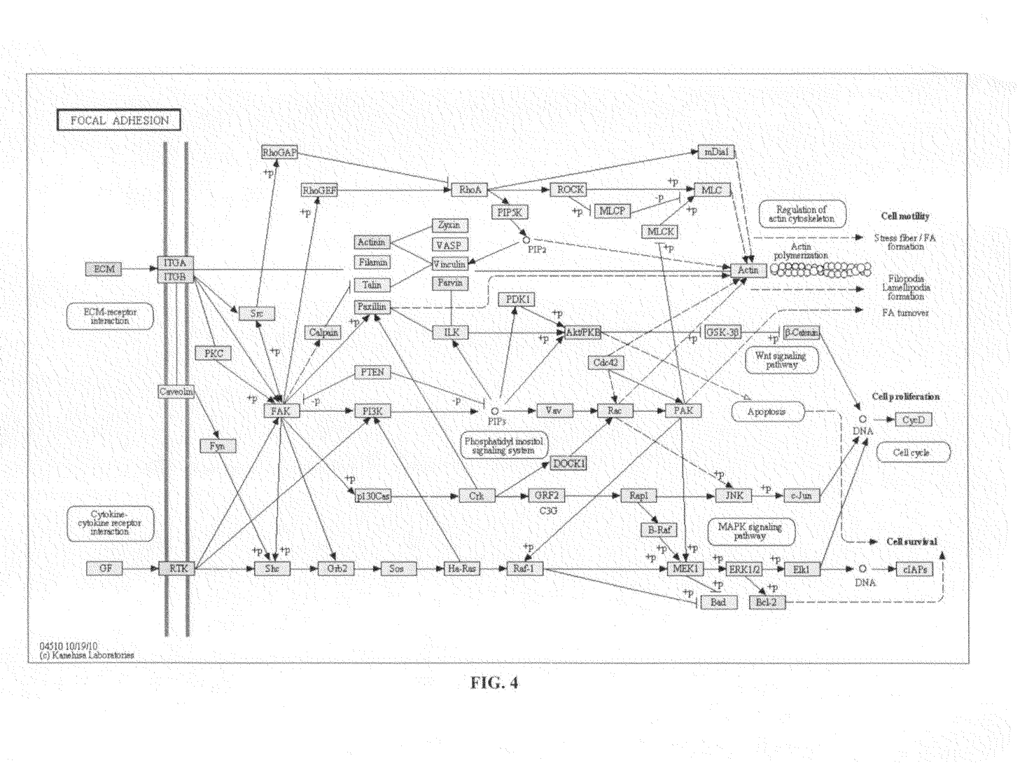

Biological pathways and gene ontologies that are enriched with differentially methylated loci were evaluated. Loci were mapped to pathways via their relationships to genes within pathways for KEGG pathways and for GO using all 1,859 differentially methylated loci identified (TABLE 1). Pathway analysis showed that interactions between cells and the matrix and cell recruitment were especially prominent in the differentially methylated group, including focal adhesion, mTOR signaling, cell adhesion molecules, leukocyte transendothelial interactions, and ECM-receptor interactions. FIG. 4 depicts the pathway ‘Focal adhesion’, which was enriched with loci that are significantly less methylated in RA. A list of differential GO terms and an analysis of hypomethylated and hypermethylated terms in RA with marginal probability>0.5 is shown in TABLE 4.

TABLE 4

| | | Marginal | |
|---|---|---|---|
| | ID | probability | Name |
| RA | GO: 0003713 | 0.99836 | Transcription coactivator activity |
| Hypomethylated | GO: 0051117 | 0.98512 | ATPase binding |
| | GO: 0031519 | 0.95603 | PcG protein complex |
| | GO: 0045944 | 0.92189 | Positive regulation of transcription |
| | | | from RNA polymerase II promoter |
| | GO: 0019897 | 0.81989 | Extrinsic to plasma membrane |
| | GO: 0008134 | 0.81119 | Transcription factor binding |
| | GO: 0015467 | 0.78847 | G-protein activated inward rectifier |
| | | | potassium channel activity |
| | GO: 0017157 | 0.76244 | Regulation of exocytosis |
| | GO: 0007369 | 0.74990 | Gastrulation |
| | GO: 0035257 | 0.70985 | Nuclear hormone receptor binding |
| | GO: 0004221 | 0.59229 | Ubiquitin thiolesterase activity |
| | GO: 0004629 | 0.55446 | Phospholipase C activity |
| | GO: 0009897 | 0.53595 | External side of plasma membrane |
| | GO: 0015629 | 0.52284 | Actin cytoskeleton |
| | GO: 0022610 | 0.51076 | Biological adhesion |
| RA | GO: 0005794 | 0.77055 | Golgi apparatus |
| Hypermethylated | GO: 0007166 | 0.76581 | Cell surface receptor linked |
| | | | signaling pathway |
| | GO: 0005488 | 0.60305 | Binding |

Of interest, the number of GO terms associated with hypomethylated DNA in RA was greater than for hypermethylated DNA (15 vs. 3). Thus, hypomethylated terms clustered with hypomethylation in RA, especially related to membrane and transcription factor biology. This analysis can be used to identify novel therapeutic targets for RA. More examples of pathways useful to identify novel therapeutic targets for RA are shown in TABLE 5.

TABLE 5

| | | | | DM | | |
|---|---|---|---|---|---|---|
| | | | | loci | Total | |
| Status | | | Enrichment | in | loci in | |
| in RA | Path group | Path name | factor | path | path | P-value |
| hypo | Cell Communication | Focal adhesion | 2.615457643 | 20 | 6226 | 0.007005624 |
| hypo | Glycan Biosynthesis | Glycosphingolipid | 9.88097044 | 5 | 412 | 0.007005624 |
| | and Metabolism | biosynthesis - lacto | | | | |
| | | and neolacto series | | | | |
| hypo | Cardiovascular | Arrhythmogenic right | 3.862390722 | 10 | 2108 | 0.007378046 |
| | Diseases | ventricular | | | | |
| | | cardiomyopathy | | | | |
| | | (ARVC) | | | | |
| hypo | Signaling Molecules | ECM-receptor | 3.303009997 | 12 | 2958 | 0.007378046 |
| | and Interaction | interaction | | | | |
| hypo | Infectious Diseases | Amoebiasis | 3.278225332 | 11 | 2732 | 0.01087908 |
| hypo | Immune System | Leukocyte | 3.339589681 | 10 | 2438 | 0.01366817 |
| | | transendothelial | | | | |
| | | migration | | | | |
| hypo | Digestive System | Protein digestion | 3.090564183 | 9 | 2371 | 0.03435163 |
| | | and absorption | | | | |
| hypo | Endocrine System | Adipocytokine | 3.505131458 | 7 | 1626 | 0.04363638 |
| | | signaling pathway | | | | |
| hypo | Signaling Molecules | Cell adhesion | 2.542760663 | 10 | 3202 | 0.06198887 |
| | and Interaction | molecules (CAMs) | | | | |
| hypo | Energy Metabolism | Nitrogen metabolism | 6.691988748 | 3 | 365 | 0.08527128 |
| hyper | Signal Transduction | ErbB signaling | 2.295320174 | 18 | 2272 | 0.0580967 |
| | | pathway | | | | |
| hyper | Infectious Diseases | African | 4.05913013 | 8 | 571 | 0.0580967 |
| | | trypanosomiasis | | | | |
| hyper | Lipid Metabolism | Primary bile acid | 5.625639088 | 4 | 206 | 0.0950006 |
| | | biosynthesis | | | | |
| hyper | Immune System | Fc epsilon RI | 2.345183916 | 13 | 1606 | 0.0950006 |
| | | signaling pathway | | | | |
| hyper | Signal Transduction | mTOR signaling | 2.295464506 | 14 | 1767 | 0.0950006 |
| | | pathway | | | | |
| hyper | Endocrine System | Adipocytokine | 2.316337866 | 13 | 1626 | 0.0950006 |
| | | signaling pathway | | | | |

### Network Analysis

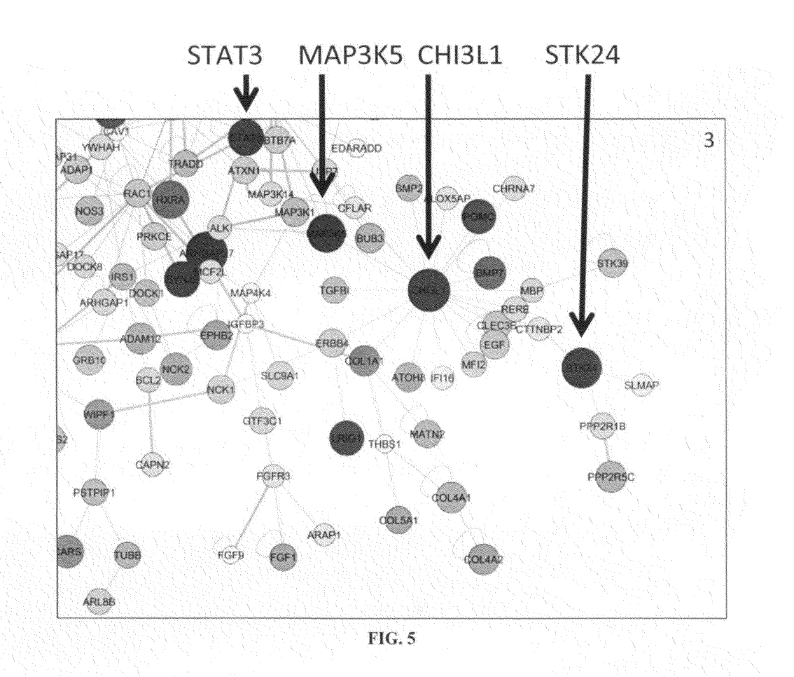

Cytoscape was used to evaluate networks affected by differential methylation and to determine likely targets for subsequent analysis. In a preliminary study, interactions between hypomethylated genes and their neighbors were evaluated. A portion of the Cytoscape analysis is shown in FIG. 5. Significant nodes included key hypomethylated loci (e.g., STAT3, MAP3K5, CHI3L1, STK24) (circle size indicates degree of hypomethylation). FIG. 5 depicts how these loci interact with each other and with multiple additional pathways, and describes how the methylated genes might regulate inflammatory responses in RA. This map shows only a fraction of the interactions and provides an unbiased view of how methylation affects synoviocyte function.

### Basal Expression of DNMTs

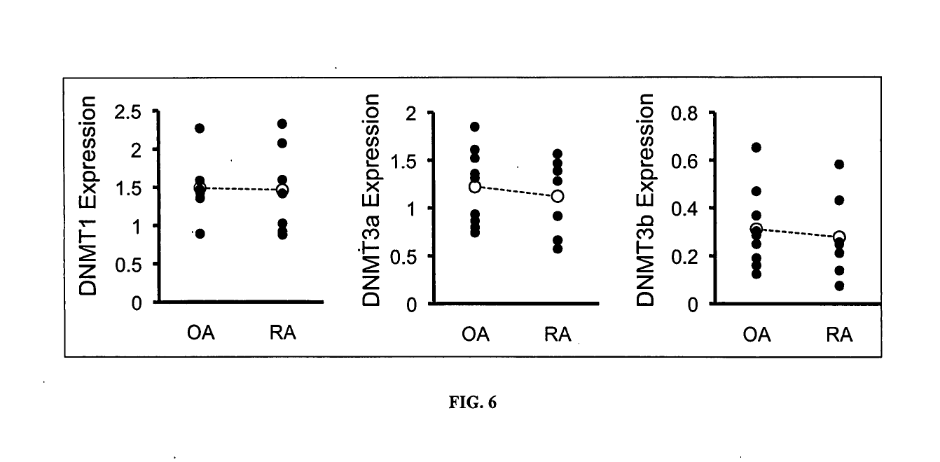

After discovering that RA FLS are differentially methylated, DNMT expression in resting cultured FLS was examined. Synoviocytes were isolated from RA and OA synovium at the time of total joint replacement. 4^{th} through 6^{th} passage cells were evaluated by qPCR. FIG. 6 depicts graphs of relative expression of DNMT1, DNMT3a, and DNMT3b in OA FLS and RA FLS. Unexpectedly, expression levels of DNMTs in RA and OA were found to be similar. In view of these findings, changes in DNMT expression in FLS was examined, and the role of cytokines, e.g., IL-1, in RA, that may contribute to a DNMT profile was examined.

Decreased DNMT1 Gene Expression after IL-1 Stimulation

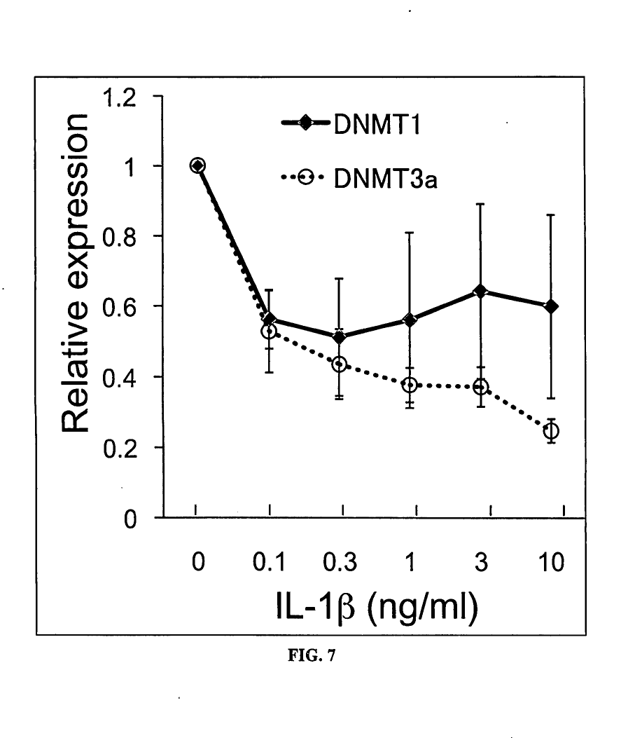

FLS were stimulated with IL-1 for 24 hr and DNMT expression was determined by qPCR. Unexpectedly, DNMT1 and DNMT3a gene expression significantly decreased after exposure to modest concentrations of IL-1 (FIG. 7). After noting that IL-1 decreased DNMT mRNA, time course experiments were performed to assess the kinetics of the effect. Cells were stimulated with 1 ng/ml of IL-1 for various time periods and qPCR was performed. DNMT mRNA levels begin to decrease within 2 to 8 hrs after exposure to the cytokine (data not shown).

### IL-1 Decreases DNMT Function in FLS

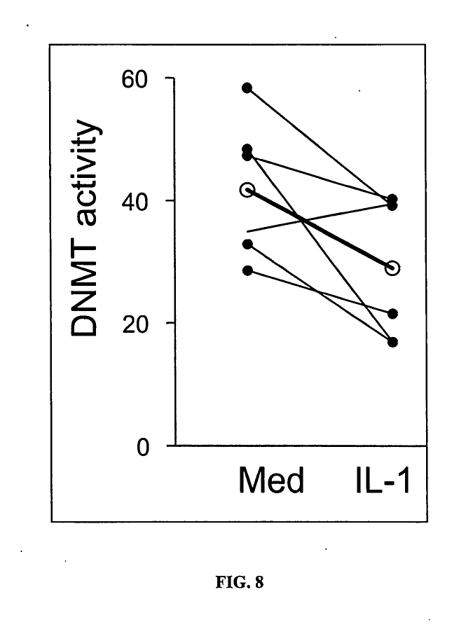

A functional assay was performed to determine if IL-1 suppresses DNA methylation activity of the DNMTs. FLS were stimulated with 1 ng/ml of IL-1 for 14 days and extracts were assayed using the DNMT Activity/Inhibition Assay (Active Motif Co., Carlsbad, Calif.), which is an ELISA-based method that measures methylation of a CpG-enriched DNA substrate. As shown in FIG. 8, a significant decline in total DNMT function (p<0.05) was observed.

### Discussion

FLS form the synovial intimal lining and play an integral role in the pathogenesis of RA by producing key cytokines, small molecule mediators, and proteases. While osteoclasts are the primary effectors of bone erosions in arthritis, FLS are responsible for cartilage damage by virtue of their ability to adhere to and invade the cartilage extracellular matrix. This capacity requires homotypic aggregation mediated by the adhesion molecule cadherin-11, which directs intimal lining formation and supports an invasive phenotype. Understanding the molecular mechanisms that regulate FLS activation could provide insights into the pathogenesis of RA and lead to novel therapeutic strategies. In the present application, the epigenetic profile of RA was evaluated by exploring a newly discovered DNA methylation signature that could potentially affect adaptive and innate immune functions, through their effects on synoviocytes and immune cells in the blood and joint.

Rheumatoid FLS exhibit a unique aggressive phenotype that contributes to the cytokine milieu and joint destruction. Functional studies suggest that RA cells are imprinted in situ and maintain these features after many passages in tissue culture. For example, RA FLS, unlike OA or normal synoviocytes, adhere to and invade cartilage explants in SCID mice. RA FLS can grow under anchorage-independent conditions, are less susceptible to contact inhibition, resistant to apoptosis. RA synoviocytes can potentially “metastasize” and transfer the invasive phenotype from one joint to another.

Several mechanisms have been implicated in this persistently aggressive phenotype, including abnormal sentrin or PTEN expression, preferential shunting of stressed cells to DNA repair rather than programmed cell death., and somatic mutations of regulatory genes. Microsatellite instability has also been identified in RA synovium, in part due to decreased DNA repair. These genetic modifications potentially serve to amplify disease severity.

While the focus on gene sequences in FLS and in disease association studies has provided important insights, other mechanisms can change cell phenotype. Epigenetics, for instance, can profoundly influence cell activation and gene expression through a variety of mechanisms, including DNA methylation, histone modification, and microRNA production. DNA methylation could be especially relevant in RA, in light of role in neoplasia and embryonic growth. Normal ontogeny relies on a carefully orchestrated sequence of DNA methylation to repress regulatory genes by methylating cytosine in CpG loci, either in promoters or in genes themselves. Methylation abnormalities have been associated with many diseases, most notably cancer where renewed expression or inappropriate suppression of genes allows cells to escape normal homeostatic controls. Hypomethylation and hypermethylation are associated with many malignancies and can contribute to transformation.

DNA methyltransferases (DNMTs) are responsible for initiating and maintaining CpG methylation in the human genome by converting cytosine to methylcytosine. In mammalian cells, DNMT1, DNMT3a, and DNMT3b are the primary enzymes responsible for CpG methylation. DNMT3a and DNMT3b mainly regulate de novo methylation while DNMT1 maintains methylation, especially during cell division. DNMT expression and DNA methylation are not immutable but are influenced by the environment and modify gene expression throughout life and even in progeny. For instance, pregnant mice fed a diet rich in methyl donors give birth to pups with increased levels of DNA methylation and increased airway reactivity in murine asthma for at least two subsequent generations.

Global hypomethylation has been described in peripheral blood mononuclear cells of patients with RA, although the specific genes involved are not known. Modest global hypomethylation was also reported in cultured RA FLS when compared to OA cells. In contrast, our studies showed that global methylation levels are similar in OA and RA FLS using two different techniques (ELISA-based and chip based). Thus, RA FLS do not appear to be hypomethylated overall, but like neoplasia, display a pattern of hypermethylated and hypomethylated genes.

The ILLUMINA chip analysis identified distinct methylation profiles of OA and RA FLS involving 1859 loci located in 1206 genes. Cluster analysis showed that the two types of FLS could be easily distinguished based solely on the methylation patters. The results were confirmed using a variety of additional analyses that reduced the possibility of random chance as an explanation. Additional analysis identified 207 genes with multiple hyper- or hypomethylated loci. Many of these genes play a key role in inflammation, matrix regulation, leukocyte recruitment and immune responses. Gene expression levels correlated with methylation status, with high expression in hypomethylated genes in RA FLS and normal expression in genes that were not differentially methylated. Given the number of influences that can potentially alter gene expression in vitro, the general concordance between methylation and mRNA levels was striking.

The data described herein provide evidence that epigenetic changes are present in RA synoviocytes and that they persist in culture. Imprinting could potentially occur before clinical disease and contribute to susceptibility. Alternatively, and perhaps more likely, the changes can be induced after initiation of synovitis. In the latter situation, the inflammatory milieu could potentially imprint synoviocytes and affect their function for many passages. Thus, local inflammation could potentially alter expression of enzymes responsible for initiating and maintaining DNA methylation. This process imprints synoviocytes, peripheral blood cells that are present in synovium for a short period of time, and immune cells, alters their behaviour, and ultimately changes the natural history of disease.

The ability to distinguish RA and control cells based solely on the DNA methylome could provide major insights into how the epigenetic profile of various tissues contribute to the pathogenesis of RA. In addition to identifying novel targets among the differentially methylated genes, it could also lead to interesting diagnostic or personalized medicine applications after sufficient data are available to correlate the methylome to phenotype.

### Example 2

### Methylation of Loci in Peripheral Blood Cells

Isolation of Genomic DNA from Peripheral Blood Cells

Approximately 6-8 ml of blood was drawn into a DB CPT tube (BD cat#362760) and inverted 10 times. Four tubes per patient were drawn. The tubes were stored at room temperature until processed. Processing was performed in less than four hours after being drawn. The tubes were spun 30 minutes at 3000 rpm. The mononuclear cell layer was carefully removed and washed in at least 10 volumes of cold PBS with 0.1% BSA (Buffer 1: PBS Invitrogen cat#14190, BSA Gemini cat#700-100P). Mononuclear cells were then spun at 1600 rpm for 10 minutes. Cells were then resuspended in 3 ml of cold PBS supplemented with 0.1% BSA and 2 mM EDTA (Buffer 2: Buffer 1 plus EDTA Invitrogen cat#15575020). Cells were then counted and divided into 2 ml eppendorf tubes as follows: 0.5 ml for T-cell isolation, 1.5 ml for B-cell isolation, 0.75 ml for monocyte isolation, and 0.25 ml for whole PBMCs. These were spun again at 1600 rpm for 10 minutes and supernatants were discarded. The whole PBMC fraction was frozen while the others continued the specific cell type separation.

Separation of PBMCs into Specific Cell Types

Cells in the PBMC preparation above were separated into B cell preparations, T cell preparations and monocyte preparations as follows. Dynabeads magnetic beads (CD19 pan B Invitrogen cat#111-43D, CD2 pan T Invitrogen cat#111-59D, CD14 monocyte Invitrogen cat#111-49D) were used for the specific cell-type isolation. The magnetic bead mixtures were added to 1 ml of cold Buffer 2 according to the number of cells (50 μl T-cell bead isolation mixture per 10⁷ cells, 25 μl B-cell bead isolation mixture per 2.5×10⁷ cells, and 25 μl monocyte isolation bead mixture per 10⁷ cells), mixed well and applied to the magnet for 3 minutes to wash the beads. Supernatants were discarded. Mononuclear cell pellets were then gently resuspended in 1.5 ml of cold buffer 2 and added to their appropriate bead isolation mixture. The bead/cell mixture was incubated at 4° C. while turning end-over-end for 20 minutes. The bead/cell mixture was then applied to the magnet for 3 minutes and supernatants were discarded. The bead/cell mixture was then washed 3 times by removing from the magnet, adding 1 ml cold Buffer 2, gently mixing, reapplying to the magnet, and discarding the supernatant. The cell/bead mixture was then frozen until DNA Isolation was performed. The DNA isolation was performed with DNeasy Blood and tissue Kit (Qiagen cat#69504). The protocol for cultured cells was followed and included the recommendation of RNase A (Qiagen cat#19101) treatment. DNA eluents were then concentrated using Amicon ultra 30K filers (Millipore cat# UFC503096). Concentrated DNA was then quantified using Quant-it Picogreen reagent (Invitrogen cat# P7589). Concentrations were then standardized to 100 ng/μl.

PBMC preparations may also be separated into other cell types, such as white blood cells, neutrophils, eosinophils, basophils, lymphocytes, plasma cells, natural killer cells, and dendritic cells using procedures such as those described above or other methods familiar to those skilled in the art. Macrophages may be separated from other cell types using methods well known in the art. In some embodiments, particular cell types can be enriched and/or isolated using a variety of methods, such methods are well known in the art and include immunological methods, fluorescent activated cell sorting (FACS) methods, and affinity chromatography methods. For example, cells such as eosinophils can be enriched/isolated using antibodies specific to specific receptors on the cell surface such as L-selectin, and VLA-4 (Sriramarao P., et al., (1994) J. Immunol. 153:4238-46, incorporated by reference in its entirety) Neutrophils may be isolated using density gradients or using antibodies specific to other cell surface receptors (Firestein G. S., et al., (1995) J. Immunol. 154:326-34, incorporated by reference in its entirety).

Analysis of Methylation States of Loci in PBMCs or Specific Cell Types Separated from PBMC Preparations in Individuals with RA or OA

Samples of genomic DNA are obtained from peripheral blood mononuclear cells or specific cell types separated from PBMC preparations as described above. The samples are obtained from individuals with RA, individuals with OA, individuals with a known prognosis for rheumatoid arthritis or osteoarthritis, individuals with a known reponse to treatment for rheumatoid arthritis or osteoarthritis and control subjects without rheumatoid arthritis, without osteoarthritis, without the known prognosis for rheumatoid arthritis or osteoarthritis, or without the known response to treatment for rheumatoid arthritis or osteoarthritis using methods described herein. The methylation states of loci for each set of genomic DNA is determined as described herein for genomic DNA from FLS cells.

Hypomethylated and hypermethylated loci in individuals with RA, individuals with OA, individuals with a known prognosis for rheumatoid arthritis or osteoarthritis, individuals with a known response to treatment for rheumatoid arthritis or osteoarthritis are identified by comparing the methylation states of the loci to the methylation states of the loci in control subjects without rheumatoid arthritis, without osteoarthritis, without the known prognosis for rheumatoid arthritis or osteoarthritis, or without the known response to treatment for rheumatoid arthritis or osteoarthritis respectively using the methods described herein for FLS cells.

In some embodiments, hypomethylated and hypermethylated loci in RA genomic DNA relative to the methylation state of loci in OA genomic DNA are identified. In other embodiments hypomethylated and hypermethylated loci in RA genomic DNA relative to the methylation state of loci in genomic DNA from control subjects without RA are identified. In some embodiments, hypomethylated and hypermethylated loci in OA genomic DNA relative to the methylation state of loci in genomic DNA are identified from control subjects without OA are identified.

Genes with multiple differentially methylated loci are analyzed as described herein for genomic DNA from FLs cells, Gene expression and methylation status are analyzed as described herein for genomic DNA from FLs cells, Pathway analyses and gene ontology analyses are performed as described herein for genomic DNA from FLs cells. Network and DNMT analyses as described herein for genomic DNA from FLs cells.

### Example 3

### Evaluation of a Subject for RA,OA, Individuals with RA, a Particular Prognosis for Rheumatoid Arthritis or Osteoarthritis, or a Particular Response to Treatment for Rheumatoid Arthritis or Osteoarthritis

Genomic DNA is obtained from a subject and the methylation states of one or more loci having differential methylation in individuals with individuals with RA, individuals with OA, individuals with a particular prognosis for rheumatoid arthritis or osteoarthritis, or individuals with a particular response to treatment for rheumatoid arthritis or osteoarthritis is determined.

In some embodiments, the methylation state of the one or more differentially methylated loci in the genomic DNA from the subject is compared with the methylation state of the one or more differentially methylated loci in normal tissue, tissue from a subject with a known prognosis, or tissue from a subject with a known response to treatment. In other embodiments, the methylation state of the one or more differentially methylated loci in the genomic DNA from the subject is compared with a methylation state of the one or more differentially methylated loci known to be indicative of RA or a lack thereof, OA or a lack thereof, a particular prognosis for rheumatoid arthritis or osteoarthritis or a lack thereof, or a particular response to treatment for rheumatoid arthritis or osteoarthritis or a lack thereof.

The genomic DNA may be obtained from any desired cell type, including the cell types listed herein. For example, the genomic DNA may be obtained from FLS cells, a peripheral blood sample or a specific cell type separated from a PBMC sample obtained from a subject. If the methylation state of the one or more differentially methylated loci is a methylation state known to be indicative of RA, a particular prognosis for rheumatoid arthritis or osteoarthritis, or a particular response to treatment for rheumatoid arthritis or osteoarthritis the subject is determined to have RA, the particular prognosis for rheumatoid arthritis or osteoarthritis, or the particular response to treatment for rheumatoid arthritis or osteoarthritis. A treatment regimen consistent with this determination may then be administered.

### Example 4

### Evaluation of a Methylation State of Loci in Control Subjects Compared to Methylation State of Loci in RA FLS and OA FLS

Genomic DNA is obtained from a subject without RA and a subject without OA and the methylation states of one or more loci having differential methylation in RA FLS and OA FLS cells, respectively, is determined.

In some embodiments, the methylation state of the one or more differentially methylated loci in RA FLS and OA FLS cells is compared to a methylation state of the one or more differentially methylated loci in the genomic DNA from a subject without RA and in the genomic DNA from a subject without OA, respectively. In some embodiments, the methylation state of loci indicative of the absence of RA or OA in a subject are determined.

The genomic DNA may be obtained from any desired cell type, including the cell types listed herein. For example, the genomic DNA may be obtained from FLS cells, a peripheral blood sample or a specific cell type separated from a PBMC sample obtained from a subject. If the methylation state of the one or more differentially methylated loci is a methylation state known to be indicative of RA, a particular prognosis for rheumatoid arthritis or osteoarthritis, or a particular response to treatment for rheumatoid arthritis or osteoarthritis the subject is determined to have RA, the particular prognosis for rheumatoid arthritis or osteoarthritis, or the particular response to treatment for rheumatoid arthritis or osteoarthritis. A treatment regimen consistent with this determination may then be administered.

### Example 5

### Evaluation of Methylation State of Loci in Peripheral Blood Mononuclear Cells (PBMCs)

The methylation states of loci in PBMCs were determined with methods substantially similar to those described herein using the ILLUMINA HumanMethylation 450 BeadChip. Differentially methylated loci (DML) were identified with average methylation differences between OA and RA of >0.10. RA/OA differential methylation was conducted using a t-test. P-values were converted to q-values to account for multiple hypothesis testing, and DML with q-values<0.25 were considered to be potential PBMC biomarkers. TABLE 8 lists about 2544 RA/OA PBMC DML with associated data including the OA-RA average methylation difference, CG identifier associated with the Illumina 450K beadchip (Locus), associated genes, and the genomic location of the potentially methylated C of the CpG (+ strand) determined using the UCSC hg19 reference genome. The CpG* column of Table 8 lists values for “chromosome:chromosome co-ordinate.”

TABLE 6

| OA − | | | SEQ |
|---|---|---|---|
| RA | Locus | Associated gene | ID No. |
| 0.39071 | cg20114732 | T | 361932 |
| 0.37096 | cg03044684 | HUNK | 62005 |
| 0.36569 | cg20824939 | | 372791 |
| 0.36469 | cg19081101 | CHI3L1 | 345293 |
| 0.36368 | cg13225830 | | 243918 |
| 0.36153 | cg27244773 | ARHGAP27; ARHGAP27; | 475250 |
| | | ARHGAP27 | |
| 0.34388 | cg02792136 | DIP2C | 56847 |
| 0.33702 | cg12557114 | WISP3; WISP3 | 233566 |
| 0.33333 | cg06043820 | ZNF592 | 119061 |
| 0.33296 | cg07804470 | STK24 | 151872 |
| 0.33037 | cg12578486 | UBAC2; UBAC2; UBAC2 | 233879 |
| 0.32914 | cg18886274 | | 342488 |
| 0.32889 | cg12651286 | MMP20 | 235219 |
| 0.32591 | cg10035831 | RPTOR; RPTOR | 191245 |
| 0.32183 | cg12476044 | EGFLAM; EGFLAM; EGFLAM; | 232241 |
| | | EGFLAM; EGFLAM | |
| 0.32154 | cg16214653 | | 298250 |
| 0.32018 | cg06550177 | | 127813 |
| 0.31828 | cg06357305 | PTGIS | 124318 |
| 0.31549 | cg27250435 | | 475334 |
| 0.31524 | cg24203758 | SH3D20 | 425444 |
| 0.31391 | cg18675043 | SH3PXD2A | 338965 |
| 0.30767 | cg11173579 | HUNK | 210693 |
| 0.30665 | cg21514164 | | 383094 |
| 0.30602 | cg21670717 | | 385687 |
| 0.30248 | cg09182085 | | 176526 |
| 0.30113 | cg09230154 | MACF1; MACF1 | 177251 |
| 0.30097 | cg27642470 | DCBLD1 | 482028 |
| 0.2993 | cg23413809 | SIX2 | 412582 |
| 0.29885 | cg07474842 | MAP3K5 | 1415804 |
| 0.29665 | cg23004031 | MGMT | 405673 |
| 0.29622 | cg02727484 | KCNMA1; KCNMA1; KCNMA1; | 55565 |
| | | KCNMA1 | |
| 0.29494 | cg07227049 | VRK2; VRK2; VRK2; VRK2; | 141396 |
| | | VRK2; VRK2 | |
| 0.28978 | cg06223539 | SHANK2 | 122219 |
| 0.28958 | cg19674091 | IDH2 | 354510 |
| 0.28897 | cg06966660 | TACC2; TACC2; TACC2; TACC2 | 136119 |
| 0.28527 | cg22778178 | | 401834 |
| 0.28511 | cg12427162 | SFT2D2 | 231416 |
| 0.28455 | cg16854917 | NCRNA00085 | 309652 |
| 0.28177 | cg03834767 | CDK14 | 77306 |
| 0.28129 | cg17820022 | PLCH2 | 324801 |
| 0.27987 | cg05040219 | GLIS1 | 100492 |
| 0.27931 | cg00807684 | RBMS3; RBMS3; RBMS3 | 17055 |
| 0.27912 | cg10536999 | NFE2L3 | 200194 |
| 0.27826 | cg22458832 | | 396980 |
| 0.2773 | cg09257635 | GJC2 | 177820 |
| 0.27706 | cg24576026 | SEPT9; SEPT9 | 431206 |
| 0.27474 | cg01999051 | BRP44L | 40997 |
| 0.27386 | cg10820084 | COL1A1 | 204937 |
| 0.27361 | cg07696516 | LIMCH1; LIMCH1; LIMCH1; | 149907 |
| | | LIMCH1; LIMCH1; LIMCH1; | |
| | | LIMCH1 | |
| 0.27349 | cg10937408 | HRNBP3 | 206779 |
| 0.2732 | cg07021447 | ACTN1; ACTN1; ACTN1 | 137188 |
| 0.27261 | cg13644549 | C7orf50; C7orf50; C7orf50 | 252081 |
| 0.27247 | cg26787787 | | 467264 |
| 0.27222 | cg10881749 | | 205937 |
| 0.27192 | cg24718015 | STAT3; STAT3; STAT3 | 433677 |
| 0.27022 | cg03780701 | ADAM32 | 76382 |
| 0.26961 | cg13735018 | SYNJ2 | 254007 |
| 0.26932 | cg19391924 | HRNBP3 | 349887 |
| 0.26888 | cg13551141 | PSMB7 | 250097 |
| 0.2674 | cg14370520 | FAM188B | 267054 |
| 0.26591 | cg01660999 | ANGPT1 | 34240 |
| 0.26415 | cg13626830 | RASGRF2 | 251743 |
| 0.26352 | cg20441502 | FXYD7; FXYD1; FXYD1 | 366782 |
| 0.26348 | cg01833196 | SH3PXD2A | 37634 |
| 0.26334 | cg00369056 | | 7795 |
| 0.26334 | cg13683667 | | 252916 |
| 0.26327 | cg19576241 | | 352883 |
| 0.26302 | cg21983607 | | 390212 |
| 0.26214 | cg03666350 | | 74131 |
| 0.26137 | cg05166871 | SKAP1; SKAP1 | 102879 |
| 0.2612 | cg15366684 | ADAMTS2; ADAMTS2 | 284322 |
| 0.26116 | cg25885684 | | 451999 |
| 0.26059 | cg17290488 | C5orf45; C5orf45 | 316572 |
| 0.25763 | cg19954341 | T | 359498 |
| 0.25735 | cg20668662 | SH3PXD2A | 370194 |
| 0.25696 | cg24657523 | MYEF2 | 432522 |
| 0.25675 | cg03537386 | COL16A1 | 71519 |
| 0.25598 | cg25616600 | | 448034 |
| 0.25566 | cg20201270 | | 363196 |
| 0.25559 | cg01229787 | | 25802 |
| 0.25515 | cg08145723 | ST14 | 158133 |
| 0.25468 | cg07878514 | INSM1; INSM1 | 153203 |
| 0.25399 | cg06189394 | LRIG1 | 121568 |
| 0.25382 | cg08830502 | | 170348 |
| 0.25298 | cg05979619 | MERTK | 117911 |
| 0.25278 | cg16402635 | CASP1; CARD16; CASP1; CASP1; | 301579 |
| | | CASP1; CASP1; CARD16 | |
| 0.25223 | cg17007374 | | 311924 |
| 0.25189 | cg26681770 | PMEPA1; PMEPA1; PMEPA1; | 465572 |
| | | PMEPA1 | |
| 0.25166 | cg04649852 | | 92984 |
| 0.25103 | cg16282024 | DERL1; DERL1 | 299433 |
| 0.25043 | cg01392518 | T | 28927 |
| 0.24945 | cg05590156 | TRIO | 110699 |
| 0.24922 | cg24174557 | TMEM49 | 425007 |
| 0.24886 | cg00225902 | LRIG1 | 4852 |
| 0.24875 | cg03401875 | ADAMTS2; ADAMTS2 | 68835 |
| 0.24859 | cg23489226 | | 413717 |
| 0.24854 | cg15745401 | P2RY6; P2RY6 | 290636 |
| 0.24785 | cg24752267 | | 434286 |
| 0.24696 | cg09527270 | POMC; POMC | 182597 |
| 0.24687 | cg00145961 | NTRK3; NTRK3; NTRK3 | 3269 |
| 0.24685 | cg15323840 | SYNJ2 | 283724 |
| 0.24679 | cg11261412 | B3GNTL1 | 212263 |
| 0.24671 | cg04723534 | C2CD2 | 94311 |
| 0.2467 | cg03771840 | TRIM15 | 76209 |
| 0.24643 | cg03472954 | PRKAR1B; PRKAR1B; PRKAR1B; | 70250 |
| | | PRKAR1B; PRKAR1B; PRKAR1B | |
| 0.24643 | cg23533513 | MYO7A; MYO7A; MYO7A | 414506 |
| 0.24628 | cg19010441 | MED12L; GPR171 | 344302 |
| 0.24604 | cg06186245 | TMEM45A | 121509 |
| 0.24529 | cg23834919 | | 419111 |
| 0.24525 | cg00037856 | | 943 |
| 0.24513 | cg27338607 | | 476928 |
| 0.24455 | cg05287372 | C3orf64 | 105151 |
| 0.24329 | cg14905634 | TRHDE | 277082 |
| 0.24298 | cg11692307 | PLCH2 | 219166 |
| 0.24275 | cg04677410 | MAML2 | 93491 |
| 0.24251 | cg16561266 | LOC146880; LOC146880 | 304457 |
| 0.24244 | cg09461792 | | 181455 |
| 0.24119 | cg00548708 | | 11644 |
| 0.24101 | cg11120154 | | 209834 |
| 0.24078 | cg14587335 | HRNBP3 | 271586 |
| 0.23936 | cg09650180 | GMEB2 | 184694 |
| 0.23799 | cg09379935 | P2RY6; P2RY6 | 180013 |
| 0.23797 | cg21680719 | FHL3 | 385790 |
| 0.23791 | cg24917131 | | 437034 |
| 0.23723 | cg12097883 | LOC146880; LOC146880 | 226317 |
| 0.23698 | cg03558269 | | 71975 |
| 0.2367 | cg08870914 | | 171157 |
| 0.23667 | cg14501219 | LOC146880; LOC146880 | 269672 |
| 0.2365 | cg09180564 | OLFM3 | 176500 |
| 0.23608 | cg02477603 | | 50448 |
| 0.23603 | cg14260695 | | 264921 |
| 0.23574 | cg08613122 | PRKAR1B; PRKAR1B; PRKAR1B; | 166544 |
| | | PRKAR1B; PRKAR1B; PRKAR1B | |
| 0.23559 | cg25577130 | CA4 | 447341 |
| 0.23548 | cg08718098 | | 168443 |
| 0.23462 | cg17896243 | | 326150 |
| 0.23435 | cg09385667 | PDZRN3 | 180096 |
| 0.2343 | cg09529093 | ANGPT1 | 182630 |
| 0.23254 | cg10960920 | PGCP | 207207 |
| 0.23124 | cg07107756 | CARD14; CARD14 | 138866 |
| 0.23111 | cg07480647 | GPR153 | 145935 |
| 0.23044 | cg11218619 | | 211494 |
| 0.23008 | cg05721365 | LOC146880; LOC146880 | 113082 |
| 0.22965 | cg21531685 | PDZRN3 | 383375 |
| 0.22946 | cg12854371 | LOC146880; LOC146880 | 238254 |
| 0.22905 | cg04028695 | | 81035 |
| 0.22873 | cg05665651 | ANKRD11 | 112125 |
| 0.22861 | cg09259235 | | 177859 |
| 0.2273 | cg15341866 | | 283990 |
| 0.22729 | cg03019360 | | 61484 |
| 0.22691 | cg19409546 | | 350194 |
| 0.22689 | cg26098164 | BMP7 | 455309 |
| 0.22559 | cg23965931 | STOX2 | 421288 |
| 0.22486 | cg27292547 | | 475989 |
| 0.22459 | cg10094238 | ARHGAP27; ARHGAP27; | 192273 |
| | | ARHGAP27 | |
| 0.22433 | cg26938118 | NEDD4L; NEDD4L; NEDD4L; | 470085 |
| | | NEDD4L; NEDD4L; NEDD4L; | |
| | | NEDD4L; NEDD4L; NEDD4L | |
| 0.22395 | cg12741645 | AEN | 236560 |
| 0.22392 | cg11189268 | ARHGAP27; ARHGAP27 | 210952 |
| 0.22268 | cg10481134 | C2orf58 | 199061 |
| 0.22247 | cg25213720 | MXD3; MXD3 | 441707 |
| 0.22232 | cg07357716 | | 143726 |
| 0.22207 | cg10975897 | JARID2 | 207446 |
| 0.22204 | cg10177197 | DHCR24 | 193879 |
| 0.22183 | cg26215428 | PKD2 | 457374 |
| 0.2215 | cg01451808 | | 30240 |
| 0.22132 | cg24186459 | | 425188 |
| 0.2211 | cg14910684 | | 277182 |
| 0.21926 | cg01040786 | | 21929 |
| 0.21871 | cg07146912 | PDZRN3 | 139702 |
| 0.21843 | cg01359469 | TRIP4 | 28275 |
| 0.2181 | cg07459478 | VOPP1 | 145466 |
| 0.21793 | cg06012804 | | 118540 |
| 0.21771 | cg25335841 | | 443510 |
| 0.21765 | cg26303870 | ACTN1; ACTN1; ACTN1 | 458957 |
| 0.21683 | cg01095004 | | 23112 |
| 0.21596 | cg13369952 | JAKMIP2 | 246290 |
| 0.21555 | cg26159368 | | 456463 |
| 0.21526 | cg10328583 | PLEKHG5; PLEKHG5; PLEKHG5; | 196553 |
| | | PLEKHG5 | |
| 0.21524 | cg22041190 | PKNOX2 | 391040 |
| 0.21505 | cg13717446 | LOC146880; LOC146880 | 253646 |
| 0.215 | cg20134287 | AUTS2; AUTS2 | 362245 |
| 0.21488 | cg16755214 | | 308115 |
| 0.21465 | cg00599219 | MEFV | 12810 |
| 0.21458 | cg13470462 | BOC | 248426 |
| 0.21391 | cg13027183 | JARID2 | 240842 |
| 0.21382 | cg14467181 | | 268995 |
| 0.21371 | cg04674201 | | 93425 |
| 0.2137 | cg16936561 | RAB11FIP5 | 310943 |
| 0.21339 | cg13393580 | MXRA8 | 246755 |
| 0.21294 | cg00813162 | ACTN1; ACTN1; ACTN1 | 17174 |
| 0.21281 | cg21687563 | TTC28 | 385895 |
| 0.21199 | cg07076816 | | 138253 |
| 0.21196 | cg10195336 | ARHGEF10 | 194213 |
| 0.21167 | cg26588688 | | 463998 |
| 0.21129 | cg14524975 | ITGB7 | 270223 |
| 0.21125 | cg05373161 | TMEM231; TMEM231; TMEM231 | 106877 |
| 0.21109 | cg10283505 | FUT4; FUT4 | 195763 |
| 0.2109 | cg27190145 | DAAM2 | 474406 |
| 0.21048 | cg23403980 | SMG6; SMG6 | 412406 |
| 0.21047 | cg10734665 | ATP10A | 203498 |
| 0.2102 | cg01071185 | | 22591 |
| 0.20996 | cg13313047 | CABLES1; CABLES1; CABLES1 | 245295 |
| 0.20986 | cg18325315 | GTF2IRD1; GTF2IRD1 | 333329 |
| 0.2097 | cg24478420 | | 429606 |
| 0.20967 | cg20489026 | KCNJ9 | 367521 |
| 0.20958 | cg09119854 | AXIN2 | 175345 |
| 0.2095 | cg02186748 | COL1A1 | 44750 |
| 0.20938 | cg08223717 | EEPD1 | 159595 |
| 0.20902 | cg17265829 | FAT4 | 316130 |
| 0.20884 | cg04158862 | KIAA0247 | 83588 |
| 0.2087 | cg01521220 | MIR1203; SKAP1; SKAP1 | 31528 |
| 0.20851 | cg20472384 | DIP2C | 367281 |
| 0.2085 | cg01322214 | C7orf31 | 27599 |
| 0.20824 | cg22972806 | LYPD6B | 405136 |
| 0.20805 | cg12531838 | DIP2C | 233187 |
| 0.20773 | cg06946797 | | 135711 |
| 0.20742 | cg11394820 | | 214445 |
| 0.2069 | cg06132400 | | 120606 |
| 0.20684 | cg13505393 | LRRC32; LRRC32 | 249183 |
| 0.20661 | cg02931762 | | 59687 |
| 0.20651 | cg23367392 | GLI3 | 411874 |
| 0.20651 | cg21699330 | NFE2L3 | 386040 |
| 0.20618 | cg09532095 | THUMPD2; THUMPD2 | 182672 |
| 0.20597 | cg02955287 | | 60173 |
| 0.20575 | cg07980854 | | 155098 |
| 0.20548 | cg21838924 | CLDN4 | 388015 |
| 0.20493 | cg09703910 | | 185605 |
| 0.20481 | cg03179435 | FRMD4A | 64565 |
| 0.20458 | cg25946605 | CYS1 | 452893 |
| 0.20455 | cg12473775 | RHOD | 232200 |
| 0.20401 | cg05467716 | LOC146880 | 108495 |
| 0.20331 | cg18476176 | CNTF; ZFP91-CNTF | 335848 |
| 0.20322 | cg18362448 | SSBP4; SSBP4 | 333911 |
| 0.20314 | cg19245980 | | 347655 |
| 0.20291 | cg27342781 | | 476987 |
| 0.20279 | cg08011255 | B3GNTL1 | 155601 |
| 0.2027 | cg25252585 | FXYD7 | 442281 |
| 0.20218 | cg08170227 | ACTN1; ACTN1; ACTN1 | 158605 |
| 0.20196 | cg01700462 | C20orf123 | 34874 |
| 0.20185 | cg08920174 | | 172096 |
| 0.20181 | cg06180729 | LDLRAD3 | 121411 |
| 0.20165 | cg03144232 | STX1B | 63872 |
| 0.20053 | cg05302100 | COL1A1 | 105438 |
| 0.20037 | cg01572653 | | 32559 |
| 0.20024 | cg00509485 | | 10852 |
| 0.20012 | cg02077068 | GPRC5B | 42649 |
| 0.19965 | cg08797704 | | 169751 |
| 0.19946 | cg25674613 | | 448920 |
| 0.19907 | cg14236519 | C7orf50; C7orf50; C7orf50 | 264409 |
| 0.19873 | cg09371059 | | 179858 |
| 0.19866 | cg07250758 | VWCE | 141804 |
| 0.19825 | cg14463068 | MYT1L | 268912 |
| 0.19821 | cg27638057 | C18orf45 | 481936 |
| 0.19814 | cg19764489 | KCNQ1; KCNQ1 | 356206 |
| 0.19799 | cg05501859 | C11orf17; C11orf17 | 109151 |
| 0.19792 | cg02108623 | PRDM1 | 43306 |
| 0.19791 | cg18229049 | NXN | 331759 |
| 0.1975 | cg15557036 | | 287330 |
| 0.19749 | cg03284879 | | 66524 |
| 0.19681 | cg23899450 | | 420132 |
| 0.19665 | cg16896313 | | 310257 |
| 0.19654 | cg11738198 | | 219981 |
| 0.19638 | cg25104234 | | 439983 |
| 0.19612 | cg08863777 | FUT4 | 171018 |
| 0.19591 | cg03143849 | CDKN1C; CDKN1C; CDKN1C | 63866 |
| 0.19551 | cg27608563 | CMIP | 481430 |
| 0.19548 | cg15601264 | | 288057 |
| 0.19518 | cg25712088 | DIP2C | 449437 |
| 0.19493 | cg14003265 | TRAF2 | 259423 |
| 0.19481 | cg16926213 | | 310774 |
| 0.19467 | cg15339026 | | 283944 |
| 0.19466 | cg16429975 | ACOT7; ACOT7 | 302121 |
| 0.19451 | cg19780308 | MAML3 | 356489 |
| 0.19435 | cg01741688 | SH3RF3 | 35665 |
| 0.19412 | cg15465501 | | 285947 |
| 0.19412 | cg08639948 | | 167048 |
| 0.19403 | cg12453623 | | 231887 |
| 0.19394 | cg19093370 | PLD6 | 345512 |
| 0.19388 | cg10331829 | SLC1A2 | 196611 |
| 0.19382 | cg01806382 | | 37024 |
| 0.1935 | cg20953052 | MIR548F2; ERBB4; ERBB4 | 374498 |
| 0.19341 | cg20917077 | CPA5; CPA5; CPA5 | 373937 |
| 0.19321 | cg16434331 | SLC39A11; SLC39A11 | 302207 |
| 0.19286 | cg06581409 | | 128362 |
| 0.1925 | cg13229078 | | 243978 |
| 0.19228 | cg07044938 | | 137615 |
| 0.19221 | cg22853713 | CASZ1; CASZ1 | 403146 |
| 0.19142 | cg23878906 | | 419768 |
| 0.19138 | cg10469520 | | 198872 |
| 0.19133 | cg24229188 | HMG20B | 425784 |
| 0.19107 | cg27185501 | | 474347 |
| 0.19077 | cg03556869 | UST | 71956 |
| 0.19047 | cg03179447 | | 64567 |
| 0.18988 | cg15457718 | | 285805 |
| 0.18981 | cg27169559 | | 474039 |
| 0.1897 | cg00660305 | PRKAR1B; PRKAR1B; PRKAR1B; | 13970 |
| | | PRKAR1B; PRKAR1B; PRKAR1B | |
| 0.1891 | cg20142501 | NXN | 362369 |
| 0.18892 | cg15298286 | C17orf72 | 283380 |
| 0.18871 | cg25954269 | | 453043 |
| 0.18845 | cg25418528 | P2RX6; P2RX6 | 444919 |
| 0.18829 | cg27132535 | | 473446 |
| 0.18828 | cg15449664 | THY1 | 285660 |
| 0.18804 | cg09109656 | | 175197 |
| 0.18788 | cg04070601 | FMNL3; FMNL3 | 81748 |
| 0.18776 | cg20882260 | CACNA1C; | 373464 |
| 0.18756 | cg18267489 | CD93 | 332434 |
| 0.1875 | cg20463302 | SH3D20 | 367133 |
| 0.18746 | cg22020227 | OLFML3 | 390677 |
| 0.18628 | cg11419575 | | 214920 |
| 0.18581 | cg23369564 | PLEKHO1 | 411901 |
| 0.18537 | cg04768998 | WIPF1; WIPF1 | 95158 |
| 0.18526 | cg06605053 | | 128746 |
| 0.18519 | cg05008070 | DACT1; DACT1 | 99885 |
| 0.18519 | cg16030113 | | 295289 |
| 0.18514 | cg00472879 | SORBS1; SORBS1; SORBS1; | 10022 |
| | | SORBS1; SORBS1; SORBS1; | |
| | | SORBS1 | |
| 0.18466 | cg14832378 | | 275849 |
| 0.18464 | cg27438889 | TACC2; TACC2; TACC2; TACC2 | 478571 |
| 0.18403 | cg04098052 | | 82284 |
| 0.184 | cg26275647 | MDFIC; MDFIC | 458453 |
| 0.18384 | cg05757757 | | 113669 |
| 0.18333 | cg01198994 | MXRA8 | 25206 |
| 0.1833 | cg16104139 | | 296436 |
| 0.18322 | cg09868374 | | 188450 |
| 0.18317 | cg17797591 | AXIN2 | 324404 |
| 0.18247 | cg00250409 | | 5354 |
| 0.18239 | cg09976669 | SWAP70 | 190212 |
| 0.18236 | cg22231400 | SLC17A7 | 393823 |
| 0.18227 | cg02351674 | | 48045 |
| 0.18188 | cg11676636 | ARHGAP22 | 218912 |
| 0.18152 | cg00831127 | EPHB2; EPHB2 | 17525 |
| 0.18147 | cg14054883 | SYK; SYK; SYK; SYK | 260571 |
| 0.1814 | cg05147262 | ENAM | 102478 |
| 0.1812 | cg21242079 | ADAMTS7 | 379397 |
| 0.18059 | cg26523175 | PDZK1IP1 | 462775 |
| 0.18031 | cg04778236 | SPSB1 | 95348 |
| 0.18026 | cg01820962 | NT5DC1 | 37371 |
| 0.18025 | cg12315995 | | 229820 |
| 0.17993 | cg17250262 | LBH | 315917 |
| 0.17965 | cg14227785 | | 264238 |
| 0.17877 | cg10882304 | TACC2; TACC2; TACC2; TACC2 | 205944 |
| 0.17828 | cg07047570 | INSM1; INSM1 | 137657 |
| 0.17808 | cg07286123 | CD93 | 142424 |
| 0.17794 | cg14089493 | | 261291 |
| 0.17787 | cg07997634 | SLC25A13; SLC25A13; SLC25A13 | 155378 |
| 0.1776 | cg18628732 | | 338265 |
| 0.17747 | cg12239557 | TACC2; TACC2 | 228662 |
| 0.17737 | cg06376402 | INPP5A | 124706 |
| 0.17726 | cg09642857 | | 184563 |
| 0.17704 | cg17099048 | COL22A1 | 313414 |
| 0.17689 | cg10819733 | | 204928 |
| 0.17667 | cg26477792 | | 462022 |
| 0.17575 | cg00373436 | TAGLN3; TAGLN3; TAGLN3 | 7897 |
| 0.17567 | cg15029402 | | 279170 |
| 0.17558 | cg19080233 | | 345285 |
| 0.17527 | cg00157016 | PFN4 | 3525 |
| 0.17525 | cg02278646 | ANO6; ANO6; PLEKHA9; ANO6 | 46492 |
| 0.17489 | cg14684457 | NFE2L3 | 273413 |
| 0.17474 | cg09125999 | DSCAML1 | 175477 |
| 0.17462 | cg04940251 | | 98587 |
| 0.17444 | cg07135032 | LMX1A | 139425 |
| 0.17437 | cg03491895 | | 70579 |
| 0.17348 | cg07459019 | | 145455 |
| 0.17324 | cg03762242 | GAS7; GAS7; GAS7 | 76010 |
| 0.17305 | cg23585686 | | 415170 |
| 0.17287 | cg00400827 | | 8476 |
| 0.17285 | cg02284273 | FRMD4A | 46611 |
| 0.17232 | cg03560973 | POMC; POMC | 72034 |
| 0.17213 | cg22304543 | | 394846 |
| 0.17212 | cg24147392 | LOC440461 | 424521 |
| 0.17206 | cg14251267 | ZBTB46 | 264704 |
| 0.1718 | cg20052079 | JARID2 | 360990 |
| 0.17144 | cg19161251 | LMO4 | 346462 |
| 0.17138 | cg01364478 | PRRX1; PRRX1 | 28365 |
| 0.17129 | cg14255700 | IPPK | 264808 |
| 0.17113 | cg07067577 | SH3D20 | 138069 |
| 0.17078 | cg27345989 | NXN | 477039 |
| 0.17062 | cg21029769 | ST14 | 375731 |
| 0.17054 | cg26821721 | KLHL29 | 467947 |
| 0.17025 | cg13890428 | | 257013 |
| 0.17003 | cg15134506 | USP54 | 280991 |
| 0.17002 | cg21142398 | GLIS1 | 377429 |
| 0.16954 | cg08533336 | USP2; USP2 | 165172 |
| 0.16947 | cg18006637 | FAM13A; FAM13A | 327818 |
| 0.16924 | cg23692500 | | 417116 |
| 0.16901 | cg01093786 | MAML2 | 23085 |
| 0.169 | cg00285394 | SQLE | 6012 |
| 0.16825 | cg20673819 | C6orf176; C6orf176 | 370286 |
| 0.16804 | cg03676578 | | 74324 |
| 0.168 | cg21946195 | ATOH8 | 389751 |
| 0.16791 | cg10346364 | LOC100130987; CLCF1; CLCF1 | 196856 |
| 0.16785 | cg08935841 | | 172361 |
| 0.16779 | cg02216727 | GJD3; GJD3 | 45257 |
| 0.16775 | cg13088556 | | 241806 |
| 0.16685 | cg20860726 | MYT1L | 373230 |
| 0.16671 | cg11927233 | NPM1; NPM1; NPM1 | 223346 |
| 0.16655 | cg17842189 | TBC1D22A | 325238 |
| 0.16649 | cg23508201 | | 414035 |
| 0.16631 | cg18772205 | KLHL6 | 340567 |
| 0.16625 | cg18402987 | | 334557 |
| 0.16616 | cg10350880 | EPHB4 | 196929 |
| 0.16606 | cg03943115 | SPRR2D | 79303 |
| 0.16531 | cg03967266 | | 79784 |
| 0.16529 | cg09099242 | MMRN2 | 175043 |
| 0.16453 | cg08302650 | LRRC24; LRRC14 | 161144 |
| 0.1644 | cg08049717 | AMOTL2 | 156270 |
| 0.16429 | cg03764092 | TBXAS1; TBXAS1; TBXAS1; | 76037 |
| | | TBXAS1; TBXAS1; TBXAS1 | |
| 0.16421 | cg07275179 | ATXN7; ATXN7 | 142217 |
| 0.16413 | cg05529123 | | 109677 |
| 0.16411 | cg14103872 | OLFML2A | 261610 |
| 0.16404 | cg19996406 | | 360145 |
| 0.16364 | cg16568084 | | 304587 |
| 0.16364 | cg13165983 | | 242911 |
| 0.16332 | cg00409696 | | 8667 |
| 0.16319 | cg11733958 | MPRIP; MPRIP | 219898 |
| 0.16308 | cg08458711 | BSND | 163781 |
| 0.16261 | cg18861112 | WDFY2 | 342032 |
| 0.16254 | cg21293455 | | 380108 |
| 0.16247 | cg06526620 | FUT4 | 127302 |
| 0.16242 | cg12303623 | USP54 | 229638 |
| 0.16225 | cg06501835 | | 126864 |
| 0.16225 | cg05243389 | MYEF2 | 104339 |
| 0.1619 | cg15372959 | TACC2; TACC2; TACC2; TACC2 | 284406 |
| 0.16187 | cg08879365 | | 171347 |
| 0.16183 | cg00802386 | | 16940 |
| 0.16175 | cg16224163 | LPP | 298398 |
| 0.16119 | cg02276070 | ARSI | 46431 |
| 0.16116 | cg00702126 | VWA1; VWA1 | 14846 |
| 0.1611 | cg04674421 | | 93433 |
| 0.16107 | cg08333931 | SH3D20 | 161705 |
| 0.16089 | cg27177158 | | 474183 |
| 0.16083 | cg04948016 | | 98755 |
| 0.1607 | cg19144071 | | 346231 |
| 0.1607 | cg10628617 | SCUBE1 | 201790 |
| 0.16063 | cg19542987 | ARMC3 | 352374 |
| 0.16049 | cg27265404 | | 475561 |
| 0.16007 | cg03756522 | | 75895 |
| 0.15976 | cg19856845 | RIN3 | 357783 |
| 0.15961 | cg09492169 | ARHGAP27; ARHGAP27 | 181962 |
| 0.15953 | cg27458177 | | 478888 |
| 0.1592 | cg03303857 | GRIK4 | 66884 |
| 0.15887 | cg11845549 | | 221989 |
| 0.15871 | cg15246712 | EBF2 | 282686 |
| 0.15867 | cg08009487 | KRT4; KRT4 | 155576 |
| 0.15857 | cg21429516 | | 381785 |
| 0.15832 | cg13552867 | KCNJ9 | 250134 |
| 0.15799 | cg22044408 | | 391096 |
| 0.1579 | cg13440947 | GFRA1; GFRA1; GFRA1 | 247730 |
| 0.15763 | cg14363469 | | 266959 |
| 0.15736 | cg08005872 | | 155513 |
| 0.1573 | cg00094585 | | 2207 |
| 0.15729 | cg04944536 | | 98671 |
| 0.15686 | cg04658243 | RPTOR; RPTOR | 93139 |
| 0.15641 | cg11454459 | | 215415 |
| 0.15627 | cg11084571 | KANK4 | 209299 |
| 0.15582 | cg26227310 | TLX1NB | 457602 |
| 0.15574 | cg05541311 | | 109868 |
| 0.15565 | cg00095431 | | 2233 |
| 0.15534 | cg08548284 | | 165445 |
| 0.15493 | cg03919650 | MEFV | 78874 |
| 0.15488 | cg05786009 | | 114191 |
| 0.15461 | cg04024827 | FLJ12825 | 80948 |
| 0.15448 | cg15653254 | | 289035 |
| 0.1544 | cg04649598 | ODZ4 | 92979 |
| 0.15426 | cg08615354 | | 166583 |
| 0.15398 | cg05909702 | | 116481 |
| 0.15394 | cg08785724 | | 169563 |
| 0.15382 | cg00990769 | MATN2; MATN2 | 20836 |
| 0.15381 | cg08734931 | FRMD4A | 168749 |
| 0.15277 | cg05277096 | PDZRN3 | 104972 |
| 0.15274 | cg25169706 | ST5; ST5; ST5 | A441040 |
| 0.15273 | cg24572745 | BOC | 431168 |
| 0.15262 | cg21176130 | HLA-E | 378075 |
| 0.15251 | cg00514353 | PCDH1 | 10956 |
| 0.15225 | cg25515269 | ITGA4 | 446437 |
| 0.15211 | cg25653204 | FPR2 | 448621 |
| 0.15196 | cg25349820 | | 443748 |
| 0.15173 | cg11401293 | COL6A1 | 214569 |
| 0.15074 | cg14036069 | NEK6; NEK6; NEK6; NEK6 | 260162 |
| 0.15059 | cg14110685 | | 261719 |
| 0.15038 | cg11673092 | ARMC3 | 218848 |
| 0.15033 | cg24098326 | TACC2; TACC2; TACC2; TACC2 | 423638 |
| 0.15015 | cg17783086 | LOC146880; LOC146880 | 324191 |
| 0.14948 | cg17332379 | LOC146880; LOC146880 | 317320 |
| 0.14904 | cg26697605 | | 465886 |
| 0.1489 | cg16637224 | | 305900 |
| 0.14883 | cg19793499 | AUTS2; AUTS2 | 356717 |
| 0.14862 | cg13698865 | COL5A1 | 253254 |
| 0.14823 | cg14955976 | | 277886 |
| 0.1482 | cg24537942 | KANK4 | 430673 |
| 0.14803 | cg22958076 | | 404890 |
| 0.14799 | cg02079741 | POMC; POMC | 42708 |
| 0.14778 | cg12055422 | | 225496 |
| 0.14773 | cg13947999 | | 258288 |
| 0.1477 | cg13121428 | TGFB3 | 242270 |
| 0.14752 | cg25651608 | | 448592 |
| 0.14738 | cg03573945 | EBF2 | 72279 |
| 0.14713 | cg07922154 | ARG2 | 154039 |
| 0.14713 | cg06516502 | | 127100 |
| 0.14685 | cg26719062 | | 466225 |
| 0.14673 | cg00020474 | | 497 |
| 0.14661 | cg07015554 | | 137042 |
| 0.1466 | cg02095937 | TRIM59 | 43034 |
| 0.14648 | cg22111167 | PTPRJ; PTPRJ | 392026 |
| 0.14639 | cg16892553 | PRR15L; PRR15L | 310202 |
| 0.14634 | cg00512602 | SH3D20 | 10920 |
| 0.14595 | cg27359636 | | 477238 |
| 0.14577 | cg23325335 | | 411144 |
| 0.14539 | cg15671996 | TRIM15 | 289301 |
| 0.14536 | cg14725580 | C6orf106; C6orf106 | 274100 |
| 0.14511 | cg13724550 | FGF1; FGF1; FGF1; FGF1; FGF1; | 253793 |
| | | FGF1; FGF1 | |
| 0.14497 | cg19399514 | RFTN1 | 349989 |
| 0.14495 | cg11790417 | | 220854 |
| 0.14482 | cg15152115 | | 281271 |
| 0.1445 | cg11474182 | CNTNAP3 | 215691 |
| 0.14435 | cg05123976 | | 101999 |
| 0.1441 | cg03009240 | FZR1 | 61309 |
| 0.14404 | cg09359810 | FRMD6 | 179634 |
| 0.14388 | cg23931919 | | 420690 |
| 0.14388 | cg10321236 | MYO7A; MYO7A; MYO7A | 196418 |
| 0.1436 | cg11222217 | CASZ1; CASZ1 | 211555 |
| 0.14337 | cg21487363 | FOXP1; FOXP1 | 382660 |
| 0.14296 | cg23522194 | PLA2G4C; PLA2G4C; PLA2G4C | 414285 |
| 0.14295 | cg26871180 | | 468843 |
| 0.14284 | cg16139517 | CNTN4 | 297000 |
| 0.14275 | cg15371815 | CRIM1 | 284394 |
| 0.14258 | cg24118856 | PPAN-P2RY11; P2RY11 | 423968 |
| 0.14258 | cg15400997 | PLXNC1 | 284893 |
| 0.14257 | cg26950531 | DPF1; DPF1; DPF1 | 470304 |
| 0.14252 | cg23270841 | PARD3B; PARD3B; PARD3B | 410288 |
| 0.1418 | cg00542975 | CLASP1; CLASP1; CLASP1 | 11524 |
| 0.14168 | cg06644998 | | 129612 |
| 0.14118 | cg04567302 | SLC44A4 | 91475 |
| 0.14106 | cg26002568 | | 453787 |
| 0.14087 | cg04289208 | LGR6; LGR6 | 86168 |
| 0.14072 | cg13085980 | PLXNC1 | 241773 |
| 0.14055 | cg18124852 | | 329967 |
| 0.14042 | cg13214162 | | 243716 |
| 0.14037 | cg07103748 | | 138784 |
| 0.14016 | cg04756252 | SLA; SLA; TG; SLA | 94952 |
| 0.13944 | cg17253459 | OLFML3 | 315956 |
| 0.1392 | cg04601228 | AUTS2; AUTS2 | 92149 |
| 0.13894 | cg10005270 | C10orf90 | 190712 |
| 0.13871 | cg13948956 | | 258313 |
| 0.1387 | cg01211852 | | 25460 |
| 0.13832 | cg01654566 | | 34124 |
| 0.13826 | cg09528501 | | 182620 |
| 0.13793 | cg21688152 | B3GNTL1 | 385901 |
| 0.13778 | cg25472296 | DOCK1 | 445774 |
| 0.13777 | cg21951457 | TACC2; TACC2; TACC2; TACC2 | 389827 |
| 0.13769 | cg08102611 | GAS7 | 157232 |
| 0.13766 | cg13008631 | IRS1 | 240565 |
| 0.13745 | cg20535425 | | 368163 |
| 0.13721 | cg04689880 | | 93734 |
| 0.13713 | cg04768479 | | 95139 |
| 0.13705 | cg20438277 | CD93 | 366734 |
| 0.13663 | cg10301879 | C6orf105; C6orf105 | 196052 |
| 0.13653 | cg19582200 | SCUBE1 | 352974 |
| 0.13647 | cg25967170 | | 453238 |
| 0.13638 | cg06620993 | SH3PXD2A | 129075 |
| 0.13588 | cg12812165 | NIN; NIN; NIN | 237654 |
| 0.13581 | cg13053992 | | 241251 |
| 0.13547 | cg14598213 | NGFR | 271799 |
| 0.13528 | cg19193956 | | 346894 |
| 0.13503 | cg06021990 | FLT1 | 118695 |
| 0.13498 | cg01715025 | RNF115 | 35160 |
| 0.13498 | cg05787209 | STX1B | 114223 |
| 0.13486 | cg14206273 | CASZ1; CASZ1 | 263709 |
| 0.13472 | cg25599473 | SMOC2; SMOC2 | 447739 |
| 0.13467 | cg23200466 | FBXW11; FBXW11; FBXW11 | 408900 |
| 0.13452 | cg09868780 | LVRN | 188458 |
| 0.13444 | cg05246303 | ITGA4 | 104393 |
| 0.13439 | cg03213259 | TRADD | 65187 |
| 0.13426 | cg07067982 | SLC43A3; SLC43A3; SLC43A3; | 138080 |
| | | SLC43A3 | |
| 0.13383 | cg24960118 | PKHD1; PKHD1 | 437734 |
| 0.13373 | cg06942124 | | 135615 |
| 0.13373 | cg06443678 | | 125849 |
| 0.13342 | cg18674487 | | 338954 |
| 0.13337 | cg11268280 | | 212378 |
| 0.13318 | cg08681473 | COL1A1 | 167753 |
| 0.13306 | cg03143046 | | 63849 |
| 0.13304 | cg06842253 | | 133582 |
| 0.13286 | cg26363935 | NWD1 | 460109 |
| 0.13282 | cg24250588 | CLDN19; CLDN19 | 426051 |
| 0.13272 | cg01579600 | | 32722 |
| 0.13255 | cg20567368 | | 368674 |
| 0.13204 | cg20054157 | SFTA1P | 361020 |
| 0.13169 | cg16052198 | FPR2 | 295658 |
| 0.13115 | cg12888521 | | 238770 |
| 0.13102 | cg21322248 | PSTPIP1 | 380448 |
| 0.13082 | cg23418965 | PKNOX2 | 412666 |
| 0.13073 | cg10193441 | HRNBP3 | 194174 |
| 0.13072 | cg25566312 | TGFBI | 447171 |
| 0.13053 | cg00334821 | LIMK1 | 7073 |
| 0.13046 | cg18132405 | LEPREL1; LEPREL1 | 330109 |
| 0.13036 | cg13893782 | PDE7B | 257077 |
| 0.13034 | cg12575928 | GAS2 | 233842 |
| 0.13018 | cg03669949 | DDX43; DDX43 | 74206 |
| 0.13012 | cg19528338 | BMP2 | 352128 |
| 0.13001 | cg15878619 | TUBB | 292734 |
| 0.1298 | cg07187808 | KCNJ9 | 140601 |
| 0.1297 | cg06641357 | | 129537 |
| 0.12946 | cg06618298 | MYO10 | 129012 |
| 0.12929 | cg04332927 | | 87001 |
| 0.12922 | cg11041060 | SMOC2; SMOC2 | 208580 |
| 0.12875 | cg19264571 | APCDD1 | 347991 |
| 0.12861 | cg00832547 | RAGE | 17556 |
| 0.12794 | cg05376469 | | 106960 |
| 0.1279 | cg10331500 | TGFA; TGFA | 196606 |
| 0.12762 | cg06983052 | LRRC8D; LRRC8D | 136433 |
| 0.12762 | cg12019475 | | 224847 |
| 0.12739 | cg16900264 | | 310326 |
| 0.12733 | cg01055696 | | 22251 |
| 0.12706 | cg00652846 | BOC | 13828 |
| 0.12699 | cg17265419 | ITGA4 | 316124 |
| 0.12692 | cg07570498 | PHC2; PHC2 | 147621 |
| 0.12659 | cg17504145 | CRYBB2 | 320177 |
| 0.12646 | cg00872354 | | 18374 |
| 0.12628 | cg08504407 | HRNBP3 | 164658 |
| 0.12625 | cg19072083 | | 345194 |
| 0.12581 | cg22660447 | C16orf7 | 399970 |
| 0.12507 | cg18169095 | LAD1 | 330752 |
| 0.12494 | cg24167667 | | 424904 |
| 0.12488 | cg02281167 | TRIM15 | 46550 |
| 0.12477 | cg18771659 | SPRR1B | 340554 |
| 0.12456 | cg13197521 | SPSB1 | 243430 |
| 0.12441 | cg23733123 | MARVELD1; MARVELD1 | 417794 |
| 0.12439 | cg19681793 | THBS2 | 354639 |
| 0.12435 | cg22373687 | | 395848 |
| 0.12435 | cg01838728 | | 37759 |
| 0.12422 | cg03083517 | COL22A1 | 62783 |
| 0.12417 | cg01149239 | MFI2; MFI2 | 24207 |
| 0.12417 | cg05593669 | | 110779 |
| 0.12379 | cg00348891 | | 7377 |
| 0.12362 | cg19125999 | C1orf172 | 345998 |
| 0.12359 | cg26221801 | C20orf195 | 457494 |
| 0.12334 | cg01325188 | BANP; BANP | 27654 |
| 0.12301 | cg08066024 | NRXN1; NRXN1 | 156540 |
| 0.12289 | cg04344730 | DAB1 | 87247 |
| 0.12282 | cg03467555 | | 70136 |
| 0.12254 | cg22124564 | AMZ1 | 392246 |
| 0.12211 | cg09882901 | | 188677 |
| 0.1218 | cg26472922 | | 461941 |
| 0.12177 | cg22810790 | CA4 | 402441 |
| 0.1217 | cg20396299 | | 366115 |
| 0.1215 | cg01399485 | CASZ1; CASZ1 | 29081 |
| 0.12144 | cg27423739 | | 478314 |
| 0.12144 | cg11857093 | | 222224 |
| 0.12129 | cg04004513 | SKAP1; SKAP1 | 80529 |
| 0.12105 | cg16731157 | RCAN3 | 307717 |
| 0.12064 | cg04757806 | FUT4 | 94983 |
| 0.12052 | cg10930901 | | 206696 |
| 0.12044 | cg09728333 | | 185957 |
| 0.12032 | cg22159835 | NRXN2; NRXN2; NRXN2 | 392801 |
| 0.12007 | cg10168635 | C2orf88; C2orf88; C2orf88; C2orf88 | 193705 |
| 0.1198 | cg12891678 | | 238813 |
| 0.11957 | cg06382546 | LRP1B | 124829 |
| 0.11923 | cg16118803 | MOG; MOG; MOG; MOG; MOG; | 296688 |
| | | MOG; MOG; MOG; MOG; MOG; | |
| | | MOG | |
| 0.11879 | cg24642523 | HSPA2 | 432315 |
| 0.11842 | cg05280131 | FAM128A; LOC150776 | 105029 |
| 0.11823 | cg23693749 | | 417137 |
| 0.11821 | cg25255713 | EVPLL | 442316 |
| 0.11814 | cg23257225 | ADORA1; ADORA1 | 410054 |
| 0.11813 | cg12536891 | CLPB | 233264 |
| 0.11771 | cg08922201 | SYT7 | 172124 |
| 0.11744 | cg16274954 | DPCR1 | 299319 |
| 0.11728 | cg04011829 | SLCO4A1 | 80684 |
| 0.11703 | cg16143578 | ROBO4; ROBO4 | 297069 |
| 0.11698 | cg09966895 | ODZ4 | 190045 |
| 0.11691 | cg24113950 | | 423878 |
| 0.11646 | cg19119609 | | 345922 |
| 0.11612 | cg05663064 | COL6A1 | 112083 |
| 0.1161 | cg19388972 | | 349838 |
| 0.11582 | cg05979400 | LOC100128731 | 117903 |
| 0.11568 | cg01311341 | KIAA1671 | 27416 |
| 0.11546 | cg06825512 | APCDD1 | 133201 |
| 0.11466 | cg26292150 | ZDHHC19 | 458748 |
| 0.1146 | cg06282247 | | 123104 |
| 0.11404 | cg17438457 | | 319053 |
| 0.11398 | cg06427611 | PARVB | 125571 |
| 0.11372 | cg11386098 | SYT7 | 214308 |
| 0.11358 | cg13188382 | | 243299 |
| 0.11315 | cg03409151 | ALDH7A1 | 68983 |
| 0.11311 | cg18771300 | RHOJ | 340543 |
| 0.11298 | cg15320349 | | 283665 |
| 0.11293 | cg03840259 | GRAP2 | 77400 |
| 0.11242 | cg03779072 | | 76349 |
| 0.11235 | cg00795584 | | 16819 |
| 0.11176 | cg06938705 | | 135549 |
| 0.11144 | cg04263215 | NDUFS2; ADAMTS4 | 85649 |
| 0.11144 | cg09296742 | | 178512 |
| 0.11107 | cg18804920 | CNTNAP2 | 341118 |
| 0.11103 | cg12835313 | CACNA2D3 | 237974 |
| 0.11083 | cg07157030 | RHOJ; RHOJ | 139948 |
| 0.11062 | cg19725553 | BLVRA | 355438 |
| 0.11045 | cg13777287 | CNKSR3 | 254763 |
| 0.11016 | cg26048630 | PRRX1; PRRX1 | 454529 |
| 0.10972 | cg02666837 | | 54361 |
| 0.10966 | cg11073796 | | 209086 |
| 0.10936 | cg04554928 | TRIM15 | 91220 |
| 0.10888 | cg17963461 | | 327141 |
| 0.1087 | cg15580417 | KCNJ9 | 287721 |
| 0.10863 | cg21602520 | BCL2; BCL2 | 384588 |
| 0.10849 | cg21817750 | MRPS27 | 387694 |
| 0.10849 | cg21306949 | | 380293 |
| 0.10796 | cg09126995 | PHC2; PHC2 | 175501 |
| 0.10783 | cg23931734 | CA12; CA12 | 420686 |
| 0.10741 | cg03679504 | LRRC2; LRRC2; TDGF1 | 74369 |
| 0.10704 | cg05272790 | TMPRSS13 | 104903 |
| 0.10702 | cg10512203 | COL9A2 | 199691 |
| 0.10677 | cg17848515 | SMPD4; SMPD4; SMPD4; SMPD4; | 325326 |
| | | SMPD4; SMPD4; SMPD4; | |
| | | FAM128B | |
| 0.10643 | cg06824523 | PLCH2 | 133178 |
| 0.10623 | cg24833464 | KRTAP3-1 | 435563 |
| 0.1062 | cg01619846 | UHRF1; UHRF1 | 33506 |
| 0.10589 | cg00089821 | | 2084 |
| 0.1058 | cg02506908 | HPD | 51094 |
| 0.10525 | cg24248680 | FAM24B | 426016 |
| 0.10495 | cg22261895 | PLBD1 | 394295 |
| 0.10453 | cg25638443 | ST8SIA1 | 448399 |
| 0.10437 | cg08770647 | ROBO4 | 169334 |
| 0.10378 | cg10663897 | | 202375 |
| 0.10325 | cg09075327 | PSD3 | 174665 |
| 0.10321 | cg11505113 | VASH2; VASH2; VASH2 | 216106 |
| 0.10291 | cg24251135 | USP38 | 426061 |
| 0.1029 | cg26824174 | OLFML3 | 467994 |
| 0.10284 | cg11912513 | | 223148 |
| 0.10264 | cg03887520 | | 78268 |
| 0.1024 | cg21674153 | B4GALT6 | 385733 |
| 0.10236 | cg17147466 | | 314205 |
| 0.10181 | cg21931864 | SH3D20 | 389527 |
| 0.10134 | cg09469870 | | 181594 |
| 0.10132 | cg06294384 | | 123314 |
| 0.10127 | cg22693493 | | 400543 |
| 0.10127 | cg05492433 | COL22A1 | 108970 |
| 0.10066 | cg06915112 | | 135102 |
| 0.10002 | cg14320626 | | 266136 |

TABLE 7

| | | | SEQ |
|---|---|---|---|
| OA − RA | Locus | Associated gene | ID No. |
| −0.10022 | cg00490976 | | 10388 |
| −0.10038 | cg00082140 | | 1884 |
| −0.10076 | cg04080530 | | 81955 |
| −0.10083 | cg25035059 | CHD7 | 438996 |
| −0.10092 | cg09944434 | | 189753 |
| −0.10117 | cg14538532 | PBX2 | 270543 |
| −0.10144 | cg27165033 | | 473953 |
| −0.10204 | cg03110300 | CPLX4 | 63236 |
| −0.10239 | cg14729148 | CUGBP2; CUGBP2; CUGBP2; | 274152 |
| | | CUGBP2 | |
| −0.10249 | cg15730121 | DLGAP4; DLGAP4; DLGAP4 | 290375 |
| −0.10256 | cg24037389 | SCARA5 | 422606 |
| −0.10268 | cg20933239 | | 374167 |
| −0.10274 | cg10627737 | COL4A2 | 201776 |
| −0.1028 | cg19006947 | PLCB3 | 344239 |
| −0.10299 | cg08613239 | | 166546 |
| −0.10305 | cg15645757 | UBE2Q1 | 288883 |
| −0.10316 | cg19056664 | CLPTM1L | 345033 |
| −0.10356 | cg03741475 | | 75606 |
| −0.10398 | cg26332797 | CTTNBP2 | 459481 |
| −0.10398 | cg07447015 | ANKRD11 | 145230 |
| −0.10414 | cg06823060 | CMIP; CMIP | 133147 |
| −0.10436 | cg05150973 | EHD1 | 102541 |
| −0.10453 | cg05587232 | C7orf50; C7orf50; C7orf50 | 110657 |
| −0.10463 | cg26949393 | AGAP3 | 470288 |
| −0.10493 | cg07266910 | ZMAT3; ZMAT3 | 142085 |
| −0.10509 | cg01993576 | SLC29A1; SLC29A1 | 40885 |
| −0.10514 | cg22907277 | C7orf50; C7orf50; C7orf50 | 404094 |
| −0.10515 | cg12544243 | GRIN3B | 233359 |
| −0.10541 | cg23078123 | GPR177 | 407010 |
| −0.10541 | cg09545918 | COL8A1; MIR548G; COL8A1 | 182887 |
| −0.1056 | cg15067032 | | 279802 |
| −0.10586 | cg24781973 | FMN1 | 434762 |
| −0.1059 | cg07576033 | C16orf11 | 147740 |
| −0.10596 | cg02256384 | | 46036 |
| −0.10602 | cg08371643 | CA4 | 162329 |
| −0.10751 | cg10985158 | NAV1 | 207637 |
| −0.10791 | cg06531624 | | 127430 |
| −0.10813 | cg25570929 | COLEC12 | 447236 |
| −0.1085 | cg00997264 | | 20997 |
| −0.10866 | cg18494399 | SLC1A2 | 336159 |
| −0.10876 | cg21406461 | IFI16 | 381503 |
| −0.10879 | cg24395452 | SPATA18 | 428258 |
| −0.109 | cg10903916 | ASAP2; ASAP2 | 206293 |
| −0.1093 | cg06330375 | TSC2; TSC2; TSC2 | 123883 |
| −0.10957 | cg00779797 | CAPN2; CAPN2 | 16482 |
| −0.11024 | cg18145505 | GREM1 | 330327 |
| −0.11042 | cg26883033 | | 469090 |
| −0.11044 | cg25334834 | VGLL4; VGLL4; VGLL4; VGLL4 | 443491 |
| −0.11048 | cg18565130 | C7orf50; C7orf50; C7orf50 | 337296 |
| −0.11085 | cg06067955 | MYPN | 119469 |
| −0.11124 | cg11147154 | FLNC; FLNC | 210249 |
| −0.11125 | cg14473823 | | 269143 |
| −0.11142 | cg18324583 | | 333319 |
| −0.11152 | cg24917359 | C1QTNF1; C1QTNF1; C1QTNF1 | 437035 |
| −0.11155 | cg22346073 | CLASP1; CLASP1; CLASP1 | 395428 |
| −0.1116 | cg04958191 | ASPH; ASPH; ASPH; ASPH; | 98926 |
| | | ASPH | |
| −0.11187 | cg12160741 | BRF1 | 227383 |
| −0.11193 | cg01821635 | | 37390 |
| −0.11205 | cg03131767 | ABCB9; ABCB9; ABCB9 | 63648 |
| −0.11209 | cg26576937 | EBF4 | 463815 |
| −0.11214 | cg15142477 | RNF165 | 281126 |
| −0.1125 | cg13893427 | | 257066 |
| −0.11262 | cg17527422 | C12orf68; C12orf68 | 320633 |
| −0.1128 | cg08642731 | C10orf71; C10orf71 | 167107 |
| −0.11281 | cg15006627 | GPRC5A | 278771 |
| −0.11292 | cg12626411 | PRG4; PRG4; PRG4; MIR548F1; | 234784 |
| | | PRG4 | |
| −0.1131 | cg22851557 | PRDM16; PRDM16 | 403103 |
| −0.11319 | cg26803803 | DNMT3A; DNMT3A; MIR1301; | 467585 |
| | | DNMT3A | |
| −0.11355 | cg18746826 | SCARA5 | 340084 |
| −0.11368 | cg26186331 | DAB2 | 456925 |
| −0.11378 | cg15696968 | KIAA1949; NRM; KIAA1949 | 289755 |
| −0.11389 | cg11014810 | SH3PXD2A | 208137 |
| −0.11393 | cg14190889 | HLA-H | 263376 |
| −0.11407 | cg17655346 | CACNA2D3 | 322379 |
| −0.11414 | cg24777564 | ADAMTS12 | 434703 |
| −0.11439 | cg04156383 | PDE4DIP; PDE4DIP; PDE4DIP; | 83534 |
| | | PDE4DIP | |
| −0.11448 | cg02296376 | MIR1286; RTN4R | 46872 |
| −0.11454 | cg00145875 | | 3263 |
| −0.11464 | cg21862126 | | 388401 |
| −0.11468 | cg12181407 | | 227738 |
| −0.11476 | cg21733013 | RAPGEF5 | 386423 |
| −0.11485 | cg25379549 | ADARB2 | 444296 |
| −0.11508 | cg07815521 | MIR641; AKT2 | 152123 |
| −0.11512 | cg03037030 | TNF | 61825 |
| −0.11515 | cg14930864 | YWHAQ | 277519 |
| −0.11516 | cg23980760 | ACCSL; ACCSL | 421559 |
| −0.11517 | cg26858414 | CDSN; PSORS1C1 | 468630 |
| −0.11524 | cg10831642 | SH3PXD2A | 205142 |
| −0.11537 | cg03901836 | C2orf57 | 78575 |
| −0.11539 | cg16705287 | POLR2E | 307214 |
| −0.11541 | cg05557262 | FAM180A | 110128 |
| −0.11544 | cg03860038 | | 77780 |
| −0.11546 | cg20989591 | C7orf50; C7orf50; C7orf50 | 375141 |
| −0.1157 | cg03627771 | GRAMD1B | 73371 |
| −0.1158 | cg02145701 | BANP; BANP | 43968 |
| −0.11616 | cg13093380 | ALK | 241873 |
| −0.11621 | cg10239022 | GPR133 | 195020 |
| −0.1167 | cg17753942 | | 323769 |
| −0.11676 | cg21540399 | PTPRG | 383531 |
| −0.11688 | cg16538359 | | 304039 |
| −0.11692 | cg01320211 | SEMA6D; SEMA6D; SEMA6D; | 27561 |
| | | SEMA6D; SEMA6D; SEMA6D | |
| −0.11693 | cg02020821 | PPP2R5C; PPP2R5C; PPP2R5C; | 41450 |
| | | PPP2R5C; PPP2R5C | |
| −0.11698 | cg06873401 | LHFPL2 | 134232 |
| −0.11705 | cg15671083 | UCN3 | 289292 |
| −0.1173 | cg00102093 | | 2393 |
| −0.11732 | cg21356710 | MFSD2B | 380867 |
| −0.11738 | cg21205282 | | 378649 |
| −0.11761 | cg07723948 | OSGIN1; OSGIN1; OSGIN1 | 150463 |
| −0.1177 | cg16500916 | | 303286 |
| −0.11772 | cg24634422 | SLC1A2 | 432160 |
| −0.11788 | cg06755262 | | 131696 |
| −0.11803 | cg24698488 | MMP14 | 433309 |
| −0.11805 | cg01675953 | MCF2L; MCF2L | 34463 |
| −0.11814 | cg15547672 | RPTOR; RPTOR | 287142 |
| −0.11827 | cg23850174 | | 419365 |
| −0.1184 | cg05429527 | | 107954 |
| −0.11845 | cg27437823 | MAP3K14 | 478553 |
| −0.11865 | cg15470365 | SORBS2; SORBS2; SORBS2; | 286032 |
| | | SORBS2; SORBS2 | |
| −0.1187 | cg24577455 | PRKCZ; PRKCZ; PRKCZ | 431245 |
| −0.11873 | cg18458739 | BCAS3; BCAS3 | 335553 |
| −0.11874 | cg11335264 | CAPZB | 213463 |
| −0.11901 | cg00961940 | TIMP2 | 20220 |
| −0.11901 | cg04712892 | NRAP; NRAP | 94119 |
| −0.11917 | cg27582168 | FANCA | 481016 |
| −0.11931 | cg17294136 | CACNA2D1 | 316631 |
| −0.11937 | cg13932122 | | 257954 |
| −0.11937 | cg10637688 | | 201971 |
| −0.11938 | cg05384139 | PITPNM2 | 107123 |
| −0.11954 | cg04554240 | ESYT3 | 91205 |
| −0.11954 | cg22429169 | ZNF335 | 396572 |
| −0.11965 | cg03917297 | | 78835 |
| −0.11967 | cg19408740 | IGSF8 | 350178 |
| −0.11986 | cg09745791 | CTRB1 | 186257 |
| −0.12011 | cg06521562 | JRK; JRK | 127195 |
| −0.12013 | cg19678029 | KCNQ1OT1; KCNQ1; KCNQ1 | 354576 |
| −0.12018 | cg09915421 | | 189251 |
| −0.12024 | cg15827092 | | 291857 |
| −0.12026 | cg25827666 | SH2D2A; SH2D2A; NTRK1; | 451242 |
| | | NTRK1; SH2D2A; SH2D2A; | |
| | | SH2D2A | |
| −0.12033 | cg14166209 | | 262818 |
| −0.12033 | cg06647382 | PDK2 | 129663 |
| −0.12099 | cg13543096 | LOC100271831; GDPD3 | 249930 |
| −0.12099 | cg06679262 | | 130241 |
| −0.12103 | cg05514932 | RETSAT | 109417 |
| −0.12105 | cg22624391 | | 399439 |
| −0.12109 | cg19480385 | | 351330 |
| −0.12113 | cg08325842 | ZFP91; ZFP91-CNTF | 161555 |
| −0.12122 | cg15368872 | KLRK1 | 284353 |
| −0.12175 | cg27313492 | WFIKKN1 | 476470 |
| −0.12203 | cg05182583 | BAT2 | 103165 |
| −0.12205 | cg22193013 | CHRNA7 | 393284 |
| −0.12211 | cg16209444 | ACOX2 | 298173 |
| −0.12214 | cg20577102 | | 368824 |
| −0.12217 | cg06326425 | GPC6 | 123816 |
| −0.1226 | cg24353272 | RRP15 | 427604 |
| −0.12271 | cg06306791 | | 123517 |
| −0.12281 | cg11253913 | CALD1; CALD1; CALD1; | 212154 |
| | | CALD1; CALD1 | |
| −0.12285 | cg08352422 | | 161993 |
| −0.12293 | cg16044109 | HEG1 | 295491 |
| −0.12298 | cg23198815 | | 408873 |
| −0.12305 | cg23922753 | | 420530 |
| −0.12305 | cg15442583 | CDGAP | 285507 |
| −0.12317 | cg02248749 | TNXB | 45883 |
| −0.12323 | cg13455257 | TMOD4 | 248067 |
| −0.12337 | cg00740389 | ITGB7 | 15652 |
| −0.12369 | cg08448751 | SEMA3G | 163578 |
| −0.1237 | cg20663448 | FILIP1 | 370104 |
| −0.12374 | cg00819141 | | 17296 |
| −0.12375 | cg00161791 | | 3633 |
| −0.12376 | cg01569083 | TNF | 32491 |
| −0.12392 | cg00254017 | MAP3K1 | 5441 |
| −0.12398 | cg24885556 | ITGA7 | 436558 |
| −0.12399 | cg21082141 | PRDM8 | 376504 |
| −0.12415 | cg01785514 | TATDN3; TATDN3; TATDN3; | 36554 |
| | | TATDN3; TATDN3 | |
| −0.12442 | cg14771877 | C17orf99 | 274788 |
| −0.12455 | cg02866149 | C20orf117 | 58258 |
| −0.12464 | cg02667577 | WIPI2; WIPI2; WIPI2; WIPI2; | 54375 |
| | | WIPI2 | |
| −0.12507 | cg19973338 | C17orf99 | 359782 |
| −0.12517 | cg13709561 | SCARA5 | 253479 |
| −0.12585 | cg14118850 | HPCAL1; HPCAL1 | 261879 |
| −0.1259 | cg03728674 | | 75338 |
| −0.12611 | cg25340361 | COMMD1 | 443580 |
| −0.12613 | cg07347019 | | 143506 |
| −0.12631 | cg16000393 | TNK2; TNK2 | 294736 |
| −0.12649 | cg02898239 | | 58930 |
| −0.12655 | cg16323245 | ZBTB20; ZBTB20 | 300173 |
| −0.12655 | cg00907661 | ZDHHC20 | 19059 |
| −0.12665 | cg23986495 | | 421674 |
| −0.12691 | cg13720395 | YWHAG | 253710 |
| −0.12696 | cg00643876 | TBC1D24 | 13650 |
| −0.12697 | cg14781826 | BPTF; BPTF | 275014 |
| −0.12748 | cg01476807 | FBXO30 | 30709 |
| −0.1277 | cg19659642 | CD9 | 354321 |
| −0.12785 | cg21455901 | CRYL1 | 382161 |
| −0.1279 | cg01876531 | PLXNA2 | 38448 |
| −0.12817 | cg03925072 | | 78966 |
| −0.12858 | cg24267275 | | 426255 |
| −0.12861 | cg10356210 | KCNK3 | 197004 |
| −0.1289 | cg12145349 | NAA40 | 227108 |
| −0.12904 | cg11820824 | S100A16 | 221475 |
| −0.12906 | cg26989053 | NTAN1 | 471015 |
| −0.12914 | cg09119494 | E2F2 | 175337 |
| −0.12918 | cg18904855 | | 342756 |
| −0.12918 | cg00572560 | | 12164 |
| −0.12919 | cg25242423 | RTBDN; RTBDN | 442118 |
| −0.12926 | cg26745520 | GRK5 | 466632 |
| −0.12934 | cg07044523 | S100A5 | 137607 |
| −0.12938 | cg10048215 | APOLD1; APOLD1 | 191451 |
| −0.12949 | cg10471743 | | 198906 |
| −0.12978 | cg00767886 | EHD4 | 16225 |
| −0.12983 | cg26562772 | DUSP10; DUSP10; DUSP10 | 463554 |
| −0.13012 | cg00877958 | | 18503 |
| −0.13016 | cg21230535 | FTCD | 379161 |
| −0.13031 | cg09664445 | KIAA0664 | 184948 |
| −0.13045 | cg06708731 | DBH | 130785 |
| −0.13094 | cg11585805 | | 217365 |
| −0.13101 | cg10626556 | KIAA0406 | 201746 |
| −0.13101 | cg09982224 | ALDH3B1 | 190313 |
| −0.13118 | cg09437523 | | 181003 |
| −0.13123 | cg06769258 | MFSD7 | 132014 |
| −0.13123 | cg05299140 | | 105372 |
| −0.1315 | cg25735648 | | 449823 |
| −0.13164 | cg20865778 | TCIRG1; TCIRG1 | 373291 |
| −0.13179 | cg12450872 | | 231834 |
| −0.13196 | cg00154554 | RERE; RERE | 3460 |
| −0.1321 | cg10006897 | PCBP3; PCBP3 | 190741 |
| −0.13211 | cg14602234 | | 271884 |
| −0.13235 | cg08917489 | EYA4; EYA4; EYA4 | 172050 |
| −0.13261 | cg24088438 | FERMT3; FERMT3; FERMT3; | 423490 |
| | | FERMT3 | |
| −0.13277 | cg07325692 | | 143137 |
| −0.13288 | cg08013109 | ULK1 | 155629 |
| −0.13289 | cg09337391 | ZMYND8; ZMYND8; ZMYND8 | 179281 |
| −0.13298 | cg12430302 | SPSB1 | 231458 |
| −0.13311 | cg22912818 | TMCO3 | 404171 |
| −0.13319 | cg08338281 | WIBG | 161768 |
| −0.13321 | cg10097651 | | 192354 |
| −0.13345 | cg08122070 | NADK | 157631 |
| −0.13393 | cg10723746 | SPPL2B; SPPL2B | 203334 |
| −0.13398 | cg21656522 | | 385493 |
| −0.134 | cg24598094 | SLC30A1 | 431596 |
| −0.13423 | cg03466066 | | 70093 |
| −0.13424 | cg19503826 | EPN1; EPN1; EPN1 | 351690 |
| −0.13442 | cg18330041 | YWHAH | 333416 |
| −0.13456 | cg25707745 | | 449371 |
| −0.13486 | cg09018904 | ANO10 | 173744 |
| −0.13517 | cg07908868 | PLXNA2 | 153755 |
| −0.13522 | cg01364674 | | 28371 |
| −0.13527 | cg24995976 | PEAR1 | 438225 |
| −0.13549 | cg17802464 | SEC14L3 | 324476 |
| −0.13562 | cg23941016 | TBC1D2B; TBC1D2B | 420855 |
| −0.13568 | cg14504636 | AP2A2 | 269745 |
| −0.13571 | cg17846466 | MCOLN2 | 325303 |
| −0.13574 | cg16950726 | NLGN2 | 311097 |
| −0.13588 | cg07466788 | SLC16A3; SLC16A3; SLC16A3 | 145619 |
| −0.13613 | cg00767135 | ODZ3 | 16210 |
| −0.13616 | cg01336162 | TGFBR3 | 27842 |
| −0.13632 | cg05007126 | SDCBP2 | 99869 |
| −0.13637 | cg12045511 | EHD4 | 225319 |
| −0.13667 | cg18028470 | PIGV | 328228 |
| −0.13671 | cg15773893 | DOCK10 | 291034 |
| −0.13693 | cg05652528 | DNMT3A; DNMT3A; DNMT3A; | 111855 |
| | | MIR1301 | |
| −0.13703 | cg19931529 | UGP2; UGP2 | 359121 |
| −0.1372 | cg22488568 | SLC9A1 | 397378 |
| −0.13723 | cg14904034 | HPCAL1; HPCAL1 | 277047 |
| −0.13747 | cg27027230 | ARID5B | 471633 |
| −0.1377 | cg06607384 | C14orf159; C14orf159; C14orf159; | 128776 |
| | | C14orf159; C14orf159 | |
| −0.13773 | cg09766355 | NPL | 186590 |
| −0.13778 | cg17015522 | C1QTNF1; C1QTNF1; C1QTNF1 | 312055 |
| −0.13789 | cg19494960 | RPTOR; RPTOR | 351529 |
| −0.13793 | cg07366462 | ADCY5 | 143859 |
| −0.138 | cg04075247 | LGALS12; LGALS12; LGALS12; | 81844 |
| | | LGALS12; LGALS12 | |
| −0.13801 | cg01174811 | PDE4D; PDE4D; PDE4D | 24728 |
| −0.13825 | cg15536489 | C7orf50; C7orf50; C7orf50 | 286952 |
| −0.13832 | cg00622170 | C4orf10; NOP14 | 13248 |
| −0.13853 | cg08743794 | KIAA1949; NRM; KIAA1949 | 168897 |
| −0.13873 | cg21370522 | TNF | 381016 |
| −0.13878 | cg06047674 | FRMD4A | 119120 |
| −0.13896 | cg12225794 | | 228457 |
| −0.13921 | cg05905731 | | 116408 |
| −0.13924 | cg03407594 | MUC6 | 68950 |
| −0.13938 | cg26605914 | SKI | 464218 |
| −0.1394 | cg07359991 | HSD3B7; HSD3B7; HSD3B7; | 143753 |
| | | SETD1A | |
| −0.13946 | cg14780449 | | 274978 |
| −0.13949 | cg16843799 | FLJ39653; FLJ39653 | 309488 |
| −0.13957 | cg26805263 | TNFRSF19; TNFRSF19 | 467622 |
| −0.13961 | cg13460740 | MGC16703; P2RX6; P2RX6 | 248181 |
| −0.13977 | cg05488681 | | 108898 |
| −0.13998 | cg17810878 | ANO6; ANO6; ANO6 | 324639 |
| −0.14009 | cg12416561 | C4orf22 | 231221 |
| −0.14014 | cg03649589 | CSGALNACT1; CSGALNACT1; | 73790 |
| | | CSGALNACT1 | |
| −0.14031 | cg07877964 | USP7 | 153196 |
| −0.14044 | cg17499955 | | 320108 |
| −0.14047 | cg17407629 | PAM; PAM; PAM; PAM | 318547 |
| −0.14048 | cg04305228 | MICAL3 | 86473 |
| −0.14066 | cg24499411 | TNS3 | 429956 |
| −0.14066 | cg17125990 | | 313833 |
| −0.14069 | cg16608018 | | 305379 |
| −0.14074 | cg15053322 | SEC14L3 | 279556 |
| −0.14077 | cg11263420 | FCGRT; FCGRT | 212295 |
| −0.14081 | cg11437140 | TIMP2 | 215182 |
| −0.14083 | cg07328796 | | 143190 |
| −0.14111 | cg00528616 | ZNRF1 | 11235 |
| −0.14116 | cg13619044 | C7orf41 | 251590 |
| −0.14119 | cg03277153 | | 66388 |
| −0.14128 | cg22321646 | LZTS1 | 395060 |
| −0.14131 | cg11462252 | SLIT3 | 215511 |
| −0.14143 | cg02711975 | SNTG2 | 55231 |
| −0.14153 | cg18890417 | RHOBTB2; RHOBTB2; | 342552 |
| | | RHOBTB2 | |
| −0.14155 | cg19704102 | | 355015 |
| −0.14158 | cg01982724 | | 40687 |
| −0.14161 | cg01506917 | TRERF1 | 31222 |
| −0.14162 | cg13868106 | | 256590 |
| −0.14164 | cg27121538 | SV2B; SV2B | 473253 |
| −0.14179 | cg18657389 | EVPL | 338738 |
| −0.14191 | cg07044115 | | 137597 |
| −0.14228 | cg26775558 | TCF7L2; TCF7L2; TCF7L2; | 467042 |
| | | TCF7L2; TCF7L2; TCF7L2 | |
| −0.14233 | cg01860954 | | 38147 |
| −0.14249 | cg23951190 | CPNE4 | 421020 |
| −0.14255 | cg20721467 | MYST4 | 371233 |
| −0.14265 | cg12138483 | CLPP | 227002 |
| −0.14272 | cg11478024 | ANKS1A | 215738 |
| −0.14274 | cg16289449 | FERMT3; FERMT3; FERMT3; | 299541 |
| | | FERMT3 | |
| −0.14274 | cg14050363 | | 260484 |
| −0.14285 | cg03905247 | NCOR2; NCOR2 | 78635 |
| −0.14288 | cg08993079 | ADAM12; ADAM12 | 173319 |
| −0.14309 | cg16224727 | FBRSL1 | 298403 |
| −0.14309 | cg06385449 | FERMT3; FERMT3 | 124889 |
| −0.1432 | cg17594351 | MYF6 | 321588 |
| −0.14328 | cg12464631 | | 232068 |
| −0.14333 | cg04244932 | | 85279 |
| −0.14351 | cg04955877 | ZHX2 | 98883 |
| −0.14362 | cg07618453 | KCNQ1; KCNQ1 | 148575 |
| −0.14384 | cg27121309 | ACER3 | 473247 |
| −0.14384 | cg14807945 | ZEB2; ZEB2 | 275475 |
| −0.14392 | cg04142988 | PCSK6; PCSK6; PCSK6; PCSK6; | 83265 |
| | | PCSK6; PCSK6; PCSK6; | |
| | | PCSK6 | |
| −0.14429 | cg00925950 | PSKH1 | 19464 |
| −0.14432 | cg21933359 | KHDRBS3 | 389559 |
| −0.14433 | cg11287400 | MITF; MITF; MITF | 212678 |
| −0.14465 | cg00926267 | MKL1 | 19470 |
| −0.14468 | cg16098340 | MIR548C; RASSF3 | 296333 |
| −0.14478 | cg08630040 | | 166875 |
| −0.14489 | cg25880242 | | 451900 |
| −0.145 | cg09706277 | CD276; CD276 | 185655 |
| −0.14524 | cg07062388 | LMBRD1 | 137963 |
| −0.14533 | cg02470874 | PPM1L | 50305 |
| −0.14552 | cg00997251 | ARHGAP26; ARHGAP26 | 20995 |
| −0.1456 | cg16996242 | ZMYM4 | 311738 |
| −0.14564 | cg12316142 | DPYSL3 | 229823 |
| −0.14568 | cg05624853 | HSD3B7; HSD3B7; HSD3B7 | 111349 |
| −0.14588 | cg05433460 | ARHGAP1; ZNF408 | 108004 |
| −0.1463 | cg01226417 | | 25743 |
| −0.14632 | cg00063979 | | 1516 |
| −0.14661 | cg00498804 | ST5; ST5; ST5 | 10580 |
| −0.14682 | cg22132509 | PPM1L | 392374 |
| −0.14711 | cg22166248 | NXPH2 | 392926 |
| −0.14724 | cg02563600 | KIAA0182; KIAA0182 | 52166 |
| −0.14756 | cg12681001 | TNF | 235670 |
| −0.14759 | cg23590159 | UBXN11; UBXN11; UBXN11 | 415243 |
| −0.14761 | cg21528927 | SLC6A12; SLC6A12; SLC6A12 | 383331 |
| −0.1477 | cg02742775 | C12orf34 | 55869 |
| −0.14778 | cg10273072 | C21orf34 | 195585 |
| −0.14801 | cg22927076 | | 404363 |
| −0.14817 | cg03070550 | HTRA1 | 62547 |
| −0.14858 | cg18791929 | MYH15 | 340884 |
| −0.14861 | cg05991539 | C6orf25; C6orf25; C6orf25; | 118154 |
| | | C6orf25; C6orf25; C6orf25 | |
| −0.14869 | cg02092181 | DOT1L | 42966 |
| −0.1488 | cg13798679 | | 255238 |
| −0.14883 | cg17073306 | MYL5; MYL5 | 312976 |
| −0.1489 | cg17015179 | PPT1; PPT1 | 312048 |
| −0.14935 | cg01343936 | | 27981 |
| −0.14948 | cg11258484 | | 212233 |
| −0.14951 | cg13971140 | LASS4 | 258695 |
| −0.14961 | cg07282201 | TBC1D10C | 142343 |
| −0.14982 | cg12424504 | MACC1 | 231361 |
| −0.14985 | cg01312762 | CCDC134 | 27446 |
| −0.15027 | cg09568355 | | 183338 |
| −0.15031 | cg08800856 | NRXN2; NRXN2 | 169806 |
| −0.15034 | cg23617530 | | 415668 |
| −0.15048 | cg02076598 | KIAA1671 | 42633 |
| −0.15058 | cg18375707 | PLCB3 | 334103 |
| −0.1507 | cg15833565 | ERI3 | 291986 |
| −0.15079 | cg04101009 | | 82356 |
| −0.15089 | cg09302355 | FERMT3; FERMT3 | 178600 |
| −0.15093 | cg06191091 | | 121594 |
| −0.1512 | cg07812957 | | 152057 |
| −0.15121 | cg11724147 | HIBADH | 219728 |
| −0.15134 | cg27402999 | SLC9A9 | 477987 |
| −0.15154 | cg02814691 | NPL | 57253 |
| −0.15199 | cg03907570 | PRKAR1B; PRKAR1B; | 78679 |
| | | PRKAR1B; PRKAR1B; | |
| | | PRKAR1B; PRKAR1B | |
| −0.15217 | cg13166875 | SEC14L3 | 242924 |
| −0.15227 | cg26048923 | TRAPPC9; TRAPPC9 | 454532 |
| −0.15243 | cg12884495 | WBP2; UNC13D | 238717 |
| −0.15272 | cg23558764 | | 414863 |
| −0.15282 | cg05648614 | ARL8B | 111782 |
| −0.15283 | cg07449758 | C6orf25; C6orf25; C6orf25; | 145282 |
| | | C6orf25; C6orf25; C6orf25 | |
| −0.15287 | cg19642408 | | 354063 |
| −0.15303 | cg14831085 | PCSK6; PCSK6; PCSK6; PCSK6; | 275823 |
| | | PCSK6; PCSK6; PCSK6; | |
| | | PCSK6 | |
| −0.15304 | cg01882991 | | 38611 |
| −0.15305 | cg20848130 | GJC2 | 373060 |
| −0.15327 | cg16032111 | PPP2R1B; PPP2R1B | 295322 |
| −0.15331 | cg12268531 | HSP90B3P | 229089 |
| −0.15337 | cg08287887 | OR7E37P | 160856 |
| −0.15355 | cg14526997 | EHMT1; EHMT1 | 270270 |
| −0.15364 | cg06569229 | FAT3 | 128143 |
| −0.15382 | cg26485376 | | 462139 |
| −0.15389 | cg15513221 | | 286669 |
| −0.15389 | cg08577912 | | 165952 |
| −0.15392 | cg05778820 | SLC9A1 | 114033 |
| −0.15397 | cg02222066 | CYTH1; CYTH1 | 45356 |
| −0.15413 | cg02112681 | C7orf50; C7orf50; C7orf50 | 43376 |
| −0.15419 | cg02913511 | CNNM4 | 59256 |
| −0.15421 | cg25368116 | | 444093 |
| −0.15435 | cg01357135 | PPM1L | 28229 |
| −0.15439 | cg26139338 | KLF12 | 456089 |
| −0.15451 | cg19351617 | SIGIRR; SIGIRR; SIGIRR | 349235 |
| −0.15453 | cg22852065 | SPATA18 | 403114 |
| −0.15478 | cg14455925 | | 268761 |
| −0.15478 | cg00594561 | DLX3 | 12719 |
| −0.15482 | cg26114961 | C6orf25; C6orf25; C6orf25; | 455644 |
| | | C6orf25; C6orf25; C6orf25 | |
| −0.15511 | cg09996076 | HSD3B7; HSD3B7; HSD3B7; | 190546 |
| | | SETD1A | |
| −0.15544 | cg07821417 | ARVCF | 152230 |
| −0.1557 | cg23991482 | LSM7 | 421773 |
| −0.15611 | cg00751386 | CTNNA2; CTNNA2 | 15883 |
| −0.15614 | cg02316852 | RPA1 | 47305 |
| −0.15619 | cg21350115 | CALCRL; CALCRL | 380795 |
| −0.15672 | cg12526091 | | 233088 |
| −0.1568 | cg02481950 | METTL9; IGSF6; METTL9 | 50548 |
| −0.157 | cg19019345 | ST5 | 344432 |
| −0.15712 | cg13552337 | NDOR1; NDOR1; NDOR1; | 250119 |
| | | NDOR1 | |
| −0.15733 | cg19321263 | TRIM26 | 348840 |
| −0.15767 | cg07506749 | | 146418 |
| −0.15773 | cg26197679 | | 457116 |
| −0.15792 | cg03680790 | STX2; STX2 | 74392 |
| −0.15802 | cg05744888 | EIF2C2; EIF2C2 | 113460 |
| −0.1583 | cg00433709 | ZNRF1 | 9174 |
| −0.15835 | cg22856279 | | 403194 |
| −0.15845 | cg15256491 | FOXK1 | 282829 |
| −0.15854 | cg20171297 | MYF6; MYF6 | 362835 |
| −0.15858 | cg11244402 | FOXO1 | 211986 |
| −0.15879 | cg14510322 | | 269863 |
| −0.15902 | cg24236839 | KDM4B | 425869 |
| −0.15907 | cg01353907 | GPR133 | 28172 |
| −0.15926 | cg23935522 | ZDHHC17 | 420759 |
| −0.15938 | cg01893068 | | 38791 |
| −0.15944 | cg20432350 | KCND3; KCND3 | 366636 |
| −0.15948 | cg07453773 | ST5; ST5; ST5 | 145358 |
| −0.15956 | cg11370814 | | 214057 |
| −0.15957 | cg07820423 | | 152210 |
| −0.15972 | cg00550928 | MGAT3; MGAT3 | 11698 |
| −0.15977 | cg16576811 | NEDD4L; NEDD4L; NEDD4L; | 304754 |
| | | NEDD4L; NEDD4L; NEDD4L; | |
| | | NEDD4L; NEDD4L; NEDD4L | |
| −0.15996 | cg03285617 | NT5E | 66533 |
| −0.1605 | cg08145262 | FRS2; FRS2 | 158124 |
| −0.16083 | cg07791897 | | 151636 |
| −0.16096 | cg13127591 | KRTAP9-9; KRTAP9-8 | 242341 |
| −0.1612 | cg00029979 | | 743 |
| −0.1613 | cg18702278 | PDE6B; PDE6B; PDE6B | 339372 |
| −0.16132 | cg25683744 | NINJ2 | 449043 |
| −0.16135 | cg13675051 | RNF19A; RNF19A | 252709 |
| −0.16155 | cg20364025 | GALNS | 365624 |
| −0.16174 | cg22649800 | OGDH; OGDH | 399813 |
| −0.16179 | cg07692929 | OLFML1 | 149840 |
| −0.16208 | cg02903929 | | 59057 |
| −0.16229 | cg10447977 | ADAMTS9 | 198519 |
| −0.1623 | cg05383524 | MGRN1; MGRN1; MGRN1; | 107108 |
| | | MGRN1 | |
| −0.16237 | cg25902666 | CSNK1G2 | 452187 |
| −0.16237 | cg07662964 | BRD1; LOC90834 | 149327 |
| −0.16243 | cg14299931 | ARHGEF7; ARHGEF7; | 265726 |
| | | ARHGEF7; ARHGEF7; | |
| | | ARHGEF7; ARHGEF7 | |
| −0.16257 | cg02017814 | NSMCE2 | 41390 |
| −0.16265 | cg01703196 | | 34921 |
| −0.16293 | cg02119955 | ITGBL1 | 43511 |
| −0.16305 | cg17987982 | | 327555 |
| −0.1631 | cg02449461 | ARRB1; ARRB1 | 49866 |
| −0.16315 | cg03928874 | LY86 | 79027 |
| −0.16339 | cg07735586 | | 150671 |
| −0.1634 | cg21089930 | DTX4 | 376611 |
| −0.16371 | cg25105578 | | 440013 |
| −0.16389 | cg04021454 | TAF12; TAF12 | 80874 |
| −0.16408 | cg21126020 | TRIM10; TRIM10 | 377162 |
| −0.16417 | cg27395226 | SRPK2 | 477869 |
| −0.16428 | cg09141623 | PRKG1; PRKG1 | 175757 |
| −0.16437 | cg16746362 | C6orf25; C6orf25; C6orf25; | 307984 |
| | | C6orf25; C6orf25; C6orf25 | |
| −0.16464 | cg03273420 | SMCR7; SMCR7; SMCR7 | 66322 |
| −0.16496 | cg04221461 | AKT3; AKT3 | 84805 |
| −0.16524 | cg13573761 | C4orf23 | 250625 |
| −0.1653 | cg17807913 | | 324584 |
| −0.16531 | cg19506253 | CYTIP | 351732 |
| −0.16538 | cg00895231 | | 18812 |
| −0.16547 | cg00671823 | GTF3C1 | 14204 |
| −0.16584 | cg23632029 | OR10A5 | 415943 |
| −0.16593 | cg04345118 | PAX8; PAX8; LOC654433; | 87257 |
| | | PAX8; PAX8; PAX8; LOC440839 | |
| −0.16595 | cg09228831 | GPRIN3 | 177227 |
| −0.16609 | cg05910124 | TRAPPC9; TRAPPC9 | 116485 |
| −0.16622 | cg15124432 | CDC34 | 280793 |
| −0.16631 | cg03199366 | | 64930 |
| −0.16651 | cg21524538 | SDCBP2 | 383268 |
| −0.16652 | cg22950982 | MAPKAPK3 | 404771 |
| −0.1666 | cg26807536 | LOC100271831; GDPD3 | 467662 |
| −0.16661 | cg16618389 | | 305550 |
| −0.16665 | cg18347607 | | 333691 |
| −0.16674 | cg24828746 | ZBTB7A | 435490 |
| −0.16708 | cg01386735 | LMBRD1 | 28819 |
| −0.16723 | cg12214366 | ANTXR2; ANTXR2 | 228267 |
| −0.16727 | cg04132452 | PPM1L | 83027 |
| −0.1674 | cg17923377 | BRD1; LOC90834 | 326503 |
| −0.1675 | cg00659931 | PCSK6; PCSK6; PCSK6; PCSK6; | 13955 |
| | | PCSK6; PCSK6; PCSK6; | |
| | | PCSK6 | |
| −0.16751 | cg01412400 | RPTOR; RPTOR | 29361 |
| −0.16763 | cg18507146 | ZNF236 | 336390 |
| −0.16773 | cg17001765 | PGBD2; PGBD2 | 311841 |
| −0.16781 | cg23683146 | | 416923 |
| −0.16815 | cg22688687 | C19orf42 | 400457 |
| −0.16825 | cg10969245 | DAGLA | 207350 |
| −0.16826 | cg15637527 | ARHGAP1 | 288736 |
| −0.16829 | cg11903920 | TCERG1L | 223003 |
| −0.1685 | cg15268191 | | 283001 |
| −0.16862 | cg03204322 | SAMD13; SAMD13; SAMD13 | 65015 |
| −0.16866 | cg09906928 | LHFPL2 | 189108 |
| −0.1688 | cg23808165 | | 418718 |
| −0.16903 | cg15594550 | ZNF696 | 287945 |
| −0.16921 | cg08462367 | | 163870 |
| −0.16922 | cg07412186 | ATXN1; ATXN1 | 144647 |
| −0.16923 | cg02988730 | AMPD2; AMPD2; AMPD2 | 60882 |
| −0.16929 | cg20090290 | AMBRA1 | 361565 |
| −0.16937 | cg25296103 | ZEB2; ZEB2 | 442878 |
| −0.1694 | cg25124489 | PRKCE | 440332 |
| −0.1695 | cg07739478 | C6orf25; C6orf25; C6orf25; | 150742 |
| | | C6orf25; C6orf25; C6orf25 | |
| −0.16956 | cg10090414 | | 192177 |
| −0.16978 | cg08937102 | NADK | 172382 |
| −0.16999 | cg10785394 | | 204327 |
| −0.17008 | cg05425699 | TNS3 | 107909 |
| −0.1701 | cg12987138 | | 240257 |
| −0.17028 | cg04558553 | UGT2B7 | 91298 |
| −0.17028 | cg21764632 | WIPI2; WIPI2; WIPI2; WIPI2; | 386908 |
| | | WIPI2 | |
| −0.17032 | cg01306151 | C16orf7 | 27295 |
| −0.17039 | cg22718891 | | 400997 |
| −0.17042 | cg21346154 | TGFA; TGFA | 380741 |
| −0.17048 | cg21241151 | | 379374 |
| −0.17049 | cg25091527 | COL4A1 | 439775 |
| −0.17072 | cg26581714 | SPATA18 | 463898 |
| −0.17077 | cg04771838 | COL4A2 | 95213 |
| −0.17083 | cg04988694 | TBCD | 99510 |
| −0.17083 | cg05697734 | | 112662 |
| −0.17096 | cg25221919 | | 441857 |
| −0.17096 | cg18071202 | LOC100292680 | 328953 |
| −0.17113 | cg19184688 | | 346756 |
| −0.17114 | cg13740185 | C10orf105; CDH23 | 254087 |
| −0.17123 | cg05801329 | | 114496 |
| −0.17126 | cg26576576 | PTPN14 | 463811 |
| −0.17157 | cg14692453 | ANKRD23 | 273553 |
| −0.17158 | cg10143416 | | 193223 |
| −0.17163 | cg05856321 | | 115416 |
| −0.17167 | cg00230120 | LOC100292680 | 4919 |
| −0.17176 | cg24152251 | | 424608 |
| −0.17178 | cg18030453 | LARS2 | 328261 |
| −0.17206 | cg25749107 | | 450067 |
| −0.17211 | cg26457569 | KANK2; KANK2 | 461638 |
| −0.17223 | cg08890627 | SEC14L1; SEC14L1; SEC14L1; | 171586 |
| | | SEC14L1; SEC14L1 | |
| −0.17237 | cg22481643 | C3orf26; FILIP1L; MIR548G; | 397283 |
| | | FILIP1L; FILIP1L | |
| −0.17247 | cg08791347 | FRMD4A | 169648 |
| −0.17248 | cg08656333 | | 167353 |
| −0.17277 | cg14121740 | | 261943 |
| −0.17277 | cg05514531 | GBE1 | 109409 |
| −0.17287 | cg00045902 | PWWP2B; PWWP2B | 1131 |
| −0.17294 | cg20584712 | BAT2 | 368914 |
| −0.17333 | cg09305096 | GULP1 | 178661 |
| −0.17333 | cg09156542 | | 176059 |
| −0.17338 | cg05660874 | SPPL2B; SPPL2B | 112045 |
| −0.17344 | cg01397912 | C19orf51; TNNI3 | 29052 |
| −0.17357 | cg19164997 | CAPN5 | 346507 |
| −0.17408 | cg26110562 | AGAP1; AGAP1 | 455549 |
| −0.17424 | cg19583934 | RASA3 | 352999 |
| −0.17429 | cg20319604 | PPM1B; PPM1B; PPM1B; | 364994 |
| | | PPM1B; PPM1B | |
| −0.17454 | cg02955940 | CORIN | 60186 |
| −0.17456 | cg05426956 | HTRA1 | 107922 |
| −0.17468 | cg00128100 | KIAA1949; NRM; KIAA1949 | 2929 |
| −0.17519 | cg01786151 | | 36566 |
| −0.17536 | cg20788482 | | 372245 |
| −0.17539 | cg04724873 | KIAA1751 | 94344 |
| −0.17547 | cg11473240 | KRT31 | 215681 |
| −0.17547 | cg10539122 | NAV1; MIR1231; NAV1 | 200240 |
| −0.17549 | cg02123534 | ADAP1 | 43564 |
| −0.17597 | cg05776861 | ARHGEF7; ARHGEF7; | 113995 |
| | | ARHGEF7; ARHGEF7; | |
| | | ARHGEF7 | |
| −0.17599 | cg01104590 | CARS; CARS; CARS; CARS | 23322 |
| −0.17623 | cg24888581 | AGAP1; AGAP1 | 436607 |
| −0.17625 | cg18805457 | TCERG1; TCERG1 | 341130 |
| −0.17669 | cg08189139 | | 158940 |
| −0.17672 | cg17644903 | ZDBF2 | 322224 |
| −0.17677 | cg20725334 | | 371301 |
| −0.1769 | cg00851627 | | 17937 |
| −0.177 | cg05891054 | STAT5B | 116105 |
| −0.17702 | cg13609694 | | 251374 |
| −0.17731 | cg19482843 | EIF2C2; EIF2C2 | 351362 |
| −0.17753 | cg19975759 | B3GNTL1 | 359813 |
| −0.17754 | cg24864413 | SLC22A1; SLC22A1 | 436118 |
| −0.1776 | cg10592521 | MEX3D | 201161 |
| −0.17783 | cg21548131 | NLGN1 | 383703 |
| −0.178 | cg02971328 | RAC1; RAC1 | 60519 |
| −0.17804 | cg04857881 | ITFG1 | 96921 |
| −0.17826 | cg16653138 | PRKCZ; PRKCZ; PRKCZ | 306192 |
| −0.17831 | cg22146612 | | 392580 |
| −0.17849 | cg17627813 | PRDM10; PRDM10; PRDM10; | 322003 |
| | | PRDM10 | |
| −0.17863 | cg24522085 | LASS6 | 430348 |
| −0.17898 | cg02374207 | MACROD1 | 48511 |
| −0.17927 | cg01286133 | APP; APP; APP; APP; APP | 26850 |
| −0.17931 | cg04644504 | SENP8; MYO9A | 92888 |
| −0.17974 | cg01923312 | SLC22A3 | 39391 |
| −0.1805 | cg25541708 | | 446789 |
| −0.1806 | cg15852553 | | 292348 |
| −0.18062 | cg18722504 | HBEGF | 339716 |
| −0.18072 | cg20440552 | | 366770 |
| −0.18085 | cg12979590 | CUGBP1 | 240142 |
| −0.18112 | cg18664915 | C7orf50; C7orf50; C7orf50 | 338829 |
| −0.18131 | cg18080303 | LAMA4; LAMA4; LAMA4; | 329086 |
| | | LAMA4; LAMA4 | |
| −0.18135 | cg12496975 | RAPGEF3; RAPGEF3; RAPGEF3 | 232622 |
| −0.18167 | cg25677376 | | 448951 |
| −0.18198 | cg06476926 | TBCD | 126410 |
| −0.18258 | cg16161418 | KIAA1949; KIAA1949 | 297331 |
| −0.18265 | cg14661139 | UBE2J2; UBE2J2; | 273045 |
| | | LOC100128842; UBE2J2; | |
| | | UBE2J2 | |
| −0.18273 | cg07930874 | SLAIN1; SLAIN1 | 154214 |
| −0.18302 | cg13912858 | SORT1 | 257507 |
| −0.18327 | cg06059810 | RUFY3; RUFY3; RUFY3; | 119317 |
| | | RUFY3; RUFY3 | |
| −0.18359 | cg01822532 | LRP5 | 37414 |
| −0.18366 | cg05059607 | PITPNC1; PITPNC1 | 100893 |
| −0.18367 | cg07547798 | NSMCE2 | 147165 |
| −0.18371 | cg04761972 | | 95031 |
| −0.18382 | cg15932980 | STX2; STX2 | 293635 |
| −0.18394 | cg17425351 | | 318837 |
| −0.18398 | cg09777237 | ELN; ELN; ELN; ELN; ELN | 186754 |
| −0.18403 | cg04068914 | MTHFSD; MTHFSD; MTHFSD; | 81718 |
| | | MTHFSD; MTHFSD; MTHFSD; | |
| | | MTHFSD; FLJ30679 | |
| −0.18411 | cg07224918 | ARHGEF10L; ARHGEF10L | 141354 |
| −0.1845 | cg24269082 | TBCD | 426275 |
| −0.18457 | cg00646241 | SHANK2 | 13697 |
| −0.18467 | cg24290996 | | 426537 |
| −0.18474 | cg13690679 | | 253081 |
| −0.18479 | cg18926409 | | 343073 |
| −0.18495 | cg19589494 | | 353114 |
| −0.18496 | cg12806353 | PLA1A | 237540 |
| −0.1853 | cg00237010 | NINJ2 | 5072 |
| −0.18559 | cg04144521 | MFSD6 | 83296 |
| −0.18565 | cg07833951 | | 152463 |
| −0.18583 | cg04056576 | PPM1L | 81465 |
| −0.18583 | cg14837896 | THBS4 | 275954 |
| −0.18588 | cg22330533 | BRD1; LOC90834 | 395190 |
| −0.18592 | cg03659680 | LYN; LYN | 73997 |
| −0.18618 | cg06795960 | ARL4D | 132561 |
| −0.1862 | cg14679230 | LIPE | 273341 |
| −0.18622 | cg00873517 | KHDRBS3 | 18398 |
| −0.18626 | cg13613044 | | 251444 |
| −0.18636 | cg20021790 | RPH3AL | 360547 |
| −0.1866 | cg08279097 | DCLRE1B | 160695 |
| −0.18672 | cg13613439 | PHYHD1; PHYHD1; PHYHD1; | 251453 |
| | | PHYHD1; PHYHD1 | |
| −0.18676 | cg25331919 | LIPG | 443459 |
| −0.18684 | cg16046833 | SNX7; SNX7 | 295544 |
| −0.18685 | cg02535674 | | 51638 |
| −0.18728 | cg27554156 | GSG1; GSG1; GSG1; GSG1 | 480508 |
| −0.18728 | cg24940885 | | 437453 |
| −0.1873 | cg02360002 | | 48201 |
| −0.18738 | cg04912273 | SLC9A1 | 98039 |
| −0.18751 | cg18321354 | CARD11 | 333265 |
| −0.18756 | cg10453419 | PCSK6; PCSK6; PCSK6; PCSK6; | 198614 |
| | | PCSK6; PCSK6; PCSK6; | |
| | | PCSK6 | |
| −0.18757 | cg04833731 | GDPD3; GDPD3; LOC100271831 | 96391 |
| −0.18789 | cg16872841 | COL4A2 | 309904 |
| −0.18796 | cg08176694 | PITPNM2 | 158725 |
| −0.1881 | cg08426898 | | 163219 |
| −0.18822 | cg02813863 | APOLD1; APOLD1 | 57236 |
| −0.1885 | cg02959282 | NCK1 | 60268 |
| −0.1885 | cg11458974 | HMOX1 | 215460 |
| −0.18885 | cg01156373 | ERBB4; ERBB4 | 24354 |
| −0.18902 | cg08405910 | NCK2; NCK2; NCK2 | 162877 |
| −0.18904 | cg06445944 | PIK3R1; PIK3R1; PIK3R1 | 125887 |
| −0.18909 | cg19623595 | | 353718 |
| −0.18909 | cg05145297 | HIP1 | 102438 |
| −0.18938 | cg11626175 | JARID2 | 218013 |
| −0.18939 | cg05310046 | THSD4 | 105621 |
| −0.18981 | cg26005086 | JAG1 | 453833 |
| −0.18991 | cg09990852 | RUFY3; RUFY3; RUFY3; | 190465 |
| | | RUFY3; RUFY3 | |
| −0.19005 | cg23413567 | FOXO1 | 412578 |
| −0.19014 | cg11815008 | CETN1 | 221367 |
| −0.19024 | cg14883993 | NFIC; NFIC | 276772 |
| −0.1905 | cg27302175 | ZSWIM6 | 476195 |
| −0.19064 | cg05647929 | CFC1B; CFC1 | 111770 |
| −0.19077 | cg00859622 | VSTM2L | 18096 |
| −0.19081 | cg25940447 | GNPDA1 | 452774 |
| −0.19085 | cg00213052 | | 4580 |
| −0.19087 | cg03057216 | VPS72; TMOD4 | 62283 |
| −0.19087 | cg01932663 | C3orf26; FILIP1L; FILIP1L | 39583 |
| −0.19118 | cg25026329 | APLP2; APLP2; APLP2; APLP2; | 438829 |
| | | APLP2; APLP2 | |
| −0.1912 | cg06758827 | TRIO | 131795 |
| −0.19133 | cg09353563 | UACA; UACA; UACA | 179511 |
| −0.19136 | cg20629021 | CPT1A; CPT1A | 369542 |
| −0.19145 | cg07823562 | CYTH1; CYTH1 | 152263 |
| −0.19146 | cg23429240 | CORO1C | 412803 |
| −0.19147 | cg03320743 | | 67233 |
| −0.19215 | cg10407488 | ARHGEF12 | 197843 |
| −0.19222 | cg19876649 | MYOM1; MYOM1 | 358154 |
| −0.19233 | cg13148151 | LIMD1 | 242630 |
| −0.19234 | cg24073770 | | 423219 |
| −0.19257 | cg08010094 | NXPH2 | 155586 |
| −0.19262 | cg01742357 | RAPGEF4; RAPGEF4 | 35676 |
| −0.19263 | cg05957190 | CHST11 | 117447 |
| −0.19286 | cg07394266 | C9orf71 | 144374 |
| −0.19326 | cg00689612 | TIMP2 | 14559 |
| −0.19327 | cg12131324 | RPTOR; RPTOR | 226898 |
| −0.19329 | cg07155381 | RPH3AL | 139908 |
| −0.1934 | cg13189271 | JARID2 | 243319 |
| −0.19379 | cg03447908 | CLEC3B | 69735 |
| −0.1938 | cg19185706 | LDB3; LDB3; LDB3 | 346776 |
| −0.19452 | cg12744888 | MYH15 | 236621 |
| −0.19464 | cg04081835 | | 81973 |
| −0.1948 | cg05297666 | ETS1 | 105349 |
| −0.19506 | cg19252218 | ARL4D | 347770 |
| −0.19508 | cg11668573 | FNIP2 | 218773 |
| −0.19509 | cg07879785 | CRYL1 | 153222 |
| −0.19518 | cg04005958 | | 80561 |
| −0.19522 | cg01617074 | SLC45A4; SLC45A4 | 33454 |
| −0.19532 | cg12560421 | | 233610 |
| −0.19534 | cg00498089 | | 10560 |
| −0.19543 | cg17622952 | | 321946 |
| −0.1955 | cg05541460 | | 109873 |
| −0.19558 | cg02073224 | MEGF6 | 42570 |
| −0.19569 | cg07983363 | PCSK6; PCSK6; PCSK6; PCSK6; | 155143 |
| | | PCSK6; PCSK6; PCSK6; | |
| | | PCSK6 | |
| −0.19582 | cg00989315 | CDCA7L; CDCA7L; CDCA7L | 20808 |
| −0.19585 | cg11634496 | | 218142 |
| −0.19592 | cg14887955 | | 276836 |
| −0.19603 | cg10345724 | C7orf50; C7orf50; C7orf50 | 196843 |
| −0.1963 | cg14002578 | GALNT12 | 259402 |
| −0.19635 | cg02670123 | ITGA6; ITGA6 | 54419 |
| −0.19673 | cg12056017 | ANKRD44 | 225506 |
| −0.19679 | cg03462604 | C2orf18 | 70013 |
| −0.1968 | cg02800733 | CD320; CD320 | 57003 |
| −0.19696 | cg18835549 | TRIM40 | 341642 |
| −0.19701 | cg08079387 | LOC90834; BRD1 | 156793 |
| −0.19706 | cg22251117 | RPH3AL | 394137 |
| −0.19709 | cg02382878 | | 48672 |
| −0.1974 | cg14710529 | ASAP2; ASAP2 | 273872 |
| −0.19767 | cg01891925 | BTC | 38772 |
| −0.19768 | cg20439101 | MAP3K1 | 366753 |
| −0.19769 | cg06709178 | INPP5B | 130792 |
| −0.19814 | cg07573020 | ANKH | 147681 |
| −0.19821 | cg08373573 | MUC6 | 162363 |
| −0.19832 | cg27396760 | CMIP; CMIP | 477898 |
| −0.19891 | cg05796178 | LZTS1 | 114384 |
| −0.19909 | cg25345520 | | 443680 |
| −0.19912 | cg21562035 | KANK2; KANK2 | 383932 |
| −0.19945 | cg06136573 | | 120675 |
| −0.19954 | cg18756557 | LOC100292680 | 340244 |
| −0.19966 | cg06049177 | GSR | 119151 |
| −0.19972 | cg24536145 | SORBS2 | 430636 |
| −0.19978 | cg16875057 | STK39 | 309942 |
| −0.20006 | cg01698392 | TIMP2 | 34835 |
| −0.2003 | cg12251803 | LRRFIP1; LRRFIP1; LRRFIP1; | 228847 |
| | | LRRFIP1; LRRFIP1 | |
| −0.20035 | cg21007971 | PLA2G6; BAIAP2L2; PLA2G6 | 375427 |
| −0.20045 | cg04142824 | ATP11A; ATP11A | 83262 |
| −0.20048 | cg00435616 | C7orf50; C7orf50; C7orf50 | 9208 |
| −0.20064 | cg04094923 | | 82221 |
| −0.20076 | cg13496793 | ZBTB34 | 249014 |
| −0.20101 | cg23659250 | BRD1; LOC90834 | 416462 |
| −0.20138 | cg15690822 | RFFL; RFFL | 289648 |
| −0.2019 | cg02019508 | ATF7; ATF7; ATF7 | 41425 |
| −0.20196 | cg08577298 | | 165938 |
| −0.20213 | cg25238480 | GALNTL4 | 442087 |
| −0.20221 | cg12294678 | NOS3 | 229476 |
| −0.20232 | cg21881338 | AACS | 388735 |
| −0.2025 | cg24901662 | PARD3 | 436800 |
| −0.20251 | cg11407540 | EGF | 214668 |
| −0.20269 | cg07069839 | | 138124 |
| −0.20308 | cg21931263 | | 389515 |
| −0.20316 | cg11561301 | C4orf6 | 216983 |
| −0.20337 | cg07795513 | ATF6 | 151706 |
| −0.20362 | cg04589673 | | 91937 |
| −0.20374 | cg06418113 | RNF165 | 125411 |
| −0.20385 | cg01471802 | TRIM6- | 30616 |
| | | TRIM34; TRIM6; TRIM6 | |
| −0.20416 | cg15884713 | NINJ2 | 292821 |
| −0.2046 | cg22043118 | TMED7- | 391069 |
| | | TICAM2; TICAM2; TMED7- | |
| | | TICAM2 | |
| −0.20502 | cg07593326 | PCCA; PCCA | 148106 |
| −0.20504 | cg12800012 | SH3PXD2A | 237419 |
| −0.20507 | cg04667640 | ZFHX3; ZFHX3 | 93314 |
| −0.20511 | cg00527307 | | 11212 |
| −0.2055 | cg13300630 | ROBO1; ROBO1; ROBO1; | 245107 |
| | | ROBO1 | |
| −0.20558 | cg25888386 | | 452027 |
| −0.2057 | cg03571277 | ACCN1 | 72232 |
| −0.20574 | cg19660729 | KIF26A | 354335 |
| −0.206 | cg13689563 | ZNF136 | 253055 |
| −0.20627 | cg27585074 | CSNK1G2 | 481080 |
| −0.20638 | cg25266816 | | 442457 |
| −0.20669 | cg15984712 | | 294427 |
| −0.2068 | cg08124461 | CGNL1 | 157691 |
| −0.20692 | cg12555652 | ITGBL1 | 233545 |
| −0.20701 | cg06707286 | PCSK6; PCSK6; PCSK6; PCSK6; | 130749 |
| | | PCSK6; PCSK6; PCSK6; | |
| | | PCSK6 | |
| −0.20713 | cg12758625 | RNF24; RNF24; RNF24 | 236809 |
| −0.20719 | cg16241878 | FRMD5 | 298677 |
| −0.20733 | cg06389019 | SLC9A3R1 | 124935 |
| −0.20737 | cg02803847 | BUB3; BUB3 | 57065 |
| −0.20737 | cg07376547 | | 144035 |
| −0.20746 | cg20625588 | MUC6 | 369496 |
| −0.20756 | cg05415840 | ARC; ARC | 107735 |
| −0.2077 | cg00311751 | TRERF1 | 6568 |
| −0.20775 | cg02465887 | DCHS2 | 50200 |
| −0.20799 | cg02392737 | ADCY9 | 48895 |
| −0.20804 | cg13043815 | C21orf63 | 241083 |
| −0.20812 | cg06232466 | CACNA1H; CACNA1H | 122343 |
| −0.20827 | cg08490246 | | 164404 |
| −0.20838 | cg05964487 | | 117593 |
| −0.20854 | cg15891850 | KLF12 | 292938 |
| −0.20874 | cg20218678 | FAM10A4 | 363445 |
| −0.20882 | cg01569709 | | 32505 |
| −0.20902 | cg15044010 | SRPK2; SRPK2 | 279419 |
| −0.20915 | cg10269431 | EGF | 195527 |
| −0.20964 | cg01064758 | TG; SLA; SLA | 22444 |
| −0.21022 | cg12597325 | STX2; STX2 | 234249 |
| −0.21045 | cg20263733 | ATP2C1; ATP2C1; ATP2C1; | 364115 |
| | | ATP2C1 | |
| −0.21084 | cg02872914 | CTBP2; CTBP2; CTBP2 | 58381 |
| −0.21116 | cg14153069 | PHYHD1; PHYHD1; PHYHD1; | 262529 |
| | | PHYHD1; PHYHD1 | |
| −0.21127 | cg21617357 | HTATIP2; HTATIP2; HTATIP2; | 384851 |
| | | HTATIP2; HTATIP2 | |
| −0.2114 | cg23723013 | KCNAB1; KCNAB1; KCNAB1 | 417619 |
| −0.21172 | cg02497428 | METTL9; IGSF6; METTL9 | 50892 |
| −0.2118 | cg12018521 | TES | 224833 |
| −0.21202 | cg02773403 | ANK3 | 56524 |
| −0.21204 | cg00765737 | COL4A2 | 16178 |
| −0.21216 | cg06432487 | | 125644 |
| −0.21225 | cg13447820 | | 247908 |
| −0.21276 | cg04470324 | FSTL4 | 89614 |
| −0.21292 | cg26295272 | FLNB; FLNB; FLNB; FLNB | 458803 |
| −0.21314 | cg05450916 | TGFBR2; TGFBR2 | 108229 |
| −0.21317 | cg04893454 | | 97666 |
| −0.21341 | cg22219255 | | 393632 |
| −0.21349 | cg23198558 | ARHGAP26; ARHGAP26 | 408866 |
| −0.21367 | cg19353947 | ZEB2; ZEB2; ZEB2 | 349264 |
| −0.21373 | cg04208996 | | 84534 |
| −0.2138 | cg02534659 | | 51624 |
| −0.21385 | cg04266607 | JAZF1 | 85718 |
| −0.21392 | cg07042532 | CREB5; CREB5; CREB5 | 137576 |
| −0.21393 | cg09130535 | ADRBK1 | 175551 |
| −0.21397 | cg24919468 | | 437059 |
| −0.21428 | cg00058644 | | 1391 |
| −0.2144 | cg06127541 | RRAS2; RRAS2 | 120526 |
| −0.21446 | cg16497661 | CKB | 303238 |
| −0.21492 | cg02712587 | EIF2AK4 | 55252 |
| −0.21494 | cg00232092 | FBXL18 | 4975 |
| −0.21509 | cg13679772 | FOXN3 | 252820 |
| −0.21555 | cg01915822 | ADCK5 | 39234 |
| −0.21558 | cg11460562 | TNPO1; TNPO1 | 215482 |
| −0.21561 | cg14375985 | CTBP2; CTBP2; CTBP2 | 267187 |
| −0.21562 | cg04267284 | PYGB | 85734 |
| −0.21585 | cg19093405 | CACNA2D3 | 345515 |
| −0.21621 | cg02668218 | | 54387 |
| −0.21671 | cg25286715 | | 442752 |
| −0.21697 | cg14772660 | SLC5A7 | 274806 |
| −0.21707 | cg01495332 | GRB10; GRB10; GRB10 | 31016 |
| −0.21758 | cg00446697 | HCCA2 | 9405 |
| −0.21821 | cg03844838 | DIP2C | 77475 |
| −0.21834 | cg25799348 | FOXK1 | 450816 |
| −0.21849 | cg08702689 | PRRX1; PRRX1 | 168159 |
| −0.21863 | cg13495373 | CREB5; CREB5; CREB5 | 248982 |
| −0.21887 | cg11165960 | | 210588 |
| −0.21906 | cg24721647 | ACSL1 | 433758 |
| −0.21922 | cg15380511 | | 284521 |
| −0.21944 | cg07739826 | OSBP | 150749 |
| −0.21962 | cg18780243 | EEF1DP3 | 340687 |
| −0.21966 | cg13289202 | ABTB2 | 244906 |
| −0.21972 | cg00684423 | GPER; C7orf50; C7orf50; GPER; | 14439 |
| | | C7orf50 | |
| −0.21994 | cg06013113 | RAB40C | 118547 |
| −0.21999 | cg18129282 | | 330053 |
| −0.22012 | cg26325949 | SYNPO2; SYNPO2; SYNPO2 | 459352 |
| −0.22027 | cg07091154 | SHANK2 | 138518 |
| −0.22031 | cg27584112 | | 481064 |
| −0.22034 | cg20203400 | TMEM182 | 363230 |
| −0.22065 | cg14125542 | FAM49A | 262013 |
| −0.22071 | cg00380924 | RPS6KA2; RPS6KA2 | 8060 |
| −0.22081 | cg18887296 | PPFIA3 | 342507 |
| −0.22107 | cg17858500 | C20orf27 | 325500 |
| −0.22142 | cg04974647 | | 99207 |
| −0.22165 | cg20334115 | PYCR2 | 365215 |
| −0.22175 | cg16523645 | ITPK1; ITPK1; ITPK1 | 303721 |
| −0.22212 | cg12525096 | | 233069 |
| −0.22213 | cg20131219 | PHLDB2; PHLDB2; PHLDB2; | 362199 |
| | | PHLDB2 | |
| −0.22244 | cg21750426 | MIRLET7C; C21orf34; C21orf34; | 386667 |
| | | MIR99A | |
| −0.22263 | cg17918796 | | 326430 |
| −0.22269 | cg14165772 | | 262803 |
| −0.22294 | cg14081251 | ST6GALNAC4; ST6GALNAC4 | 261114 |
| −0.22318 | cg01824625 | EPHA3; EPHA3 | 37465 |
| −0.22327 | cg20080845 | BRD1; LOC90834 | 361454 |
| −0.2235 | cg15016481 | LRRFIP1; LRRFIP1; LRRFIP1; | 278955 |
| | | LRRFIP1; LRRFIP1 | |
| −0.22429 | cg04112058 | SHCBP1 | 82595 |
| −0.22434 | cg17879450 | MACF1 | 325874 |
| −0.2244 | cg12399224 | ZBTB46 | 230928 |
| −0.22443 | cg22909677 | ARMC2 | 404123 |
| −0.22448 | cg25874123 | MYOM1; MYOM1 | 451828 |
| −0.22451 | cg18720326 | ADAMTS2; ADAMTS2 | 339675 |
| −0.2246 | cg16230121 | APBB1; APBB1 | 298473 |
| −0.22463 | cg21848191 | RAD51L1; RAD51L1; RAD51L1 | 388170 |
| −0.22463 | cg19026231 | GALK1 | 344545 |
| −0.22483 | cg07080231 | | 138324 |
| −0.22528 | cg17531430 | BIN1; BIN1; BIN1; BIN1; BIN1; | 320696 |
| | | BIN1; BIN1; BIN1; BIN1; BIN1 | |
| −0.22531 | cg07807373 | | 151942 |
| −0.22568 | cg09038971 | CHST11 | 174071 |
| −0.22576 | cg16624521 | | 305662 |
| −0.22583 | cg10031769 | CREM; CREM; CREM; CREM; | 191189 |
| | | CREM; CREM; CREM; CREM; | |
| | | CREM; CREM; CREM; | |
| | | CREM; CREM; CREM; CREM; | |
| | | CREM; CREM; CREM; | |
| | | CREM; CREM | |
| −0.22674 | cg15853771 | | 292370 |
| −0.22677 | cg05242915 | | 104329 |
| −0.22727 | cg09264140 | FMNL1 | 177950 |
| −0.22741 | cg24977338 | RGPD8; RGPD5 | 437964 |
| −0.22749 | cg27469810 | CETN1 | 479043 |
| −0.22788 | cg00653025 | | 13831 |
| −0.22788 | cg05285759 | TBCK; TBCK; TBCK; TBCK | 105125 |
| −0.22818 | cg12642431 | UACA; UACA | 235055 |
| −0.22826 | cg09169413 | P11 | 176276 |
| −0.22856 | cg23079012 | | 407020 |
| −0.2289 | cg23904501 | PTPRS; PTPRS; PTPRS; PTPRS | 420216 |
| −0.22901 | cg07631435 | ZBTB16; ZBTB16 | 148813 |
| −0.22976 | cg13752933 | DAPK1 | 254331 |
| −0.22984 | cg19173502 | MIRLET7C; C21orf34; C21orf34 | 346603 |
| −0.22995 | cg21502482 | SPATA7; SPATA7 | 382912 |
| −0.22997 | cg03144446 | | 63874 |
| −0.23001 | cg01164141 | | 24508 |
| −0.23003 | cg27314273 | PTPRG | 476489 |
| −0.23018 | cg13879937 | EHD4 | 256817 |
| −0.23035 | cg11168687 | ACSL1 | 210627 |
| −0.23037 | cg22905866 | | 404070 |
| −0.23047 | cg19788250 | APP; APP; APP; APP; APP | 356630 |
| −0.23055 | cg19478371 | PPP2R5C; PPP2R5C; PPP2R5C; | 351294 |
| | | PPP2R5C; PPP2R5C | |
| −0.23081 | cg08153404 | | 158265 |
| −0.23098 | cg11945474 | | 223676 |
| −0.23103 | cg07470830 | | 145712 |
| −0.23116 | cg09537792 | ANKRD11 | 182760 |
| −0.23122 | cg17880118 | COL4A1 | 325886 |
| −0.2315 | cg07468585 | | 145653 |
| −0.23177 | cg00108038 | LOC100292680 | 2509 |
| −0.23183 | cg23560676 | NSMCE2 | 414881 |
| −0.23213 | cg11063877 | LARP4; LARP4; LARP4; LARP4; | 208898 |
| | | LARP4; LARP4; LARP4 | |
| −0.23228 | cg19735406 | COL4A2 | 355626 |
| −0.23258 | cg09259797 | | 177870 |
| −0.23286 | cg05314142 | SLC22A1; SLC22A1 | 105694 |
| −0.23299 | cg06519061 | PTPN14 | 127144 |
| −0.23315 | cg26983411 | | 470886 |
| −0.23331 | cg01417625 | LAMC2; LAMC2 | 29483 |
| −0.23345 | cg06112923 | STEAP2; STEAP2; STEAP2 | 120290 |
| −0.23386 | cg22416916 | SLC22A1; SLC22A1 | 396454 |
| −0.23403 | cg10492417 | FANCA | 199244 |
| −0.23409 | cg02540833 | SHANK2 | 51760 |
| −0.2342 | cg09329079 | TMEM184A | 179139 |
| −0.23446 | cg08135718 | CUX1; CUX1; CUX1 | 157939 |
| −0.23468 | cg20724690 | CAB39; CAB39; CAB39 | 371287 |
| −0.23494 | cg20192387 | RPS6KA2; RPS6KA2 | 363087 |
| −0.23519 | cg09169739 | GFPT2 | 176289 |
| −0.23558 | cg22396663 | MAD1L1; MAD1L1; MAD1L1 | 396208 |
| −0.23597 | cg02923628 | | 59493 |
| −0.23632 | cg03109660 | RELL1; RELL1 | 63223 |
| −0.23648 | cg06562964 | RELL1; RELL1 | 128035 |
| −0.23857 | cg17133045 | AKT3; AKT3 | 313972 |
| −0.23937 | cg03606098 | NADK | 72963 |
| −0.23938 | cg18771195 | RPH3AL | 340542 |
| −0.23963 | cg10013121 | TRERF1 | 190859 |
| −0.23979 | cg02781060 | | 56673 |
| −0.23996 | cg22117968 | | 392140 |
| −0.2401 | cg11520719 | ARID1B; ARID1B; ARID1B | 216320 |
| −0.24011 | cg04126956 | TRAPPC9; TRAPPC9 | 82912 |
| −0.24087 | cg01312265 | TCERG1L | 27436 |
| −0.24102 | cg09873409 | PLXNA4; PLXNA4 | 188543 |
| −0.24159 | cg00089451 | | 2073 |
| −0.24161 | cg27446233 | PFKM; PFKM; PFKM; PFKM; | 478685 |
| | | PFKM | |
| −0.24161 | cg07251141 | ADAM12; ADAM12 | 141811 |
| −0.24173 | cg02773698 | FCHSD2 | 56533 |
| −0.2419 | cg11028872 | | 208387 |
| −0.24214 | cg06865642 | BRD1; LOC90834 | 134034 |
| −0.24269 | cg18000216 | ERC2 | 327710 |
| −0.24279 | cg14558716 | ATF1 | 270966 |
| −0.24303 | cg06165742 | FOXP2; FOXP2; FOXP2 | 121155 |
| −0.24317 | cg07491828 | MKL2 | 146139 |
| −0.24321 | cg05495611 | EXT2; EXT2 | 109044 |
| −0.24432 | cg00264129 | HBEGF | 5659 |
| −0.24487 | cg20539449 | | 368227 |
| −0.24494 | cg07877629 | | 153192 |
| −0.24502 | cg00026412 | ANK3 | 645 |
| −0.24505 | cg25089903 | NINJ2 | 439753 |
| −0.24567 | cg23708624 | CCDC146; FGL2 | 417387 |
| −0.24585 | cg25838968 | PLXNA2 | 451397 |
| −0.24656 | cg02724461 | GALNS | 55487 |
| −0.24687 | cg23669855 | LYSMD4 | 416659 |
| −0.24737 | cg19547694 | BRD1; LOC90834 | 352451 |
| −0.24757 | cg13962170 | | 258544 |
| −0.24861 | cg17798857 | UACA; UACA | 324425 |
| −0.24871 | cg09741789 | FBN1 | 186193 |
| −0.24879 | cg04721825 | PRKCA | 94264 |
| −0.24908 | cg01556677 | SEMA3C | 32186 |
| −0.24945 | cg19450056 | | 350853 |
| −0.24949 | cg25141441 | RORA; RORA; RORA; RORA | 440625 |
| −0.24973 | cg04265492 | BAI2 | 85697 |
| −0.2506 | cg03870329 | | 77949 |
| −0.25077 | cg25206802 | SACS | 441606 |
| −0.25093 | cg25483191 | TRIM55; TRIM55; TRIM55; | 445943 |
| | | TRIM55 | |
| −0.25124 | cg08535361 | NTN4 | 165209 |
| −0.25143 | cg04425710 | ESCO2; CCDC25 | 88827 |
| −0.25168 | cg08464954 | | 163934 |
| −0.25182 | cg13661832 | | 252443 |
| −0.25199 | cg10259111 | GNG2 | 195382 |
| −0.25214 | cg24331722 | | 427189 |
| −0.25249 | cg26529437 | ITGB8 | 462893 |
| −0.25283 | cg25706447 | PIGV | 449350 |
| −0.25296 | cg26131670 | SLC4A4; SLC4A4 | 455944 |
| −0.25313 | cg11274148 | RPTOR; RPTOR | 212468 |
| −0.25324 | cg14571004 | GALNT12 | 271226 |
| −0.25333 | cg26306638 | HBEGF | 459010 |
| −0.25362 | cg08412699 | ITGB8 | 162975 |
| −0.25409 | cg12208381 | CSNK1G2 | 228165 |
| −0.25453 | cg11611676 | | 217803 |
| −0.25457 | cg13665060 | | 252509 |
| −0.25472 | cg16784943 | ITPK1; ITPK1; ITPK1 | 308597 |
| −0.25501 | cg24534767 | | 430608 |
| −0.25576 | cg03778207 | ME3; ME3; ME3 | 76334 |
| −0.25579 | cg09088836 | | 174888 |
| −0.25665 | cg26543192 | | 463160 |
| −0.2568 | cg14457429 | | 268785 |
| −0.25731 | cg16834726 | DCTN2 | 309390 |
| −0.25741 | cg14523881 | VASH2; VASH2; VASH2 | 270202 |
| −0.25788 | cg05208056 | NCK2; NCK2; NCK2 | 103597 |
| −0.25817 | cg07525751 | SLC6A3 | 146753 |
| −0.25823 | cg03855388 | CPT1A; CPT1A | 77697 |
| −0.25832 | cg22308453 | DPYSL2 | 394900 |
| −0.25865 | cg07911905 | CHST11 | 153817 |
| −0.25865 | cg05196076 | FBXO31; FBXO31 | 103393 |
| −0.25949 | cg18371976 | TRERF1 | 334048 |
| −0.25957 | cg16878214 | PLEKHF2 | 309991 |
| −0.25958 | cg20655877 | | 369984 |
| −0.26099 | cg21197233 | ARHGAP20 | 378484 |
| −0.26155 | cg15568998 | AGMAT | 287517 |
| −0.26178 | cg27511861 | | 479747 |
| −0.26219 | cg12564175 | ESCO2; CCDC25 | 233667 |
| −0.26237 | cg16090790 | | 296212 |
| −0.2632 | cg12552626 | CCDC146; FGL2 | 233502 |
| −0.26416 | cg11072779 | ANXA4 | 209068 |
| −0.26569 | cg22352504 | HAS1 | 395515 |
| −0.26608 | cg07263759 | | 142020 |
| −0.26615 | cg18022344 | METTL9; IGSF6; METTL9 | 328111 |
| −0.2662 | cg24033633 | | 422524 |
| −0.26675 | cg05310185 | TACC2; TACC2; TACC2; TACC2 | 105623 |
| −0.26683 | cg02606505 | NKTR | 53079 |
| −0.26684 | cg05899338 | ZNF239; ZNF239; ZNF239 | 116275 |
| −0.26725 | cg06385757 | PRKAR1B; PRKAR1B; | 124893 |
| | | PRKAR1B; PRKAR1B; | |
| | | PRKAR1B; PRKAR1B | |
| −0.26763 | cg14178899 | ERRFI1 | 263095 |
| −0.2679 | cg03372385 | LRRC16A | 68299 |
| −0.26797 | cg25072766 | CARS; CARS; CARS; CARS | 439518 |
| −0.26865 | cg20608783 | BAI2 | 369275 |
| −0.26917 | cg07776993 | ADCK5 | 151365 |
| −0.27071 | cg01295631 | | 27066 |
| −0.27154 | cg06836480 | DHRS9; DHRS9; DHRS9 | 133449 |
| −0.27159 | cg01957599 | FAM13AOS | 40119 |
| −0.2728 | cg17478992 | SLC12A8 | 319759 |
| −0.27286 | cg12884466 | MRPL3 | 238716 |
| −0.27379 | cg13200640 | | 243476 |
| −0.27391 | cg12619262 | | 234658 |
| −0.27459 | cg26551026 | CABLES1; CABLES1 | 463331 |
| −0.27512 | cg03078520 | MICAL3; MICAL3; MICAL3 | 62698 |
| −0.27559 | cg19295451 | PCSK6; PCSK6; PCSK6; PCSK6; | 348451 |
| | | PCSK6; PCSK6; PCSK6; | |
| | | PCSK6 | |
| −0.2759 | cg01359165 | | 28268 |
| −0.27611 | cg03389789 | FBXW8; FBXW8 | 68597 |
| −0.27642 | cg27199697 | DLX3 | 474550 |
| −0.27828 | cg19995595 | ITPK1; ITPK1; ITPK1 | 360131 |
| −0.2796 | cg19600494 | | 353349 |
| −0.28011 | cg22801134 | PRICKLE2 | 402255 |
| −0.28065 | cg20140333 | | 362329 |
| −0.28088 | cg00101350 | MGAT3; MGAT3 | 2375 |
| −0.28176 | cg10784341 | PHACTR2; PHACTR2 | 204295 |
| −0.28206 | cg23338380 | CUX1; CUX1; CUX1 | 411370 |
| −0.28216 | cg22122925 | ERRFI1 | 392216 |
| −0.28241 | cg06235645 | SEMA6D; SEMA6D; SEMA6D; | 122412 |
| | | SEMA6D; SEMA6D; SEMA6D | |
| −0.28268 | cg16474725 | CLIC5; CLIC5 | 302879 |
| −0.28421 | cg14054582 | | 260566 |
| −0.2851 | cg19926144 | DIP2C | 359006 |
| −0.28525 | cg26214026 | ATXN2 | 457353 |
| −0.28725 | cg18030133 | PHACTR2; PHACTR2; | 328254 |
| | | PHACTR2; PHACTR2 | |
| −0.28771 | cg22716599 | DNM3; DNM3 | 400952 |
| −0.28822 | cg00745735 | MYPN | 15768 |
| −0.28884 | cg02071553 | MIR216B | 42524 |
| −0.28893 | cg01756899 | | 35949 |
| −0.28929 | cg05928904 | | 116850 |
| −0.28962 | cg01598421 | TG | 33081 |
| −0.28997 | cg02131142 | TBC1D9B; TBC1D9B | 43723 |
| −0.29156 | cg03412557 | RUNDC2A | 69042 |
| −0.29166 | cg17947992 | | 326928 |
| −0.29212 | cg00855685 | SEMA5A | 18020 |
| −0.29231 | cg08672203 | PPAP2A; PPAP2A | 167602 |
| −0.29315 | cg17613911 | STK32A | 321844 |
| −0.29506 | cg23666374 | AUTS2; AUTS2; AUTS2 | 416583 |
| −0.29533 | cg16666745 | GALNT10 | 306478 |
| −0.2964 | cg26826631 | CD55; CD55 | 468043 |
| −0.29748 | cg17919396 | ALDH1A3 | 326446 |
| −0.29758 | cg01574912 | | 32628 |
| −0.29814 | cg14334389 | SPARCL1; SPARCL1 | 266428 |
| −0.29818 | cg04304338 | ANKRD33B | 86451 |
| −0.29932 | cg02847703 | BTC | 57849 |
| −0.30181 | cg23155767 | GPR1; GPR1 | 408172 |
| −0.30344 | cg09819635 | KHDRBS3 | 187501 |
| −0.30363 | cg25131079 | ADAMTS2; ADAMTS2 | 440427 |
| −0.30642 | cg12754854 | PHYHD1; PHYHD1 | 236759 |
| −0.30675 | cg08943696 | GPR133 | 172510 |
| −0.30689 | cg03983715 | PRMT7 | 80095 |
| −0.30824 | cg09287328 | | 178354 |
| −0.30861 | cg03713317 | CYFIP1; CYFIP1 | 74995 |
| −0.31349 | cg23666829 | TBCD | 416590 |
| −0.31884 | cg26253868 | | 458047 |
| −0.32034 | cg03700279 | PREPL; PREPL; PREPL; PREPL; | 74742 |
| | | PREPL; PREPL; PREPL | |
| −0.32126 | cg12894142 | | 238844 |
| −0.32278 | cg16689609 | UTRN | 306875 |
| −0.32343 | cg12359895 | LOC100216001; LOC338588 | 230357 |
| −0.32409 | cg19244380 | AMBRA1; AMBRA1 | 347620 |
| −0.32631 | cg01928411 | LARP4; LARP4; LARP4; LARP4; | 39500 |
| | | LARP4; LARP4; LARP4 | |
| −0.32706 | cg25254949 | | 442307 |
| −0.32836 | cg23159678 | NOVA1; NOVA1; NOVA1 | 408237 |
| −0.33031 | cg11520439 | LOC286367 | 216313 |
| −0.33087 | cg11937508 | GPR1; GPR1 | 223523 |
| −0.33212 | cg12205433 | TES; TES | 228120 |
| −0.33433 | cg18901940 | MYPN; MYPN | 342719 |
| −0.34043 | cg10362527 | GPR1; GPR1 | 197111 |
| −0.34108 | cg14570632 | RXRA | 271222 |
| −0.34414 | cg03361973 | SEMA6D; SEMA6D; SEMA6D; | 68105 |
| | | SEMA6D; SEMA6D; SEMA6D | |
| −0.34417 | cg05073720 | PHLPP1 | 101171 |
| −0.34459 | cg16087541 | TES; TES | 296173 |
| −0.34847 | cg09835421 | PRMT7 | 187811 |
| −0.34934 | cg21161187 | SLC6A3 | 377787 |
| −0.35195 | cg02666566 | | 54356 |
| −0.35418 | cg18262591 | CPT1A; CPT1A | 332336 |
| −0.37588 | cg16808481 | TES; TES | 309027 |
| −0.38211 | cg14604584 | | 271938 |
| −0.40768 | cg05110241 | PRMT7 | 101782 |
| −0.41512 | cg20071744 | TRAPPC9; TRAPPC9 | 361314 |
| −0.43386 | cg15853299 | TBCD | 292363 |

TABLE 8

| | Associated | | | |
|---|---|---|---|---|
| OA − RA | gene | CpG* | Locus | SEQ ID No. |
| −0.603 | LIMS1 | 2: 109251064 | cg10879348 | 205889 |
| −0.603 | | 15:97311928 | cg04875706 | 97278 |
| −0.572 | | 17:35161825 | cg11331837 | 213402 |
| −0.563 | ACOT11 | 1:55014160 | cg01802772 | 36934 |
| −0.552 | GNAS | 20:57463991 | cg09885502 | 188727 |
| −0.527 | | 11:28642652 | cg26387458 | 460494 |
| −0.511 | HDAC11 | 3:13555664 | cg26845082 | 468403 |
| −0.506 | | 3:22422855 | cg20979384 | 374956 |
| −0.484 | | 1:204556835 | cg13232075 | 244024 |
| −0.478 | AP2A2 | 11:1001560 | cg16999994 | 311801 |
| −0.463 | ALS2CR12 | 2:202163482 | cg07227024 | 141394 |
| −0.463 | | 4:82273176 | cg07454933 | 145378 |
| −0.456 | CPO | 2:207803547 | cg17600943 | 321671 |
| −0.455 | FAM19A5 | 22:48896579 | cg11019791 | 208228 |
| −0.452 | FHIT | 3:60420416 | cg16140565 | 297019 |
| −0.443 | | 6:75775380 | cg15706692 | 289960 |
| −0.439 | ATP11A | 13:113539522 | cg21463262 | 382273 |
| −0.435 | PDHA2 | 4:96760945 | cg10590622 | 201120 |
| −0.433 | CNTNAP5 | 2:124971544 | cg01013522 | 21327 |
| −0.432 | COLEC11 | 2:3675155 | cg10724632 | 203346 |
| −0.432 | RD3 | 1:211667751 | cg22481673 | 397284 |
| −0.424 | RBM28 | 7:127950724 | cg03119308 | 63402 |
| −0.423 | | 13:21896301 | cg13730219 | 253914 |
| −0.423 | | 8:142283564 | cg04123498 | 82844 |
| −0.421 | MYOCD | 17:12562530 | cg14192979 | 263428 |
| −0.421 | SLFN12 | 17:33734664 | cg20086657 | 361521 |
| −0.417 | | 2:150845309 | cg15958422 | 293993 |
| −0.409 | IFT43; TGFB3 | 14:76443578 | cg21193926 | 378403 |
| −0.405 | FAM101A; NCOR2; | 12:124810173 | cg13127920 | 242348 |
| | ZNF664- | | | |
| | FAM101A | | | |
| −0.403 | SLC2A9 | 4:10009916 | cg21795255 | 387383 |
| −0.403 | ATXN7L1 | 7:105306555 | cg11957130 | 223895 |
| −0.403 | TRIM64C | 11:49073835 | cg06445586 | 125882 |
| −0.400 | HLA-A | 6:29906977 | cg26739327 | 466540 |
| −0.394 | SCN4B | 11:118022607 | cg22251955 | 394153 |
| −0.392 | LINC00254 | 16:1939295 | cg10051493 | 191486 |
| −0.391 | MIR4457; TERT | 5:1300309 | cg14597862 | 271789 |
| −0.390 | | 14:22279816 | cg16423096 | 302000 |
| −0.384 | RPL27A; SNORA3; | 11:8714324 | cg15828613 | 291887 |
| | SNORA45; | | | |
| | ST5 | | | |
| −0.382 | MXRA7 | 17:74679597 | cg03810198 | 76895 |
| −0.378 | CD82 | 11:44649802 | cg21974656 | 390131 |
| −0.377 | BPIFA2 | 20:31755943 | cg05840533 | 115130 |
| −0.377 | CCDC109B | 4:110524473 | cg17082366 | 313142 |
| −0.375 | TRIM24 | 7:138229989 | cg12743416 | 236587 |
| −0.374 | RPTOR | 17:78935070 | cg17906851 | 326284 |
| −0.371 | KDM2B | 12:121893769 | cg07741840 | 150784 |
| −0.370 | HCG4B | 6:29895204 | cg26121931 | 455779 |
| −0.369 | NSMCE4A | 10:123731471 | cg19360212 | 349382 |
| −0.369 | NMNAT3 | 3:139397901 | cg15295200 | 283344 |
| −0.366 | | 12:25454990 | cg25134647 | 440489 |
| −0.365 | | 6:55778951 | cg11290949 | 212735 |
| −0.361 | MRPL23 | 11:1985287 | cg06258939 | 122771 |
| −0.351 | | 4:8569129 | cg06991380 | 136538 |
| −0.350 | CDH4; MIR1257 | 20:60520624 | cg16423285 | 302003 |
| −0.349 | MAST2 | 1:46420220 | cg22337626 | 395319 |
| −0.346 | KIAA1908; PSMG3 | 7:1616229 | cg23403836 | 412403 |
| −0.346 | SUMO3; UBE2G2 | 21:46220161 | cg00272795 | 5810 |
| −0.337 | OR10K1 | 1:158435277 | cg01463139 | 30452 |
| −0.337 | SLCO2B1 | 11:74861433 | cg20704148 | 370892 |
| −0.329 | SPATS2L | 2:201289756 | cg17174466 | 314712 |
| −0.327 | SFT2D3; WDR33 | 2:128453335 | cg03738707 | 75556 |
| −0.325 | HLA-DRB1 | 6:32555411 | cg00598125 | 12786 |
| −0.323 | CCHCR1; PSORS1C2 | 6:31107186 | cg07414487 | 144678 |
| −0.322 | HLA-A | 6:29913343 | cg19585676 | 353041 |
| −0.320 | LOC100628307 | 14:80858152 | cg07572984 | 147668 |
| −0.318 | PSORS1C3 | 6:31148606 | cg01982166 | 40677 |
| −0.313 | PTGER3; ZRANB2- | 1:71511514 | cg11418303 | 214891 |
| | AS1 | | | |
| −0.313 | | 10:131209556 | cg02372404 | 48486 |
| −0.310 | | 7:158750985 | cg00413089 | 8734 |
| −0.307 | HCG4B | 6:29894152 | cg25644740 | 448500 |
| −0.305 | | 2:5605864 | cg23233416 | 409551 |
| −0.305 | SLC47A2 | 17:19620263 | cg08102564 | 157230 |
| −0.302 | SYS1; SYS1- | 20:44007674 | cg17811452 | 324650 |
| | DBNDD2; TP53TG5 | | | |
| −0.301 | COL4A2 | 13:111115558 | cg07618759 | 148582 |
| −0.300 | CDCA8; EPHA10 | 1:38180356 | cg06437931 | 125736 |
| −0.300 | RPTOR | 17:78809403 | cg20502501 | 367732 |
| −0.299 | NDRG4 | 16:58534681 | cg00758881 | 16033 |
| −0.296 | ITIH1; ITIH3 | 3:52828628 | cg05393861 | 107345 |
| −0.296 | DPM2; FAM102A | 9:130705120 | cg01681863 | 34567 |
| −0.295 | TTC34 | 1:2710376 | cg21783012 | 387180 |
| −0.292 | HCG4B | 6:29895175 | cg23681866 | 416899 |
| −0.290 | HCG4B | 6:29895187 | cg15411272 | 285048 |
| −0.288 | PSMD9; WDR66 | 12:122356390 | cg21171335 | 377993 |
| −0.286 | BOC | 3:112927486 | cg27187580 | 474373 |
| −0.286 | MCF2L | 13:113688132 | cg15388766 | 284663 |
| −0.285 | B4GALNT4 | 11:364752 | cg25497175 | 446150 |
| −0.283 | | 8:92570895 | cg11420142 | 214927 |
| −0.281 | | 7:158751184 | cg12744031 | 236605 |
| −0.279 | | 13:27077539 | cg03531388 | 71371 |
| −0.278 | CASP7 | 10:115465924 | cg01128042 | 23762 |
| −0.278 | HCG4B | 6:29895260 | cg24838316 | 435648 |
| −0.278 | | 2:69440951 | cg02444957 | 49778 |
| −0.277 | OR51A2; OR51A4 | 11:4976263 | cg25641451 | 448448 |
| −0.274 | FRMD4A | 10:13972210 | cg07629776 | 148782 |
| −0.274 | | 22:25571929 | cg19707653 | 355076 |
| −0.272 | ATHL1; NLRP6 | 11:285037 | cg22280068 | 394534 |
| −0.269 | SORBS2 | 4:186549051 | cg09120722 | 175360 |
| −0.267 | | 11:5384517 | cg26567385 | 463630 |
| −0.265 | MRPL44; WDFY1 | 2:224813276 | cg15176306 | 281664 |
| −0.265 | | 19:29218101 | cg04546413 | 91043 |
| −0.264 | | 10:47964834 | cg16435686 | 302231 |
| −0.264 | | 7:158750244 | cg12954512 | 239779 |
| −0.263 | | 16:14380714 | cg03940883 | 79274 |
| −0.262 | B4GALT6 | 18:29205358 | cg11986743 | 224388 |
| −0.261 | FBRSL1 | 12:133078117 | cg23836570 | 419138 |
| −0.259 | LOC440905 | 2:130795582 | cg18480627 | 335932 |
| −0.257 | SLC27A1 | 19:17600122 | cg02707854 | 55128 |
| −0.257 | | 5:54117761 | cg25997988 | 453719 |
| −0.257 | ATOH1 | 4:94759825 | cg05893845 | 116159 |
| −0.256 | LOC100289473 | 20:1757570 | cg03299990 | 66803 |
| −0.252 | COL18A1 | 21:46921982 | cg05638648 | 111601 |
| −0.251 | | 7:1923695 | cg12169700 | 227539 |
| −0.250 | C1orf86; PRKCZ | 1:2121039 | cg26227225 | 457598 |
| −0.248 | | 5:31106255 | cg12908908 | 239081 |
| −0.247 | FAM157B | 9:141105974 | cg04824555 | 96218 |
| −0.247 | | 7:1976457 | cg03075889 | 62651 |
| −0.246 | SLC6A3 | 5:1420305 | cg07664579 | 149363 |
| −0.245 | ADAMTS2 | 5:178565001 | cg01387905 | 28837 |
| −0.243 | | 7:136832262 | cg05364766 | 106693 |
| −0.241 | | 19:13276344 | cg19225953 | 347327 |
| −0.241 | PSMD9; WDR66 | 12:122356400 | cg22618164 | 399325 |
| −0.239 | | 4:9479622 | cg01394167 | 28957 |
| −0.239 | | 12:125411922 | cg07877257 | 153188 |
| −0.238 | | 7:158750607 | cg00815399 | 17228 |
| −0.238 | RNF213 | 17:78264297 | cg11606607 | 217718 |
| −0.237 | TMEM39B | 1:32535981 | cg10312513 | 196231 |
| −0.234 | LPIN1 | 2:11888753 | cg00523161 | 11124 |
| −0.234 | SGK110 | 19:56061334 | cg18795732 | 340965 |
| −0.232 | LOC154822; VIPR2 | 7:158821175 | cg25909532 | 452273 |
| −0.231 | | 7:158750384 | cg11945929 | 223689 |
| −0.229 | | 1:161410551 | cg22730047 | 401169 |
| −0.228 | PLEC | 8:145003776 | cg18000391 | 327713 |
| −0.227 | | 17:80849463 | cg14195178 | 263473 |
| −0.226 | HOXA3; HOXA4; | 7:27170394 | cg19142026 | 346203 |
| | LOC100133311 | | | |
| −0.225 | | 6:16962712 | cg19300401 | 348504 |
| −0.225 | FAM66A | 8:12237626 | cg10206627 | 194443 |
| −0.223 | HTATIP2 | 11:20384377 | cg26155681 | 456393 |
| −0.221 | AP2A2 | 11:970389 | cg12893697 | 238836 |
| −0.220 | COL4A2 | 13:111111172 | cg19218082 | 347212 |
| −0.218 | PAPLN | 14:73712902 | cg14834903 | 275901 |
| −0.218 | OSBPL5 | 11:3174092 | cg06120313 | 120406 |
| −0.217 | C1orf86; PRKCZ | 1:2120985 | cg20300514 | 364680 |
| −0.217 | | 7:1367727 | cg23896876 | 420086 |
| −0.216 | EGLN2; MIA- | 19:41307778 | cg21653913 | 385460 |
| | RAB4B; RAB4B; | | | |
| | RAB4B- | | | |
| | EGLN2 | | | |
| −0.216 | | 13:80755568 | cg00750481 | 15862 |
| −0.216 | | 5:176099987 | cg11583351 | 217324 |
| −0.215 | | 5:125171542 | cg12599971 | 234303 |
| −0.215 | MYO1D | 17:31149877 | cg01050010 | 22131 |
| −0.213 | DPYSL4; JAKMIP3 | 10:134001728 | cg10144198 | 193247 |
| −0.213 | | 19:21863715 | cg26562263 | 463546 |
| −0.213 | NDRG4 | 16:58533979 | cg27113419 | 473118 |
| −0.212 | BMP4 | 14:54410717 | cg16720578 | 307533 |
| −0.210 | | 5:134388224 | cg03827835 | 77177 |
| −0.209 | C1orf86; PRKCZ | 1:2121349 | cg05337761 | 106096 |
| −0.209 | SFT2D3; WDR33 | 2:128453445 | cg11059159 | 208817 |
| −0.208 | APOA1; APOC3; | 11:116708413 | cg09558982 | 183152 |
| | SIK3 | | | |
| −0.207 | SFT2D3; WDR33 | 2:128453484 | cg25277809 | 442618 |
| −0.206 | | 7:158750417 | cg10079374 | 191993 |
| −0.205 | | 10:43522343 | cg08461752 | 163848 |
| −0.205 | LASP1 | 17:37024042 | cg20826740 | 372827 |
| −0.204 | NSUN4 | 1:46807113 | cg17806798 | 324558 |
| −0.204 | | 1:149145082 | cg16661522 | 306375 |
| −0.204 | PTP4A3 | 8:142427117 | cg04687040 | 93680 |
| −0.204 | | 5:92414398 | cg18783429 | 340738 |
| −0.203 | FLJ37453 | 1:16154831 | cg21385522 | 381217 |
| −0.203 | | 12:132869069 | cg05948955 | 117271 |
| −0.202 | RADIL | 7:4854126 | cg08799816 | 169789 |
| −0.202 | | 1:34615965 | cg21440084 | 381930 |
| −0.201 | ARHGEF40; C14orf176; | 14:21567333 | cg00968545 | 20358 |
| | ZNF219 | | | |
| −0.200 | | 7:1948755 | cg16178271 | 297641 |
| −0.199 | TNS1 | 2:218707806 | cg17588704 | 321484 |
| −0.198 | HLA-C | 6:31241294 | cg05338672 | 106118 |
| −0.197 | SLC27A1 | 19:17599784 | cg17132030 | 313955 |
| −0.196 | | 11:19699299 | cg18593660 | 337773 |
| −0.196 | | 16:1797050 | cg07869343 | 153029 |
| −0.195 | GAS2L2; RASL10B | 17:34067305 | cg23633026 | 415965 |
| −0.195 | | 7:158790006 | cg09640425 | 184513 |
| −0.195 | LRRC27 | 10:134150581 | cg25076597 | 439582 |
| −0.194 | LOC440905 | 2:130795350 | cg25180228 | 441163 |
| −0.194 | ZC3H12D | 6:149785754 | cg24131442 | 424218 |
| −0.193 | CES1 | 16:55867072 | cg03744383 | 75666 |
| −0.193 | ACSF3 | 16:89152443 | cg00324979 | 6844 |
| −0.192 | | 16:56000067 | cg00991179 | 20841 |
| −0.192 | AP2A2 | 11:970664 | cg06064525 | 119407 |
| −0.192 | CHTOP; S100A1; | 1:153599704 | cg11343894 | 213623 |
| | S100A13 | | | |
| −0.190 | DBNL; PGAM2; | 7:44104860 | cg07075347 | 138233 |
| | POLM | | | |
| −0.189 | MYO10 | 5:16895511 | cg02288345 | 46701 |
| −0.189 | | 19:29218262 | cg14983838 | 278359 |
| −0.189 | EIF2D | 1:206786180 | cg21936179 | 389599 |
| −0.189 | FN3K; TBCD | 17:80708632 | cg02398342 | 49038 |
| −0.188 | TM7SF4 | 8:105342491 | cg04554929 | 91221 |
| −0.187 | NXN | 17:707141 | cg20403644 | 366219 |
| −0.187 | NFATC1 | 18:77292443 | cg06675417 | 130167 |
| −0.186 | KIAA1751 | 1:1886501 | cg06090818 | 119880 |
| −0.185 | LOC100507055 | 12:133180238 | cg26621607 | 464484 |
| −0.185 | SLC9A3 | 5:502291 | cg14137381 | 262235 |
| −0.184 | CLDN22; CLDN24; | 4:184250312 | cg14241748 | 264522 |
| | WWC2 | | | |
| −0.184 | MMD2 | 7:5000111 | cg02276823 | 46453 |
| −0.184 | COL5A1 | 9:137691430 | cg13889467 | 257004 |
| −0.184 | | 6:30000187 | cg01915885 | 39235 |
| −0.184 | SLC47A2 | 17:19627822 | cg12434897 | 231546 |
| −0.183 | KRTAP4-7 | 17:39240789 | cg23827950 | 418999 |
| −0.182 | | 19:29218302 | cg12756686 | 236783 |
| −0.182 | GAB2 | 11:78052863 | cg24769381 | 434565 |
| −0.182 | | 13:33854227 | cg15405588 | 284964 |
| −0.182 | | 13:112978703 | cg19891452 | 358388 |
| −0.180 | C11orf53 | 11:111148753 | cg27649396 | 482130 |
| −0.179 | | 19:29218774 | cg03161606 | 64225 |
| −0.178 | ADAP1; COX19 | 7:1003645 | cg08735705 | 168762 |
| −0.178 | MTG1; PAOX | 10:135202877 | cg06080948 | 119697 |
| −0.177 | NEUROG3 | 10:71339632 | cg02330874 | 47587 |
| −0.177 | C10orf96 | 10:118084587 | cg02721176 | 55417 |
| −0.177 | BAI1 | 8:143546858 | cg02048922 | 41986 |
| −0.176 | | 8:119750651 | cg26711508 | 466113 |
| −0.176 | GPR50 | X:150351493 | cg20071203 | 361302 |
| −0.175 | LARS2 | 3:45429626 | cg13510812 | 249280 |
| −0.175 | TUBG2 | 17:40808068 | cg24417155 | 428624 |
| −0.175 | OR12D2 | 6:29364799 | cg25786333 | 450628 |
| −0.174 | RASA3 | 13:114872606 | cg27086157 | 472626 |
| −0.174 | C21orf128; UMODL1 | 21:43529162 | cg02489157 | 50718 |
| −0.174 | MYT1L | 2:2119533 | cg26383138 | 460416 |
| −0.174 | | 10:125751386 | cg00502035 | 10662 |
| −0.174 | | 7:102134668 | cg17207545 | 315260 |
| −0.173 | NRG1 | 8:32579253 | cg20127899 | 362151 |
| −0.173 | CHTOP; S100A1; | 1:153599671 | cg13946767 | 258256 |
| | S100A13 | | | |
| −0.173 | APOA1BP; IQGAP3; | 1:156551787 | cg09319828 | 178942 |
| | TTC24 | | | |
| −0.172 | ATP11A | 13:113413587 | cg08484560 | 164316 |
| −0.171 | NKX2-3 | 10:101287609 | cg10947831 | 206964 |
| −0.171 | | 5:171057282 | cg10881242 | 205925 |
| −0.171 | ADAP1; COX19 | 7:1003710 | cg12575883 | 233841 |
| −0.171 | CRAMP1L; HN1L | 16:1722957 | cg04245870 | 85306 |
| −0.170 | ADAMTS7 | 15:79107452 | cg23450377 | 413109 |
| −0.170 | PCSK6 | 15:101932559 | cg27370471 | 477437 |
| −0.170 | | 19:1737123 | cg08692111 | 167944 |
| −0.170 | C5orf38; IRX2 | 5:2743124 | cg18803147 | 341088 |
| −0.169 | BAI1 | 8:143581481 | cg26953288 | 470357 |
| −0.169 | STK32B | 4:5374425 | cg14422932 | 268145 |
| −0.168 | | X:152584950 | cg03201165 | 64963 |
| −0.168 | POU5F1; PSORS1C3 | 6:31146845 | cg20073472 | 361344 |
| −0.168 | NDRG4 | 16:58534708 | cg04484415 | 89876 |
| −0.168 | FAM20C | 7:194798 | cg12042135 | 225245 |
| −0.167 | EDN1 | 6:12292676 | cg27565517 | 480713 |
| −0.167 | C1QTNF8 | 16:1152511 | cg06394109 | 125022 |
| −0.167 | KLK5; KLK6 | 19:51456308 | cg00768359 | 16234 |
| −0.166 | ADAP1 | 7:961457 | cg06589239 | 128495 |
| −0.166 | | 11:134892703 | cg21757617 | 386803 |
| −0.166 | HOXA3; HOXA4; | 7:27170892 | cg14359292 | 266882 |
| | HOXA5; LOC100133311 | | | |
| −0.165 | LCP1 | 13:46757415 | cg06477663 | 126420 |
| −0.165 | SPAG11A | 8:7720259 | cg23703303 | 417310 |
| −0.165 | | 19:21767566 | cg19051117 | 344954 |
| −0.165 | OR10R2 | 1:158450237 | cg04132017 | 83014 |
| −0.165 | LOC284661 | 1:4481995 | cg20252997 | 363933 |
| −0.164 | FHOD3 | 18:34327498 | cg21367586 | 380971 |
| −0.164 | PCDHB4 | 5:140501812 | cg06048354 | 119137 |
| −0.164 | HLA-DQB2 | 6:32729174 | cg04418355 | 88678 |
| −0.164 | | 6:28447087 | cg26968378 | 470632 |
| −0.163 | BAI1 | 8:143580965 | cg09544050 | 182856 |
| −0.163 | LOC440905 | 2:130794753 | cg25239986 | 442097 |
| −0.163 | SPINK5 | 5:147473666 | cg04829448 | 96297 |
| −0.163 | ZNF718 | 4:124342 | cg12717203 | 236185 |
| −0.163 | TIAM2 | 6:155464712 | cg24207161 | 425489 |
| −0.162 | PACS2 | 14:105830721 | cg19499452 | 351610 |
| −0.162 | MCTS1 | X:119738550 | cg00357087 | 7563 |
| −0.162 | RASA3 | 13:114900021 | cg06611532 | 128857 |
| −0.161 | | 4:187984958 | cg19632603 | 353880 |
| −0.160 | ITGA11 | 15:68713677 | cg02454536 | 49961 |
| −0.160 | ACAP1; KCTD11; | 17:7253720 | cg02676175 | 54546 |
| | TMEM95 | | | |
| −0.160 | HAAO | 2:43023945 | cg14631114 | 272427 |
| −0.160 | WWP2 | 16:69830414 | cg03424554 | 69244 |
| −0.160 | PGRMC2 | 4:129212177 | cg02096220 | 43042 |
| −0.159 | SHISA6 | 17:11142733 | cg23474049 | 413465 |
| −0.159 | | 6:28447107 | cg12584458 | 233995 |
| −0.159 | ZP2 | 16:21222939 | cg18249108 | 332126 |
| −0.159 | CLCNKA; HSPB7 | 1:16349079 | cg08231696 | 159747 |
| −0.158 | SPINK7 | 5:147699718 | cg08859406 | 170938 |
| −0.158 | GNAS | 20:57463503 | cg11480267 | 215775 |
| −0.158 | STX2 | 12:131303247 | cg11011512 | 208090 |
| −0.158 | ADCK4 | 19:41208803 | cg07615236 | 148518 |
| −0.158 | | 6:148540308 | cg21796170 | 387391 |
| −0.158 | NDRG4 | 16:58534157 | cg05725404 | 113149 |
| −0.157 | ANXA9; CERS2 | 1:150954826 | cg13320146 | 245435 |
| −0.157 | SAMD3 | 6:130504413 | cg24048806 | 422810 |
| −0.157 | TCL6 | 14:96129574 | cg05093429 | 101497 |
| −0.157 | | 6:55761410 | cg17273096 | 316241 |
| −0.156 | B3GNTL1 | 17:81008971 | cg17652174 | 322327 |
| −0.156 | RNF219 | 13:79234251 | cg01819759 | 37343 |
| −0.156 | GGNBP2; MYO19; | 17:34891843 | cg02097479 | 43078 |
| | PIGW | | | |
| −0.156 | | 7:79964738 | cg22110839 | 392010 |
| −0.156 | HOXA3; HOXA4; | 7:27170412 | cg11410718 | 214730 |
| | LOC100133311 | | | |
| −0.155 | | 6:58777827 | cg26951520 | 470325 |
| −0.155 | KIRREL3- | 11:126872863 | cg17273911 | 316252 |
| | AS3 | | | |
| −0.155 | NRXN1 | 2:51260399 | cg00345522 | 7288 |
| −0.155 | | 14:106025958 | cg27055313 | 472099 |
| −0.155 | | 4:60001000 | cg11281224 | 212579 |
| −0.154 | | 4:9479947 | cg12006118 | 224679 |
| −0.154 | IL12RB1 | 19:18170383 | cg26642774 | 464790 |
| −0.154 | | 1:22599443 | cg07371337 | 143948 |
| −0.154 | ZNF845 | 19:53828996 | cg22256944 | 394221 |
| −0.154 | | 13:112705636 | cg01896130 | 38843 |
| −0.154 | HOTAIR; HOXC12 | 12:54354590 | cg05573844 | 110417 |
| −0.154 | | 13:61758710 | cg15508935 | 286618 |
| −0.154 | MICA | 6:31382027 | cg20144487 | 362399 |
| −0.153 | | 6:156983263 | cg05282260 | 105071 |
| −0.153 | MIR1283- | 19:54199538 | cg26325497 | 459342 |
| | 1; MIR518B; MIR518F; | | | |
| | MIR519B; | | | |
| | MIR519C; | | | |
| | MIR520A; MIR520B; | | | |
| | MIR523; | | | |
| | MIR525; MIR526A1; | | | |
| | MIR526B | | | |
| −0.153 | MKX | 10:28030033 | cg13806489 | 255420 |
| −0.153 | | 10:53639124 | cg06976598 | 136324 |
| −0.153 | CHTOP; S100A1; | 1:153600156 | cg07898899 | 153567 |
| | S100A13 | | | |
| −0.152 | | 11:24346161 | cg05202300 | 103488 |
| −0.152 | LOC728613 | 5:1641766 | cg08769156 | 169313 |
| −0.152 | LOC100506388; | 17:184106 | cg15996459 | 294655 |
| | RPH3AL | | | |
| −0.151 | PALMD | 1:100111849 | cg24603803 | 431699 |
| −0.151 | HCG4B | 6:29895116 | cg15671450 | 289296 |
| −0.151 | | 8:88760082 | cg05893029 | 116145 |
| −0.150 | GPR158 | 10:25620462 | cg01006048 | 21182 |
| −0.150 | | 14:95155784 | cg16462006 | 302671 |
| −0.150 | PALM2; PALM2- | 9:112562314 | cg13690280 | 253070 |
| | AKAP2 | | | |
| −0.150 | LOC100507055; | 12:133185615 | cg18833933 | 341613 |
| | P2RX2 | | | |
| −0.150 | IGF2; IGF2- | 11:2154132 | cg11717189 | 219595 |
| | AS; INS- | | | |
| | IGF2; MIR483 | | | |
| −0.149 | MYO16 | 13:109833854 | cg10697882 | 202881 |
| −0.149 | SUMO3 | 21:46239952 | cg22595657 | 398990 |
| −0.149 | EME2; MRPS34; | 16:1822124 | cg09540102 | 182804 |
| | NME3; SPSB3 | | | |
| −0.149 | TTLL10 | 1:1118157 | cg16366262 | 300931 |
| −0.149 | | 7:158269409 | cg17429772 | 318912 |
| −0.149 | | 12:133003907 | cg04741728 | 94673 |
| −0.148 | SHANK2 | 11:70493931 | cg05794931 | 114366 |
| −0.148 | CRTAC1 | 10:99734513 | cg19421526 | 350393 |
| −0.148 | PXDN | 2:1734344 | cg12760299 | 236833 |
| −0.148 | | 6:28447115 | cg21903646 | 389050 |
| −0.148 | GGNBP1; LINC00336 | 6:33561099 | cg04329454 | 86936 |
| −0.148 | CLGN | 4:141317067 | cg22901919 | 403995 |
| −0.147 | COL11A2; RXRB | 6:33158020 | cg16507569 | 303394 |
| −0.147 | | 7:93960063 | cg01827933 | 37531 |
| −0.147 | LHX6 | 9:124983482 | cg13832372 | 255929 |
| −0.147 | ISL1 | 5:50688913 | cg26293019 | 458765 |
| −0.147 | DBNL; PGAM2; | 7:44105050 | cg16627090 | 305718 |
| | POLM | | | |
| −0.147 | | 3:193587490 | cg21197336 | 378486 |
| −0.147 | | 14:107095056 | cg07958169 | 154668 |
| −0.147 | SNED1 | 2:241975140 | cg09991306 | 190473 |
| −0.147 | HOXA3; HOXA4; | 7:27170755 | cg03724423 | 75252 |
| | HOXA5; LOC100133311 | | | |
| −0.147 | SEPT3; WBP2NL | 22:42395067 | cg22189786 | 393238 |
| −0.147 | | 2:208541770 | cg03716125 | 75048 |
| −0.146 | | 5:66541588 | cg11553311 | 216848 |
| −0.146 | KTN1; KTN1- | 14:56046098 | cg16907514 | 310470 |
| | AS1 | | | |
| −0.146 | FAM116B; PLXNB2 | 22:50753068 | cg20435485 | 366698 |
| −0.146 | HOXA3; HOXA4; | 7:27170388 | cg07317062 | 142981 |
| | LOC100133311 | | | |
| −0.145 | HLA-A | 6:29911295 | cg09535358 | 182715 |
| −0.145 | ARNTL | 11:13302098 | cg13286116 | 244869 |
| −0.145 | MSH5; MSH5- | 6:31731881 | cg03392100 | 68641 |
| | SAPCD1; SAPCD1; | | | |
| | VWA7 | | | |
| −0.145 | IL31; LRRC43 | 12:122651623 | cg14874216 | 276626 |
| −0.145 | MSH5; MSH5- | 6:31734192 | cg13187827 | 243288 |
| | SAPCD1; SAPCD1; | | | |
| | VWA7 | | | |
| −0.144 | SERPINB6 | 6:2972435 | cg08155050 | 158293 |
| −0.144 | DLGAP2 | 8:1455764 | cg10636144 | 201951 |
| −0.144 | SAMD11 | 1:869368 | cg13692836 | 253138 |
| −0.144 | RPH3AL | 17:146926 | cg04172345 | 83843 |
| −0.144 | | 1:228743235 | cg12437277 | 231594 |
| −0.143 | | 17:79455736 | cg09841001 | 187897 |
| −0.143 | C11orf53; C11orf92 | 11:111155021 | cg03554573 | 71909 |
| −0.143 | ATXN1 | 6:16413624 | cg11291305 | 212741 |
| −0.143 | FEM1A | 19:4785374 | cg14818248 | 275598 |
| −0.143 | HLA-A | 6:29912713 | cg18349863 | 333730 |
| −0.143 | TP73 | 1:3600879 | cg17471939 | 319626 |
| −0.143 | | 1:2863815 | cg25137787 | 440554 |
| −0.143 | ARNT2 | 15:80824318 | cg19708986 | 355104 |
| −0.143 | | 13:99084682 | cg03040740 | 61905 |
| −0.143 | | 6:28446840 | cg01792601 | 36700 |
| −0.142 | LRRC15 | 3:194091167 | cg07048348 | 137676 |
| −0.142 | | 1:37231474 | cg21952052 | 389839 |
| −0.142 | DFNA5 | 7:24742552 | cg25709790 | 449401 |
| −0.142 | PACS2 | 14:105830606 | cg12425861 | 231390 |
| −0.142 | INPP5K; LOC100306951; | 17:1412950 | cg07185664 | 140562 |
| | PITPNA | | | |
| −0.141 | LOC100506388; | 17:184131 | cg24871089 | 436253 |
| | RPH3AL | | | |
| −0.141 | DDX58 | 9:32525315 | cg14286514 | 265460 |
| −0.141 | | 7:158789849 | cg19418458 | 350346 |
| −0.141 | ARHGAP30; PVRL4 | 1:161042063 | cg00862398 | 18157 |
| −0.141 | IKZF3; ZPBP2 | 17:38024146 | cg16810031 | 309049 |
| −0.141 | | 17:21416281 | cg00007215 | 167 |
| −0.141 | | 8:23605601 | cg03324961 | 67319 |
| −0.141 | PDE6B | 4:645781 | cg01132407 | 23844 |
| −0.140 | HHIPL1 | 14:100130465 | cg18678107 | 339014 |
| −0.140 | MTG1; PAOX | 10:135202906 | cg24644436 | 432348 |
| −0.140 | CES1 | 16:55866757 | cg03880033 | 78129 |
| −0.139 | TLX1NB | 10:102872719 | cg17034508 | 312397 |
| −0.139 | PCDHB17; PCDHB6 | 5:140530410 | cg23247945 | 409860 |
| −0.139 | TMEM100 | 17:53800484 | cg18040354 | 328429 |
| −0.139 | PRKG2 | 4:82075193 | cg22772235 | 401742 |
| −0.139 | SEL1L2 | 20:13830102 | cg17971895 | 327285 |
| −0.139 | | 5:74908142 | cg19683494 | 354666 |
| −0.139 | | 2:131010647 | cg27624826 | 481683 |
| −0.139 | HIPK4; PRX | 19:40904691 | cg24923931 | 437130 |
| −0.138 | SCN7A | 2:167350883 | cg14771658 | 274784 |
| −0.138 | | 15:98196247 | cg19459332 | 350984 |
| −0.137 | PMPCA; SDCCAG3; | 9:139299357 | cg14108380 | 261681 |
| | SNAPC4 | | | |
| −0.137 | | 16:62984521 | cg02248103 | 45873 |
| −0.137 | CYB5D2; ZZEF1 | 17:4043867 | cg04417708 | 88669 |
| −0.137 | CST11; CSTL1 | 20:23433608 | cg11657817 | 218579 |
| −0.137 | CTC1; LINC00324 | 17:8124867 | cg20250341 | 363887 |
| −0.137 | CNR2; FUCA1 | 1:24195143 | cg24792360 | 434918 |
| −0.137 | PKNOX2 | 11:125170859 | cg23418965 | 412666 |
| −0.137 | PAX2 | 10:102592366 | cg03286031 | 66537 |
| −0.137 | TEX2 | 17:62341028 | cg18312782 | 333101 |
| −0.137 | | 2:226913713 | cg05293897 | 105270 |
| −0.136 | TMEM72-AS1 | 10:45374906 | cg20638675 | 369712 |
| −0.136 | HLA-DQB2 | 6:32729130 | cg07180897 | 140474 |
| −0.136 | ALK | 2:29751968 | cg01412762 | 29371 |
| −0.136 | | 19:29218732 | cg12667521 | 235503 |
| −0.136 | TOX2 | 20:42548586 | cg20889774 | 373555 |
| −0.136 | RECK | 9:36036514 | cg13392078 | 246714 |
| −0.136 | MIR593 | 7:127720749 | cg20426275 | 366531 |
| −0.136 | ODZ4 | 11:78948859 | cg12642289 | 235052 |
| −0.136 | MTERF | 7:91510405 | cg16284459 | 299467 |
| −0.136 | RBFOX3 | 17:77302162 | cg08223357 | 159591 |
| −0.136 | | 18:44770781 | cg24371990 | 427906 |
| −0.136 | CACNA1C | 12:2696048 | cg08166587 | 158536 |
| −0.136 | DAB1 | 1:58089001 | cg00026909 | 659 |
| −0.135 | | 6:156983304 | cg08839808 | 170533 |
| −0.135 | MTG1; PAOX | 10:135202839 | cg17143376 | 314138 |
| −0.135 | PIP5K1A; VPS72 | 1:151170427 | cg24720336 | 433732 |
| −0.135 | ANKS3; C16orf71; | 16:4793770 | cg00605362 | 12928 |
| | ZNF500 | | | |
| −0.135 | | 11:91950460 | cg20506843 | 367791 |
| −0.135 | RTN4RL1 | 17:1835482 | cg23676314 | 416771 |
| −0.135 | | 2:10984512 | cg00442205 | 9317 |
| −0.134 | ZDHHC13 | 11:19138045 | cg00161556 | 3630 |
| −0.134 | PAPLN | 14:73712491 | cg09636214 | 184437 |
| −0.134 | CTRB2 | 16:75242114 | cg08467866 | 163985 |
| −0.133 | ZNF718 | 4:125112 | cg10197305 | 194260 |
| −0.133 | IGF2; IGF2- | 11:2154113 | cg13165070 | 242895 |
| | AS; INS- | | | |
| | IGF2; MIR483 | | | |
| −0.133 | | 2:38099030 | cg02752932 | 56067 |
| −0.133 | TACC2 | 10:123873748 | cg23828763 | 419014 |
| −0.133 | | 7:115116310 | cg04544267 | 91006 |
| −0.133 | CES1 | 16:55866997 | cg23196985 | 408839 |
| −0.133 | CCDC105 | 19:15121385 | cg18739061 | 339959 |
| −0.133 | | 19:43203368 | cg24333150 | 427217 |
| −0.132 | FXYD2; FXYD6- | 11:117696743 | cg11820026 | 221463 |
| | FXYD2 | | | |
| −0.132 | DAB2IP | 9:124363848 | cg21234082 | 379229 |
| −0.132 | LOC100289187 | 7:99195819 | cg12290671 | 229416 |
| −0.132 | OCA2 | 15:28014040 | cg15194100 | 281887 |
| −0.132 | NAV1 | 1:201644923 | cg24253904 | 426103 |
| −0.132 | SLC24A6 | 12:113773298 | cg12583184 | 233973 |
| −0.131 | | 3:42265641 | cg18812386 | 341259 |
| −0.131 | GREB1; MIR4429 | 2:11680020 | cg11534680 | 216555 |
| −0.131 | TXNRD1 | 12:104676774 | cg15775217 | 291055 |
| −0.131 | CCBL2; RBMXL1 | 1:89459529 | cg11309454 | 213030 |
| −0.131 | GPR12 | 13:27338713 | cg14339397 | 266524 |
| −0.131 | | 9:34810228 | cg13923371 | 257761 |
| −0.131 | IFI44; IFI44L | 1:79114976 | cg07107453 | 138862 |
| −0.130 | | 5:171057196 | cg07871442 | 153079 |
| −0.130 | OVCH2 | 11:7709186 | cg25833922 | 451333 |
| −0.130 | PSMD11 | 17:30770961 | cg09309269 | 178739 |
| −0.130 | DEFA4; DEFA6 | 8:6783521 | cg02932321 | 59694 |
| −0.130 | TEPP | 16:58016655 | cg01479664 | 30752 |
| −0.130 | IL28B | 19:39737768 | cg04722177 | 94275 |
| −0.130 | IL20RA | 6:137366545 | cg08823985 | 170226 |
| −0.130 | PA2G4; RPL41; | 12:56509603 | cg18500368 | 336269 |
| | ZC3H10 | | | |
| −0.129 | | 14:26338282 | cg16725552 | 307629 |
| −0.129 | ATHL1; NLRP6 | 11:281516 | cg06432119 | 125636 |
| −0.129 | GIMAP4 | 7:150264767 | cg08960815 | 172781 |
| −0.129 | KLHL29 | 2:23839710 | cg01387450 | 28832 |
| −0.129 | | 12:114701413 | cg10233830 | 194930 |
| −0.129 | | 2:62521325 | cg22770637 | 401725 |
| −0.129 | ALK | 2:30142677 | cg00643897 | 13652 |
| −0.129 | SEMA5B | 3:122712271 | cg11160908 | 210504 |
| −0.129 | TTF2 | 1:117604726 | cg19319037 | 348805 |
| −0.129 | C20orf54 | 20:741798 | cg19037007 | 344707 |
| −0.129 | | 12:69725435 | cg22375663 | 395880 |
| −0.129 | | 8:41585554 | cg17182837 | 314877 |
| −0.128 | | 3:118144311 | cg20207443 | 363278 |
| −0.128 | LINC00299 | 2:8444004 | cg18854004 | 341911 |
| −0.128 | | 11:106239635 | cg01152624 | 24270 |
| −0.128 | GNAS; GNAS- | 20:57431202 | cg09437522 | 181002 |
| | AS1 | | | |
| −0.128 | | 13:112978683 | cg20923605 | 374019 |
| −0.128 | MITF | 3:69915302 | cg03831180 | 77231 |
| −0.128 | PTN | 7:137029097 | cg15284177 | 283213 |
| −0.128 | | 1:149155792 | cg27554973 | 480528 |
| −0.127 | ENTPD1; LOC728558 | 10:97624891 | cg09217157 | 177016 |
| −0.127 | ZFPM1 | 16:88536950 | cg05183538 | 103174 |
| −0.127 | CCDC64B; HCFC1R1; | 16:3079953 | cg07063883 | 137997 |
| | THOC6; | | | |
| | TNFRSF12A | | | |
| −0.127 | APOA1BP; IQGAP3; | 1:156551873 | cg01010328 | 21258 |
| | TTC24 | | | |
| −0.127 | NRG1 | 8:32076032 | cg23964820 | 421269 |
| −0.127 | MTG1; PAOX | 10:135203102 | cg18563886 | 337268 |
| −0.127 | | 13:112984840 | cg27260867 | 475484 |
| −0.127 | AKAP8; AKAP8L | 19:15488902 | cg22765626 | 401667 |
| −0.127 | | 18:37379468 | cg15612221 | 288285 |
| −0.127 | | 17:21416258 | cg10674575 | 202559 |
| −0.127 | | 13:100583872 | cg07223206 | 141319 |
| −0.126 | KLK1; KLK15 | 19:51334890 | cg26325285 | 459337 |
| −0.126 | GALNT9; LOC100130238 | 12:132859608 | cg10173694 | 193804 |
| −0.126 | ATG4C | 1:63249199 | cg24082680 | 423369 |
| −0.126 | | 7:102330635 | cg06322601 | 123757 |
| −0.126 | | 3:73723546 | cg02131018 | 43720 |
| −0.126 | HIPK4; PRX | 19:40904851 | cg00478479 | 10159 |
| −0.126 | GNAT1; SLC38A3 | 3:50242325 | cg15335139 | 283889 |
| −0.126 | CYP4F30P | 2:131451149 | cg14204395 | 263669 |
| −0.126 | | 16:88804051 | cg26748794 | 466680 |
| −0.126 | HUNK | 21:33242495 | cg15636421 | 288718 |
| −0.125 | KLHL29 | 2:23840282 | cg17129809 | 313908 |
| −0.125 | MEIS1 | 2:66657913 | cg10835286 | 205203 |
| −0.125 | NUP98; PGAP2 | 11:3817849 | cg19247588 | 347686 |
| −0.125 | MIR4287; SCARA5 | 8:27735178 | cg04214878 | 84655 |
| −0.125 | SPINK7 | 5:147699892 | cg13022905 | 240779 |
| −0.125 | PTH1R | 3:46936655 | cg02527881 | 51488 |
| −0.125 | GIMAP4 | 7:150264987 | cg00323915 | 6821 |
| −0.125 | PTHLH | 12:28120054 | cg20389635 | 366021 |
| −0.125 | EIF2D | 1:206786174 | cg16966520 | 311311 |
| −0.124 | | 7:27475596 | cg10070328 | 191836 |
| −0.124 | | 1:161409970 | cg00975746 | 20506 |
| −0.124 | KCNK10 | 14:88791298 | cg02883668 | 58609 |
| −0.124 | | 15:34788847 | cg10767425 | 203961 |
| −0.124 | | 8:49044542 | cg11491002 | 215895 |
| −0.124 | DLGAP4 | 20:35064637 | cg01463540 | 30456 |
| −0.124 | HSP90B3P | 1:92099858 | cg04090794 | 82132 |
| −0.124 | NOSIP; PRRG2 | 19:50084626 | cg21913888 | 389236 |
| −0.124 | ISL1 | 5:50692281 | cg24549912 | 430883 |
| −0.124 | STC2 | 5:172751331 | cg01984743 | 40717 |
| −0.124 | | 2:72079276 | cg07258910 | 141932 |
| −0.123 | PKD1L2 | 16:81254169 | cg03406106 | 68931 |
| −0.123 | ANO4 | 12:101187093 | cg25027699 | 438862 |
| −0.123 | TRIM21 | 11:4415203 | cg07244098 | 141703 |
| −0.123 | MRPL38; TRIM65 | 17:73892097 | cg01453052 | 30258 |
| −0.123 | BTN3A3 | 6:26442290 | cg12537337 | 233273 |
| −0.123 | LOC100129620; | 1:99469634 | cg24373253 | 427922 |
| | LPPR5 | | | |
| −0.123 | FYN | 6:112165062 | cg26846943 | 468428 |
| −0.123 | ASB1 | 2:239334832 | cg08773314 | 169376 |
| −0.123 | FAM163A | 1:179713788 | cg14748380 | 274436 |
| −0.122 | KIAA1143; KIF15 | 3:44802604 | cg09333631 | 179217 |
| −0.122 | MAP3K7 | 6:91297516 | cg18683523 | 339091 |
| −0.122 | | 4:9382953 | cg02136252 | 43830 |
| −0.122 | | 5:141616065 | cg24749559 | 434242 |
| −0.122 | | 15:101389733 | cg07035436 | 137452 |
| −0.122 | | 5:72510846 | cg26653990 | 465004 |
| −0.122 | | 17:78955599 | cg11521079 | 216324 |
| −0.122 | | 2:227476235 | cg08982961 | 173162 |
| −0.122 | LRRC27 | 10:134150542 | cg22586726 | 398883 |
| −0.122 | EXOC7; GALR2; | 17:74073876 | cg07909178 | 153763 |
| | SRP68; ZACN | | | |
| −0.121 | TAF3 | 10:7859768 | cg09994553 | 190530 |
| −0.121 | CDK12; MED1 | 17:37608096 | cg15445000 | 285571 |
| −0.121 | HLA- | 6:33043849 | cg22483030 | 397307 |
| | DPA1; HLA- | | | |
| | DPB1 | | | |
| −0.121 | NUBP1 | 16:10837135 | cg09079882 | 174735 |
| −0.121 | TSLP | 5:110407538 | cg15739437 | 290539 |
| −0.121 | PSMD9; WDR66 | 12:122356598 | cg21016266 | 375557 |
| −0.121 | DOK7 | 4:3487436 | cg15652666 | 289019 |
| −0.121 | OR6B3; PRR21 | 2:240985618 | cg10906826 | 206344 |
| −0.121 | FBLN7 | 2:112898400 | cg09982942 | 190323 |
| −0.120 | CXCR7 | 2:237478664 | cg03626672 | 73351 |
| −0.120 | MBTPS2 | X:21858304 | cg21195120 | 378430 |
| −0.120 | NLRP3; OR2B11 | 1:247616686 | cg14830002 | 275801 |
| −0.120 | CTAGE11P | 13:75814496 | cg17109374 | 313605 |
| −0.120 | | 5:177395755 | cg11264997 | 212321 |
| −0.120 | GAS7; RCVRN | 17:9805074 | cg26853458 | 468546 |
| −0.120 | GAD2 | 10:26531942 | cg23052437 | 406538 |
| −0.120 | LCE2A | 1:152670843 | cg13697387 | 253231 |
| −0.120 | SPDYC | 11:64936920 | cg23079866 | 407032 |
| −0.119 | SPTBN2 | 11:66487938 | cg04883026 | 97440 |
| −0.119 | HOTTIP; HOXA11- | 7:27236674 | cg01363170 | 28338 |
| | AS1; HOXA13 | | | |
| −0.119 | SYS1; SYS1- | 20:43991402 | cg00385441 | 8134 |
| | DBNDD2 | | | |
| −0.119 | ZNF876P | 4:204382 | cg00569896 | 12104 |
| −0.119 | CCDC71; KLHDC8B | 3:49203081 | cg18590502 | 337716 |
| −0.119 | GRAMD2 | 15:72448319 | cg12252008 | 228855 |
| −0.119 | | 16:76228294 | cg06827491 | 133242 |
| −0.119 | LOC116437 | 12:131645153 | cg07374109 | 143989 |
| −0.119 | CACNG4 | 17:64973939 | cg23968154 | 421320 |
| −0.119 | NAV1 | 1:201619295 | cg04179952 | 83989 |
| −0.119 | | 12:58934471 | cg01919941 | 39316 |
| −0.119 | | 13:21893289 | cg21970626 | 390084 |
| −0.119 | ANXA9; FAM63A | 1:150969592 | cg21859421 | 388363 |
| −0.119 | | 1:10895517 | cg17480077 | 319783 |
| −0.118 | LRRC27 | 10:134150489 | cg21370255 | 381012 |
| −0.118 | POTEA | 8:43147397 | cg26969150 | 470647 |
| −0.118 | HOXA3; HOXA4; | 7:27170313 | cg04317399 | 86720 |
| | LOC100133311 | | | |
| −0.118 | C1orf86; PRKCZ | 1:2121449 | cg08200543 | 159156 |
| −0.118 | | 8:81143546 | cg16745033 | 307963 |
| −0.118 | | 7:136362483 | cg11678250 | 218935 |
| −0.118 | SPIN2B | X:57147918 | cg12074688 | 225883 |
| −0.118 | | 8:144152376 | cg17001448 | 311828 |
| −0.118 | ASPHD1; KCTD13; | 16:29912700 | cg10093648 | 192261 |
| | SEZ6L2 | | | |
| −0.118 | EIF2D | 1:206786178 | cg16639185 | 305935 |
| −0.118 | PXN | 12:120688744 | cg22564731 | 398621 |
| −0.118 | MIR4757; OSR1 | 2:19553950 | cg00954878 | 20061 |
| −0.118 | PROX1 | 1:214152805 | cg26054764 | 454613 |
| −0.117 | CDH3 | 16:68691561 | cg09821218 | 187538 |
| −0.117 | LPP | 3:188115336 | cg23651889 | 416316 |
| −0.117 | | 5:72666838 | cg11316517 | 213156 |
| −0.117 | DSCR3 | 21:38630506 | cg18380783 | 334168 |
| −0.117 | | 7:136190456 | cg03439301 | 69540 |
| −0.117 | TMEM100 | 17:53800554 | cg03233426 | 65601 |
| −0.117 | PSMD11 | 17:30770932 | cg20190559 | 363067 |
| −0.117 | | 5:171057078 | cg11620716 | 217943 |
| −0.117 | FN3K; TBCD | 17:80708513 | cg00809820 | 17098 |
| −0.117 | SECTM1 | 17:80285515 | cg05744064 | 113450 |
| −0.117 | NCOA1 | 2:24806444 | cg23648923 | 416268 |
| −0.117 | LOC100128398; | 19:58515046 | cg09577391 | 183474 |
| | ZNF606 | | | |
| −0.117 | LOC100289187 | 7:99195788 | cg24024660 | 422355 |
| −0.117 | | 16:77535527 | cg06794141 | 132525 |
| −0.117 | | 2:192792315 | cg01836906 | 37718 |
| −0.117 | LELP1 | 1:153175842 | cg00298977 | 6302 |
| −0.117 | ODF1 | 8:103556749 | cg27000960 | 471198 |
| −0.117 | GCK | 7:44229319 | cg16476432 | 302901 |
| −0.117 | | 11:2211939 | cg19586845 | 353065 |
| −0.117 | HCG4; LOC554223 | 6:29761905 | cg25193571 | 441364 |
| −0.117 | CTNND2 | 5:11588971 | cg01937840 | 39702 |
| −0.116 | MYOM2 | 8:1994478 | cg20219329 | 363455 |
| −0.116 | | 15:30337018 | cg14398295 | 267622 |
| −0.116 | RASAL1 | 12:113574368 | cg16890739 | 310176 |
| −0.116 | | 15:36699121 | cg02763009 | 56272 |
| −0.116 | BIRC7; FLJ16779; | 20:61878032 | cg12649455 | 235197 |
| | MIR3196; | | | |
| | NKAIN4 | | | |
| −0.116 | | 7:1250246 | cg07034004 | 137422 |
| −0.116 | | 5:170763642 | cg23036405 | 406232 |
| −0.116 | NCR1 | 19:55417647 | cg12952132 | 239747 |
| −0.116 | | 1:22591361 | cg13048466 | 241145 |
| −0.115 | GLP1R | 6:39015339 | cg19772011 | 356340 |
| −0.115 | RGMA | 15:93615146 | cg21079003 | 376460 |
| −0.115 | PGM5P2 | 9:69148624 | cg18861917 | 342052 |
| −0.115 | ZBED4 | 22:50250283 | cg20618087 | 369398 |
| −0.115 | LARS2 | 3:45429742 | cg04459086 | 89409 |
| −0.115 | TACC2 | 10:123886914 | cg26111575 | 455575 |
| −0.115 | ST8SIA2 | 15:92939006 | cg08289525 | 160900 |
| −0.115 | ANO7 | 2:242147702 | cg00255732 | 5474 |
| −0.115 | LRRC34; LRRIQ4 | 3:169531864 | cg10994914 | 207829 |
| −0.115 | | 14:88097221 | cg26252794 | 458035 |
| −0.115 | DGKB | 7:14415317 | cg03229767 | 65553 |
| −0.114 | LRRC27 | 10:134150690 | cg02535060 | 51631 |
| −0.114 | LOC100131138 | 12:111374169 | cg09792970 | 187023 |
| −0.114 | CNNM2 | 10:104676907 | cg15439196 | 285445 |
| −0.114 | CCNYL1 | 2:208572165 | cg18757817 | 340266 |
| −0.114 | PRRX1 | 1:170631847 | cg22693806 | 400548 |
| −0.114 | | 11:44489577 | cg03708694 | 74912 |
| −0.114 | DKFZp434L192 | 7:56563820 | cg21216010 | 378877 |
| −0.114 | NRP1 | 10:33612892 | cg17425818 | 318845 |
| −0.114 | TFCP2 | 12:51570174 | cg21570277 | 384054 |
| −0.114 | LHX6 | 9:124982834 | cg17930194 | 326612 |
| −0.114 | MMP17 | 12:132317128 | cg01830463 | 37580 |
| −0.114 | LRRC27 | 10:134150567 | cg07119315 | 139090 |
| −0.114 | | 8:67317506 | cg02934300 | 59732 |
| −0.114 | DBNL; PGAM2; | 7:44105721 | cg21088488 | 376592 |
| | POLM | | | |
| −0.114 | | 1:113425263 | cg10541466 | 200276 |
| −0.114 | AGAP1 | 2:236774164 | cg03785646 | 76466 |
| −0.114 | | 7:73424891 | cg09076650 | 174691 |
| −0.114 | | 6:1449399 | cg18182981 | 331002 |
| −0.114 | NDUFA12 | 12:95397704 | cg22141781 | 392510 |
| −0.114 | HOXA3; HOXA4; | 7:27170880 | cg04321618 | 86813 |
| | HOXA5; LOC100133311 | | | |
| −0.113 | MIR4283- | 7:63085215 | cg10945855 | 206928 |
| | 1; MIR4283-2 | | | |
| −0.113 | PPIL2 | 22:22020076 | cg04983276 | 99379 |
| −0.113 | | 2:72079413 | cg24516106 | 430249 |
| −0.113 | | 10:125222790 | cg11653519 | 218499 |
| −0.113 | | 7:21209781 | cg08701183 | 168131 |
| −0.113 | MTG1; PAOX | 10:135202522 | cg10902549 | 206273 |
| −0.113 | NR2F1 | 5:92927796 | cg26067259 | 454804 |
| −0.113 | | 2:218048915 | cg00549040 | 11649 |
| −0.113 | GRK4 | 4:3043527 | cg01807241 | 37046 |
| −0.113 | TNXB | 6:32066446 | cg14011987 | 259623 |
| −0.113 | SNED1 | 2:241975756 | cg26707709 | 466046 |
| −0.113 | | 6:28446794 | cg03350138 | 67869 |
| −0.113 | C6orf48; HSPA1B; | 6:31806478 | cg17794996 | 324365 |
| | SNORD48; | | | |
| | SNORD52 | | | |
| −0.113 | CBX7 | 22:39545030 | cg18708252 | 339475 |
| −0.113 | UPF2 | 10:12085641 | cg00513208 | 339475 |
| −0.113 | | 17:80860250 | cg04497611 | 90135 |
| −0.112 | DMD | X:31285129 | cg06746884 | 131521 |
| −0.112 | FGF4 | 11:69587473 | cg26765567 | 466900 |
| −0.112 | | 6:30419373 | cg03507875 | 70857 |
| −0.112 | | 19:29217858 | cg25267487 | 442467 |
| −0.112 | | 16:84953424 | cg01765164 | 36115 |
| −0.112 | DGKG | 3:185912486 | cg18172135 | 330799 |
| −0.112 | | 13:111451972 | cg22731513 | 401204 |
| −0.112 | FZD1 | 7:90895894 | cg08714590 | 168374 |
| −0.112 | CLCN7 | 16:1524255 | cg08008072 | 155549 |
| −0.112 | CRX | 19:48324309 | cg06945747 | 135695 |
| −0.112 | | 1:91227319 | cg08895590 | 171690 |
| −0.112 | KALRN | 3:124232190 | cg06690085 | 130439 |
| −0.112 | | 9:128799328 | cg13946341 | 258251 |
| −0.112 | IL34; MTSS1L | 16:70688171 | cg02602352 | 52989 |
| −0.112 | TMEM171 | 5:72431891 | cg01170694 | 24642 |
| −0.112 | | 17:42706084 | cg14836839 | 275934 |
| −0.112 | RASGEF1C | 5:179634671 | cg12408990 | 231108 |
| −0.112 | DOK7 | 4:3486116 | cg00752263 | 15898 |
| −0.112 | ANAPC2; GRIN1; | 9:140062197 | cg03206445 | 65043 |
| | LRRC26; | | | |
| | MIR3621 | | | |
| −0.111 | OR6Q1; OR9Q1 | 11:57792403 | cg18886347 | 342489 |
| −0.111 | BAI1 | 8:143580823 | cg05758861 | 113684 |
| −0.111 | | 2:121280002 | cg00314115 | 6610 |
| −0.111 | MUC22 | 6:30980847 | cg27292220 | 475981 |
| −0.111 | AGAP1 | 2:236773924 | cg15981851 | 294380 |
| −0.111 | | 6:55774999 | cg19318364 | 348791 |
| −0.111 | ITIH1; ITIH3 | 3:52824283 | cg18404041 | 334584 |
| −0.111 | PDE6B | 4:648752 | cg17643796 | 322207 |
| −0.111 | TRIM7 | 5:180614858 | cg25606201 | 447878 |
| −0.111 | | 8:141490124 | cg15842218 | 292164 |
| −0.111 | IPMK | 10:59949191 | cg09039685 | 174085 |
| −0.111 | CCDC129 | 7:31685368 | cg02943305 | 59917 |
| −0.111 | | 5:1645640 | cg11628972 | 218045 |
| −0.111 | CDHR3 | 7:105596715 | cg27099486 | 472857 |
| −0.111 | DCX | X:110654011 | cg11031647 | 208426 |
| −0.111 | PLA2G4D | 15:42371967 | cg02760164 | 56222 |
| −0.111 | ANKRD34C | 15:79576704 | cg14577319 | 271361 |
| −0.110 | | 1:161391807 | cg00056433 | 1344 |
| −0.110 | FHL3; SF3A3 | 1:38460950 | cg05202204 | 103487 |
| −0.110 | | 5:72485634 | cg14727987 | 274140 |
| −0.110 | | 11:41481578 | cg24638253 | 432227 |
| −0.110 | | 6:27521330 | cg26264769 | 458241 |
| −0.110 | | 5:177592761 | cg25087499 | 439729 |
| −0.110 | | 14:103783717 | cg10559742 | 200586 |
| −0.110 | ARL4C | 2:235406291 | cg05308656 | 105595 |
| −0.110 | HHIP; LOC646576 | 4:145566595 | cg02524475 | 51415 |
| −0.110 | C11orf9 | 11:61545187 | cg07136108 | 139453 |
| −0.110 | PCDHA1; PCDHA10; | 5:140238079 | cg18906028 | 342772 |
| | PCDHA11; | | | |
| | PCDHA2; | | | |
| | PCDHA3; PCDHA4; | | | |
| | PCDHA5; | | | |
| | PCDHA6; | | | |
| | PCDHA7; PCDHA8; | | | |
| | PCDHA9 | | | |
| −0.110 | LOC151171 | 2:239463229 | cg10182060 | 193967 |
| −0.110 | | 14:22385890 | cg16138400 | 296975 |
| −0.110 | MAGIX; PLP2; | X:49027884 | cg22366498 | 395712 |
| | PRICKLE3 | | | |
| −0.110 | COL1A1 | 17:48288538 | cg06769994 | 132032 |
| −0.110 | FAM70A | X:119443941 | cg26583344 | 463923 |
| −0.110 | CXCR2 | 2:219001517 | cg10819992 | 204935 |
| −0.110 | | 16:66162203 | cg26798936 | 467489 |
| −0.110 | CCHCR1; PSORS1C2 | 6:31105872 | cg03034365 | 61773 |
| −0.110 | | 3:36675685 | cg12870891 | 238511 |
| −0.110 | DEFB135; DEFB136 | 8:11838843 | cg12807628 | 237566 |
| −0.110 | MDC1; TUBB | 6:30684572 | cg10925640 | 206607 |
| −0.110 | CLDN4; WBSCR27 | 7:73256416 | cg03781731 | 76399 |
| −0.110 | SNRPN | 15:25123381 | cg08560373 | 165668 |
| −0.109 | FBXL16; METRN | 16:757149 | cg07644125 | 149038 |
| −0.109 | EIF2D | 1:206786170 | cg21155316 | 377647 |
| −0.109 | | 8:135726252 | cg00057272 | 1361 |
| −0.109 | | 13:112979998 | cg20620751 | 369432 |
| −0.109 | DHRS7 | 14:60629192 | cg06898279 | 134753 |
| −0.109 | EFS; IL25 | 14:23835595 | cg17205324 | 315223 |
| −0.109 | PALMD | 1:100111727 | cg07960762 | 154721 |
| −0.109 | | 10:79470169 | cg19094530 | 345528 |
| −0.109 | ARHGEF6 | X:135863582 | cg01134144 | 23879 |
| −0.109 | DUS3L | 19:5799340 | cg20467412 | 367204 |
| −0.109 | RGMA | 15:93633408 | cg04697454 | 93855 |
| −0.109 | VWA5B1 | 1:20608736 | cg16713551 | 307396 |
| −0.109 | | 14:106791268 | cg14042931 | 260352 |
| −0.109 | | 19:1673498 | cg10697737 | 202878 |
| −0.109 | GPR4; OPA3 | 19:46097075 | cg16620032 | 305585 |
| −0.109 | LOC388630 | 1:48452740 | cg07202054 | 140866 |
| −0.108 | HK3; UNC5A | 5:176314628 | cg06783121 | 132285 |
| −0.108 | | 6:12342070 | cg03519011 | 71096 |
| −0.108 | C12orf73; TDG | 12:104350162 | cg22927789 | 404375 |
| −0.108 | CDH22 | 20:44882514 | cg25558663 | 447055 |
| −0.108 | | 6:18752614 | cg00664117 | 14044 |
| −0.108 | ARHGEF25; B4GALNT1; | 12:58011837 | cg00700412 | 14808 |
| | DTX3; | | | |
| | SLC26A10 | | | |
| −0.108 | SHANK2 | 11:70564414 | cg13080721 | 241684 |
| −0.108 | LOC154449 | 6:170571686 | cg18698681 | 339306 |
| −0.108 | KCNH2 | 7:150644459 | cg01559770 | 32264 |
| −0.108 | TMEM132D | 12:130384734 | cg08261450 | 160299 |
| −0.108 | DUOX1; DUOXA1 | 15:45427034 | cg14918338 | 277309 |
| −0.108 | DNAJB13 | 11:73661250 | cg02070028 | 42486 |
| −0.108 | PSMD7 | 16:74331618 | cg09313046 | 178793 |
| −0.108 | LGR6 | 1:202172778 | cg04811114 | 95994 |
| −0.108 | | 13:69557960 | cg18586262 | 337639 |
| −0.108 | | 1:201316444 | cg26233753 | 457728 |
| −0.108 | | 1:37139265 | cg20745381 | 371614 |
| −0.108 | | 10:121837310 | cg10087780 | 192116 |
| −0.108 | | 15:98196234 | cg08064292 | 156507 |
| −0.107 | GUCY2E; LRRC32 | 11:76381800 | cg19752094 | 355941 |
| −0.107 | LOC440910 | 2:132057004 | cg07051654 | 137739 |
| −0.107 | NAPSA | 19:50861262 | cg12273319 | 229159 |
| −0.107 | TRIM69 | 15:45028270 | cg10439456 | 198368 |
| −0.107 | CHTOP; S100A1; | 1:153599831 | cg02331910 | 47613 |
| | S100A13 | | | |
| −0.107 | | 5:132938694 | cg19005955 | 344221 |
| −0.107 | ACOT11; FAM151A | 1:55096622 | cg22619910 | 399357 |
| −0.107 | CRYBA2; FEV; | 2:219851181 | cg18664514 | 338819 |
| | LOC151300 | | | |
| −0.107 | C10orf96 | 10:118084504 | cg13836550 | 255997 |
| −0.107 | SORCS3 | 10:106749483 | cg26349484 | 459830 |
| −0.107 | ZEB2; ZEB2- | 2:145273345 | cg04640885 | 92836 |
| | AS1 | | | |
| −0.107 | GALNT6 | 12:51786489 | cg15127250 | 280846 |
| −0.107 | | 3:137187558 | cg24821307 | 435362 |
| −0.107 | ADORA1 | 1:203086869 | cg18811731 | 341246 |
| −0.107 | OR1J4; OR1N1 | 9:125289357 | cg14598387 | 271804 |
| −0.107 | CARTPT | 5:71012177 | cg11927345 | 223348 |
| −0.107 | HCRT; KCNH4 | 17:40330907 | cg06514003 | 127062 |
| −0.107 | SNRPN | 15:25093366 | cg02171545 | 44484 |
| −0.107 | C2orf28; CAD; | 2:27434262 | cg14242246 | 264531 |
| | SLC5A6 | | | |
| −0.107 | HOXA3; HOXA4; | 7:27170552 | cg16651126 | 306141 |
| | LOC100133311 | | | |
| −0.107 | | 15:20562413 | cg03344283 | 67729 |
| −0.106 | PLA2G7 | 6:46703670 | cg12854458 | 238255 |
| −0.106 | MAG | 19:35790951 | cg25443560 | 445295 |
| −0.106 | HLA-DRB1 | 6:32552022 | cg10765922 | 203924 |
| −0.106 | FAM83H; MAPK15 | 8:144801088 | cg05626536 | 111385 |
| −0.106 | | 3:193987757 | cg16282892 | 299443 |
| −0.106 | POU4F2 | 4:147573410 | cg05710982 | 112882 |
| −0.106 | DKFZp434J0226 | 19:46712075 | cg00537984 | 11421 |
| −0.106 | GPR31 | 6:167571630 | cg25285053 | 442729 |
| −0.106 | PCDHA1; PCDHA10; | 5:140248610 | cg25024993 | 438807 |
| | PCDHA11; | | | |
| | PCDHA12; | | | |
| | PCDHA2; PCDHA3; | | | |
| | PCDHA4; | | | |
| | PCDHA5; | | | |
| | PCDHA6; PCDHA7; | | | |
| | PCDHA8; | | | |
| | PCDHA9 | | | |
| −0.106 | SGPP2 | 2:223288637 | cg11300809 | 212887 |
| −0.106 | DLC1 | 8:13373090 | cg00933411 | 19610 |
| −0.106 | PRSS55 | 8:10386222 | cg10657228 | 202268 |
| −0.106 | ZIC1; ZIC4 | 3:147120248 | cg13897134 | 257142 |
| −0.106 | DKFZp434L192 | 7:56563742 | cg17876758 | 325825 |
| −0.106 | C11orf10; C11orf9 | 11:61548003 | cg19617373 | 353600 |
| −0.106 | | 6:99075228 | cg05593122 | 110762 |
| −0.106 | | 14:104787388 | cg05210671 | 103662 |
| −0.106 | HHLA2 | 3:108041508 | cg24769830 | 434575 |
| −0.106 | LOC100506012; | 19:46999055 | cg03969996 | 79829 |
| | PNMAL2 | | | |
| −0.106 | BEX5 | X:101411275 | cg03993934 | 80323 |
| −0.106 | | 15:98196333 | cg11023328 | 208285 |
| −0.105 | ZIC5 | 13:100605441 | cg10523198 | 199939 |
| −0.105 | TIGIT | 3:114009756 | cg05943254 | 117166 |
| −0.105 | CTNND2 | 5:11589049 | cg17415451 | 318667 |
| −0.105 | MGC27382 | 1:78693899 | cg22559013 | 398527 |
| −0.105 | DLC1 | 8:13372483 | cg08768218 | 169300 |
| −0.105 | F10; F7 | 13:113783990 | cg25796986 | 450780 |
| −0.105 | SOX2-OT | 3:181417613 | cg06283493 | 123126 |
| −0.105 | | 13:54898133 | cg18817396 | 341362 |
| −0.105 | KIAA1143; KIF15 | 3:44802566 | cg17546649 | 320901 |
| −0.105 | CRYAA | 21:44593708 | cg21895314 | 388915 |
| −0.105 | FOXS1 | 20:30437731 | cg05366561 | 106738 |
| −0.105 | FIBIN | 11:27015592 | cg02565993 | 52210 |
| −0.105 | CLC | 19:40228820 | cg04764056 | 95048 |
| −0.105 | | 15:84974881 | cg06813100 | 132909 |
| −0.105 | FBXO47 | 17:37123767 | cg10600889 | 201304 |
| −0.105 | C14orf23; FOXG1 | 14:29240992 | cg03472898 | 70248 |
| −0.105 | STK10 | 5:171541827 | cg11334870 | 213456 |
| −0.105 | HLA- | 6:33041697 | cg00798281 | 16870 |
| | DPA1; HLA- | | | |
| | DPB1 | | | |
| −0.105 | P2RX5 | 17:3598755 | cg13375828 | 246413 |
| −0.105 | LOC100506012; | 19:46998383 | cg06484000 | 126528 |
| | PNMAL2 | | | |
| −0.105 | CMBL | 5:10308785 | cg26878318 | 468990 |
| −0.105 | MYO7A | 11:76903134 | cg24852442 | 435899 |
| −0.105 | DKFZp434L192 | 7:56555482 | cg21720373 | 386268 |
| −0.105 | | 22:50482250 | cg10146330 | 193299 |
| −0.105 | PKD1L2 | 16:81254209 | cg07091758 | 138538 |
| −0.104 | | 2:75136334 | cg12269972 | 229112 |
| −0.104 | | 6:30433749 | cg07017214 | 137090 |
| −0.104 | SLC38A4 | 12:47226005 | cg08005660 | 155507 |
| −0.104 | XPNPEP1 | 10:111683631 | cg17201651 | 315167 |
| −0.104 | MYH11 | 16:15931580 | cg03737209 | 75526 |
| −0.104 | FLOT1; TUBB | 6:30696029 | cg22024120 | 390747 |
| −0.104 | | 5:170763941 | cg26497768 | 462323 |
| −0.104 | KCNK3 | 2:26916575 | cg20955989 | 374545 |
| −0.104 | TRIM31 | 6:30080230 | cg17048834 | 312621 |
| −0.104 | ABCA7; HMHA1 | 19:1064938 | cg26576206 | 463804 |
| −0.104 | PRKCB | 16:23850404 | cg26562691 | 463553 |
| −0.104 | GAPDHS; LOC100506469; | 19:36024313 | cg25681177 | 449001 |
| | SBSN | | | |
| −0.104 | CDSN; PSORS1C1; | 6:31096204 | cg03880216 | 78131 |
| | PSORS1C2 | | | |
| −0.104 | MORC2; MORC2- | 22:31316184 | cg26278151 | 458506 |
| | AS1 | | | |
| −0.104 | PCDHB5 | 5:140515736 | cg15120855 | 280719 |
| −0.104 | | 6:68849256 | cg09531389 | 182666 |
| −0.104 | WNT10A | 2:219763642 | cg16582803 | 304901 |
| −0.104 | | 14:56471704 | cg05314124 | 105693 |
| −0.104 | MMRN2; SNCG | 10:88717623 | cg07436701 | 145067 |
| −0.104 | | 1:31280493 | cg25204088 | 441555 |
| −0.104 | RFTN2 | 2:198540621 | cg08373610 | 162365 |
| −0.104 | C1QTNF8 | 16:1152474 | cg04004158 | 80525 |
| −0.104 | LOC100507463; | 6:32805692 | cg26685246 | 465648 |
| | PSMB8; TAP1; | | | |
| | TAP2 | | | |
| −0.104 | THOC2 | X:122867073 | cg08100265 | 157187 |
| −0.104 | PDLIM4 | 5:131589552 | cg14815361 | 275561 |
| −0.103 | HHATL; KBTBD5 | 3:42727489 | cg25129124 | 440393 |
| −0.103 | LOC84931 | 2:121223847 | cg21406402 | 381501 |
| −0.103 | MACROD2 | 20:15177509 | cg09937190 | 189621 |
| −0.103 | FAM163A | 1:179713828 | cg24460126 | 429325 |
| −0.103 | LOC100506012; | 19:46988034 | cg14128773 | 262082 |
| | PNMAL2 | | | |
| −0.103 | ABHD8; DDA1; | 19:17420304 | cg10664184 | 202380 |
| | MRPL34 | | | |
| −0.103 | SLC9A3 | 5:501938 | cg22572362 | 398700 |
| −0.103 | C1QTNF8; LOC146336; | 16:1133074 | cg03327829 | 67387 |
| | SSTR5 | | | |
| −0.103 | HCN4 | 15:73639105 | cg11831431 | 221705 |
| −0.103 | | 3:27638612 | cg14143723 | 262354 |
| −0.103 | BST2 | 19:17516712 | cg01329005 | 27706 |
| −0.103 | | 4:115649889 | cg14855679 | 276322 |
| −0.103 | TMEM136 | 11:120187999 | cg09120934 | 175365 |
| −0.103 | IRS4 | X:107980846 | cg21111568 | 376933 |
| −0.103 | | 7:55072042 | cg13490352 | 248855 |
| −0.103 | NCKAP5 | 2:134326172 | cg18984282 | 343881 |
| −0.103 | NCR1 | 19:55417496 | cg17804112 | 324505 |
| −0.103 | FHL3; SF3A3 | 1:38461687 | cg17077180 | 313040 |
| −0.102 | SEMA5B | 3:122712224 | cg04830808 | 96325 |
| −0.102 | SLCO1B1 | 12:21324585 | cg04579507 | 91754 |
| −0.102 | | 22:39323204 | cg07123855 | 139180 |
| −0.102 | | 5:134881662 | cg12613020 | 234541 |
| −0.102 | | 2:220352476 | cg20456243 | 367005 |
| −0.102 | BAI1 | 8:143581070 | cg25473866 | 445797 |
| −0.102 | PCDHB17; PCDHB6 | 5:140528785 | cg26866168 | 468766 |
| −0.102 | GABBR2 | 9:101192792 | cg14523238 | 270180 |
| −0.102 | | 5:74908170 | cg00601450 | 12858 |
| −0.102 | OCA2 | 15:28053339 | cg24943998 | 437498 |
| −0.102 | DPF1 | 19:38721070 | cg23480284 | 413571 |
| −0.102 | TRAPPC12; TSSC1 | 2:3383257 | cg12447832 | 231782 |
| −0.102 | FGF8; NPM3 | 10:103532798 | cg16079364 | 296042 |
| −0.102 | CHRND; PRSS56 | 2:233384489 | cg25571880 | 447246 |
| −0.102 | APCDD1 | 18:10456115 | cg14623989 | 272309 |
| −0.102 | | X:119151476 | cg22956922 | 404868 |
| −0.102 | | 5:88991074 | cg04117076 | 82698 |
| −0.102 | | 2:131010522 | cg13302869 | 245147 |
| −0.102 | | 13:19243756 | cg08628006 | 166844 |
| −0.102 | GCM2; SYCP2L | 6:10881782 | cg18187244 | 331085 |
| −0.102 | | 7:158243632 | cg23859303 | 419493 |
| −0.102 | C16orf91; CCDC154 | 16:1478718 | cg09615056 | 184112 |
| −0.102 | LRRC10B; MIR4488; | 11:61273282 | cg15263021 | 282930 |
| | SYT7 | | | |
| −0.102 | FBXO7 | 22:32870150 | cg21023126 | 375623 |
| −0.102 | | 13:112985463 | cg17655970 | 322387 |
| −0.102 | | 15:74060182 | cg26579616 | 463866 |
| −0.102 | | 6:28781594 | cg00176718 | 3961 |
| −0.102 | PLK5 | 19:1542897 | cg00392196 | 8275 |
| −0.102 | | 6:156983315 | cg00269725 | 5770 |
| −0.102 | LOC100506776 | 7:38403507 | cg05460050 | 108373 |
| −0.102 | | 5:15385947 | cg00105403 | 2447 |
| −0.102 | CKMT1A | 15:43984922 | cg26247942 | 457947 |
| −0.102 | | 1:14462810 | cg16164607 | 297377 |
| −0.102 | CDH22 | 20:44838981 | cg07296387 | 142575 |
| −0.102 | | 10:133156015 | cg09005971 | 173513 |
| −0.101 | CHRDL1 | X:110038954 | cg07653640 | 149177 |
| −0.101 | | 6:79339334 | cg11074783 | 209107 |
| −0.101 | C1orf159 | 1:1052600 | cg10126324 | 192906 |
| −0.101 | RINL; SIRT2 | 19:39360313 | cg02711397 | 55211 |
| −0.101 | ZFP41 | 8:144339106 | cg19504605 | 351705 |
| −0.101 | | 16:85981336 | cg10274029 | 195601 |
| −0.101 | | 11:2212225 | cg08241307 | 159919 |
| −0.101 | | 15:98196022 | cg00744413 | 15733 |
| −0.101 | | 5:9119047 | cg17608082 | 321764 |
| −0.101 | DPYSL5 | 2:27068081 | cg08806184 | 169893 |
| −0.101 | GP6 | 19:55526208 | cg27154418 | 473807 |
| −0.101 | C16orf61; CENPN | 16:81039227 | cg08265926 | 160397 |
| −0.101 | LOC100129620; | 1:99471295 | cg03593336 | 72666 |
| | LPPR5 | | | |
| −0.101 | HLA- | 6:33040610 | cg20617328 | 369385 |
| | DPA1; HLA- | | | |
| | DPB1 | | | |
| −0.101 | CD3EAP; ERCC1; | 19:45901453 | cg08884571 | 369385 |
| | PPP1R13L | | | |
| −0.101 | NALCN | 13:102060580 | cg22191606 | 393258 |
| −0.101 | MGLL | 3:127473936 | cg15694422 | 289708 |
| −0.101 | | 2:121279886 | cg23405152 | 412432 |
| −0.101 | DNAH17; PGS1 | 17:76417995 | cg22290140 | 394680 |
| −0.101 | PALM2; PALM2- | 9:112593847 | cg13893012 | 257062 |
| | AKAP2 | | | |
| −0.101 | CHAT; SLC18A3 | 10:50816262 | cg04730768 | 94454 |
| −0.101 | CCBL2; RBMXL1 | 1:89459658 | cg23627354 | 415857 |
| −0.101 | | 21:10888524 | cg19433175 | 350575 |
| −0.101 | WDSUB1 | 2:160142643 | cg00168785 | 3783 |
| −0.101 | CYP26B1 | 2:72357937 | cg15160709 | 281412 |
| −0.101 | | 6:74965323 | cg11654562 | 218523 |
| −0.101 | AAAS; SP7 | 12:53715148 | cg23032316 | 406156 |
| −0.101 | ZNF718 | 4:124693 | cg15792688 | 291287 |
| −0.101 | | 11:120387302 | cg06995068 | 136607 |
| −0.101 | | 7:147500722 | cg17372101 | 317966 |
| −0.101 | | 2:3044737 | cg11902863 | 222986 |
| −0.101 | | 14:77519601 | cg13576669 | 250680 |
| −0.101 | PDE11A | 2:178770927 | cg04628174 | 92633 |
| −0.101 | CCDC64B; HCFC1R1; THOC6; | 16:3079708 | cg07868599 | 153015 |
| | TNFRSF12A | | | |
| −0.101 | HLA- | 6:33044510 | cg00750366 | 15857 |
| | DPA1; HLA- | | | |
| | DPB1 | | | |
| −0.101 | ZNF767 | 7:149323101 | cg08744726 | 168908 |
| −0.100 | LOC100506012; | 19:46999224 | cg11410682 | 214729 |
| | PNMAL2 | | | |
| −0.100 | SDC3 | 1:31368797 | cg00658151 | 13923 |
| −0.100 | CILP2; NDUFA13; | 19:19643534 | cg09935271 | 189575 |
| | YJEFN3 | | | |
| −0.100 | HOXD3; LOC401022 | 2:177028804 | cg10304824 | 196084 |
| −0.100 | PCDHGA1; PCDHGA10; PCDHGA11; PCDHGA12; PCDHGA2; | 5:140807225 | cg21929183 | 389472 |
| | PCDHGA3; | | | |
| | PCDHGA4; | | | |
| | PCDHGA5; | | | |
| | PCDHGA6; | | | |
| | PCDHGA7; | | | |
| | PCDHGA8; PCDHGA9; | | | |
| | PCDHGB1; | | | |
| | PCDHGB2; | | | |
| | PCDHGB3; | | | |
| | PCDHGB4; | | | |
| | PCDHGB5; | | | |
| | PCDHGB6; PCDHGB7; | | | |
| | PCDHGB8P | | | |
| −0.100 | SCN7A | 2:167350889 | cg06038864 | 118973 |
| −0.100 | CEBPZ; LOC100505876; | 2:37424015 | cg23687288 | 417012 |
| | SULT6B1 | | | |
| −0.100 | | 15:101389354 | cg25878441 | 451879 |
| 0.100 | | 20:690915 | cg12338552 | 230066 |
| 0.100 | KIRREL3- | 11:126873181 | cg08668551 | 167552 |
| | AS3 | | | |
| 0.100 | MLNR | 13:49795541 | cg06476591 | 126402 |
| 0.100 | ZNF542 | 19:56879662 | cg03146949 | 63935 |
| 0.100 | KY | 3:134369987 | cg12307306 | 229684 |
| 0.100 | TMEM200B | 1:29449356 | cg00866556 | 18256 |
| 0.100 | ERICH1 | 8:687384 | cg01200640 | 25242 |
| 0.100 | HOXA5; HOXA6; | 7:27187372 | cg04265576 | 85698 |
| | HOXA7; LOC100133311 | | | |
| 0.100 | LOC386758; ZNF582 | 19:56904901 | cg09568464 | 183339 |
| 0.100 | ISM1 | 20:13200973 | cg06262436 | 122823 |
| 0.100 | | 10:100109635 | cg23078268 | 407013 |
| 0.100 | AQPEP | 5:115298720 | cg25044651 | 439130 |
| 0.100 | AJAP1 | 1:4714672 | cg12475759 | 232236 |
| 0.100 | | 15:78112847 | cg09369863 | 179831 |
| 0.100 | HOXC4 | 12:54441509 | cg10366797 | 197193 |
| 0.100 | C7orf50; CYP2W1 | 7:1029594 | cg22505411 | 397678 |
| 0.100 | SLC22A3 | 6:160769116 | cg07883823 | 153297 |
| 0.100 | CDH8 | 16:62070086 | cg06831576 | 133330 |
| 0.100 | CCDC164; EPT1 | 2:26625047 | cg13255216 | 244382 |
| 0.100 | GHSR | 3:172167445 | cg04851268 | 96789 |
| 0.100 | | 2:213402433 | cg08042975 | 156156 |
| 0.100 | TCHH | 1:152081002 | cg05523911 | 109583 |
| 0.100 | ERBB2 | 17:37856378 | cg26111030 | 455565 |
| 0.100 | | 9:13278626 | cg14655855 | 272946 |
| 0.100 | EPGN; MTHFD2L | 4:75174080 | cg07643762 | 149031 |
| 0.100 | ISL2; SCAPER | 15:76634887 | cg02245004 | 45800 |
| 0.100 | UGGT2 | 13:96705123 | cg08797194 | 169739 |
| 0.100 | TBL1XR1 | 3:176827739 | cg22160300 | 392814 |
| 0.100 | LMO3 | 12:16762831 | cg05355757 | 106496 |
| 0.101 | | 17:25676440 | cg07143451 | 139627 |
| 0.101 | MYF5; MYF6 | 12:81110706 | cg14067873 | 260821 |
| 0.101 | EVX2; HOXD13 | 2:176948693 | cg01749491 | 35793 |
| 0.101 | OR7G3 | 19:9238958 | cg03494634 | 70624 |
| 0.101 | PON2; PON3 | 7:95025905 | cg15927196 | 293537 |
| 0.101 | TP73; WRAP73 | 1:3567986 | cg24073122 | 423203 |
| 0.101 | | 1:148854572 | cg11818631 | 221440 |
| 0.101 | ASCL2 | 11:2292000 | cg13930892 | 257925 |
| 0.101 | CNGB1 | 16:57915850 | cg07029201 | 137333 |
| 0.101 | HTR4 | 5:148033708 | cg12825070 | 237836 |
| 0.101 | C1orf201; NIPAL3 | 1:24741723 | cg21298759 | 380184 |
| 0.101 | | 7:158286570 | cg23618588 | 415687 |
| 0.101 | | 5:4942612 | cg11468819 | 215620 |
| 0.101 | | 5:56595244 | cg19985056 | 359969 |
| 0.101 | FLJ23152 | 6:17102678 | cg05312353 | 105662 |
| 0.101 | TCHH | 1:152080543 | cg04938315 | 98550 |
| 0.101 | DCC | 18:49868477 | cg01839464 | 37773 |
| 0.101 | FAM155A | 13:108519752 | cg06446668 | 125897 |
| 0.101 | LOC285548; | 4:13546151 | cg27438798 | 478567 |
| | NKX3-2 | | | |
| 0.101 | | 6:29818300 | cg22473097 | 397165 |
| 0.101 | PSKH2 | 8:87082023 | cg20982735 | 375026 |
| 0.101 | UTP14A | X:129065551 | cg02699127 | 54944 |
| 0.101 | FAM169A | 5:74161231 | cg07222505 | 141302 |
| 0.101 | PLS1 | 3:142314493 | cg00063773 | 1512 |
| 0.101 | RUNX1T1 | 8:93115504 | cg01806520 | 37028 |
| 0.101 | | 2:133106819 | cg12792250 | 237266 |
| 0.101 | LOC654342 | 2:91848218 | cg24829460 | 435500 |
| 0.101 | | 5:2915236 | cg21081803 | 376494 |
| 0.101 | THRB | 3:24536252 | cg02333852 | 47654 |
| 0.101 | SNAP91 | 6:84418789 | cg16334314 | 300384 |
| 0.101 | SGK1 | 6:134497247 | cg03400131 | 68781 |
| 0.101 | LOC729852; | 7:7726202 | cg26804611 | 467608 |
| | RPA3 | | | |
| 0.101 | ILF2; NPR1 | 1:153651568 | cg04484579 | 89880 |
| 0.101 | LOC643714; TOX3 | 16:52581551 | cg02709321 | 55162 |
| 0.101 | EYA4 | 6:133562475 | cg20787173 | 372221 |
| 0.101 | DBNDD2; PIGT | 20:44035850 | cg03172947 | 64447 |
| 0.101 | PAK3 | X:110187421 | cg06455967 | 126034 |
| 0.101 | HCN1 | 5:45695922 | cg06498267 | 126802 |
| 0.101 | ZIC4 | 3:147111120 | cg27606499 | 481395 |
| 0.101 | MAGI2-AS3 | 7:79082531 | cg14592474 | 271684 |
| 0.101 | WTIP | 19:34972358 | cg11869770 | 222476 |
| 0.101 | UCP1 | 4:141490153 | cg19346645 | 349167 |
| 0.101 | COL23A1 | 5:178017853 | cg26996818 | 471137 |
| 0.101 | ZC3H12C | 11:109964976 | cg11758932 | 220367 |
| 0.101 | SYT9 | 11:7273498 | cg08185661 | 158874 |
| 0.101 | | 8:41004167 | cg09091657 | 174922 |
| 0.101 | | 14:28733613 | cg01840183 | 37791 |
| 0.101 | CDK6 | 7:92442568 | cg05278074 | 104986 |
| 0.101 | FLJ42875; PRDM16 | 1:2990490 | cg17001566 | 311833 |
| 0.102 | | 7:53286906 | cg10502121 | 199449 |
| 0.102 | IQGAP3; TTC24 | 1:156542103 | cg12262564 | 229022 |
| 0.102 | DEAF1; DRD4 | 11:640606 | cg20411756 | 366324 |
| 0.102 | | 12:29303160 | cg23787937 | 418459 |
| 0.102 | FOXF1; LOC400550 | 16:86548895 | cg08589354 | 166151 |
| 0.102 | | 18:44787492 | cg18074954 | 329008 |
| 0.102 | KANK3 | 19:8398300 | cg17490089 | 319937 |
| 0.102 | PRDM12 | 9:133534961 | cg13465477 | 248295 |
| 0.102 | GLRA1 | 5:151304817 | cg25352328 | 443801 |
| 0.102 | GRIA2 | 4:158141570 | cg00699993 | 14796 |
| 0.102 | ADNP2; LOC100130522; | 18:77905119 | cg18973878 | 343754 |
| | PARD6G | | | |
| 0.102 | TBL1X | X:9432900 | cg10839723 | 205305 |
| 0.102 | LOC255480; TBX5 | 12:114845868 | cg21907579 | 389107 |
| 0.102 | TDRD5 | 1:179555658 | cg17138852 | 314056 |
| 0.102 | GPR176 | 15:40212781 | cg17497271 | 320059 |
| 0.102 | CHST3 | 10:73724697 | cg19731268 | 355545 |
| 0.102 | SNAP91 | 6:84418659 | cg15950068 | 293877 |
| 0.102 | PDXK | 21:45147292 | cg02253535 | 45973 |
| 0.102 | | 5:168728149 | cg15927720 | 293550 |
| 0.102 | PRDM13 | 6:100061111 | cg17302155 | 316788 |
| 0.102 | DLL4 | 15:41219238 | cg16069079 | 295915 |
| 0.102 | PDGFRA | 4:55100025 | cg24799620 | 435038 |
| 0.102 | FAM190A | 4:91049007 | cg17275700 | 316281 |
| 0.102 | FUT9 | 6:96463959 | cg25114913 | 440174 |
| 0.102 | MOXD2P; PRSS58 | 7:141951023 | cg12970830 | 240011 |
| 0.102 | HPS4; SRRD | 22:26875563 | cg27087377 | 472647 |
| 0.102 | | 17:25676386 | cg05977781 | 117874 |
| 0.102 | | 15:96905554 | cg00221709 | 4765 |
| 0.102 | RARRES1 | 3:158450208 | cg03269060 | 66248 |
| 0.102 | KDR | 4:55991701 | cg25722465 | 449597 |
| 0.102 | SOX17 | 8:55370579 | cg26059468 | 454690 |
| 0.102 | | 14:95330880 | cg18939596 | 343247 |
| 0.102 | DLC1 | 8:13134166 | cg20536983 | 368192 |
| 0.102 | | 2:44312696 | cg08613504 | 166550 |
| 0.102 | ESR1 | 6:152085619 | cg17741339 | 323560 |
| 0.102 | CPLX2 | 5:175299035 | cg06833732 | 133382 |
| 0.102 | | 21:42218932 | cg09880551 | 188645 |
| 0.102 | CCDC108; CRYBA2; | 2:219858146 | cg24374738 | 427940 |
| | FEV; MIR375 | | | |
| 0.102 | FBXL15; NFKB2; | 10:104170217 | cg26089160 | 455163 |
| | PSD | | | |
| 0.102 | PTPRZ1 | 7:121513695 | cg10948929 | 206989 |
| 0.102 | | 5:87979504 | cg12991050 | 240324 |
| 0.102 | RNF180 | 5:63461566 | cg16485558 | 303034 |
| 0.102 | ZDBF2 | 2:207140303 | cg00410106 | 8675 |
| 0.103 | CACNA1B | 9:140917413 | cg11827453 | 221623 |
| 0.103 | CUL4B | X:119694554 | cg22267947 | 394373 |
| 0.103 | DUOX1; DUOXA1 | 15:45422054 | cg24505618 | 430070 |
| 0.103 | RUNX1T1 | 8:93114294 | cg07622493 | 148653 |
| 0.103 | GBX2 | 2:237078733 | cg07014673 | 137017 |
| 0.103 | HOXD4; MIR10B | 2:177014949 | cg27160395 | 473893 |
| 0.103 | GLRB | 4:157997363 | cg09688546 | 185354 |
| 0.103 | DENND4C | 9:19298547 | cg19584733 | 353015 |
| 0.103 | CASC4 | 15:44579510 | cg00093643 | 2177 |
| 0.103 | | 6:9399379 | cg20876760 | 373416 |
| 0.103 | GBX2 | 2:237076171 | cg20804191 | 372465 |
| 0.103 | LY6H | 8:144240945 | cg00831710 | 17537 |
| 0.103 | | 6:28853021 | cg24867279 | 436176 |
| 0.103 | ALKBH7; CLPP | 19:6362787 | cg16848221 | 309550 |
| 0.103 | | 5:1107837 | cg22172143 | 393020 |
| 0.103 | ZNF729 | 19:22469860 | cg15413034 | 285081 |
| 0.103 | TP73; WRAP73 | 1:3567412 | cg16823083 | 309243 |
| 0.103 | KBTBD11 | 8:1949342 | cg25021970 | 438735 |
| 0.103 | KLF16 | 19:1851995 | cg11434705 | 215145 |
| 0.103 | PEX5L | 3:179670323 | cg18765712 | 340436 |
| 0.103 | NFIX | 19:13107252 | cg06197482 | 121707 |
| 0.103 | | 2:9235972 | cg04575202 | 91657 |
| 0.103 | PCDHGA1; PCDHGA2 | 5:140712459 | cg21722795 | 386305 |
| 0.103 | | 1:32237923 | cg04878000 | 97320 |
| 0.103 | | 12:130422172 | cg01448115 | 30160 |
| 0.103 | RBFOX3 | 17:77454437 | cg25258515 | 442358 |
| 0.103 | EPB41L3 | 18:5543945 | cg16304950 | 299805 |
| 0.103 | | 13:112759355 | cg15415452 | 285112 |
| 0.103 | PPYR1 | 10:47083426 | cg02394955 | 48953 |
| 0.103 | ANKRD20A8P | 2:95522902 | cg08721908 | 168505 |
| 0.103 | C2orf65 | 2:74813369 | cg02102114 | 43172 |
| 0.103 | MIR137; MIR2682 | 1:98519101 | cg09896412 | 188935 |
| 0.103 | NR1D1; THRA | 17:38255871 | cg09161455 | 176141 |
| 0.103 | RPTOR | 17:78815421 | cg02363653 | 48276 |
| 0.103 | LOC100505806; | 5:9545976 | cg11011938 | 208099 |
| | SNORD123 | | | |
| 0.103 | GRIN2A | 16:10051500 | cg06880615 | 134389 |
| 0.103 | | 16:85369038 | cg05877550 | 115832 |
| 0.103 | CAPN1; SPDYC | 11:64949878 | cg25363789 | 444004 |
| 0.103 | | 4:1407989 | cg26860935 | 468675 |
| 0.103 | THSD7A | 7:11871690 | cg26748945 | 466682 |
| 0.103 | NEURL; SH3PXD2A | 10:105344174 | cg16311536 | 299928 |
| 0.103 | FREM3 | 4:144621385 | cg04514249 | 90444 |
| 0.103 | CRNDE; IRX5 | 16:54965084 | cg02056682 | 42152 |
| 0.104 | POLR1B; TTL | 2:113298655 | cg12937159 | 239513 |
| 0.104 | PRKD1 | 14:30396845 | cg08608974 | 166464 |
| 0.104 | LAIR1 | 19:54876795 | cg20117742 | 361985 |
| 0.104 | CNTNAP2 | 7:145814152 | cg16254309 | 298913 |
| 0.104 | CRMP1 | 4:5894930 | cg15448975 | 285648 |
| 0.104 | ASIP | 20:32857151 | cg09775533 | 186730 |
| 0.104 | RBFOX1 | 16:6623787 | cg04671611 | 93373 |
| 0.104 | PCDHA1; PCDHA2; | 5:140207609 | cg16987900 | 311601 |
| | PCDHA3; | | | |
| | PCDHA4; | | | |
| | PCDHA5; PCDHA6; | | | |
| | PCDHA7 | | | |
| 0.104 | TGIF1 | 18:3452302 | cg12899157 | 238913 |
| 0.104 | ADRBK2 | 22:25961523 | cg15154002 | 281307 |
| 0.104 | FBP1 | 9:97402452 | cg14211930 | 263861 |
| 0.104 | GABRG2 | 5:161494500 | cg08566437 | 165741 |
| 0.104 | SMAD2 | 18:45458243 | cg03294491 | 66702 |
| 0.104 | SHISA6 | 17:11143830 | cg08169394 | 158589 |
| 0.104 | ADAMTSL3 | 15:84322946 | cg14230666 | 264293 |
| 0.104 | TRIM67 | 1:231296818 | cg08855395 | 170825 |
| 0.104 | RBP4 | 10:95361223 | cg25793377 | 450725 |
| 0.104 | EDIL3 | 5:83679941 | cg07201620 | 140856 |
| 0.104 | C10orf114; MIR1915 | 10:21789124 | cg00580562 | 12369 |
| 0.104 | CAMK1G | 1:209759145 | cg14034757 | 260152 |
| 0.104 | NET1 | 10:5488500 | cg14426660 | 268226 |
| 0.104 | ACTL6B | 7:100253914 | cg08572611 | 165841 |
| 0.104 | TEKT1 | 17:6734940 | cg20663042 | 370096 |
| 0.104 | LOC100271702 | 12:2030223 | cg08621624 | 166700 |
| 0.104 | OCRL | X:128674222 | cg27633753 | 481846 |
| 0.104 | ANKRD53; TEX261 | 2:71205891 | cg18006568 | 327816 |
| 0.104 | RAB32 | 6:146864752 | cg23833452 | 419083 |
| 0.104 | NCAM1 | 11:112833773 | cg12040830 | 225223 |
| 0.104 | C3orf26; FILIP1L | 3:99595145 | cg22092811 | 391783 |
| 0.104 | | 14:95331232 | cg00814780 | 17210 |
| 0.104 | LOC100507043 | 14:96342365 | cg01718322 | 35254 |
| 0.104 | CPLX1 | 4:789505 | cg12792086 | 237263 |
| 0.104 | LINC00327 | 13:24062873 | cg08381620 | 162504 |
| 0.104 | OLFM2 | 19:10024282 | cg06911487 | 135026 |
| 0.104 | FLJ41350; LBX1 | 10:102987257 | cg12646649 | 235126 |
| 0.104 | DUSP27 | 1:167090974 | cg17801546 | 324462 |
| 0.104 | PCDHA1; PCDHA10; | 5:140346199 | cg25500080 | 446199 |
| | PCDHA11; | | | |
| | PCDHA12; | | | |
| | PCDHA13; | | | |
| | PCDHA2; PCDHA3; | | | |
| | PCDHA4; | | | |
| | PCDHA5; | | | |
| | PCDHA6; PCDHA7; | | | |
| | PCDHA8; | | | |
| | PCDHA9; | | | |
| | PCDHAC1; PCDHAC2 | | | |
| 0.104 | ABCC3; CACNA1G | 17:48712370 | cg00081975 | 1883 |
| 0.104 | ZBTB16 | 11:113953621 | cg20268992 | 364195 |
| 0.104 | TMEM179 | 14:105070864 | cg10281977 | 195739 |
| 0.104 | ASB10; GBX1 | 7:150870036 | cg12649727 | 235202 |
| 0.105 | | 11:79151811 | cg09673208 | 185113 |
| 0.105 | CA10 | 17:50235965 | cg22702328 | 400704 |
| 0.105 | SOX17 | 8:55371013 | cg08044097 | 156179 |
| 0.105 | PIWIL1 | 12:130822603 | cg24838063 | 435641 |
| 0.105 | SLC7A14 | 3:170303721 | cg21884231 | 388773 |
| 0.105 | GRIN2D; GRWD1 | 19:48946564 | cg04419610 | 88694 |
| 0.105 | STK24 | 13:99218657 | cg17176619 | 314752 |
| 0.105 | FAM159B | 5:63986733 | cg14004073 | 259441 |
| 0.105 | LOC728723; SNORA47; | 5:76373719 | cg03163459 | 64262 |
| | ZBED3 | | | |
| 0.105 | ZNF536 | 19:30866192 | cg03758150 | 75925 |
| 0.105 | PCDHA1; PCDHA10; | 5:140346394 | cg21537235 | 383480 |
| | PCDHA11; | | | |
| | PCDHA12; | | | |
| | PCDHA13; | | | |
| | PCDHA2; PCDHA3; | | | |
| | PCDHA4; | | | |
| | PCDHA5; | | | |
| | PCDHA6; PCDHA7; | | | |
| | PCDHA8; | | | |
| | PCDHA9; | | | |
| | PCDHAC1; PCDHAC2 | | | |
| 0.105 | | 11:43569269 | cg13201808 | 243496 |
| 0.105 | GLDN | 15:51634039 | cg09495418 | 182011 |
| 0.105 | UGGT1 | 2:128953231 | cg11873311 | 222541 |
| 0.105 | TBX3 | 12:115129446 | cg10297792 | 195985 |
| 0.105 | GPR123 | 10:134902278 | cg07779444 | 151406 |
| 0.105 | IRX1 | 5:3592435 | cg19814981 | 357062 |
| 0.105 | HOTAIRM1; HOXA1; | 7:27142682 | cg09871315 | 188506 |
| | HOXA2; | | | |
| | HOXA3 | | | |
| 0.105 | PLK2 | 5:57756539 | cg07479092 | 145902 |
| 0.105 | TBX3 | 12:115123875 | cg18573327 | 337428 |
| 0.105 | ACTN2 | 1:236849994 | cg20482698 | 367438 |
| 0.105 | LOC388499; LRRC8E | 19:7953281 | cg27615578 | 481540 |
| 0.105 | THRB | 3:24536889 | cg26146027 | 456200 |
| 0.105 | GRIA2 | 4:158141542 | cg10410142 | 197887 |
| 0.105 | FN3K; FN3KRP | 17:80693076 | cg03077331 | 62673 |
| 0.105 | OPA3; VASP | 19:46032528 | cg26958124 | 470450 |
| 0.105 | HOXD1; LOC401022 | 2:177053274 | cg03450948 | 69796 |
| 0.105 | VWC2 | 7:49813088 | cg01893212 | 38794 |
| 0.105 | | 1:241520331 | cg13849378 | 256225 |
| 0.105 | DLX6-AS1 | 7:96622709 | cg18502142 | 336304 |
| 0.105 | | 4:180979631 | cg12950911 | 239731 |
| 0.105 | DNAH8 | 6:38683221 | cg03166753 | 64319 |
| 0.105 | NETO1 | 18:70536461 | cg13644317 | 252077 |
| 0.105 | FLJ12825; HOXC4 | 12:54454163 | cg04024827 | 80948 |
| 0.105 | | 6:125855124 | cg02612397 | 53198 |
| 0.105 | TP73; WRAP73 | 1:3567646 | cg07382920 | 144177 |
| 0.105 | | 4:165304540 | cg21300373 | 380200 |
| 0.105 | PRDM8 | 4:81118588 | cg26299084 | 458882 |
| 0.106 | ASIP | 20:32857072 | cg16655240 | 306231 |
| 0.106 | TBX18 | 6:85474093 | cg02106850 | 43270 |
| 0.106 | THBD | 20:23030250 | cg09279949 | 178245 |
| 0.106 | HOXD8; LOC100506783 | 2:177004015 | cg18516557 | 336519 |
| 0.106 | AQPEP | 5:115298704 | cg22010052 | 390518 |
| 0.106 | WNT3A | 1:228248013 | cg26841013 | 468320 |
| 0.106 | C14orf23; FOXG1 | 14:29236008 | cg02681442 | 54626 |
| 0.106 | COL4A1; COL4A2 | 13:110959428 | cg06659727 | 129920 |
| 0.106 | LOC100192378; | 8:77590322 | cg09938511 | 189649 |
| | ZFHX4 | | | |
| 0.106 | MAGI2-AS3 | 7:79083753 | cg07448060 | 145248 |
| 0.106 | CHRDL1 | X:110039536 | cg00619443 | 13187 |
| 0.106 | FAM174B | 15:93199037 | cg25864024 | 451730 |
| 0.106 | CNPY1 | 7:155301734 | cg05944137 | 117184 |
| 0.106 | PIGZ | 3:196695320 | cg23484358 | 413640 |
| 0.106 | WNT7B | 22:46372742 | cg17179568 | 314808 |
| 0.106 | TMPRSS3; UBASH3A | 21:43824071 | cg00134539 | 3054 |
| 0.106 | EPHA5; LOC100144602 | 4:66534218 | cg23193365 | 408770 |
| 0.106 | DGKI | 7:137125473 | cg13734282 | 253993 |
| 0.106 | UNC5C | 4:96470286 | cg11723848 | 219719 |
| 0.106 | | 10:35256135 | cg16738929 | 307860 |
| 0.106 | NRG1 | 8:32504919 | cg14663451 | 273089 |
| 0.106 | HOTTIP; HOXA13 | 7:27246799 | cg25720795 | 449560 |
| 0.106 | LMF1; SOX8 | 16:1030619 | cg05520409 | 109522 |
| 0.106 | CRMP1 | 4:5894923 | cg07963234 | 154775 |
| 0.106 | HOTAIR; HOXC12; | 12:54349169 | cg26931862 | 469970 |
| | HOXC13 | | | |
| 0.106 | FOXI2 | 10:129535186 | cg25539045 | 446751 |
| 0.106 | | 1:41350014 | cg13472192 | 248463 |
| 0.106 | | 6:39740405 | cg14173968 | 262984 |
| 0.106 | | 20:690693 | cg02217786 | 45276 |
| 0.106 | PRSS3 | 9:33750687 | cg14369648 | 267044 |
| 0.106 | ZNF362 | 1:33721234 | cg03767970 | 76126 |
| 0.106 | FGFR1OP; MIR3939 | 6:167409842 | cg02634327 | 53695 |
| 0.106 | PCDH8 | 13:53419963 | cg07847863 | 152703 |
| 0.106 | HS6ST3 | 13:96742421 | cg08612100 | 166526 |
| 0.106 | ARMS2; HTRA1 | 10:124220367 | cg09576143 | 183453 |
| 0.106 | | 3:64430940 | cg11236526 | 211855 |
| 0.106 | CHST9 | 18:24765490 | cg21895526 | 388922 |
| 0.106 | C14orf132 | 14:96505722 | cg16539629 | 304057 |
| 0.106 | | 5:72596646 | cg01050429 | 22138 |
| 0.106 | | 8:61823275 | cg06011086 | 118514 |
| 0.106 | ST6GAL2 | 2:107502679 | cg19412467 | 350247 |
| 0.106 | CHAT; SLC18A3 | 10:50818299 | cg11389172 | 214354 |
| 0.106 | SLC6A1 | 3:11033866 | cg00375819 | 7950 |
| 0.107 | CLEC2L | 7:139208852 | cg08832906 | 170404 |
| 0.107 | CCDC27; TP73- | 1:3663435 | cg10634619 | 201918 |
| | AS1 | | | |
| 0.107 | ZNF90 | 19:20188803 | cg03317517 | 67181 |
| 0.107 | GBP1 | 1:89523325 | cg11083157 | 209272 |
| 0.107 | OSR2 | 8:99961874 | cg01581084 | 32753 |
| 0.107 | KCNA7; NTF4 | 19:49575475 | cg26923490 | 469807 |
| 0.107 | ISL1 | 5:50678925 | cg01587896 | 32872 |
| 0.107 | NEFH | 22:29876945 | cg23353952 | 411659 |
| 0.107 | | 7:149744935 | cg15058253 | 279644 |
| 0.107 | DDX25; HYLS1; | 11:125774406 | cg19792599 | 356708 |
| | PUS3 | | | |
| 0.107 | CBWD1; FOXD4 | 9:113530 | cg01237333 | 25956 |
| 0.107 | MIR1253 | 17:2659081 | cg08457011 | 163741 |
| 0.107 | TAS2R1 | 5:9623366 | cg01984802 | 40719 |
| 0.107 | TBX18 | 6:85474090 | cg07028914 | 137326 |
| 0.107 | ARL5C; CACNB1 | 17:37321631 | cg20555674 | 368474 |
| 0.107 | CNTNAP2 | 7:145813413 | cg05749717 | 113523 |
| 0.107 | | 2:165698949 | cg21639287 | 385195 |
| 0.107 | DNAJC6 | 1:65775570 | cg26885220 | 469135 |
| 0.107 | LILRA2 | 19:55084269 | cg16893868 | 310220 |
| 0.107 | CRNDE; IRX5 | 16:54965100 | cg06191898 | 121610 |
| 0.107 | PRKAR1B | 7:751962 | cg12441126 | 231659 |
| 0.107 | ARHGEF10; | 8:1772116 | cg22880677 | 403671 |
| | MIR596 | | | |
| 0.107 | | 5:143978290 | cg02030403 | 41637 |
| 0.107 | CBLN4 | 20:54579084 | cg01729827 | 35479 |
| 0.107 | HOXD10; HOXD8; | 2:176989349 | cg09173768 | 176359 |
| | HOXD9 | | | |
| 0.107 | MIR196A1 | 17:46713962 | cg13769834 | 254628 |
| 0.107 | CRAMP1L | 16:1665094 | cg06810647 | 132860 |
| 0.107 | ANGPT1 | 8:108511174 | cg09443479 | 181117 |
| 0.107 | DRD1 | 5:174871289 | cg18190187 | 331134 |
| 0.107 | | 1:227729906 | cg11846112 | 221996 |
| 0.107 | TGFBI | 5:135364556 | cg03625177 | 73315 |
| 0.107 | TRMT2B | X:100307142 | cg11708328 | 219456 |
| 0.107 | HOXD4; MIR10B | 2:177014966 | cg12215739 | 228288 |
| 0.107 | | X:39548267 | cg11712793 | 219521 |
| 0.107 | DEAF1; DRD4; | 11:637035 | cg23501406 | 413927 |
| | SCT | | | |
| 0.107 | ZNF578 | 19:52956683 | cg14582763 | 271490 |
| 0.107 | KITLG | 12:88974161 | cg25741452 | 449928 |
| 0.107 | BIRC5; TMEM235 | 17:76228233 | cg09473315 | 181656 |
| 0.107 | DMRTA2 | 1:50884480 | cg10512745 | 199703 |
| 0.107 | TMEM132C | 12:128752040 | cg04475027 | 89694 |
| 0.107 | INPP5A; NKX6-2 | 10:134600675 | cg15304699 | 283459 |
| 0.107 | FGF8; MGEA5; | 10:103536342 | cg25209842 | 441648 |
| | NPM3 | | | |
| 0.108 | SLC22A20 | 11:64993335 | cg23930334 | 420666 |
| 0.108 | DIP2C | 10:601816 | cg06279296 | 123066 |
| 0.108 | PRKD1 | 14:30396515 | cg01588388 | 32879 |
| 0.108 | EYA4 | 6:133562332 | cg01328892 | 27704 |
| 0.108 | PTPRM | 18:7566781 | cg16474170 | 302867 |
| 0.108 | FAM129C | 19:17650009 | cg12585966 | 234024 |
| 0.108 | CD247 | 1:167409054 | cg06900257 | 134801 |
| 0.108 | DRD5 | 4:9783206 | cg04597433 | 92065 |
| 0.108 | ISCA2; LTBP2 | 14:74959018 | cg26195881 | 457091 |
| 0.108 | HOXC4; HOXC5; | 12:54408091 | cg14569644 | 271205 |
| | HOXC6; HOXC8 | | | |
| 0.108 | FAM5C | 1:190447290 | cg23010538 | 405785 |
| 0.108 | LRP12 | 8:105601696 | cg00662122 | 14007 |
| 0.108 | SFRP1 | 8:41166990 | cg24319902 | 427005 |
| 0.108 | | 13:24270087 | cg10885151 | 205995 |
| 0.108 | SVEP1 | 9:113341525 | cg13606569 | 251311 |
| 0.108 | | 3:181444563 | cg04859466 | 96950 |
| 0.108 | GABRA2 | 4:46391392 | cg25145765 | 440699 |
| 0.108 | KLF16 | 19:1851882 | cg13382072 | 246526 |
| 0.108 | CATSPER2P1; | 15:44035561 | cg15437748 | 285428 |
| | PDIA3 | | | |
| 0.108 | TRAF3IP2; TRAF3IP2- | 6:111926749 | cg15772366 | 291005 |
| | AS1 | | | |
| 0.108 | PCDHA1; PCDHA10; | 5:140345966 | cg04454086 | 89312 |
| | PCDHA11; | | | |
| | PCDHA12; | | | |
| | PCDHA13; | | | |
| | PCDHA2; PCDHA3; | | | |
| | PCDHA4; | | | |
| | PCDHA5; | | | |
| | PCDHA6; PCDHA7; | | | |
| | PCDHA8; | | | |
| | PCDHA9; | | | |
| | PCDHAC1; PCDHAC2 | | | |
| 0.108 | VWC2 | 7:49813486 | cg18206027 | 331413 |
| 0.108 | | 6:157264715 | cg16455339 | 302565 |
| 0.108 | FAM110C | 2:46465 | cg14255256 | 264798 |
| 0.108 | ZNF148 | 3:125076480 | cg22895305 | 403903 |
| 0.108 | ADAMTS16 | 5:5139874 | cg16508480 | 303412 |
| 0.108 | LOC100506757; | X:133678716 | cg19112977 | 345819 |
| | MGC16121; | | | |
| | MIR424; MIR450A1; | | | |
| | MIR450A2; | | | |
| | MIR450B; | | | |
| | MIR503; MIR542 | | | |
| 0.108 | SNX9 | 6:158243833 | cg26845300 | 468408 |
| 0.108 | TMEM181 | 6:158957433 | cg00590036 | 12606 |
| 0.108 | | 3:75661206 | cg24573207 | 431170 |
| 0.108 | PSKH2 | 8:87082014 | cg04470072 | 89611 |
| 0.108 | C10orf140; MLLT10 | 10:21814313 | cg03891590 | 78352 |
| 0.108 | MIR1258; ZNF385B | 2:180726035 | cg24575676 | 431204 |
| 0.108 | UBD | 6:29521501 | cg20723129 | 371254 |
| 0.108 | | 13:112759386 | cg21054521 | 376149 |
| 0.108 | NTNG2 | 9:135118406 | cg14408447 | 267814 |
| 0.108 | PTPRT | 20:41818356 | cg13168820 | 242965 |
| 0.109 | SOX1 | 13:112722333 | cg15466862 | 285977 |
| 0.109 | RSPO1 | 1:38100467 | cg13448605 | 247923 |
| 0.109 | FGF8; MGEA5; | 10:103536348 | cg06169131 | 121199 |
| | NPM3 | | | |
| 0.109 | HOXB2; HOXB3 | 17:46618919 | cg21780393 | 387140 |
| 0.109 | SYT7 | 11:61335254 | cg03200166 | 64948 |
| 0.109 | SPATA18 | 4:52918471 | cg17031543 | 312329 |
| 0.109 | C3orf25; MBD4 | 3:129147397 | cg17468185 | 319549 |
| 0.109 | SRCIN1 | 17:36718549 | cg20090283 | 361564 |
| 0.109 | LOC400657 | 18:72263688 | cg20276402 | 364322 |
| 0.109 | C14orf23; FOXG1 | 14:29237480 | cg16804284 | 308960 |
| 0.109 | GABRG2 | 5:161494515 | cg09220361 | 177083 |
| 0.109 | | 8:72468820 | cg24809973 | 435221 |
| 0.109 | | 7:87974722 | cg27221053 | 474880 |
| 0.109 | NXPH2 | 2:139537745 | cg10989763 | 207734 |
| 0.109 | SGPL1 | 10:72594465 | cg09140723 | 175742 |
| 0.109 | FGF18 | 5:170848123 | cg01392179 | 28917 |
| 0.109 | ZNF300 | 5:150284796 | cg21228005 | 379112 |
| 0.109 | ZNF329; ZSCAN18 | 19:58629828 | cg03145999 | 63909 |
| 0.109 | LOC255480 | 12:114852091 | cg01184975 | 24931 |
| 0.109 | LPHN3 | 4:62383028 | cg16225703 | 298417 |
| 0.109 | | 7:156409058 | cg16771407 | 308382 |
| 0.109 | UNCX | 7:1271236 | cg18629009 | 338270 |
| 0.109 | | 6:28945500 | cg24126967 | 424128 |
| 0.109 | MSX1 | 4:4860061 | cg15092343 | 280260 |
| 0.109 | SOX1 | 13:112721789 | cg02547394 | 51873 |
| 0.109 | CHST8 | 19:34112991 | cg19594305 | 353222 |
| 0.109 | BMP4 | 14:54413548 | cg27302539 | 476206 |
| 0.109 | FUT9 | 6:96463902 | cg01758512 | 35982 |
| 0.109 | | 2:240153778 | cg25746499 | 450027 |
| 0.109 | CCDC50; UTS2D | 3:191047240 | cg25235397 | 442049 |
| 0.109 | PAX1 | 20:21686282 | cg08448701 | 163575 |
| 0.110 | UBQLN2 | X:56589768 | cg02587153 | 52704 |
| 0.110 | | 10:13425015 | cg18675735 | 338977 |
| 0.110 | | 18:6414958 | cg22058122 | 391310 |
| 0.110 | CSMD1 | 8:4852036 | cg12258042 | 228965 |
| 0.110 | ASPG; MIR203; | 14:104583067 | cg23960324 | 421202 |
| | MIR3545 | | | |
| 0.110 | RACGAP1 | 12:50426901 | cg19796532 | 356768 |
| 0.110 | | 13:20875915 | cg14472366 | 269103 |
| 0.110 | | 14:95330984 | cg19453488 | 350906 |
| 0.110 | ZNF385D | 3:21792857 | cg26077378 | 454997 |
| 0.110 | MBNL2 | 13:98002933 | cg03372334 | 68296 |
| 0.110 | ENO4 | 10:118608698 | cg01035689 | 21828 |
| 0.110 | CRHBP | 5:76249502 | cg04306063 | 86503 |
| 0.110 | ODZ1 | X:124097806 | cg24761295 | 434425 |
| 0.110 | | 6:78359965 | cg10264012 | 195459 |
| 0.110 | | 7:4921827 | cg04816699 | 96082 |
| 0.110 | MDGA1 | 6:37616598 | cg03062252 | 62373 |
| 0.110 | GJD2 | 15:35046760 | cg21053529 | 376133 |
| 0.110 | RLF; TMCO2; | 1:40714009 | cg06776907 | 132164 |
| | ZMPSTE24 | | | |
| 0.110 | HS3ST6 | 16:1968232 | cg14936269 | 277598 |
| 0.110 | DKFZp434J0226 | 19:46713473 | cg21629821 | 385058 |
| 0.110 | ANO1 | 11:69934109 | cg05846633 | 115247 |
| 0.110 | KIAA1755 | 20:36889364 | cg14003035 | 259413 |
| 0.110 | CORIN; NFXL1 | 4:47840011 | cg26018901 | 454082 |
| 0.110 | GLT1D1 | 12:129337897 | cg04054313 | 81425 |
| 0.110 | | 9:13279482 | cg06202228 | 121794 |
| 0.110 | CADM1 | 11:115069145 | cg05125455 | 102021 |
| 0.110 | DPYS | 8:105479058 | cg10303487 | 196069 |
| 0.110 | KDM2B | 12:122019080 | cg17452615 | 319270 |
| 0.110 | FOXI2 | 10:129535509 | cg09614415 | 184092 |
| 0.110 | ARHGEF4 | 2:131673894 | cg12910906 | 239117 |
| 0.110 | CPNE7 | 16:89654085 | cg10499753 | 199387 |
| 0.110 | CBLN1 | 16:49315302 | cg08464190 | 163914 |
| 0.110 | ADAM33; ATRN; | 20:3641641 | cg25164624 | 440992 |
| | GFRA4 | | | |
| 0.110 | LIMK2 | 22:31644160 | cg15504662 | 286549 |
| 0.110 | FLT3 | 13:28673762 | cg26472910 | 461940 |
| 0.110 | BANK1 | 4:102712397 | cg14855874 | 276324 |
| 0.110 | | 12:34500585 | cg19457237 | 350958 |
| 0.110 | KDM2B | 12:122018897 | cg09151742 | 175965 |
| 0.111 | | 2:130763641 | cg08857994 | 170896 |
| 0.111 | PAX6 | 11:31819380 | cg26029734 | 454240 |
| 0.111 | HMGCLL1 | 6:55443868 | cg06401021 | 125152 |
| 0.111 | ZNF578 | 19:52956832 | cg25763393 | 450297 |
| 0.111 | TRPC4 | 13:38444134 | cg15696906 | 289754 |
| 0.111 | PLEKHA6 | 1:204256846 | cg25407979 | 444757 |
| 0.111 | C5orf38; IRX2 | 5:2743601 | cg26195356 | 457077 |
| 0.111 | EP400; PUS1 | 12:132433838 | cg26147845 | 456245 |
| 0.111 | MLLT3 | 9:20339790 | cg21222426 | 379016 |
| 0.111 | CTTNBP2 | 7:117513348 | cg17112089 | 313639 |
| 0.111 | EFHD1 | 2:233499460 | cg06842071 | 133579 |
| 0.111 | TMEM131 | 2:98613076 | cg02995664 | 61036 |
| 0.111 | DLX5; DLX6; DLX6- | 7:96647021 | cg23262036 | 410160 |
| | AS1 | | | |
| 0.111 | RAX | 18:56940333 | cg06676637 | 130191 |
| 0.111 | COLEC12 | 18:500979 | cg19461621 | 351026 |
| 0.111 | MIR618 | 12:81331486 | cg23441441 | 412989 |
| 0.111 | | 7:64407743 | cg24002887 | 421987 |
| 0.111 | ST6GALNAC5 | 1:77334045 | cg23243867 | 409762 |
| 0.111 | MIR618 | 12:81331469 | cg17061244 | 312814 |
| 0.111 | LAMA1 | 18:7117680 | cg07846220 | 152681 |
| 0.111 | LOC100506012; | 19:46975062 | cg21988244 | 390260 |
| | PNMAL1 | | | |
| 0.111 | FGF12 | 3:192127330 | cg16376000 | 301109 |
| 0.111 | | 4:85403915 | cg25174438 | 441100 |
| 0.111 | GALR1 | 18:74961968 | cg20872937 | 373368 |
| 0.111 | LRIT1; RGR | 10:86001254 | cg14034858 | 260156 |
| 0.111 | LAG3; PTMS | 12:6888035 | cg11429292 | 215067 |
| 0.111 | PDE1C | 7:32110523 | cg14604568 | 271937 |
| 0.111 | ZNF503; ZNF503- | 10:77156218 | cg20659435 | 370034 |
| | AS2 | | | |
| 0.111 | CDH8 | 16:62069337 | cg02172312 | 44501 |
| 0.111 | ISL1 | 5:50678913 | cg17412886 | 318628 |
| 0.111 | THRB | 3:24537469 | cg08123277 | 157665 |
| 0.111 | HOXD10; HOXD11; | 2:176972068 | cg05500840 | 109127 |
| | HOXD12 | | | |
| 0.111 | ADAMTS16 | 5:5140003 | cg03209854 | 65125 |
| 0.111 | | 2:200524012 | cg27211284 | 474729 |
| 0.111 | SLC16A2 | X:73641955 | cg06805513 | 132754 |
| 0.111 | HOXC4; HOXC5; | 12:54424964 | cg10005224 | 190710 |
| | HOXC6; MIR615 | | | |
| 0.111 | PTPRZ1 | 7:121513058 | cg05931096 | 116896 |
| 0.111 | SOX17 | 8:55370434 | cg15186181 | 281779 |
| 0.112 | ZNF536 | 19:30866194 | cg06000994 | 118339 |
| 0.112 | | 1:17199104 | cg20168927 | 362799 |
| 0.112 | STAC2 | 17:37381475 | cg14328457 | 266291 |
| 0.112 | CLMP | 11:123008408 | cg12995113 | 240381 |
| 0.112 | CADPS | 3:62860224 | cg13514230 | 249338 |
| 0.112 | TAPBPL; VAMP1 | 12:6572482 | cg22129323 | 392333 |
| 0.112 | | 13:112849184 | cg20681688 | 370443 |
| 0.112 | OR51A2; OR51A4 | 11:4977924 | cg07589078 | 148007 |
| 0.112 | | 11:110627909 | cg04297660 | 86337 |
| 0.112 | DNMT3A | 2:25474327 | cg10749994 | 203678 |
| 0.112 | | 19:23185714 | cg24911113 | 436942 |
| 0.112 | CCT8L2; TPTEP1 | 22:17083482 | cg00816224 | 17246 |
| 0.112 | SLC35F3 | 1:234040781 | cg09424759 | 180776 |
| 0.112 | EPHB2; MIR4684 | 1:23038177 | cg27573841 | 480859 |
| 0.112 | HOXC8; HOXC9 | 12:54398518 | cg08254359 | 160184 |
| 0.112 | PRDM8 | 4:81118794 | cg06307913 | 123528 |
| 0.112 | | 22:33454324 | cg05206884 | 103569 |
| 0.112 | CR1L | 1:207842927 | cg20264068 | 364122 |
| 0.112 | DNAAF2; LRR1; | 14:50085410 | cg24149364 | 424560 |
| | MGAT2; RPL36AL | | | |
| 0.112 | MAPK14 | 6:36076933 | cg20559215 | 368537 |
| 0.112 | ISL2 | 15:76630209 | cg23513966 | 414134 |
| 0.112 | F10; PROZ | 13:113807384 | cg10067723 | 191777 |
| 0.112 | C1orf229; ZNF124; | 1:247275344 | cg23695504 | 417170 |
| | ZNF669 | | | |
| 0.112 | HOOK1 | 1:60280809 | cg23996071 | 421845 |
| 0.112 | ZNF83 | 19:53142304 | cg17132967 | 313971 |
| 0.112 | TACR1 | 2:75292272 | cg14054283 | 260559 |
| 0.112 | | 15:29862568 | cg13942157 | 258175 |
| 0.112 | | 4:165305050 | cg10453719 | 198622 |
| 0.112 | SPAG6 | 10:22634218 | cg12377139 | 230587 |
| 0.112 | GRM6 | 5:178422071 | cg23003783 | 405665 |
| 0.112 | AQP12A; AQP12B | 2:241623436 | cg08049592 | 156267 |
| 0.112 | | 16:15219673 | cg04381865 | 87943 |
| 0.112 | KIAA0182 | 16:85648263 | cg07301329 | 142673 |
| 0.112 | HOXC4; HOXC5; | 12:54413384 | cg15772924 | 291010 |
| | HOXC6; HOXC8 | | | |
| 0.112 | TCF15 | 20:591083 | cg25984671 | 453527 |
| 0.112 | C5orf38; IRX2 | 5:2751550 | cg18371475 | 334040 |
| 0.113 | ST6GAL2 | 2:107503672 | cg21649258 | 385387 |
| 0.113 | PTPRT | 20:41818574 | cg27625055 | 481684 |
| 0.113 | PRKCG | 19:54402455 | cg02849693 | 57883 |
| 0.113 | | 8:41754181 | cg17331296 | 317296 |
| 0.113 | SOX11 | 2:5836231 | cg18897632 | 342660 |
| 0.113 | | 17:55213656 | cg11030264 | 208410 |
| 0.113 | DPP6 | 7:153584597 | cg14523847 | 270200 |
| 0.113 | NLRP14; ZNF214 | 11:7041549 | cg22867063 | 403403 |
| 0.113 | | 6:28921805 | cg22489957 | 397412 |
| 0.113 | AMPD3; MIR4485; | 11:10531596 | cg18489034 | 336073 |
| | MTRNR2L8; | | | |
| | RNF141 | | | |
| 0.113 | PALLD | 4:169525923 | cg24784416 | 434791 |
| 0.113 | KHDRBS2 | 6:62996119 | cg26715952 | 466174 |
| 0.113 | PTGER2 | 14:52780158 | cg21502048 | 382905 |
| 0.113 | CTNNA2 | 2:79739863 | cg08119884 | 157578 |
| 0.113 | PRKAR1B | 7:751833 | cg00356183 | 7541 |
| 0.113 | GPR125 | 4:22517094 | cg08141192 | 158041 |
| 0.113 | SLC6A17 | 1:110693237 | cg11885396 | 222730 |
| 0.113 | FAT1 | 4:187645120 | cg27475132 | 479115 |
| 0.113 | KIAA0748 | 12:55371892 | cg24536818 | 430655 |
| 0.113 | TRIM31 | 6:30071394 | cg23642250 | 416154 |
| 0.113 | DPP6 | 7:153584582 | cg06495961 | 126761 |
| 0.113 | | 8:142276422 | cg25461290 | 445593 |
| 0.113 | MRPS15; OSCP1 | 1:36912487 | cg26030770 | 454254 |
| 0.113 | EPPK1 | 8:144952929 | cg18153977 | 330494 |
| 0.113 | | 3:184231385 | cg15718583 | 290162 |
| 0.113 | HAND2; NBLA00301 | 4:174453166 | cg15234400 | 282488 |
| 0.113 | EGLN3 | 14:34420292 | cg24665290 | 432653 |
| 0.113 | HOXD1; LOC401022 | 2:177053297 | cg19180624 | 346696 |
| 0.113 | LINC00472 | 6:72130209 | cg10039188 | 191305 |
| 0.113 | DMRTA2 | 1:50886949 | cg25191628 | 441334 |
| 0.113 | KIT | 4:55524199 | cg10087973 | 192117 |
| 0.114 | | 15:87892167 | cg02693157 | 54811 |
| 0.114 | NXPH1 | 7:8482235 | cg12597389 | 234250 |
| 0.114 | CACNG6; CACNG8; | 19:54486058 | cg02574502 | 52401 |
| | MIR935 | | | |
| 0.114 | HOXD11; HOXD12; | 2:176964720 | cg03964958 | 79739 |
| | HOXD13 | | | |
| 0.114 | | 7:127910927 | cg08234689 | 159796 |
| 0.114 | SPAG6 | 10:22634226 | cg18247055 | 332095 |
| 0.114 | FAM104B | X:55187561 | cg15824287 | 291790 |
| 0.114 | FOXQ1 | 6:1313211 | cg26422161 | 461072 |
| 0.114 | | 11:91957992 | cg07603330 | 148311 |
| 0.114 | UTF1; VENTX | 10:135050619 | cg25577463 | 447348 |
| 0.114 | ARL9; SRP72 | 4:57371607 | cg06922635 | 135243 |
| 0.114 | MACROD2; SEL1L2 | 20:13976143 | cg06571075 | 128176 |
| 0.114 | CNN3; LOC729970 | 1:95393096 | cg18344652 | 333645 |
| 0.114 | DUSP27 | 1:167090757 | cg11236550 | 211856 |
| 0.114 | MEGF6 | 1:3447348 | cg17163527 | 314497 |
| 0.114 | | 20:12809929 | cg12505343 | 232775 |
| 0.114 | CHST8 | 19:34112683 | cg27058486 | 472166 |
| 0.114 | PUS1; ULK1 | 12:132410932 | cg24221398 | 425685 |
| 0.114 | C14orf23; FOXG1 | 14:29244278 | cg21195256 | 378436 |
| 0.114 | | 1:88928467 | cg02664812 | 54315 |
| 0.114 | SLC44A2 | 19:10713016 | cg05567294 | 110272 |
| 0.114 | LOC100129794 | 14:37641462 | cg25051529 | 439230 |
| 0.114 | CRNDE; IRX5 | 16:54966471 | cg16642791 | 305999 |
| 0.114 | STAT3; STAT5A | 17:40464780 | cg25500196 | 446200 |
| 0.114 | FGF18 | 5:170878209 | cg02125316 | 43605 |
| 0.114 | | 7:127775630 | cg19871348 | 358070 |
| 0.114 | | 7:158380884 | cg22282410 | 394574 |
| 0.114 | | 16:1048069 | cg11978890 | 224277 |
| 0.114 | RGAG4 | X:71352048 | cg03056321 | 62257 |
| 0.114 | ZNF83 | 19:53142146 | cg25793430 | 450728 |
| 0.114 | FRYL; SLC10A4; | 4:48492304 | cg14249348 | 264663 |
| | ZAR1 | | | |
| 0.115 | HES6; LOC151174; | 2:239140182 | cg23497383 | 413854 |
| | LOC643387 | | | |
| 0.115 | | 6:28911938 | cg25005112 | 438404 |
| 0.115 | RBFOX1 | 16:6070188 | cg08015447 | 155664 |
| 0.115 | CHST8 | 19:34113214 | cg06399302 | 125117 |
| 0.115 | PRKG1 | 10:52752476 | cg11175150 | 210715 |
| 0.115 | CRNDE; IRX5 | 16:54965142 | cg09641689 | 184540 |
| 0.115 | EMX2; EMX2OS | 10:119303225 | cg15373239 | 284408 |
| 0.115 | ATOH1 | 4:94749865 | cg00335712 | 7088 |
| 0.115 | DSC3 | 18:28622474 | cg14719951 | 274021 |
| 0.115 | IDH1; PIKFYVE | 2:209128590 | cg06177665 | 121341 |
| 0.115 | CAMKK2; P2RX4 | 12:121678558 | cg09297514 | 178526 |
| 0.115 | GABRB3 | 15:27018037 | cg18026588 | 328201 |
| 0.115 | GNA15 | 19:3136430 | cg01032675 | 21764 |
| 0.115 | DPP6 | 7:153749736 | cg18940047 | 343252 |
| 0.115 | | 7:52156489 | cg18793404 | 340916 |
| 0.115 | KCNK18 | 10:118976677 | cg27455890 | 478852 |
| 0.115 | UPK3A | 22:45680879 | cg07021644 | 137193 |
| 0.115 | SLC6A2 | 16:55689865 | cg10362591 | 197113 |
| 0.115 | | 4:104918078 | cg05123845 | 101996 |
| 0.115 | PON2; PON3 | 7:95025836 | cg08828819 | 170325 |
| 0.115 | PROX1 | 1:214160860 | cg17954152 | 327012 |
| 0.115 | THRB | 3:24536562 | cg24120841 | 424012 |
| 0.115 | CDK5R2 | 2:219826870 | cg27638196 | 481940 |
| 0.115 | TMEM17 | 2:62732892 | cg10848792 | 205426 |
| 0.115 | RBFOX1 | 16:6533163 | cg14642432 | 272661 |
| 0.115 | PSMD14; TBR1 | 2:162270993 | cg08184652 | 158859 |
| 0.115 | FKBP5 | 6:35654363 | cg03546163 | 71700 |
| 0.115 | LOC100188947 | 10:93058376 | cg17402294 | 318468 |
| 0.115 | KIF26B | 1:245820634 | cg11424312 | 214996 |
| 0.115 | ELAVL4 | 1:50513766 | cg05697849 | 112668 |
| 0.115 | KIAA0748 | 12:55376897 | cg25486399 | 445985 |
| 0.115 | VAX1 | 10:118896755 | cg11398452 | 214514 |
| 0.115 | RFX8 | 2:102091269 | cg12291728 | 229438 |
| 0.115 | | 6:28677112 | cg14234426 | 264370 |
| 0.115 | MTNR1A | 4:187476455 | cg25107254 | 440048 |
| 0.115 | ID2 | 2:8817010 | cg21946006 | 389750 |
| 0.115 | HTR1A | 5:63257847 | cg09698471 | 185520 |
| 0.115 | SORCS3 | 10:106401319 | cg10778841 | 204179 |
| 0.116 | | 14:74100987 | cg14527456 | 270289 |
| 0.116 | FLRT2 | 14:85996499 | cg01711160 | 35095 |
| 0.116 | SHANK1 | 19:51198381 | cg07060551 | 137922 |
| 0.116 | ALLC; COLEC11 | 2:3699195 | cg13452994 | 248021 |
| 0.116 | GRM6 | 5:178421711 | cg12483476 | 232367 |
| 0.116 | | 3:22413960 | cg06062984 | 119382 |
| 0.116 | LRRC36; TPPP3; | 16:67427339 | cg07355841 | 143680 |
| | ZDHHC1 | | | |
| 0.116 | HOTAIR; HOXC12 | 12:54355476 | cg19188855 | 346817 |
| 0.116 | TACSTD2 | 1:59042275 | cg24851854 | 435890 |
| 0.116 | NPY2R | 4:156130016 | cg21512644 | 383071 |
| 0.116 | KIRREL3-AS3 | 11:126873379 | cg00373707 | 7906 |
| 0.116 | HS6ST3 | 13:96743087 | cg27176138 | 474164 |
| 0.116 | UBD | 6:29521228 | cg15814717 | 291599 |
| 0.116 | CBWD2; FOXD4L1 | 2:114256781 | cg10602757 | 201345 |
| 0.116 | HES6; LOC151174; | 2:239140024 | cg13957874 | 258474 |
| | LOC643387 | | | |
| 0.116 | OPRK1 | 8:54164296 | cg13405783 | 247011 |
| 0.116 | NKX2- | 14:36992233 | cg04224064 | 84867 |
| | 1; SFTA3 | | | |
| 0.116 | CCNA1 | 13:37005273 | cg24297049 | 426624 |
| 0.116 | | 2:91671535 | cg02109405 | 43319 |
| 0.116 | | 15:32607923 | cg00943360 | 19827 |
| 0.116 | PTPRG | 3:61547130 | cg09472360 | 181640 |
| 0.116 | GLRB | 4:157998201 | cg10146199 | 193292 |
| 0.116 | C1orf53; LHX9 | 1:197875080 | cg12578211 | 233875 |
| 0.116 | NPTX2 | 7:98246006 | cg08315202 | 161360 |
| 0.116 | GDF7 | 2:20871195 | cg10553204 | 200490 |
| 0.116 | | 8:61835848 | cg06445016 | 125876 |
| 0.116 | PRMT8 | 12:3600106 | cg27316886 | 476555 |
| 0.116 | ADAMTS16 | 5:5139878 | cg04136610 | 83123 |
| 0.116 | ZNF503; ZNF503- | 10:77163383 | cg18510056 | 336418 |
| | AS2 | | | |
| 0.116 | | 6:28911902 | cg13293729 | 244984 |
| 0.116 | DMRT1; DMRT3 | 9:971480 | cg07173635 | 140295 |
| 0.116 | | 6:170686343 | cg01061164 | 22368 |
| 0.116 | | 5:4942469 | cg18101138 | 329470 |
| 0.116 | FBN2 | 5:127874466 | cg25532099 | 446652 |
| 0.116 | PPM1L | 3:160475090 | cg04056576 | 81465 |
| 0.116 | 11-Mar | 5:16179210 | cg03705912 | 74857 |
| 0.116 | SOX11 | 2:5837057 | cg15989068 | 294511 |
| 0.116 | INTU | 4:128544255 | cg16654911 | 306224 |
| 0.116 | FAM20C | 7:193262 | cg08782337 | 169501 |
| 0.116 | SLC15A3 | 11:60719947 | cg26980692 | 470832 |
| 0.116 | RBFOX1 | 16:6068835 | cg19378133 | 349684 |
| 0.116 | | 14:95826734 | cg05162306 | 102773 |
| 0.116 | SOX1 | 13:112717244 | cg09469566 | 181584 |
| 0.117 | PRPH | 12:49689463 | cg14029489 | 260043 |
| 0.117 | MLXIPL | 7:73038859 | cg10092878 | 192247 |
| 0.117 | ATP4A | 19:36048943 | cg19715414 | 355233 |
| 0.117 | MANEAL; YRDC | 1:38261057 | cg24433287 | 428889 |
| 0.117 | KCNA4 | 11:30038677 | cg15310492 | 283522 |
| 0.117 | | 3:54119792 | cg03494612 | 70623 |
| 0.117 | MMP17 | 12:132312461 | cg03570417 | 72216 |
| 0.117 | ADAT1; KARS | 16:75661691 | cg01921773 | 39352 |
| 0.117 | FIGLA | 2:71017541 | cg22650617 | 399823 |
| 0.117 | CNPY1 | 7:155324691 | cg09090582 | 174907 |
| 0.117 | BDNF | 11:27740876 | cg05733135 | 113285 |
| 0.117 | LINC00467 | 1:211590292 | cg20802616 | 372441 |
| 0.117 | OXTR | 3:8813493 | cg08449558 | 163593 |
| 0.117 | DNAJB13 | 11:73668626 | cg20737388 | 371474 |
| 0.117 | SLC34A2 | 4:25657365 | cg19766441 | 356236 |
| 0.117 | LINC00461; MIR9-2 | 5:87968528 | cg14636534 | 272541 |
| 0.117 | PIWIL1 | 12:130822286 | cg13861644 | 256478 |
| 0.117 | DSC3 | 18:28622593 | cg15439862 | 285459 |
| 0.117 | UBD | 6:29521046 | cg18342026 | 333604 |
| 0.117 | GABRA2 | 4:46391929 | cg23160016 | 408245 |
| 0.117 | DUSP8; MOB2 | 11:1575607 | cg05280206 | 105032 |
| 0.117 | PHACTR2 | 6:143998958 | cg26846959 | 468429 |
| 0.117 | FAM47E; SCARB2 | 4:77134471 | cg19050061 | 344935 |
| 0.117 | SPG7 | 16:89600764 | cg08905080 | 171867 |
| 0.117 | BBX | 3:107244746 | cg02321062 | 47394 |
| 0.117 | ALDH1L1 | 3:125899370 | cg01566526 | 32429 |
| 0.117 | | 3:4856199 | cg08998950 | 173407 |
| 0.117 | LTBP3 | 11:65325158 | cg08965235 | 172854 |
| 0.118 | | 16:85203635 | cg00942219 | 19808 |
| 0.118 | PIPOX | 17:27369780 | cg06144905 | 120806 |
| 0.118 | LMNB2 | 19:2446487 | cg18087306 | 329215 |
| 0.118 | GUSBP4 | 6:58263193 | cg17976157 | 327360 |
| 0.118 | ISL1 | 5:50679149 | cg08734740 | 168746 |
| 0.118 | BAALC; C8orf56 | 8:104153627 | cg18168101 | 330737 |
| 0.118 | | 1:65534742 | cg20301308 | 364692 |
| 0.118 | | 9:136474735 | cg03262554 | 66122 |
| 0.118 | ZC3H8 | 2:113012627 | cg27242549 | 475215 |
| 0.118 | ZNF148 | 3:125076233 | cg12508343 | 232821 |
| 0.118 | MGC45800 | 4:183064388 | cg00053393 | 1279 |
| 0.118 | NRG2; PSD2 | 5:139223811 | cg08682153 | 167769 |
| 0.118 | HOXA5; HOXA6; | 7:27187269 | cg06237983 | 122446 |
| | HOXA7; LOC100133311 | | | |
| 0.118 | CADPS | 3:62861055 | cg15957055 | 293976 |
| 0.118 | KCNQ1DN | 11:2891082 | cg25203481 | 441543 |
| 0.118 | ZFPM2 | 8:106332119 | cg10868817 | 205750 |
| 0.118 | | 3:171543413 | cg11639615 | 218225 |
| 0.118 | PXDN | 2:1748112 | cg12164282 | 227454 |
| 0.118 | TIGD7; ZNF75A | 16:3355553 | cg05540369 | 109851 |
| 0.118 | SP8 | 7:20824770 | cg27618240 | 481572 |
| 0.118 | | 15:96905729 | cg11157253 | 210450 |
| 0.118 | ADRA1D | 20:4202378 | cg04293085 | 86242 |
| 0.118 | LOC100130298 | 8:61880392 | cg04882213 | 97419 |
| 0.118 | DYNLT1; SYTL3 | 6:159068210 | cg27278961 | 475791 |
| 0.118 | PCDHA1; PCDHA2; | 5:140201656 | cg11781306 | 220669 |
| | PCDHA3; | | | |
| | PCDHA4; | | | |
| | PCDHA5; PCDHA6 | | | |
| 0.119 | DPP6 | 7:153584416 | cg22620221 | 399362 |
| 0.119 | ZNF468 | 19:53361164 | cg08531263 | 165137 |
| 0.119 | CNTNAP5 | 2:124782831 | cg08790440 | 169635 |
| 0.119 | FBN2 | 5:127873106 | cg17564775 | 321142 |
| 0.119 | CDH8 | 16:62070184 | cg01718742 | 35267 |
| 0.119 | COX19; CYP2W1 | 7:1018969 | cg01823585 | 37440 |
| 0.119 | LPHN2 | 1:82267931 | cg02243785 | 45777 |
| 0.119 | LOC100130155; | 8:65290484 | cg07792478 | 151647 |
| | MIR124-2 | | | |
| 0.119 | LHX8 | 1:75595970 | cg22694818 | 400568 |
| 0.119 | LOC339529; ZNF238 | 1:244213616 | cg19698993 | 354930 |
| 0.119 | OLFML2A | 9:127531952 | cg14046477 | 260419 |
| 0.119 | EPHA1; LOC285965 | 7:143108841 | cg16618979 | 305561 |
| 0.119 | AZU1; LPPR3; | 19:822086 | cg21928701 | 389466 |
| | MIR3187 | | | |
| 0.119 | OSBPL3 | 7:25005464 | cg07110009 | 138907 |
| 0.119 | | 7:52155829 | cg11342198 | 213584 |
| 0.119 | PREX2 | 8:68864769 | cg06617456 | 128991 |
| 0.119 | CHRNA4 | 20:61992494 | cg01743841 | 35695 |
| 0.119 | ISLR2; LOC283731 | 15:74425552 | cg01178680 | 24810 |
| 0.119 | UNC5D | 8:35092823 | cg08000065 | 155421 |
| 0.119 | PRKAR1B | 7:751830 | cg27331241 | 476807 |
| 0.119 | MAP7 | 6:136847359 | cg18421949 | 334865 |
| 0.119 | EPHA6 | 3:96533511 | cg11410023 | 214721 |
| 0.119 | HOXA5; HOXA6; | 7:27188364 | cg19816811 | 357110 |
| | HOXA7; LOC100133311 | | | |
| 0.119 | PDGFC | 4:157892765 | cg22451233 | 396868 |
| 0.119 | HCG4B | 6:29895005 | cg01519225 | 31476 |
| 0.119 | ATP2C2 | 16:84402160 | cg08974450 | 173017 |
| 0.119 | | 4:187037318 | cg05379002 | 107021 |
| 0.119 | TRIM71 | 3:32860178 | cg12338417 | 230063 |
| 0.119 | POU6F2 | 7:39125198 | cg02367316 | 48362 |
| 0.119 | RBP1 | 3:139258295 | cg12497564 | 232636 |
| 0.119 | EFS; IL25 | 14:23834909 | cg13011388 | 240610 |
| 0.119 | MYRIP | 3:39851788 | cg07892785 | 153449 |
| 0.119 | RAB38 | 11:87908783 | cg01568784 | 32481 |
| 0.119 | TBX4 | 17:59532306 | cg14823851 | 275680 |
| 0.119 | TOX2 | 20:42544794 | cg03011535 | 61343 |
| 0.120 | DLC1 | 8:13134144 | cg03304380 | 66890 |
| 0.120 | SNED1 | 2:241938436 | cg08233654 | 159775 |
| 0.120 | KIF25 | 6:168436019 | cg12003941 | 224653 |
| 0.120 | C1orf35; MRPL55 | 1:228297864 | cg22163199 | 392875 |
| 0.120 | CTNNA2 | 2:79739278 | cg18806140 | 341143 |
| 0.120 | ALOX5 | 10:45869702 | cg27243389 | 475225 |
| 0.120 | NAALAD2 | 11:89867976 | cg21500966 | 382886 |
| 0.120 | NLRP2 | 19:55477755 | cg17760849 | 323892 |
| 0.120 | AIM1 | 6:106957995 | cg20796467 | 372352 |
| 0.120 | ZFPM2 | 8:106331995 | cg15449956 | 285663 |
| 0.120 | ISL1 | 5:50679290 | cg11711420 | 219504 |
| 0.120 | C3orf15 | 3:119422161 | cg08145590 | 158129 |
| 0.120 | | 4:82965250 | cg04643822 | 92882 |
| 0.120 | FGFR1 | 8:38323059 | cg13278539 | 244735 |
| 0.120 | SRRM4 | 12:119591747 | cg27010159 | 471336 |
| 0.120 | UGP2 | 2:64069682 | cg19915305 | 358803 |
| 0.120 | GLI4 | 8:144361073 | cg11240062 | 211921 |
| 0.120 | TOX2 | 20:42544792 | cg13459498 | 248157 |
| 0.120 | AQP4; CHST9- | 18:24445692 | cg12445775 | 231741 |
| | AS1 | | | |
| 0.120 | NKX6-1 | 4:85418265 | cg12076463 | 225925 |
| 0.121 | PCDHGA12; PCDHGC3; | 5:140871385 | cg24619378 | 431928 |
| | PCDHGC4; | | | |
| | PCDHGC5 | | | |
| 0.121 | HOXA10; HOXA10- | 7:27204894 | cg27009703 | 471328 |
| | HOXA9; HOXA7; | | | |
| | HOXA9; LOC100133311; | | | |
| | MIR196B | | | |
| 0.121 | DLG5 | 10:79622515 | cg11063088 | 208886 |
| 0.121 | KDM2B | 12:122019076 | cg15234492 | 282489 |
| 0.121 | IRF6 | 1:209979733 | cg21951975 | 389837 |
| 0.121 | ACTL6B | 7:100254149 | cg16024950 | 295200 |
| 0.121 | ONECUT2 | 18:55095177 | cg05735180 | 113314 |
| 0.121 | WDR17 | 4:176987020 | cg08095852 | 157102 |
| 0.121 | CLEC14A | 14:38725312 | cg08139247 | 158007 |
| 0.121 | HOXD4; MIR10B | 2:177014555 | cg19519747 | 352000 |
| 0.121 | UTRN | 6:144608500 | cg15785898 | 291201 |
| 0.121 | MFSD7; MYL5 | 4:682834 | cg24693053 | 433212 |
| 0.121 | GABRA5 | 15:27112430 | cg10652393 | 202197 |
| 0.121 | PLSCR2 | 3:146187706 | cg02128651 | 43681 |
| 0.121 | PENK | 8:57358590 | cg21694941 | 385984 |
| 0.121 | FBN2 | 5:127873283 | cg05209584 | 103634 |
| 0.121 | PAX1 | 20:21686293 | cg01783070 | 36501 |
| 0.121 | | 18:6414602 | cg08336641 | 161747 |
| 0.121 | NRG1 | 8:31497464 | cg24946597 | 437530 |
| 0.121 | FBXO39; XAF1 | 17:6679564 | cg02374745 | 48515 |
| 0.121 | WNT2 | 7:116963259 | cg07697895 | 149945 |
| 0.121 | | 8:142413013 | cg06830360 | 133304 |
| 0.121 | SNORA76; SNORD104; | 17:62222808 | cg20969675 | 374778 |
| | TEX2 | | | |
| 0.121 | EHBP1; LOC100132215; | 2:63275602 | cg27344587 | 477023 |
| | OTX1 | | | |
| 0.121 | IRX1 | 5:3599686 | cg01294808 | 27051 |
| 0.121 | CADM2 | 3:85008587 | cg13331200 | 245621 |
| 0.121 | FAT1 | 4:187647690 | cg12769506 | 236972 |
| 0.121 | | 10:131762052 | cg11757357 | 220344 |
| 0.122 | ADAM12 | 10:128077307 | cg05237641 | 104212 |
| 0.122 | CLVS2 | 6:123317569 | cg08194879 | 159033 |
| 0.122 | | 12:127940654 | cg16065021 | 295854 |
| 0.122 | SLC6A3 | 5:1446208 | cg14502484 | 269687 |
| 0.122 | ASPG; MIR203; | 14:104583071 | cg03401997 | 68837 |
| | MIR3545 | | | |
| 0.122 | CCDC146 | 7:76747831 | cg02901065 | 58990 |
| 0.122 | ZFP42 | 4:188916581 | cg15718581 | 290161 |
| 0.122 | | 2:225306901 | cg10193763 | 194181 |
| 0.122 | HOXD10; HOXD11; | 2:176972581 | cg24633978 | 432153 |
| | HOXD12 | | | |
| 0.122 | ENTPD6 | 20:25177340 | cg00926318 | 19472 |
| 0.122 | TSHZ1; ZADH2 | 18:72917387 | cg22088248 | 391726 |
| 0.122 | POLG | 15:89879190 | cg24365117 | 427793 |
| 0.122 | | 7:43291318 | cg02964324 | 60381 |
| 0.122 | NETO1 | 18:70534298 | cg23883696 | 419843 |
| 0.122 | POU4F1 | 13:79170283 | cg24221648 | 425692 |
| 0.122 | GNAS; GNAS- | 20:57427650 | cg02890368 | 58752 |
| | AS1 | | | |
| 0.122 | PDE2A | 11:72301724 | cg26268145 | 458304 |
| 0.122 | ARL9; SRP72 | 4:57372356 | cg14775423 | 274866 |
| 0.122 | HLCS | 21:38353114 | cg22777062 | 401811 |
| 0.122 | TP73; WRAP73 | 1:3568210 | cg20677901 | 370380 |
| 0.122 | | 2:190192866 | cg25550823 | 446916 |
| 0.122 | SNX31 | 8:101661788 | cg10715223 | 203178 |
| 0.122 | ARHGEF10; MIR596 | 8:1771973 | cg10964367 | 207265 |
| 0.122 | B3GNT4; DIABLO; | 12:122688787 | cg23445859 | 413050 |
| | LRRC43 | | | |
| 0.122 | TRPC4 | 13:38444227 | cg16409955 | 301745 |
| 0.122 | KCNA1 | 12:5020693 | cg08832227 | 170388 |
| 0.122 | EXT2 | 11:44118094 | cg24662154 | 432585 |
| 0.123 | ADAMTSL3 | 15:84363061 | cg14598478 | 271807 |
| 0.123 | STK32B | 4:5053504 | cg08323075 | 161506 |
| 0.123 | DKFZp434J0226 | 19:46713446 | cg21100328 | 376752 |
| 0.123 | RNF180 | 5:63461803 | cg09839635 | 187877 |
| 0.123 | CYB561D2; NPRL2; | 3:50383175 | cg00460268 | 9723 |
| | RASSF1; | | | |
| | TMEM115; ZMYND10 | | | |
| 0.123 | RNF207; RPL22 | 1:6269448 | cg25286333 | 442745 |
| 0.123 | | 17:2627661 | cg12187586 | 227838 |
| 0.123 | P2RY12 | 3:151058783 | cg13229867 | 243994 |
| 0.123 | DUSP4 | 8:29210592 | cg05128656 | 102084 |
| 0.123 | HTR4 | 5:148034045 | cg04091563 | 82145 |
| 0.123 | TMEM164 | X:109246494 | cg04926827 | 98346 |
| 0.123 | C1QL1 | 17:43044660 | cg24042242 | 422692 |
| 0.123 | PCDHGA1; PCDHGA2 | 5:140711852 | cg05580809 | 110540 |
| 0.123 | VSTM5 | 11:93583693 | cg14291919 | 265581 |
| 0.123 | ZFP2; ZNF454 | 5:178367827 | cg24843380 | 435721 |
| 0.123 | KLHDC10 | 7:129781152 | cg07026433 | 137279 |
| 0.123 | SLIT2 | 4:20257590 | cg19940312 | 359283 |
| 0.123 | IRF6 | 1:209979665 | cg22029157 | 390830 |
| 0.123 | | 11:71350975 | cg13009111 | 240573 |
| 0.123 | PITX1 | 5:134367394 | cg23064601 | 406786 |
| 0.123 | PLSCR2 | 3:146187740 | cg21569635 | 384039 |
| 0.124 | SKAP2 | 7:26897685 | cg03730533 | 75381 |
| 0.124 | CABYR; TTC39C | 18:21719433 | cg26349976 | 459840 |
| 0.124 | TSHZ1; ZADH2 | 18:72916776 | cg02750262 | 56023 |
| 0.124 | ZNF835 | 19:57183118 | cg08146323 | 158144 |
| 0.124 | TBX15 | 1:119535928 | cg24035245 | 422569 |
| 0.124 | OSGIN1 | 16:83986941 | cg07978099 | 155051 |
| 0.124 | PRKD1 | 14:30396444 | cg10501704 | 199443 |
| 0.124 | SCRT2 | 20:644758 | cg12590600 | 234112 |
| 0.124 | TRPM4 | 19:49713828 | cg10951975 | 207052 |
| 0.124 | | 5:53564 | cg22807585 | 402379 |
| 0.124 | DUOX2; DUOXA1; | 15:45405103 | cg04222582 | 84830 |
| | DUOXA2 | | | |
| 0.124 | | 14:99584667 | cg23683254 | 416925 |
| 0.124 | FEZF1; LOC154860 | 7:121940216 | cg27393010 | 477821 |
| 0.124 | NELF; PNPLA7 | 9:140348828 | cg14384158 | 267359 |
| 0.124 | PTPN13 | 4:87515467 | cg15543566 | 287079 |
| 0.124 | KCTD14; NDUFC2- | 11:77734206 | cg16769152 | 308350 |
| | KCTD14 | | | |
| 0.124 | GBX2 | 2:237076811 | cg21426003 | 381745 |
| 0.124 | PON2; PON3 | 7:95025829 | cg08520743 | 164919 |
| 0.124 | CPEB1 | 15:83316223 | cg27578811 | 480950 |
| 0.124 | NTM | 11:131780492 | cg15617814 | 288391 |
| 0.124 | ATP9A | 20:50385371 | cg01900832 | 38931 |
| 0.124 | | 13:36705475 | cg25724283 | 449620 |
| 0.124 | | 10:21462441 | cg17426273 | 318854 |
| 0.124 | ALX1 | 12:85674296 | cg07780095 | 151415 |
| 0.124 | | 6:106441441 | cg03942932 | 79300 |
| 0.125 | HECW1 | 7:43152254 | cg08039116 | 156084 |
| 0.125 | | 8:61823027 | cg20904475 | 373766 |
| 0.125 | PRDM8 | 4:81118188 | cg03463411 | 70030 |
| 0.125 | INSR | 19:7294093 | cg26532951 | 462966 |
| 0.125 | HOXD4; MIR10B | 2:177014959 | cg17104824 | 313519 |
| 0.125 | PON2; PON3 | 7:95025740 | cg23230584 | 409500 |
| 0.125 | TFAP4 | 16:4324280 | cg00021532 | 522 |
| 0.125 | CRNDE; IRX5 | 16:54965087 | cg05870072 | 115671 |
| 0.125 | | 14:103673450 | cg26579556 | 463863 |
| 0.125 | NXPH2 | 2:139537824 | cg18150439 | 330428 |
| 0.125 | | 13:42002627 | cg25532201 | 446653 |
| 0.125 | KLHL4 | X:86772364 | cg15554694 | 287280 |
| 0.125 | MALL; NPHP1 | 2:110873396 | cg01615704 | 33421 |
| 0.125 | HAAO | 2:43020264 | cg17246140 | 315873 |
| 0.125 | DPP6 | 7:153584609 | cg27032232 | 471703 |
| 0.125 | LHFPL4 | 3:9594385 | cg04061117 | 81564 |
| 0.125 | EGFL7 | 9:139551542 | cg13900767 | 257221 |
| 0.125 | COX16; SYNJ2BP; | 14:70827109 | cg13099429 | 241974 |
| | SYNJ2BP- | | | |
| | COX16 | | | |
| 0.125 | LHX6 | 9:124990456 | cg13817952 | 255647 |
| 0.125 | KCNK16; KCNK17 | 6:39281885 | cg08315770 | 161367 |
| 0.125 | CCT8L2; TPTEP1 | 22:17082770 | cg14505585 | 269757 |
| 0.125 | FAM135B | 8:139509672 | cg23294090 | 410690 |
| 0.125 | GDA | 9:74764428 | cg21170041 | 377969 |
| 0.126 | IL1RAPL1 | X:28604661 | cg06927864 | 135328 |
| 0.126 | CIDEA | 18:12254551 | cg07204280 | 140912 |
| 0.126 | NPAS3 | 14:33403862 | cg08117309 | 157524 |
| 0.126 | ENPP2 | 8:120650994 | cg02534163 | 51618 |
| 0.126 | | 7:156400711 | cg16561657 | 304460 |
| 0.126 | STXBP5L | 3:120627406 | cg05293820 | 105268 |
| 0.126 | CNTN1 | 12:41086680 | cg14625113 | 272330 |
| 0.126 | IRF6 | 1:209979283 | cg25192855 | 441351 |
| 0.126 | CAST | 5:96038614 | cg24742349 | 434137 |
| 0.126 | FBN2 | 5:127874463 | cg11965976 | 224052 |
| 0.126 | ATXN2 | 12:112035212 | cg08273772 | 160571 |
| 0.126 | PDE4D | 5:59189120 | cg03323696 | 67297 |
| 0.126 | TOX2 | 20:42544663 | cg06779449 | 132219 |
| 0.126 | BCOR | X:40005177 | cg12737514 | 236481 |
| 0.126 | KIAA1530 | 4:1366597 | cg23921777 | 420508 |
| 0.126 | CXCL2 | 4:74965262 | cg23244559 | 409786 |
| 0.126 | MIR1469; NR2F2 | 15:96884187 | cg07016184 | 137060 |
| 0.126 | EGLN2; RAB4B- | 19:41319790 | cg01282508 | 26784 |
| | EGLN2 | | | |
| 0.126 | TMEM132C | 12:128751460 | cg03530754 | 71358 |
| 0.126 | PDZRN4 | 12:41582349 | cg21926708 | 389441 |
| 0.126 | EFCAB1 | 8:49647734 | cg08258526 | 160240 |
| 0.126 | PIN4 | X:71401384 | cg23905908 | 420234 |
| 0.126 | RP11- | 1:111813690 | cg07197785 | 140790 |
| | 165H20.1 | | | |
| 0.126 | LOC645249 | 7:156814491 | cg00024086 | 585 |
| 0.126 | ATHL1; IFITM5; | 11:288305 | cg08829299 | 170335 |
| | NLRP6 | | | |
| 0.126 | GALR1 | 18:74961966 | cg03659519 | 73996 |
| 0.126 | | 1:2738746 | cg10105681 | 192507 |
| 0.126 | RGAG4 | X:71351980 | cg20347665 | 365414 |
| 0.126 | RNF180 | 5:63461574 | cg14591786 | 271667 |
| 0.127 | EOMES | 3:27772805 | cg11642106 | 218279 |
| 0.127 | HOXB9; MIR196A1 | 17:46711017 | cg23690166 | 417069 |
| 0.127 | ARMC5; TGFB1I1 | 16:31484032 | cg10995380 | 207836 |
| 0.127 | PON2; PON3 | 7:95025855 | cg04685170 | 93636 |
| 0.127 | PRSS3 | 9:33749216 | cg22410499 | 396383 |
| 0.127 | CPNE4 | 3:131246097 | cg08151596 | 158229 |
| 0.127 | GABRG3 | 15:27216529 | cg11651237 | 218458 |
| 0.127 | B4GALNT2 | 17:47209827 | cg20233029 | 363633 |
| 0.127 | MSX1 | 4:4864488 | cg21538208 | 383498 |
| 0.127 | ARHGEF4 | 2:131673771 | cg09015973 | 173696 |
| 0.127 | PCDHA1; PCDHA10; | 5:140261663 | cg02051771 | 42046 |
| | PCDHA11; | | | |
| | PCDHA12; | | | |
| | PCDHA13; PCDHA2; | | | |
| | PCDHA3; | | | |
| | PCDHA4; | | | |
| | PCDHA5; PCDHA6; | | | |
| | PCDHA7; | | | |
| | PCDHA8; | | | |
| | PCDHA9 | | | |
| 0.127 | ASPG; MIR203; | 14:104583048 | cg13673833 | 252679 |
| | MIR3545 | | | |
| 0.127 | MAGI2-AS3 | 7:79083507 | cg02523844 | 51402 |
| 0.127 | IRX2 | 5:2739602 | cg21573200 | 384101 |
| 0.127 | NUP210 | 3:13431720 | cg17336044 | 317388 |
| 0.128 | | 2:132088801 | cg14001664 | 259383 |
| 0.128 | CCDC130; MRI1 | 19:13875137 | cg14272688 | 265161 |
| 0.128 | | 2:71115370 | cg10954469 | 207096 |
| 0.128 | ZNF347; ZNF415 | 19:53635967 | cg18301583 | 332927 |
| 0.128 | SNED1 | 2:241938321 | cg21384492 | 381197 |
| 0.128 | VSTM2A | 7:54614477 | cg06584494 | 128412 |
| 0.128 | MEIS2 | 15:37388540 | cg00691830 | 14602 |
| 0.128 | NLRP14; ZNF214 | 11:7041930 | cg17228942 | 315646 |
| 0.128 | KCNV1 | 8:110988043 | cg23291534 | 410667 |
| 0.128 | MAGI2-AS3 | 7:79081666 | cg06809544 | 132847 |
| 0.128 | FBXO39; XAF1 | 17:6679348 | cg00376544 | 7966 |
| 0.128 | PTF1A | 10:23481176 | cg22746058 | 401390 |
| 0.128 | TAL1 | 1:47691160 | cg10810752 | 204761 |
| 0.128 | FBRSL1 | 12:133137479 | cg03621470 | 73255 |
| 0.128 | CCDC36; LOC646498 | 3:49236946 | cg03540175 | 71588 |
| 0.128 | CXXC4 | 4:105412800 | cg22604213 | 399113 |
| 0.128 | MSX1 | 4:4864902 | cg11078084 | 209175 |
| 0.128 | FLJ45983; GATA3 | 10:8097641 | cg17124583 | 313810 |
| 0.128 | ADRA1B | 5:159343549 | cg07011172 | 136949 |
| 0.128 | UBD | 6:29521272 | cg22762215 | 401614 |
| 0.128 | TGFBI | 5:135364568 | cg15146621 | 281189 |
| 0.128 | CHST8 | 19:34112825 | cg26565021 | 463587 |
| 0.128 | JPH1 | 8:75234269 | cg23599843 | 415405 |
| 0.128 | ACSS1 | 20:25039707 | cg03321508 | 67252 |
| 0.128 | BASP1; LOC285696 | 5:17218778 | cg14306451 | 265851 |
| 0.128 | | 11:79148502 | cg05454619 | 108297 |
| 0.128 | NLRP14; ZNF214 | 11:7041752 | cg11838299 | 221836 |
| 0.128 | | 2:9235898 | cg20610452 | 369302 |
| 0.129 | PCDHA1; PCDHA10; | 5:140346236 | cg12832313 | 237924 |
| | PCDHA11; | | | |
| | PCDHA12; | | | |
| | PCDHA13; PCDHA2; | | | |
| | PCDHA3; | | | |
| | PCDHA4; | | | |
| | PCDHA5; PCDHA6; | | | |
| | PCDHA7; | | | |
| | PCDHA8; | | | |
| | PCDHA9; PCDHAC1; | | | |
| | PCDHAC2 | | | |
| 0.129 | C8orf42 | 8:495736 | cg05142982 | 102391 |
| 0.129 | | 11:70962935 | cg11263364 | 212293 |
| 0.129 | CDH12 | 5:21751337 | cg23404351 | 412413 |
| 0.129 | LOC100507055; | 12:133195094 | cg00058329 | 1387 |
| | P2RX2; POLE | | | |
| 0.129 | CCDC164; EPT1 | 2:26624934 | cg14273450 | 265175 |
| 0.129 | ISM1 | 20:13200929 | cg22588144 | 398900 |
| 0.129 | EGR2 | 10:64578919 | cg22903908 | 404041 |
| 0.129 | GSDMD; ZC3H3 | 8:144626190 | cg09422696 | 180737 |
| 0.129 | KCNK9 | 8:140715802 | cg21415530 | 381621 |
| 0.129 | | 3:94243571 | cg16274205 | 299307 |
| 0.129 | UNC80 | 2:210636539 | cg24938830 | 437420 |
| 0.129 | CABLES2; RPS21 | 20:60958108 | cg14168305 | 262866 |
| 0.129 | | 11:124324477 | cg03252499 | 65933 |
| 0.129 | FAT1 | 4:187645144 | cg24410535 | 428521 |
| 0.129 | PPP1R14A; SPINT2 | 19:38747378 | cg02571816 | 52340 |
| 0.130 | ASIP | 20:32856898 | cg05267394 | 104806 |
| 0.130 | CCDC144A | 17:16593885 | cg02879122 | 58511 |
| 0.130 | CRISPLD1 | 8:75896720 | cg15953602 | 293925 |
| 0.130 | KAL1 | X:8700172 | cg18396832 | 334461 |
| 0.130 | CELF1 | 11:47528955 | cg13308137 | 245225 |
| 0.130 | HOXC4; HOXC5; | 12:54404315 | cg13545297 | 249972 |
| | HOXC6; HOXC8; | | | |
| | HOXC9 | | | |
| 0.130 | DOCK1; FAM196A | 10:128994608 | cg25984344 | 453521 |
| 0.130 | LOC100507266 | 4:4577273 | cg07914772 | 153870 |
| 0.130 | KCNS2 | 8:99440279 | cg14486338 | 269395 |
| 0.130 | | 5:3093070 | cg20428720 | 366575 |
| 0.130 | HOXA10; HOXA10- | 7:27204981 | cg26521404 | 462738 |
| | HOXA9; HOXA7; | | | |
| | HOXA9; LOC100133311; | | | |
| | MIR196B | | | |
| 0.130 | RNF180 | 5:63461582 | cg06776999 | 132168 |
| 0.130 | MTRNR2L4; OR2C1 | 16:3415077 | cg09999114 | 190605 |
| 0.130 | PENK | 8:57358312 | cg12877723 | 238608 |
| 0.130 | NETO1 | 18:70534767 | cg18448581 | 335334 |
| 0.130 | TTC40 | 10:134650467 | cg25403174 | 444670 |
| 0.130 | ASXL3 | 18:31158234 | cg24880056 | 436440 |
| 0.130 | DEAF1; DRD4; | 11:637038 | cg15861585 | 292487 |
| | SCT | | | |
| 0.130 | GLT1D1 | 12:129337910 | cg06419750 | 125449 |
| 0.130 | LOC643714; TOX3 | 16:52580987 | cg26817217 | 467846 |
| 0.131 | UNCX | 7:1265851 | cg15591276 | 287897 |
| 0.131 | RARRES1 | 3:158450251 | cg14226182 | 264214 |
| 0.131 | PRDM8 | 4:81118500 | cg19409579 | 350194 |
| 0.131 | DQX1; PCGF1; | 2:74742786 | cg19656282 | 354267 |
| | TLX2 | | | |
| 0.131 | STL | 6:125283245 | cg24822696 | 435377 |
| 0.131 | CPNE4 | 3:131756545 | cg12981577 | 240178 |
| 0.131 | UBD | 6:29521407 | cg03402459 | 68848 |
| 0.131 | HLCS | 21:38362725 | cg14785464 | 275096 |
| 0.131 | SHROOM2 | X:9753940 | cg26318441 | 459244 |
| 0.131 | FTSJ2; NUDT1; | 7:2289054 | cg17467898 | 319545 |
| | SNX8 | | | |
| 0.131 | PCDHB10; PCDHB16; | 5:140562738 | cg21081817 | 376495 |
| | PCDHB7; | | | |
| | PCDHB8; | | | |
| | PCDHB9 | | | |
| 0.131 | NOS1 | 12:117798954 | cg04883903 | 97454 |
| 0.131 | CHL1 | 3:238615 | cg07746943 | 150879 |
| 0.131 | SGPP2 | 2:223289277 | cg00043095 | 1074 |
| 0.131 | TUSC1 | 9:25678015 | cg13985784 | 259065 |
| 0.131 | NKX2-8 | 14:37051417 | cg10530851 | 200070 |
| 0.132 | FRYL; SLC10A4; | 4:48492225 | cg22773661 | 401769 |
| | ZAR1 | | | |
| 0.132 | LOC402160; RNF4 | 4:2464050 | cg21722242 | 386297 |
| 0.132 | CHL1 | 3:239207 | cg21145524 | 377484 |
| 0.132 | | 18:73167671 | cg12975230 | 240067 |
| 0.132 | SALL1 | 16:51188697 | cg00683332 | 14419 |
| 0.132 | FAM19A5 | 22:48972349 | cg06273376 | 122999 |
| 0.132 | GRM6 | 5:178422128 | cg00582971 | 12427 |
| 0.132 | RTBDN | 19:12936512 | cg07583894 | 147890 |
| 0.132 | HOXA10- | 7:27192656 | cg23263937 | 410186 |
| | HOXA9; HOXA5; | | | |
| | HOXA6; HOXA7; | | | |
| | HOXA9; | | | |
| | LOC100133311 | | | |
| 0.132 | TMEM45B | 11:129685751 | cg27274706 | 475714 |
| 0.132 | NRK | X:105066526 | cg07674139 | 149532 |
| 0.132 | NRG1 | 8:31497829 | cg26267340 | 458285 |
| 0.132 | NAALAD2 | 11:89867653 | cg01577414 | 32669 |
| 0.132 | SMTNL2 | 17:4488345 | cg07888347 | 153363 |
| 0.132 | CHRNB1; FGF11; | 17:7341641 | cg25563256 | 447126 |
| | TMEM102 | | | |
| 0.132 | NEFM | 8:24771326 | cg07552803 | 147256 |
| 0.132 | HMGCLL1 | 6:55443757 | cg11907729 | 223067 |
| 0.132 | WNT2B | 1:113051846 | cg27181253 | 474270 |
| 0.132 | RADIL | 7:4869981 | cg00469380 | 9930 |
| 0.132 | NRXN1 | 2:50574196 | cg14875171 | 276641 |
| 0.133 | CTNNA2 | 2:79740208 | cg04670857 | 93356 |
| 0.133 | DOCK1; FAM196A | 10:128994605 | cg12204732 | 228111 |
| 0.133 | | 12:132146956 | cg24533007 | 430572 |
| 0.133 | CBLN2 | 18:70209432 | cg13478228 | 248599 |
| 0.133 | PIEZO2 | 18:11149470 | cg03686593 | 74481 |
| 0.133 | NELL1 | 11:20690957 | cg17371081 | 317950 |
| 0.133 | SLC34A2 | 4:25657381 | cg03738352 | 75550 |
| 0.133 | | 3:74663796 | cg00735667 | 15563 |
| 0.133 | | 5:132947501 | cg23236554 | 409620 |
| 0.133 | LRRC4C | 11:40315392 | cg17949440 | 326949 |
| 0.133 | CHST9 | 18:24765485 | cg16933181 | 310893 |
| 0.133 | | 6:168147405 | cg07147204 | 139710 |
| 0.134 | CNTNAP2 | 7:145813417 | cg11592503 | 217489 |
| 0.134 | ENTPD2; NPDC1 | 9:139943146 | cg14024893 | 259944 |
| 0.134 | MAGI2-AS3 | 7:79083445 | cg04652097 | 93018 |
| 0.134 | TACR3 | 4:104640662 | cg18538958 | 336883 |
| 0.134 | NXNL2 | 9:91150537 | cg14347199 | 266666 |
| 0.134 | COX6B2; FAM71E2; | 19:55866087 | cg20106459 | 361820 |
| | IL11; SUV420H2 | | | |
| 0.134 | DPRXP4; RNF135 | 17:29297391 | cg01347228 | 28034 |
| 0.134 | GPR6 | 6:110300573 | cg26252167 | 458027 |
| 0.134 | MAN1C1 | 1:25944514 | cg10706230 | 203035 |
| 0.135 | TJP1 | 15:30114474 | cg19965221 | 359674 |
| 0.135 | SSPO | 7:149484798 | cg16516272 | 303562 |
| 0.135 | C3orf25; MBD4 | 3:129147541 | cg14779951 | 274962 |
| 0.135 | | 13:50701501 | cg23104954 | 407495 |
| 0.135 | LRP3 | 19:33685817 | cg10276227 | 195631 |
| 0.135 | HOXD10; HOXD8; | 2:176994448 | cg14473102 | 269120 |
| | HOXD9; | | | |
| | LOC100506783 | | | |
| 0.135 | SNAP91 | 6:84418433 | cg24190603 | 425243 |
| 0.135 | ISL1 | 5:50679147 | cg16318053 | 300061 |
| 0.135 | PCDHA1; PCDHA10; | 5:140261653 | cg19876814 | 358160 |
| | PCDHA11; | | | |
| | PCDHA12; | | | |
| | PCDHA13; PCDHA2; | | | |
| | PCDHA3; | | | |
| | PCDHA4; | | | |
| | PCDHA5; PCDHA6; | | | |
| | PCDHA7; | | | |
| | PCDHA8; | | | |
| | PCDHA9 | | | |
| 0.135 | SSTR1 | 14:38678420 | cg18443253 | 335246 |
| 0.136 | KCNQ4 | 1:41248881 | cg16294363 | 299640 |
| 0.136 | DUSP27 | 1:167090646 | cg17104258 | 313508 |
| 0.136 | | 4:170696239 | cg18912209 | 342859 |
| 0.136 | CXCL5 | 4:74864165 | cg27109129 | 473022 |
| 0.136 | LHX8 | 1:75602412 | cg08146483 | 158147 |
| 0.136 | CCR6 | 6:167559913 | cg16338365 | 300452 |
| 0.136 | KIF17; SH2D5 | 1:21044101 | cg09793172 | 187028 |
| 0.136 | ANKRD26P3; | 13:19918586 | cg06722407 | 131065 |
| | LINC00421 | | | |
| 0.136 | ZFP42 | 4:188916875 | cg10242602 | 195098 |
| 0.136 | UBD | 6:29521430 | cg15818307 | 291669 |
| 0.136 | HOXD1; LOC401022 | 2:177053292 | cg05099387 | 101608 |
| 0.136 | ISM1 | 20:13200944 | cg14060111 | 260677 |
| 0.136 | NMBR | 6:142409507 | cg20747266 | 371647 |
| 0.137 | | 7:1287065 | cg12560987 | 233619 |
| 0.137 | STL | 6:125283953 | cg10621825 | 201643 |
| 0.137 | FAM181B | 11:82444798 | cg09617579 | 184158 |
| 0.137 | DACH2 | X:85402643 | cg16259355 | 299014 |
| 0.137 | | 13:21872234 | cg11317459 | 213174 |
| 0.137 | IRX4 | 5:1882885 | cg14507560 | 269807 |
| 0.137 | FGD3 | 9:95737070 | cg14263098 | 264963 |
| 0.137 | | 7:157477846 | cg04207084 | 84494 |
| 0.137 | AATF; LHX1 | 17:35300010 | cg19760241 | 356113 |
| 0.137 | HES6; LOC151174; | 2:239149859 | cg24127874 | 424152 |
| | LOC643387; | | | |
| | PER2 | | | |
| 0.137 | HOXD4; MIR10B | 2:177014962 | cg00014998 | 368 |
| 0.137 | C5orf63 | 5:126409308 | cg21113900 | 376968 |
| 0.137 | SLC16A11; SLC16A13 | 17:6946059 | cg19639560 | 354003 |
| 0.137 | ZBTB44 | 11:130185651 | cg14482569 | 269324 |
| 0.137 | ARHGEF4 | 2:131674140 | cg07815799 | 152125 |
| 0.137 | DAXX; TAPBP; | 6:33280424 | cg26083458 | 455084 |
| | ZBTB22 | | | |
| 0.137 | DPP10; LOC389023 | 2:115919829 | cg01718116 | 35249 |
| 0.137 | OR2H1 | 6:29427519 | cg05111645 | 101800 |
| 0.137 | HAS1; LINC00085 | 19:52206667 | cg17519645 | 320492 |
| 0.137 | TGFBI | 5:135364580 | cg09873933 | 188549 |
| 0.138 | NAALAD2 | 11:89867637 | cg16886581 | 310118 |
| 0.138 | | 14:95331190 | cg03459776 | 69950 |
| 0.138 | FOXF2 | 6:1390622 | cg12221475 | 228386 |
| 0.138 | THRB | 3:24537050 | cg27526665 | 479998 |
| 0.138 | | 7:156400779 | cg18584424 | 337608 |
| 0.138 | MIR3663 | 10:118922493 | cg24234899 | 425848 |
| 0.138 | PITX1 | 5:134364387 | cg00396667 | 8373 |
| 0.138 | DRD5 | 4:9783198 | cg03045635 | 62021 |
| 0.138 | SIK3 | 11:116857794 | cg09458566 | 181396 |
| 0.138 | | 13:109147921 | cg03994318 | 80330 |
| 0.138 | IGF2BP1 | 17:47075880 | cg02745847 | 55935 |
| 0.138 | SKINTL | 1:48559803 | cg08871545 | 171173 |
| 0.138 | LMX1A | 1:165204796 | cg09118258 | 175320 |
| 0.138 | GATA6 | 18:19756877 | cg12615165 | 234584 |
| 0.138 | CYB561D2; NPRL2; | 3:50383079 | cg20881888 | 373460 |
| | RASSF1; | | | |
| | TMEM115; ZMYND10 | | | |
| 0.138 | NXNL1; TMEM221 | 19:17559217 | cg18380550 | 334165 |
| 0.138 | C12orf53 | 12:6818384 | cg23877043 | 419727 |
| 0.138 | TAL1 | 1:47691158 | cg10380348 | 197448 |
| 0.139 | ALPK3 | 15:85360664 | cg20318166 | 364967 |
| 0.139 | VSTM2A | 7:54614620 | cg06139908 | 120728 |
| 0.139 | | 15:26108391 | cg03419058 | 69144 |
| 0.139 | OPCML | 11:132813563 | cg18710784 | 339517 |
| 0.139 | HOXA5; HOXA6; | 7:27187560 | cg14044640 | 260386 |
| | HOXA7; LOC100133311 | | | |
| 0.139 | | 6:27525678 | cg20494374 | 367603 |
| 0.139 | KDM1A; MIR3115 | 1:23361764 | cg25002551 | 438359 |
| 0.139 | RLF | 1:40626599 | cg18646240 | 338570 |
| 0.139 | NRSN2 | 20:328035 | cg21192095 | 378373 |
| 0.139 | MSX1 | 4:4864430 | cg24840099 | 435675 |
| 0.139 | FLJ16779; NKAIN4 | 20:61886079 | cg08111446 | 157414 |
| 0.139 | DUSP4 | 8:29210154 | cg13635007 | 251899 |
| 0.139 | MAGI2-AS3 | 7:79083509 | cg24021956 | 422308 |
| 0.139 | H2AFY | 5:134735637 | cg15613420 | 288307 |
| 0.139 | PLD3 | 19:40871823 | cg08360726 | 162147 |
| 0.140 | KIRREL3; ST3GAL4 | 11:126286828 | cg12804791 | 237513 |
| 0.140 | ASIP | 20:32856846 | cg16440058 | 302309 |
| 0.140 | NKAIN3 | 8:63161609 | cg15858239 | 292441 |
| 0.140 | SMAD3 | 15:67396487 | cg19193595 | 346888 |
| 0.140 | ALPK3 | 15:85360655 | cg25895948 | 452116 |
| 0.140 | C8orf83 | 8:93977794 | cg16560711 | 304445 |
| 0.140 | MSX1 | 4:4864532 | cg09748975 | 186296 |
| 0.140 | ACTN3; CTSF | 11:66326798 | cg05475524 | 108647 |
| 0.141 | DPP6 | 7:153584873 | cg14564076 | 271091 |
| 0.141 | CTNNA2 | 2:79740185 | cg19707040 | 355066 |
| 0.141 | SOX1 | 13:112721325 | cg24604013 | 431705 |
| 0.141 | DOCK1; FAM196A | 10:128994297 | cg06525651 | 127283 |
| 0.141 | SLC6A18; SLC6A19 | 5:1222847 | cg04248937 | 85368 |
| 0.141 | C14orf23; FOXG1 | 14:29236898 | cg07489048 | 146092 |
| 0.141 | NANS | 9:100819928 | cg13527921 | 249630 |
| 0.141 | | 11:44545144 | cg13759632 | 254444 |
| 0.141 | LRP12 | 8:105601036 | cg24098951 | 423650 |
| 0.141 | COL23A1 | 5:178004091 | cg08475953 | 164148 |
| 0.141 | ASGR1 | 17:7077709 | cg03975755 | 79935 |
| 0.141 | | 11:133837265 | cg23152235 | 408116 |
| 0.141 | SIM2 | 21:38070671 | cg26938272 | 470089 |
| 0.141 | SCN2B; SCN4B | 11:118023765 | cg18718634 | 339649 |
| 0.141 | FZD5 | 2:208631684 | cg15714846 | 290094 |
| 0.141 | DOCK1 | 10:128593946 | cg23665778 | 416569 |
| 0.141 | INPP5A; NKX6-2 | 10:134600701 | cg17996619 | 327663 |
| 0.141 | ONECUT2 | 18:55095249 | cg15222899 | 282298 |
| 0.141 | ZDHHC4 | 7:6616423 | cg19149522 | 346313 |
| 0.141 | OR10AD1 | 12:48592721 | cg23748340 | 417996 |
| 0.142 | NLRP2 | 19:55477759 | cg19689876 | 354758 |
| 0.142 | NXPH2 | 2:139537845 | cg00804628 | 16987 |
| 0.142 | FAIM3; IL24 | 1:207082828 | cg06528575 | 127352 |
| 0.142 | ZC3H3 | 8:144599125 | cg13071618 | 241523 |
| 0.142 | FAM84A | 2:14775046 | cg05602862 | 110952 |
| 0.142 | FAM84A | 2:14773973 | cg16846645 | 309527 |
| 0.142 | HMX3 | 10:124897048 | cg15396686 | 284826 |
| 0.142 | RNF207; RPL22 | 1:6269260 | cg15731815 | 290408 |
| 0.142 | LOC100188947 | 10:93058549 | cg04285822 | 86104 |
| 0.142 | HOOK1 | 1:60280812 | cg15900248 | 293091 |
| 0.142 | DRD5 | 4:9783196 | cg23847712 | 419333 |
| 0.142 | CNTN4 | 3:2140256 | cg04222358 | 84827 |
| 0.142 | | 3:159818742 | cg07617768 | 148563 |
| 0.142 | GLRB | 4:157997367 | cg16269431 | 299215 |
| 0.143 | | 2:68917482 | cg18670846 | 338907 |
| 0.143 | | 8:41624841 | cg07162571 | 140077 |
| 0.143 | MTUS2 | 13:30055092 | cg05122437 | 101967 |
| 0.143 | FAM110B | 8:59058254 | cg13247663 | 244256 |
| 0.143 | | 2:92027943 | cg27311294 | 476418 |
| 0.143 | TNR | 1:175568559 | cg24932457 | 437299 |
| 0.143 | TNIP3 | 4:122078348 | cg26832294 | 468154 |
| 0.143 | C5orf63 | 5:126409007 | cg10200291 | 194305 |
| 0.143 | BCOR | X:40012100 | cg06548617 | 127783 |
| 0.143 | | 11:32008961 | cg20019985 | 360529 |
| 0.143 | OCRL | X:128674224 | cg05032353 | 100321 |
| 0.143 | CHST8 | 19:34113010 | cg16190732 | 297843 |
| 0.143 | UBD | 6:29520774 | cg08065408 | 156530 |
| 0.143 | PCDHGA1; PCDHGA10; | 5:140787430 | cg07613945 | 148493 |
| | PCDHGA2; | | | |
| | PCDHGA3; | | | |
| | PCDHGA4; | | | |
| | PCDHGA5; | | | |
| | PCDHGA6; | | | |
| | PCDHGA7; PCDHGA8; | | | |
| | PCDHGA9; | | | |
| | PCDHGB1; | | | |
| | PCDHGB2; | | | |
| | PCDHGB3; | | | |
| | PCDHGB4; | | | |
| | PCDHGB5; PCDHGB6; | | | |
| | PCDHGB7 | | | |
| 0.143 | SYT15 | 10:46970625 | cg09373037 | 179902 |
| 0.143 | FOXI2 | 10:129535669 | cg07918545 | 153959 |
| 0.144 | LOC649133; POU2F3 | 11:120110882 | cg26357587 | 459979 |
| 0.144 | CSMD1 | 8:4849827 | cg23495279 | 413823 |
| 0.144 | OSTalpha; ZDHHC19 | 3:195934355 | cg26292150 | 458748 |
| 0.144 | PITX1 | 5:134363877 | cg04223420 | 84848 |
| 0.144 | PDZRN4 | 12:41582362 | cg16896079 | 310254 |
| 0.144 | SH2D4B | 10:82295864 | cg15642854 | 288829 |
| 0.144 | UNC80 | 2:210636560 | cg04100532 | 82347 |
| 0.144 | CYB561D2; NPRL2; | 3:50383118 | cg11270070 | 212403 |
| | RASSF1; | | | |
| | TMEM115; ZMYND10 | | | |
| 0.144 | DPP6 | 7:153749756 | cg15549927 | 287181 |
| 0.144 | HCN1 | 5:45695643 | cg18273840 | 332528 |
| 0.144 | LHX8 | 1:75602167 | cg08272731 | 160551 |
| 0.145 | UBD | 6:29521420 | cg22305167 | 394855 |
| 0.145 | ADAMTS20 | 12:43945892 | cg19619405 | 353636 |
| 0.145 | HAGH | 16:1863053 | cg00935504 | 19660 |
| 0.145 | ISM1 | 20:13200931 | cg04432319 | 88934 |
| 0.145 | ANKS1B | 12:99288805 | cg03609960 | 73052 |
| 0.145 | LHX3 | 9:139085579 | cg04455058 | 89330 |
| 0.145 | | 6:10382103 | cg20906291 | 373785 |
| 0.145 | FAM110B | 8:59058273 | cg12615766 | 234593 |
| 0.145 | CYP1A1 | 15:75018852 | cg20004910 | 360290 |
| 0.145 | CACNB2 | 10:18429703 | cg02456226 | 50000 |
| 0.145 | SNTG2 | 2:1134065 | cg16519574 | 303633 |
| 0.145 | DPYS | 8:105478855 | cg16783744 | 308581 |
| 0.145 | | 19:57618136 | cg18384778 | 334232 |
| 0.145 | | 2:200524189 | cg20711639 | 371065 |
| 0.145 | HLA- | 6:33053608 | cg01991743 | 40852 |
| | DPA1; HLA- | | | |
| | DPB1 | | | |
| 0.146 | GNG12; LOC100289178 | 1:68299409 | cg13184736 | 243235 |
| 0.146 | NAALAD2 | 11:89867679 | cg18540157 | 336897 |
| 0.146 | | 1:241520286 | cg14156405 | 262603 |
| 0.146 | | 18:4455337 | cg25627226 | 448224 |
| 0.146 | PLSCR2 | 3:146187126 | cg24092307 | 423558 |
| 0.146 | MAD2L1BP; RSPH9 | 6:43612908 | cg17536595 | 320768 |
| 0.146 | GALNT10 | 5:153569246 | cg13372456 | 246337 |
| 0.146 | KCNA4 | 11:30038675 | cg08490115 | 164399 |
| 0.146 | | 7:158380886 | cg27392792 | 477814 |
| 0.146 | KY | 3:134369661 | cg24675998 | 432827 |
| 0.146 | IRF6 | 1:209979624 | cg09509183 | 182272 |
| 0.147 | | 7:156400881 | cg27483342 | 479249 |
| 0.147 | ZSCAN1 | 19:58545333 | cg27391267 | 477779 |
| 0.147 | NAALAD2 | 11:89867704 | cg21817737 | 387693 |
| 0.147 | NID1 | 1:236228741 | cg07157830 | 139963 |
| 0.147 | TCHH | 1:152079713 | cg16689481 | 306872 |
| 0.147 | ZBTB44 | 11:130185670 | cg05227350 | 103986 |
| 0.147 | AGAP1 | 2:236412034 | cg11045331 | 208636 |
| 0.147 | KDM2B | 12:122019006 | cg07793808 | 151670 |
| 0.147 | KIF12 | 9:116860650 | cg14326196 | 266248 |
| 0.148 | FAM150A | 8:53477881 | cg09442654 | 181101 |
| 0.148 | ADAP1 | 7:968552 | cg05904016 | 116371 |
| 0.148 | DOCK1; FAM196A | 10:128994644 | cg03129384 | 63592 |
| 0.148 | MAML2 | 11:95969587 | cg05270552 | 104870 |
| 0.148 | SP8 | 7:20824932 | cg23093589 | 407294 |
| 0.148 | | 12:119212248 | cg22125805 | 392264 |
| 0.148 | ZSCAN18 | 19:58609618 | cg22721334 | 401040 |
| 0.148 | FOSB | 19:45976195 | cg12542255 | 233333 |
| 0.148 | ZSCAN18 | 19:58609602 | cg25784220 | 450602 |
| 0.148 | GALR1 | 18:74962672 | cg06360427 | 124375 |
| 0.148 | | 5:168728081 | cg07136998 | 139473 |
| 0.148 | ANKRD18A; FAM201A | 9:38622559 | cg21189438 | 378326 |
| 0.148 | CSGALNACT2; | 10:43633030 | cg07178550 | 140402 |
| | RET | | | |
| 0.148 | | 13:53775410 | cg13974192 | 258762 |
| 0.149 | DIAPH1; PCDHGA12; | 5:140892822 | cg08860070 | 170948 |
| | PCDHGC3; | | | |
| | PCDHGC4; | | | |
| | PCDHGC5 | | | |
| 0.149 | DNAAF2; LRR1; | 14:50084155 | cg22857670 | 403218 |
| | MGAT2; RPL36AL | | | |
| 0.149 | MACROD2; SEL1L2 | 20:13976096 | cg01552272 | 32076 |
| 0.149 | MYH13 | 17:10220829 | cg13984289 | 259022 |
| 0.149 | | 22:20284604 | cg00192046 | 4240 |
| 0.149 | CCDC148; PKP4 | 2:159312727 | cg14426788 | 268232 |
| 0.149 | | 1:23544995 | cg19743168 | 355780 |
| 0.150 | GLRB | 4:157997360 | cg22065614 | 391410 |
| 0.150 | | 16:51671357 | cg07302051 | 142686 |
| 0.150 | ZFP42 | 4:188916943 | cg14189571 | 263343 |
| 0.150 | UBD | 6:29520752 | cg11935738 | 223492 |
| 0.150 | CPSF3; IAH1 | 2:9614225 | cg08047252 | 156223 |
| 0.150 | GP9 | 3:128787213 | cg20264543 | 364127 |
| 0.150 | CCR6 | 6:167559851 | cg02524205 | 51410 |
| 0.150 | EFCAB1 | 8:49647809 | cg11469098 | 215629 |
| 0.150 | IRX4 | 5:1876397 | cg14451382 | 268670 |
| 0.150 | ARHGAP23 | 17:36666642 | cg15825321 | 291821 |
| 0.150 | CLEC14A | 14:38724648 | cg16404157 | 301618 |
| 0.150 | CACNA2D3 | 3:54155663 | cg18143243 | 330278 |
| 0.151 | BTG4; C11orf88; | 11:111383624 | cg23211240 | 409137 |
| | MIR34B; MIR34C | | | |
| 0.151 | GTF3A; MTIF3 | 13:28016624 | cg26772116 | 467001 |
| 0.151 | | 16:49499006 | cg01517680 | 31431 |
| 0.151 | CTTN | 11:70262058 | cg16629523 | 305772 |
| 0.151 | CCDC106; U2AF2; | 19:56159485 | cg08496742 | 164543 |
| | ZNF580; | | | |
| | ZNF581 | | | |
| 0.152 | DQX1; PCGF1; | 2:74743009 | cg07203423 | 140895 |
| | TLX2 | | | |
| 0.152 | KCNIP1 | 5:169960825 | cg10931884 | 206704 |
| 0.152 | MAL2 | 8:120220856 | cg02225716 | 45426 |
| 0.152 | GDF6 | 8:97173034 | cg21859781 | 388371 |
| 0.152 | HOXC4; HOXC5; | 12:54424203 | cg13826247 | 255805 |
| | HOXC6; MIR615 | | | |
| 0.152 | NCAM1 | 11:112831501 | cg21727486 | 386364 |
| 0.152 | ZNF503; ZNF503- | 10:77156697 | cg25023298 | 438769 |
| | AS2 | | | |
| 0.152 | MDGA1 | 6:37616803 | cg24442454 | 429044 |
| 0.152 | SHROOM2 | X:9754927 | cg05782106 | 114103 |
| 0.152 | | 4:1515011 | cg08480901 | 164253 |
| 0.153 | NXPH2 | 2:139537826 | cg14923640 | 277390 |
| 0.153 | BDNF | 11:27740813 | cg13974632 | 258776 |
| 0.153 | | 3:74663689 | cg24346629 | 427487 |
| 0.153 | | 4:1400189 | cg24496475 | 429912 |
| 0.153 | | 14:97058777 | cg11871960 | 222513 |
| 0.154 | ATP2C2 | 16:84402158 | cg07507755 | 146441 |
| 0.154 | | 7:32358540 | cg27511599 | 479742 |
| 0.154 | INTU | 4:128544375 | cg26114043 | 455622 |
| 0.154 | PNLIPRP2 | 10:118387207 | cg18699744 | 339327 |
| 0.154 | WIPI2 | 7:5271480 | cg26660312 | 465112 |
| 0.154 | GALNTL6 | 4:172734431 | cg22153181 | 392677 |
| 0.154 | MSI2 | 17:55362836 | cg07249730 | 141786 |
| 0.154 | LIN9; MIXL1 | 1:226411057 | cg18564989 | 337293 |
| 0.154 | GPR125 | 4:22520573 | cg13702087 | 253322 |
| 0.155 | MMP16 | 8:89340139 | cg12818557 | 237751 |
| 0.155 | 11-Mar | 5:16179660 | cg09017434 | 173718 |
| 0.155 | | 5:143978357 | cg11527001 | 216436 |
| 0.155 | ADAMTS16 | 5:5140001 | cg22954449 | 404825 |
| 0.155 | PITX1 | 5:134365728 | cg18606375 | 337970 |
| 0.155 | | 10:109674526 | cg00157668 | 3542 |
| 0.155 | TSHZ1; ZADH2 | 18:72916393 | cg11568697 | 217092 |
| 0.155 | | 2:240135921 | cg18121901 | 329904 |
| 0.155 | LYPD1; NCKAP5 | 2:133427612 | cg25571189 | 447240 |
| 0.155 | NAALAD2 | 11:89867809 | cg12403137 | 230998 |
| 0.155 | HOXC8; HOXC9 | 12:54396990 | cg22679316 | 400266 |
| 0.155 | ANKRD36B | 2:98206733 | cg23224356 | 409392 |
| 0.156 | NKAIN3 | 8:63161589 | cg08855288 | 170823 |
| 0.156 | RPS7P5 | 1:240161445 | cg21854408 | 388273 |
| 0.156 | | 12:116354837 | cg20169823 | 362815 |
| 0.156 | GABRA5 | 15:27112902 | cg03462380 | 70010 |
| 0.156 | UNCX | 7:1267228 | cg11715858 | 219570 |
| 0.156 | TNIP3 | 4:122078167 | cg10061361 | 191660 |
| 0.156 | CDH11 | 16:65157428 | cg07537692 | 146986 |
| 0.156 | DNAJB6 | 7:157192974 | cg25039694 | 439065 |
| 0.156 | DOCK1; FAM196A | 10:128994432 | cg03242819 | 65770 |
| 0.156 | ADAMTSL1 | 9:18514052 | cg14470762 | 269062 |
| 0.157 | CPNE9; MTMR14 | 3:9745610 | cg02741521 | 55842 |
| 0.157 | KIF25 | 6:168436099 | cg18319852 | 333241 |
| 0.157 | PIN4 | X:71401206 | cg26578373 | 463841 |
| 0.157 | ANKRD36BP2 | 2:89065379 | cg02646394 | 53977 |
| 0.157 | PXDN | 2:1748132 | cg25181651 | 441181 |
| 0.158 | GPR78 | 4:8582227 | cg18901644 | 342716 |
| 0.158 | ADAMTS16 | 5:5139797 | cg16670809 | 306536 |
| 0.158 | | 18:6414974 | cg18556788 | 337142 |
| 0.158 | GRM7 | 3:6903019 | cg27199820 | 474552 |
| 0.158 | CBWD2; FOXD4L1 | 2:114261369 | cg23850283 | 419371 |
| 0.158 | DPP10; LOC389023 | 2:115919006 | cg03753331 | 75826 |
| 0.158 | MIR4634 | 5:174178693 | cg12398645 | 230921 |
| 0.159 | SEMA3C | 7:80548456 | cg03823904 | 77109 |
| 0.159 | DHRS4L2 | 14:24476559 | cg10926857 | 206629 |
| 0.159 | WBSCR17 | 7:70597058 | cg02300154 | 46944 |
| 0.159 | | 11:2367787 | cg20805368 | 372479 |
| 0.159 | NTM | 11:131781257 | cg19717586 | 355276 |
| 0.159 | FLJ31485; FZD10 | 12:130647004 | cg21885046 | 388782 |
| 0.160 | CBLN1 | 16:49316197 | cg03389653 | 68591 |
| 0.160 | GBX2 | 2:237076815 | cg19761848 | 356155 |
| 0.160 | BCOR | X:40035961 | cg00016934 | 407 |
| 0.160 | HS3ST2 | 16:22825046 | cg09191731 | 176695 |
| 0.160 | ZNF148 | 3:125076372 | cg02970696 | 60506 |
| 0.160 | PROZ | 13:113819073 | cg00898013 | 18847 |
| 0.160 | | 2:130971164 | cg08566455 | 165743 |
| 0.160 | | 7:32467657 | cg18961681 | 343589 |
| 0.160 | | 7:152621979 | cg08202754 | 159199 |
| 0.161 | FEZF1; LOC154860 | 7:121946036 | cg10485664 | 199138 |
| 0.161 | NKAIN3 | 8:63161579 | cg01906015 | 39032 |
| 0.161 | PROCA1; RAB34; | 17:27044629 | cg21816330 | 387671 |
| | RPL23A; | | | |
| | SNORD42A; SNORD42B; | | | |
| | SNORD4A; | | | |
| | SNORD4B; | | | |
| | TLCD1 | | | |
| 0.161 | DOCK1 | 10:128593922 | cg06128198 | 120545 |
| 0.161 | HTR4 | 5:148033882 | cg11438011 | 215195 |
| 0.161 | PITX1 | 5:134363973 | cg07274716 | 142211 |
| 0.161 | UHRF1BP1 | 6:34760872 | cg07306190 | 142760 |
| 0.161 | IRX4 | 5:1887300 | cg07565505 | 147521 |
| 0.161 | SOX17 | 8:55370429 | cg11214140 | 211422 |
| 0.161 | GRHL2 | 8:102505986 | cg03650674 | 73811 |
| 0.162 | ABCB11; G6PC2 | 2:169769616 | cg05703053 | 112745 |
| 0.162 | | 18:6729810 | cg02824202 | 57399 |
| 0.162 | GALR1 | 18:74962000 | cg17911318 | 326333 |
| 0.162 | KCNA4 | 11:30038672 | cg15044957 | 279439 |
| 0.162 | CNTNAP2 | 7:145813421 | cg21126583 | 377176 |
| 0.162 | NID1 | 1:236228744 | cg04837025 | 96465 |
| 0.162 | THRB | 3:24536474 | cg20985755 | 375077 |
| 0.162 | COBL | 7:51384371 | cg12988534 | 240269 |
| 0.162 | GDF7 | 2:20870362 | cg07755735 | 151051 |
| 0.162 | ARHGAP33; C19orf55 | 19:36265700 | cg18805164 | 341126 |
| 0.163 | | 8:143070187 | cg06573787 | 128217 |
| 0.163 | ERG | 21:40033163 | cg12884169 | 238711 |
| 0.163 | PON2; PON3 | 7:95025736 | cg07121856 | 139138 |
| 0.163 | HOXD10; HOXD11; | 2:176972563 | cg11546137 | 216732 |
| | HOXD12 | | | |
| 0.164 | NSUN4 | 1:46814106 | cg15580309 | 287719 |
| 0.164 | CYB561D2; NPRL2; | 3.50383167 | cg09682796 | 185261 |
| | RASSF1; | | | |
| | TMEM115; ZMYND10 | | | |
| 0.164 | CHL1 | 3:239665 | cg10603275 | 201352 |
| 0.164 | MYOG; PPFIA4 | 1:203045044 | cg00805880 | 17008 |
| 0.165 | GNAI1 | 7:79764178 | cg25744767 | 449994 |
| 0.165 | LOC649133; POU2F3 | 11:120110887 | cg17176573 | 314750 |
| 0.165 | 11-Mar | 5:16179633 | cg23479922 | 413567 |
| 0.165 | PSMB9; TAP1 | 6:32829303 | cg05062939 | 100976 |
| 0.165 | TMEM229A | 7:123673331 | cg17025835 | 312249 |
| 0.165 | CEND1; PDDC1; | 11:783889 | cg01741372 | 35661 |
| | SLC25A22 | | | |
| 0.166 | DOCK1 | 10:128593943 | cg22665692 | 400049 |
| 0.166 | ATP6V1A | 3:113502832 | cg19924544 | 358964 |
| 0.166 | C6orf147; KHDC1 | 6:74024628 | cg02447304 | 49827 |
| 0.166 | | 22:43168851 | cg24694833 | 433260 |
| 0.167 | FAT1 | 4:187645118 | cg01244944 | 26097 |
| 0.167 | LILRB3 | 19:54728037 | cg23679434 | 416846 |
| 0.167 | SYT14 | 1:210111561 | cg19922137 | 358920 |
| 0.167 | ZNF844 | 19:12175935 | cg26827373 | 468055 |
| 0.167 | EVX2; HOXD12; | 2:176957842 | cg04415176 | 88629 |
| | HOXD13 | | | |
| 0.167 | | 10:131762462 | cg07045241 | 137620 |
| 0.167 | KLF8 | X:56259243 | cg06457357 | 126059 |
| 0.168 | C5orf63 | 5:126408756 | cg14340928 | 266559 |
| 0.168 | MAGI2-AS3 | 7:79083408 | cg22280038 | 394533 |
| 0.168 | | 4:21950567 | cg00688962 | 14541 |
| 0.169 | SHISA6 | 17:11144043 | cg13330559 | 245613 |
| 0.169 | ARSG; SLC16A6 | 17:66287032 | cg11879514 | 222640 |
| 0.169 | RNF180 | 5:63461654 | cg17370163 | 317926 |
| 0.169 | DACH1 | 13:72439698 | cg05510902 | 109333 |
| 0.169 | | 7:55516724 | cg03357999 | 68033 |
| 0.169 | EPB41L3 | 18:5543801 | cg00027083 | 665 |
| 0.170 | | 11:67499431 | cg02666610 | 54357 |
| 0.170 | CCT8L2; TPTEP1 | 22:17082772 | cg01660911 | 34237 |
| 0.171 | UBTD1 | 10:99278449 | cg10989317 | 207724 |
| 0.171 | | 18:6414976 | cg14352983 | 266773 |
| 0.171 | WBSCR17 | 7:70597065 | cg03044249 | 61987 |
| 0.171 | MYO16 | 13:109352806 | cg24702069 | 433391 |
| 0.171 | ANKRD36 | 2:97778927 | cg12623145 | 234729 |
| 0.171 | | 7:152622022 | cg15818671 | 291676 |
| 0.171 | SP8 | 7:20824706 | cg01003961 | 21140 |
| 0.172 | CTTNBP2 | 7:117513101 | cg05951860 | 117342 |
| 0.172 | NAT2 | 8:18244861 | cg12174804 | 227617 |
| 0.172 | WBSCR17 | 7:70597921 | cg07143083 | 139618 |
| 0.172 | THRB | 3:24536478 | cg13790603 | 255057 |
| 0.172 | | 16:85368973 | cg02418439 | 49379 |
| 0.173 | UBD | 6:29521356 | cg21632158 | 385088 |
| 0.173 | ADAMTSL3 | 15:84322584 | cg04138185 | 83155 |
| 0.173 | RAB32 | 6:146864885 | cg26252281 | 458030 |
| 0.174 | CLEC14A | 14:38724675 | cg05057720 | 100847 |
| 0.174 | DOCK1; FAM196A | 10:128994603 | cg03070297 | 62543 |
| 0.174 | MDGA1 | 6:37618009 | cg26720545 | 466246 |
| 0.174 | BAIAP2L1 | 7:98029266 | cg08684580 | 167809 |
| 0.174 | LRRC4C | 11:40314978 | cg19849428 | 357643 |
| 0.174 | | 8:7079106 | cg08498719 | 164579 |
| 0.174 | FAM43B | 1:20879547 | cg12768681 | 236962 |
| 0.174 | SEMA5B | 3:122694286 | cg07733481 | 150639 |
| 0.175 | SGPP2 | 2:223289306 | cg07357987 | 143728 |
| 0.175 | | 8:76320150 | cg26220018 | 457454 |
| 0.175 | CDH2 | 18:25757710 | cg14312538 | 265980 |
| 0.175 | CR1L | 1:207842917 | cg17822947 | 324851 |
| 0.175 | NOVA1 | 14:27067372 | cg20478129 | 367360 |
| 0.176 | CACNA1B | 9:140917431 | cg13610307 | 251390 |
| 0.176 | TRAF3 | 14:103367591 | cg10920224 | 206529 |
| 0.176 | IRX1 | 5:3596207 | cg10530883 | 200071 |
| 0.176 | BTBD3 | 20:11871384 | cg23944804 | 420919 |
| 0.177 | | 10:111216962 | cg16664405 | 306435 |
| 0.177 | | 10:125751413 | cg20250269 | 363883 |
| 0.178 | IRS2 | 13:110438578 | cg02320481 | 47382 |
| 0.178 | EVX2; HOXD13 | 2:176948732 | cg15133351 | 280970 |
| 0.178 | PKDCC | 2:42275740 | cg12630082 | 234855 |
| 0.179 | IL5RA | 3:3152916 | cg21594961 | 384489 |
| 0.179 | C5orf63 | 5:126408806 | cg17848407 | 325324 |
| 0.179 | GDF7 | 2:20870812 | cg14780466 | 274979 |
| 0.179 | HOXA10; HOXA10- | 7:27206073 | cg15506609 | 286583 |
| | HOXA9; HOXA7; | | | |
| | HOXA9; MIR196B | | | |
| 0.179 | TMCC3 | 12:94995915 | cg09162806 | 176158 |
| 0.179 | LOC91149; RAPGEF4 | 2:173600676 | cg00997611 | 21009 |
| 0.179 | PHACTR1 | 6:12749978 | cg13246235 | 244239 |
| 0.180 | GCK; MYL7 | 7:44184403 | cg03812172 | 76924 |
| 0.181 | | 9:98981500 | cg21167269 | 377918 |
| 0.181 | GDF7 | 2:20870087 | cg09074113 | 174642 |
| 0.181 | KCNE1 | 21:35832028 | cg07321776 | 143076 |
| 0.181 | KCNK2 | 1:215256262 | cg18794839 | 340940 |
| 0.181 | GPR123 | 10:134910540 | cg13750264 | 254277 |
| 0.182 | FTSJ2; NUDT1; | 7:2289097 | cg12017558 | 224816 |
| | SNX8 | | | |
| 0.182 | KLF16 | 19:1852127 | cg07355926 | 143681 |
| 0.182 | | 2:154143768 | cg18771570 | 340550 |
| 0.183 | | 6:77563066 | cg21291856 | 380079 |
| 0.184 | | 17:54858770 | cg07973125 | 154969 |
| 0.184 | FGF12 | 3:192127457 | cg08002883 | 155463 |
| 0.185 | ILDR1 | 3:121714668 | cg20356878 | 365522 |
| 0.185 | SLC17A3 | 6:25882590 | cg03517284 | 71065 |
| 0.186 | FHOD3 | 18:34325827 | cg07495405 | 146206 |
| 0.186 | C1orf106 | 1:200860534 | cg00909514 | 19113 |
| 0.186 | HOXA5; HOXA6; | 7:27187502 | cg03529432 | 71326 |
| | HOXA7; LOC100133311 | | | |
| 0.186 | SH2D4B | 10:82295502 | cg08506606 | 164700 |
| 0.186 | KNDC1; UTF1; | 10:135044114 | cg09053680 | 174307 |
| | VENTX | | | |
| 0.186 | GSC2 | 22:19137874 | cg23774356 | 418306 |
| 0.186 | CLCA1 | 1:86968087 | cg19807991 | 356943 |
| 0.187 | SCTR | 2:120281999 | cg07176264 | 140350 |
| 0.187 | PITX1 | 5:134363562 | cg02037307 | 41774 |
| 0.187 | FTSJ2; NUDT1; | 7:2288716 | cg13550401 | 250077 |
| | SNX8 | | | |
| 0.188 | | 8:7543899 | cg12174280 | 227610 |
| 0.189 | CLCA1 | 1:86968543 | cg04896168 | 97717 |
| 0.190 | NAALAD2 | 11:89867911 | cg14304817 | 265815 |
| 0.190 | LHX6 | 9:124989294 | cg21469772 | 382370 |
| 0.190 | KLF16 | 19:1852165 | cg05006231 | 99848 |
| 0.191 | CLCA1 | 1:86968255 | cg26829098 | 468093 |
| 0.191 | HLA-A | 6:29910755 | cg11808100 | 221232 |
| 0.192 | | 19:22444593 | cg03692651 | 74585 |
| 0.192 | SERTM1 | 13:37248244 | cg09907509 | 189121 |
| 0.193 | CNTN4 | 3:2140699 | cg06598836 | 128644 |
| 0.193 | | 15:29862517 | cg04283162 | 86040 |
| 0.193 | | 4:56023751 | cg10360906 | 197083 |
| 0.193 | KIAA1217; PRINS | 10:24535410 | cg04122385 | 82818 |
| 0.194 | C5orf63 | 5:126409211 | cg10812634 | 204796 |
| 0.194 | LOC91149; RAPGEF4 | 2:173600362 | cg14379854 | 267268 |
| 0.195 | ZNF471 | 19:57019069 | cg00674365 | 14254 |
| 0.195 | AFG3L1P; DBNDD1 | 16:90070038 | cg16380389 | 301186 |
| 0.195 | SH2D4B | 10:82296072 | cg09981929 | 190306 |
| 0.196 | C21orf67; ITGB2; | 21:46349496 | cg02464073 | 50169 |
| | LOC100505746 | | | |
| 0.196 | NPTX2 | 7:98246633 | cg18952796 | 343455 |
| 0.197 | LHX6 | 9:124990276 | cg00142257 | 3190 |
| 0.197 | GDF7 | 2:20871401 | cg04082016 | 81976 |
| 0.197 | CR1L | 1:207842833 | cg03408135 | 68966 |
| 0.198 | DKFZP686I15217; | 6:2991147 | cg06195525 | 121679 |
| | NQO2 | | | |
| 0.199 | PCDH7 | 4:30723855 | cg26133769 | 455996 |
| 0.199 | IFITM1; IFITM3 | 11:320956 | cg14885742 | 276796 |
| 0.199 | GNAS; GNAS- | 20:57414274 | cg23732978 | 417791 |
| | AS1 | | | |
| 0.201 | NAALAD2 | 11:89867819 | cg05500015 | 109117 |
| 0.202 | CPNE4 | 3:131246020 | cg01966816 | 40359 |
| 0.204 | EFHA2 | 8:16885000 | cg12097222 | 226305 |
| 0.204 | CRMP1 | 4:5894691 | cg03544320 | 71665 |
| 0.205 | UNC5D | 8:35092876 | cg06010588 | 118507 |
| 0.206 | WTIP | 19:34972145 | cg10771931 | 204037 |
| 0.207 | DNAH2; EFNB3; | 17:7613764 | cg22098780 | 391853 |
| | WRAP53 | | | |
| 0.207 | CR1L | 1:207843084 | cg21106486 | 376853 |
| 0.207 | CR1; CR2 | 1:207669922 | cg10021878 | 191017 |
| 0.207 | | 13:21872349 | cg14456004 | 268764 |
| 0.207 | LOC645249 | 7:156814480 | cg09858188 | 188213 |
| 0.208 | | 13:50701619 | cg01803928 | 36963 |
| 0.208 | GALR1 | 18:74962133 | cg03502002 | 70747 |
| 0.210 | | 1:239550283 | cg13985485 | 259057 |
| 0.210 | | 9:96623674 | cg13679303 | 252812 |
| 0.210 | DRD5 | 4:9783192 | cg26296488 | 458826 |
| 0.210 | NETO1 | 18:70535005 | cg18651870 | 338661 |
| 0.211 | MDGA1 | 6:37617124 | cg00807871 | 17056 |
| 0.212 | FAM75C1 | 9:90531866 | cg21265404 | 379726 |
| 0.213 | TNS3 | 7:47621692 | cg06352730 | 124250 |
| 0.214 | | 2:16484904 | cg23905015 | 420220 |
| 0.214 | | 7:32358064 | cg13207630 | 243593 |
| 0.216 | PROZ | 13:113819100 | cg18105134 | 329535 |
| 0.216 | TSHZ1; ZADH2 | 18:72917101 | cg18449964 | 335368 |
| 0.217 | C5orf63 | 5:126409310 | cg15405432 | 284960 |
| 0.218 | ZNF468 | 19:53361448 | cg15721243 | 290213 |
| 0.219 | FAT1 | 4:187644739 | cg16248329 | 298797 |
| 0.220 | CTTNBP2 | 7:117513835 | cg16913250 | 310581 |
| 0.220 | HES6; LOC151174; | 2:239139911 | cg06102419 | 120125 |
| | LOC643387 | | | |
| 0.221 | MAL2 | 8:120220882 | cg06822816 | 133139 |
| 0.222 | C5orf63 | 5:126409227 | cg02304092 | 47036 |
| 0.224 | LHX6 | 9:124988720 | cg04282082 | 86021 |
| 0.226 | HCG4; LOC554223 | 6:29759078 | cg11036359 | 208507 |
| 0.229 | C5orf63 | 5:126409061 | cg18710053 | 339507 |
| 0.229 | GPR78 | 4:8582249 | cg01829241 | 37555 |
| 0.229 | LHX6 | 9:124989337 | cg13571460 | 250561 |
| 0.230 | C5orf63 | 5:126409198 | cg22905511 | 404065 |
| 0.231 | | 2:154143677 | cg07563363 | 147470 |
| 0.233 | FCRLA | 1:161675579 | cg04888234 | 97564 |
| 0.233 | FAM160B1 | 10:116634877 | cg14964115 | 278020 |
| 0.235 | PM20D1 | 1:205819251 | cg14893161 | 276892 |
| 0.235 | GDF7 | 2:20871279 | cg07021052 | 137180 |
| 0.237 | ANKRD27; RGS9BP | 19:33165484 | cg27413643 | 478153 |
| 0.238 | MDGA1 | 6:37617864 | cg20053110 | 361003 |
| 0.240 | | 18:6414978 | cg17688525 | 322822 |
| 0.244 | ATP8B5P; RUSC2 | 9:35490515 | cg15271616 | 283055 |
| 0.244 | GDF7 | 2:20871002 | cg10687131 | 202734 |
| 0.248 | | 2:181987552 | cg20685672 | 370518 |
| 0.252 | ARHGAP33; C19orf55 | 19:36264029 | cg16569309 | 304609 |
| 0.252 | LHX6 | 9:124990165 | cg03363289 | 68128 |
| 0.253 | GDF7 | 2:20870211 | cg05481257 | 108752 |
| 0.253 | SH2D4B | 10:82295723 | cg24440658 | 429013 |
| 0.254 | GDF7 | 2:20869434 | cg27141850 | 473604 |
| 0.254 | TSNARE1 | 8:143376371 | cg24849373 | 435844 |
| 0.254 | AGAP1 | 2:236579469 | cg03451670 | 69806 |
| 0.255 | | 1:41350183 | cg08870646 | 171150 |
| 0.255 | LHX6 | 9:124989915 | cg13862711 | 256498 |
| 0.256 | IGF1R | 15:99193247 | cg03437748 | 69502 |
| 0.256 | SDK1 | 7:4118583 | cg22535849 | 398182 |
| 0.257 | PM20D1 | 1:205819492 | cg24503407 | 430027 |
| 0.258 | CLCA1 | 1:86968184 | cg10760651 | 203838 |
| 0.259 | WDR17 | 4:176987009 | cg11923920 | 223292 |
| 0.261 | ACAN | 15:89354338 | cg02401352 | 49110 |
| 0.266 | TSHZ1; ZADH2 | 18:72917163 | cg03972071 | 79876 |
| 0.269 | MDGA1 | 6:37617956 | cg24796644 | 434985 |
| 0.272 | C5orf63 | 5:126409153 | cg27097438 | 472827 |
| 0.275 | C11orf67; RSF1 | 11:77532707 | cg22741595 | 401343 |
| 0.275 | | 6:167195910 | cg06330797 | 123893 |
| 0.276 | COL9A1 | 6:71022072 | cg23974730 | 421453 |
| 0.276 | INPP5A | 10:134411480 | cg12501287 | 232697 |
| 0.277 | PM20D1 | 1:205818956 | cg17178900 | 314798 |
| 0.277 | FAM181A; FAM181A- | 14:94385395 | cg20022541 | 360557 |
| | AS1 | | | |
| 0.279 | | 3:120151283 | cg06378561 | 124751 |
| 0.280 | LOC255411 | 12:49113738 | cg00007810 | 182 |
| 0.280 | CHAF1B | 21:37761363 | cg22871721 | 403496 |
| 0.281 | ASB1 | 2:239330383 | cg13295614 | 245013 |
| 0.282 | TNFSF8 | 9:117694183 | cg14361804 | 266932 |
| 0.284 | DISC1 | 1:231820076 | cg07134368 | 139412 |
| 0.285 | DIP2C | 10:593275 | cg06051619 | 119189 |
| 0.286 | ULK4P1; ULK4P2 | 15:30861172 | cg23804921 | 418678 |
| 0.287 | TPO | 2:1426786 | cg01957222 | 40112 |
| 0.288 | PM20D1 | 1:205819463 | cg07167872 | 140186 |
| 0.290 | PM20D1 | 1:205819179 | cg14159672 | 262672 |
| 0.290 | LOC200772 | 2:241896910 | cg06193597 | 121647 |
| 0.291 | PM20D1 | 1:205819406 | cg11965913 | 224049 |
| 0.292 | PPP2R2B | 5:146253830 | cg10926851 | 206628 |
| 0.294 | LOC100133461 | 4:3683268 | cg03965172 | 79742 |
| 0.297 | AVL9; LSM5 | 7:32526065 | cg26856631 | 468599 |
| 0.300 | PM20D1 | 1:205819088 | cg26354017 | 459920 |
| 0.300 | MIR182; MIR183; | 7:129412730 | cg16069065 | 295914 |
| | MIR96 | | | |
| 0.301 | SCN1A | 2:166930521 | cg00881894 | 18574 |
| 0.305 | RPSAP58; ZNF681 | 19:23941859 | cg25958450 | 453100 |
| 0.305 | CCDC130; MRI1 | 19:13875111 | cg25755428 | 450171 |
| 0.317 | PML | 15:74345103 | cg11399582 | 214535 |
| 0.318 | C1orf109; CDCA8 | 1:38156652 | cg06917450 | 135153 |
| 0.324 | CELF4 | 18:34917603 | cg20094343 | 361624 |
| 0.325 | CLEC4F | 2:71033156 | cg02479782 | 50493 |
| 0.343 | EHMT2; NEU1; | 6:31838544 | cg20370184 | 365714 |
| | SLC44A4 | | | |
| 0.344 | NCOR2 | 12:124827315 | cg23878260 | 419752 |
| 0.349 | PCCA | 13:100989375 | cg20536971 | 368191 |
| 0.351 | ANKRD26P3; | 13:19918525 | cg08801017 | 169810 |
| | LINC00421 | | | |
| 0.355 | PPYR1 | 10:47083392 | cg11861387 | 222311 |
| 0.358 | | 5:5887642 | cg26690407 | 465769 |
| 0.363 | | 13:79694063 | cg24941469 | 437468 |
| 0.366 | ALLC | 2:3753297 | cg05971102 | 117729 |
| 0.378 | MIR3663 | 10:118931160 | cg17723206 | 323281 |
| 0.388 | SERPINB9 | 6:2888212 | cg19577958 | 352915 |
| 0.399 | NCOR2 | 12:124831017 | cg07474670 | 145798 |
| 0.404 | | 14:22370643 | cg12819537 | 237764 |
| 0.408 | SLC37A1 | 21:43989949 | cg08407901 | 162897 |
| 0.413 | MRPL1 | 4:78852682 | cg15897435 | 293045 |
| 0.417 | C7orf26; ZNF853 | 7:6653510 | cg08693140 | 167967 |
| 0.431 | | 14:22361282 | cg16702660 | 307151 |
| 0.432 | ITIH4; MUSTN1; | 3:52869263 | cg24629711 | 432072 |
| | TMEM110; TMEM110- | | | |
| | MUSTN1 | | | |
| 0.442 | MAGI2-AS3 | 7:79083056 | cg14464244 | 268938 |
| 0.586 | PPP1R16B | 20:37438823 | cg18674961 | 338963 |

*UCSC hg19 Cytosine Coordinate of CpG (+strand)

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

The following references are incorporated herein by reference in their entireties.

### REFERENCES

1 Firestein G S. Evolving concepts of rheumatoid arthritis. Nature, 423:356-361, 2003
2 Lee differentially methylated, Kiener H P, Agarwal S K, Noss E H, Watts G F, Chisaka O, Takeichi M, Brenner M B. 2007. Cadherin-11 in synovial lining formation and pathology in arthritis. Science. 315:1006
3 Kiener H P, Niederreiter B, Lee differentially methylated, Jimenez-Boj E, Smolen J S, Brenner M B. Cadherin 11 promotes invasive behavior of fibroblast-like synoviocytes. Arthritis Rheum. 2009 May; 60(5):1305-10
4 Firestein, G S. Invasive fibroblast-like synoviocytes in rheumatoid arthritis: Passive responders or transformed aggressors? Arthritis Rheum 39:1781-1790, 1996
5 Müller-Ladner U, Kriegsmann J, Franklin B N, Matsumoto S, Geiler T, Gay R E, Gay S. Synovial fibroblasts of patients with rheumatoid arthritis attach to and invade normal human cartilage when engrafted into SCID mice. Am J Pathol. 1996 November; 149(5):1607-15
6 Lafyatis R, Remmers E F, Roberts A B, Yocum D E, Sporn M B, Wilder R L. 1989. Anchorage-independent growth of synoviocytes from arthritic and normal joints. Stimulation by exogenous platelet-derived growth factor and inhibition by transforming growth factor-beta and retinoids. J Clin Invest. 83:1267
7 Baier A, Meineckel I, Gay S, Pap T. Apoptosis in rheumatoid arthritis. Curr Opin Rheumatol. 2003 May; 15(3):274-9
8 Lefèvre S, Knedla A, Tennie C, Kampmann A, Wunrau C, Dinser R, Korb A, Schnäker E M, Tamer I H, Robbins P D, Evans C H, Stürz H, Steinmeyer J, Gay S, Schölmerich J, Pap T, Müller-Ladner U, Neumann E. Synovial fibroblasts spread rheumatoid arthritis to unaffected joints. Nat Med. 2009 December; 15(12):1414-20
9 Pap T, Franz J K, Hummel K M, Jeisy E, Gay R, Gay S. Activation of synovial fibroblasts in rheumatoid arthritis: lack of Expression of the tumour suppressor PTEN at sites of invasive growth and destruction. Arthritis Res. 2000; 2(1):59-64
10 Franz J K, Pap T, Hummel K M, Nawrath M, Aicher W K, Shigeyama Y, Müller-Ladner U, Gay R E, Gay S. Expression of sentrin, a novel antiapoptotic molecule, at sites of synovial invasion in rheumatoid arthritis. Arthritis Rheum. 2000 March; 43(3):599-607
11 You X, Boyle D L, Hammaker D, Firestein G S. PUMA-mediated apoptosis in fibroblast-like synoviocytes does not require p53. Arthritis Res Ther. 2006; 8(6):R157
12 Firestein G S, F Echeverri, M Yeo, N J Zvaifler, and D R Green. Somatic mutations in the p53 tumor suppressor gene in rheumatoid arthritis synovium. Proc Natl Acad Sci USA, 94: 10895-10900, 1997
13 Igarashi H, Hashimoto J, Tomita T, Yoshikawa H, Ishihara K. TP53 mutations coincide with the ectopic expression of activation-induced cytidine deaminase in the fibroblast-like synoviocytes derived from a fraction of patients with rheumatoid arthritis. Clin Exp Immunol. 2010 Jul. 1; 161(1):71-80
14 Inazuka M, Tahira T, Horiuchi T, Harashima S, Sawabe T, Kondo M, Miyahara H, Hayashi K. Analysis of p53 tumour suppressor gene somatic mutations in rheumatoid arthritis synovium. Rheumatology (Oxford). 2000 March; 39(3):262-6
15 Rème T, Travaglio A, Gueydon E, Adla L, Jorgensen C, Sany J. Mutations of the p53 tumour suppressor gene in erosive rheumatoid synovial tissue. Clin Exp Immunol. 1998 February; 111(2):353-8
16 Bang H, Egerer K, Gauliard A, Lüthke K, Rudolph P E, Fredenhagen G, Berg W, Feist E, Burmester G R. Mutation and citrullination modifies vimentin to a novel autoantigen for rheumatoid arthritis. Arthritis Rheum. 2007 August; 56(8):2503-11
17 Da Sylva T R, Connor A, Mburu Y, Keystone E, Wu G E. Somatic mutations in the mitochondria of rheumatoid arthritis synoviocytes. Arthritis Res Ther. 2005; 7(4):R844-51
19 Lee S-H, D K Chang, A Goel, C R Boland, W Bugbee, D L Boyle, and G S Firestein. Microsatellite instability and suppressed DNA repair enzyme expression in rheumatoid arthritis. J Immunol, 170:2214-20, 2003
19 Christophersen N S, Helin K. Epigenetic control of embryonic stem cell fate. J Exp Med. Oct. 25, 2010; 207(11):2287-95
20 Kulis M, Esteller M. DNA methylation and cancer. Adv Genet. 2010; 70:27-56
21 Ren J, Singh B N, Huang Q, Li Z, Gao Y, Mishra P, Hwa Y L, Li J, Dowdy S C, Jiang S W. DNA hypermethylation as a chemotherapy target. Cell Signal. Feb. 21, 2011. [Epub ahead of print]
22 Turek-Plewa J, Jagodzinski P P. The role of mammalian DNA methyltransferases in the regulation of gene expression. Cell Mol Biol Lett. 2005; 10(4):631-47
23 Hollingsworth J W, Maruoka S, Boon K, Garantziotis S, Li Z, Tomfohr J, Bailey N, Potts E N, Whitehead G, Brass differentially methylated, Schwartz D A. In utero supplementation with methyl donors enhances allergic airway disease in mice. J Clin Invest. 2008 October; 118(10):3462-9
24 Miller R L. Prenatal maternal diet affects asthma risk in offspring. J Clin Invest. 2008. 118:3265-8
25 Liu C C, Fang T J, Ou T T, Wu C C, Li R N, Lin Y C, Lin C H, Tsai W C, Liu H W, Yen J H. Global DNA methylation, DNMT1, and MBD2 in patients with rheumatoid arthritis. Immunol Lett. Mar. 30, 2011; 135(1-2):96-9. Epub Oct. 16, 2010
26 Karouzakis E, Gay R E, Michel B A, Gay S, Neidhart M. DNA hypomethylation in rheumatoid arthritis synovial fibroblasts. Arthritis Rheum. 2009 December; 60(12):3613-22
27 Lee B H et al. Procainamide is a specific inhibitor of DNA methyltransferase 1. J Biol Chem. Dec. 9, 2005; 280(49):40749-56
28 Schipper R G et al. Polyamines and DNA methylation in childhood leukaemia. Biochem Soc Trans. 2007 April; 35(Pt 2):331-529 Fu L. H. et al., Methylation status of the IL-10 gene promoter in the peripheral blood mononuclear cells of rheumatoid arthritis patients. Yi Chuan. 2007 November; 29(11):1357-61.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising determining whether at least 2 nucleic acid loci or at least 2 genes in a sample from said subject have methylation states indicative of rheumatoid arthritis, osteoarthritis, a rheumatoid arthritis or osteoarthritis prognosis, or a response to treatment for rheumatoid arthritis or osteoarthritis.

2. The method of claim 1, further comprising comparing the methylation states of the at least 2 loci or at least 2 genes in the sample from said subject with the methylation states of the loci or the genes in normal tissue, tissue from a subject without said known rheumatoid arthritis or osteoarthritis prognosis, or tissue from a subject without said known response to treatment for rheumatoid arthritis or osteoarthritis.

3. The method of claim 1, wherein an increase or decrease in the extent of methylation of at least 2 loci or at least 2 genes compared to the extent of methylation of the loci or genes in normal tissue, tissue from a subject without said known rheumatoid arthritis or osteoarthritis prognosis, or tissue from a subject without said known response to treatment for rheumatoid arthritis or osteoarthritis is indicative of the presence or absence of rheumatoid arthritis, osteoarthritis, a rheumatoid arthritis or osteoarthritis prognosis, or response to treatment for rheumatoid arthritis or osteoarthritis for the subject.

4. The method of claim 1, wherein the methylation states of said at least 2 loci or at least 2 genes is determined in a cell from said subject selected from the group consisting of fibroblast, synoviocyte, fibroblast-like synoviocyte, macrophage, and peripheral blood cell.

5. The method of claim 4, wherein the fibroblast-like synoviocyte is selected from the group consisting of rheumatoid arthritis fibroblast-like synoviocyte and osteoarthritis fibroblast-like synoviocyte

6. The method of claim 4, wherein the peripheral blood cell is selected from the group consisting of white blood cell, neutrophil, eosinophil, basophil, lymphocyte, B cell, plasma cell, T cell, natural killer cell, monocyte, and dendritic cell.

7. The method of claim 1, wherein the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

8. The method of claim 1, wherein the at least 2 loci or at least 2 genes are selected from the group consisting of SEQ ID NO.s 1-485512, the loci listed in TABLE 6, TABLE 7, or TABLE 8, and the genes listed in TABLE 3.

9. The method of claim 1, wherein the methylation states of at least 5 nucleic acid loci or at least 5 genes in said sample are determined.

10. The method of claim 1, wherein the subject is a human.

11. A method for diagnosing rheumatoid arthritis or osteoarthritis, determining a rheumatoid arthritis or osteoarthritis prognosis, or determining or predicting a response to treatment for rheumatoid arthritis or osteoarthritis in a subject comprising:
determining the methylation states of a plurality of nucleic acid loci or the methylation states of a plurality of genes in a sample of said subject to obtain a methylation profile; and
determining whether said methylation profile is indicative of rheumatoid arthritis, osteoarthritis, a rheumatoid arthritis or osteoarthritis prognosis, or a response to treatment for rheumatoid arthritis or osteoarthritis.

12. The method of claim 11, further comprising comparing the methylation state of the plurality of nucleic acid loci or the methylation state of the plurality of genes in the sample from said subject with the methylation state of the plurality of nucleic acid loci or the methylation state of the plurality of genes in normal tissue, tissue from a subject without said known rheumatoid arthritis or osteoarthritis prognosis, or tissue from a subject without said known response to treatment for rheumatoid arthritis or osteoarthritis.

13. The method of claim 12, wherein an increase or decrease in the extent of methylation of plurality of nucleic acid loci or of the methylation state of the plurality of genes compared to the extent of methylation of the plurality of nucleic acid loci or the extent of methylation of the plurality of genes in normal tissue, tissue from a subject without said known rheumatoid arthritis or osteoarthritis prognosis, or tissue from a subject without said known response to treatment for rheumatoid arthritis or osteoarthritis is indicative of the presence or absence of rheumatoid arthritis, osteoarthritis, a rheumatoid arthritis or osteoarthritis prognosis, or response to treatment for rheumatoid arthritis or osteoarthritis for the subject.

14. The method of claim 11, wherein the methylation state of said plurality of nucleic acid loci or said plurality of genes is determined in in a cell from said subject selected from the group consisting of fibroblast, synoviocyte, fibroblast-like synoviocyte, macrophage and peripheral blood cell.

15. The method of claim 14, wherein the fibroblast-like synoviocyte is selected from the group consisting of rheumatoid arthritis fibroblast-like synoviocyte and osteoarthritis fibroblast-like synoviocyte

16. The method of claim 14, wherein the peripheral blood cell is selected from the group consisting of white blood cell, neutrophil, eosinophil, basophil, lymphocyte, B cell, plasma cell, T cell, natural killer cell, monocyte, and dendritic cell.

17. The method of claim 11, wherein the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

18. The method of claim 11, wherein the plurality of nucleic acid loci or the plurality of genes is selected from the group consisting of SEQ ID NO.s 1-485512, the loci listed in TABLE 6, TABLE 7, or TABLE 8, and the genes listed in TABLE 3.

19. The method of claim 11, wherein the methylation states of at least 5 nucleic acid loci or at least 5 genes in said sample are determined.

20. The method of claim 11, wherein the subject is a human.

21. A method of identifying a methylation profile indicative of rheumatoid arthritis, osteoarthritis, a rheumatoid arthritis or osteoarthritis prognosis, or a response to treatment for rheumatoid arthritis or osteoarthritis comprising:
determining the methylation states of a plurality of nucleic acid loci or the methylation states of a plurality of genes in a sample from a test subject with rheumatoid arthritis, osteoarthritis, a particular rheumatoid arthritis or osteoarthritis prognosis, or a particular response to treatment for rheumatoid arthritis or osteoarthritis;
determining the methylation states of said plurality of nucleic acid loci or said plurality of genes in a sample from a control subject without rheumatoid arthritis, without osteoarthritis, without said rheumatoid arthritis or osteoarthritis prognosis or without said response to treatment for rheumatoid arthritis or osteoarthritis; and
identifying loci or genes which are hypermethylated or hypomethylated in said sample from said test subject relative to said sample from said control subject to identify said methylation profile indicative of rheumatoid arthritis, osteoarthritis a rheumatoid arthritis or osteoarthritis prognosis, or a response to treatment for rheumatoid arthritis or osteoarthritis.

22. The method of claim 21, further comprising storing data representing said loci or said genes which are hypermethylated or hypomethylated in said sample from said test subject relative to said sample from said control subject on a non-transitory computer readable medium.

23. The method of claim 22, further comprising comparing the methylation states of a plurality of nucleic acid loci or the methylation states of a plurality of genes in a sample from a test subject with rheumatoid arthritis, osteoarthritis, a particular rheumatoid arthritis or osteoarthritis prognosis, or a particular response to treatment for rheumatoid arthritis or osteoarthritis with the methylation states of said plurality of nucleic acid loci or the methylation states of said plurality of genes in a sample from a control subject without rheumatoid arthritis, without osteoarthritis, without said rheumatoid arthritis or osteoarthritis prognosis or without said response to treatment for rheumatoid arthritis or osteoarthritis.

24. The method of claim 21, wherein the methylation state of said plurality of nucleic acid loci or said plurality of genes in a sample from said test subject or said control subject is determined in a cell from said subject or said control subject selected from the group consisting of fibroblast, synoviocyte, fibroblast-like synoviocyte, macrophage, and peripheral blood cell.

25. The method of claim 24, wherein the fibroblast-like synoviocyte is selected from the group consisting of rheumatoid arthritis fibroblast-like synoviocyte and osteoarthritis fibroblast-like synoviocyte

26. The method of claim 24, wherein the peripheral blood cell is selected from the group consisting of white blood cell, neutrophil, eosinophil, basophil, lymphocyte, B cell, plasma cell, T cell, natural killer cell, monocyte, and dendritic cell.

27. The method of claim 21, wherein the sample is selected from the group consisting of a tissue sample, a frozen tissue sample, a biopsy specimen, a surgical specimen, a cytological specimen, whole blood, bone marrow, cerebral spinal fluid, peritoneal fluid, lymph fluid, serum, plasma, urine, stool, and nipple aspirate.

28. The method of claim 21, wherein the methylation states of at least 5 nucleic acid loci or at least 5 genes are determined.

29. The method of claim 21, wherein the subject is a human.

30. A method of ameliorating rheumatoid arthritis or osteoarthritis comprising:
evaluating the methylation status of a plurality of human nucleic acid loci in a nucleic acid sample from a human subject having symptoms of rheumatoid arthritis or osteoarthritis, wherein said loci comprise at least 5 loci or at least 5 genes selected from the group consisting of SEQ ID NO.s 1-485512, the loci listed in TABLE 6, TABLE 7, or TABLE 8, and the genes listed in TABLE 3; and
administering a treatment for rheumatoid arthritis or osteoarthritis if said at least 5 loci or said at least 5 genes have a methylation status indicative of rheumatoid arthritis or osteoarthritis.
